(19) United States
(12) Patent Application Publication
(10) Pub. No.: US 2026/0063647 A1
(43) Pub. Date: Mar. 5, 2026

(54) COMBINATIONS OF BIOMARKERS FOR METHODS FOR DETECTING TRISOMY 21

(71) Applicant: ROSETTA SIGNALING LABORATORIES, LLC, PHOENIX, AZ (US)

(72) Inventors: Carl Philip Weiner, Phoneix, AZ (US); Yafeng Dong, Overland Park, KS (US)

(21) Appl. No.: 19/388,492

(22) Filed: Nov. 13, 2025

Related U.S. Application Data
(63) Continuation of application No. 17/203,534, filed on Mar. 16, 2021, now Pat. No. 12,474,355.

(51) Int. Cl. G01N 33/68; G01N 33/53

(52) U.S. Cl. CPC G01N 33/6896; G01N 33/5308; G01N 2800/387

(57) ABSTRACT

Methods for detecting a Group of Biomarkers is provided herein. The translation profile of the Group of Biomarkers can be used for determining whether a subject, such as a fetus, has Down syndrome The methods include detecting one or more specific groups of biomarkers in a biological sample, and determining whether the expression of the biomarkers is altered when compared to expression of the biomarkers in one or more subjects that do not have trisomy 21 (e.g., a transcriptional standard). The biological sample can be a blood sample, and the biomarkers are cell free plasma RNAs.

### CROSS-REFERENCE TO RELATED APPLICATIONS

This application is a continuation of U.S. application Ser. No. 17/203,534 filed Mar. 16, 2021, which is a continuation-in-part of U.S. application Ser. No. 14/361,895 filed May 30, 2014, which is the § 371 U.S. National Stage of International Application No. PCT/US2012/067328, filed Nov. 30, 2012, which claims the benefit of U.S. Provisional Application No. 61/565,761, filed Dec. 1, 2011, wherein each are incorporated by reference herein in their entireties.

### SEQUENCE LISTING

This application contains a Sequence Listing which has been submitted in XML format via Patent Center and is hereby incorporated by reference in its entirety. The XML copy, created on Nov. 13, 2025, is named W2460.10002US04 and is 3,254,129 bytes in size.

### BACKGROUND

Trisomy 21, also referred to as Down Syndrome and Mongolism, is the result of a chromosomal abnormality. A human cell has two types of chromosomes. One type is the autosomal chromosomes (chromosomes 1-22), and the other type is the sex chromosome (the X and Y chromosomes). In a normal human cell there are 46 chromosomes, and they are present in the cell as 23 pairs. Thus each normal human cell has two of each autosomal chromosomes (two copies of chromosome 1, two copies of chromosome 2, etc.) and one pair of sex chromosomes (an X and a Y chromosome for a male, or two X chromosomes for a female). A karyotype of a normal male is referred to as 46XY, and that of a normal female is 46XX. The chromosomal abnormality in a person having trisomy 21 is an extra chromosome 21. The karyotype of a male having trisomy 21 is 47XY+21, and the karyotype of a female having trisomy 21 is 47XX+21.

Trisomy 21 is typically caused by a meiotic nondisjunction event. With nondisjunction, a gamete (either a sperm or egg cell) is produced with an extra copy of the chromosome 21; thus, the gamete has 24 and not the normal 23 chromosomes. When combined with a normal gamete from the other parent, the resulting embryo has 47 chromosomes, with three copies of chromosome 21. An analogous process accounts for most cases of trisomy 13 and 18. Trisomy 21 is the cause of approximately 95% of Down syndromes, with 88% resulting from nondisjunction in the maternal gamete and 8% from nondisjunction in the paternal gamete. The actual Down syndrome “critical region” encompasses chromosome bands 21q22.1-q22.3.

Since trisomy 21 is usually caused by nondisjunction in the gametes prior to conception, all cells in the resulting conceptus are affected. However, when some of the cells in the body are normal and other cells have trisomy 21, it is called mosaic Down syndrome (46,XX/47,XX,+21). This can occur in one of main two ways: in the first, a nondisjunctional event occurs in a normal embryo during an early cell division results in a fraction of the cells having trisomy 21; in the second, a Down syndrome embryo secondary to nondisjunction loses the extra 21 chromosome with the result of a normal cell line. There is considerable variability in the fraction of trisomy 21 cells among mosaic trisomy 21, both as a whole and among tissues. Mosaicism causes 1-2% of Down syndrome.

Down syndrome is a random event. There is no evidence that it is due to parental behavior (other than age) or environmental factors. In 2006, the Centers for Disease Control and Prevention estimated the overall rate for Down syndrome in the United States was one per 733 live births (5429 new cases per year). Approximately 95% of these are trisomy 21. Down syndrome occurs in all ethnic groups and among all economic classes.

Maternal age affects the probability of conceiving a fetus with Down syndrome. At maternal age 20 to 24, the probability is one in 1562; at age 35 to 39 the probability is one in 214, and above age 45 the probability is one in 19 (Huether et al., 1998, J Med Genet 35 (6): 482-90. doi:10.1136/jmg.35.6.482. PMID 9643290). Although the probability increases with advancing maternal age, 80% of children with Down syndrome are born to women under the age of 35, reflecting the overall fecundity of that age group. Recent information suggests paternal age, especially beyond 42 years increases the risk of Down syndrome too.

A diagnostic test for Down syndrome is typically preceded by some form of screening test (history, ultrasound or blood protein measurement) as the current methods for the definitive diagnosis involves an invasive test-amniocentesis, chorionic villus sampling or percutaneous umbilical cord blood sampling (PUBS)—that is then offered to families who are screen positive, i.e. have an increased likelihood of having a fetus with Down syndrome. In the United States, the American College of Obstetrics and Gynecology guidelines recommend non-invasive screening and invasive testing be offered to all women, regardless of their age, and most likely all physicians follow these guidelines. This recommendation is supported by some insurance plans that only reimburse invasive testing if the woman is >34 years old or if she has received a high-risk score from a non-invasive screening test. Amniocentesis, CVS and PUBS are invasive procedures that involve inserting instruments into the uterus, and therefore carry a risk of causing fetal injury or pregnancy loss. The risks of a loss from CVS and amniocentesis are often quoted as 1% and 0.5% respectively. In all likelihood, the fetuses lost as a result are otherwise normal.

In the past, several common non-invasive screening tests were the main method to identify a fetus at high risk for Down syndrome. These tests are typically performed in the late 1^{st} or early 2^{nd} trimester. Due to the nature of screening, each test has a significant chance of a false positive that is suggesting the fetus has Down syndrome when in fact, the fetus does not. Screen positive tests must be verified by an invasive procedure before a diagnosis of Down syndrome is made. More recently, these tests have been supplanted by a noninvasive screening method based on testing the fetal DNA in maternal blood often referred to as NIPS-noninvasive prenatal screening using plasma cell free DNA. Using one of several methods, the fetal DNA is isolated and probed to determine the presence of T21, T13, and T18 along with the Y chromosome. Common screening procedures for Down syndrome are given in Table A.

TABLE A
First and Second trimester Down syndrome screens

| | Weeks | | False | |
|---|---|---|---|---|
| | gestation | Detection | positive | |
| Screen | performed | rate | rate | Description |
| Quad screen | 15-20 | 81% | 5% | Measures the maternal serum a feto |
| | | | | protein, estriol, human chorionic |
| | | | | gonadotropin (hCG) and inhibin-α. |
| Nuchal | 10-13.5 | 85% | 5% | Uses ultrasound to measure Nuchal |
| translucency/free | | | | Translucency (NT) plus free β hCG |
| β/PAPP-A (1^{st} | | | | and PAPP-A. |
| Trimester | | | | |
| Combined Test) | | | | |
| Integrated Test | 10-13.5 | 95% | 5% | The Integrated test uses both the 1^{st} |
| | and | | | Trimester Combined test and the 2^{nd} |
| | 15-20 | | | Trimester Quad test to yield a more |
| | | | | accurate screening result. |
| NIPS | 9-40 | 99% | ≤1% | Fetal DNA derived from a maternal |
| | | | | plasma sample |

Inaccuracies may result from undetected multiple fetuses (rare with adequate ultrasound testing), incorrect dating of the pregnancy, normal variation in the ultrasound measurements or maternal protein levels that constitute the screen, or operator error. These ultrasound measurements and protein levels overlap in the healthy and disease groups broadly in all instances.

Confirmation of screen positive status requires a fetal karyotype that should be accomplished by amniocentesis or chorionic villus sampling (CVS). Amniocentesis involves taking amniotic fluid from the amniotic sac, and CVS a placental biopsy, each to obtain fetal cells. The laboratory work can take several weeks but will detect over 99.8% of all numerical chromosomal problems with a very low false positive rate; however, these methods of confirmation have the risk of miscarriage of a healthy fetus. As a result, many patients chose to rely on the NIPS result in light of its high detection rate and low false positive rate and avoid the risk of a pregnancy loss from an invasive procedure.

As noted, the focus of noninvasive detection of Down syndrome has been on the separation and testing of fetal DNA found in the mother's peripheral blood. A few investigators have tried to use ribonucleic acid (RNA) based technologies with little applicable success. RNA is present in all living cells and is a reflection of how cell DNA is being used. Its many roles include acting as a messenger carrying instructions from DNA for controlling the synthesis of proteins, or to modify the transcribing of DNA or the translation of some RNAs.

### SUMMARY OF THE INVENTION

In some embodiments, a method can include: obtaining a plasma sample from a human subject, wherein the human subject is a pregnant female; obtaining cell free nucleic acids from the plasma sample; detecting in the cell free nucleic acids the presence of a combination of nucleic acid biomarkers comprising: ATP50, ICOSLG, DOP1B, PKNOX1, COL6A1, and GART, wherein the detecting comprises: contacting the cell free nucleic acids with primers or probes that are complementary to the nucleic acid biomarkers in the combination of nucleic acid biomarkers, and detecting hybridization between the primers or probes and the combination of nucleic acid biomarkers. In some aspects, the combination of nucleic acid biomarkers further comprises: ENSG00000199633 F2, hsa-mir-5481, hsa-mir-26b, hsa-mir-450b and ENSG00000212363.

In some embodiments, a method can include: obtaining a plasma sample from a human subject, wherein the human subject is a pregnant female; obtaining cell free nucleic acids from the plasma sample; detecting in the cell free nucleic acids the presence of a combination of nucleic acid biomarkers comprising: ENSG00000199633 F2, hsa-mir-548I, hsa-mir-26b, hsa-mir-450b, ENSG00000212363, and GART, wherein the detecting comprises: contacting the cell free nucleic acids with primers or probes that are complementary to the nucleic acid biomarkers in the combination of nucleic acid biomarkers, and detecting hybridization between the primers or probes and the combination of nucleic acid biomarkers. In some aspects, the combination of nucleic acid biomarkers further comprises: ATP50, ICOSLG, DOP1B, PKNOXI, and COL6A1.

In some embodiments, the combination of nucleic acid biomarkers further comprises: RASGRP4, FAM20A, NEK9, ABCCI, SORBS2; TMPRSS2, DSCAM, ERG, ICOSLG, C21orf33, ADAMTS5, CXADR, NCAM2, UBASH3A, PFKL, CHODL, CYYR1, SLC19A1, PRDM15; COL6A1; and ABCG1.

In some embodiments, the combination of nucleic acid biomarkers further comprises: ENSG00000199633 F2, ENSG00000207147 F2, hsa-let-7d F1, hsa-mir-569 F1, hsa-mir-548I, ENSG00000201980, ENSG00000202231, hsa-mir-216b, hsa-mir-98, hsa-mir-26b, hsa-mir-581 F1, hsa-mir-450b, ENSG00000212363, ENSG00000199282, hsa-mir-523, hsa-mir-376a-2/1 F2, ENSG00000199856 F1, and HBII-276 F2.

In some embodiments, the nucleic acid biomarkers are RNA.

In some embodiments, the methods include detecting in the cell free nucleic acids the presence of a normalization nucleic acid.

In some embodiments, the method includes: obtaining a plasma sample from a second human subject, wherein the second human subject is a pregnant female carrying a fetus without trisomy 21; obtaining a second cell free nucleic acid sample from the plasma sample; and detecting in the second cell free nucleic acid sample the presence of the combination of nucleic acid biomarkers. In some aspects, the method can include: quantitating the amount of each nucleic acid biomarker in the cell free nucleic acids from the pregnant female; and quantitating the amount of each nucleic acid biomarker in the second cell free nucleic acid sample from the second pregnant female.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Examples of normalization sequences. Peptidylprolyl isomerase A (SEQ ID NO: 1); snRNA:U6:96Aa (SEQ ID NO: 2); snRNA:U6:96Ab (SEQ ID NO: 3); and snRNA:U6:96Ac (SEQ ID NO: 4).

FIGS. 2A-1 to 2A-72 and 2B-1 to 2B-2. Examples of T21 Biomarkers Selection: There are 3,143 mRNA biomarkers (FIGS. 2A-1 to 2A-72) and 98 noncoding small RNA sequences (miRNA plus other small RNAs such as snoRNA) biomarkers (FIGS. 2B-1 to 2B-2) selected from around 25,000 genes using Affymetrix microarray technique. FIGS. 2A-1 to 2A-72 and 2B-1 to 2B-2 are collectively referred to as FIG. 2 herein, where FIGS. 2A-1 to 2A-72 are collectively referred to as FIG. 2A and FIGS. 2B-1 to 2B-2 are collectively referred to as FIG. 2B.

FIGS. 3-1 to 3-255. Examples of 3,143 T21 mRNA biomarker sequences. Each biomarker includes the following: SEQ ID NO, Chromosome location, Gene Symbol, Gene Accession Number, fold-change, and sequence. FIGS. 3-1 to 3-255 are collectively referred to as FIG. 3.

FIGS. 4-1 to 4-7. Examples of 98 T21 noncoding small RNA biomarker sequences (miRNA plus other small RNAs such as snoRNA). Each biomarker includes the following: SEQ ID NO, Chromosome location, Gene Symbol, Gene Accession Number, fold-change, and sequence. FIGS. 4-1 to 4-7 are collectively referred to as FIG. 4.

FIGS. 5A-50. Examples of 15 T21 mRNA biomarkers confirmed by Real-time PCR in 10 affected pregnancies. The X-axis is the subject number. The figures represent a graphic illustration of marker expression in trisomy 21 (the squares) compared to the normal range for chromosomally normal fetuses. The dotted lines demarcate the 95% confidence interval for normal. FIGS. 5A-50 are collectively referred to as FIG. 5.

FIG. 6 shows that the maternal age in Normal (euploid fetus) women and those with a Trisomy 21 fetus.

FIG. 7A shows the Mean RNA expression of a 54 cell free RNAs subset (Group).

FIG. 7B shows the Mean RNA expression for the group of 54 cell free RNA markers for the Trisomy 21 (n=50) (Y axis) plotted against mean expression of the same marker in the Normal subjects (n=948).

FIGS. 8A-8I show the ROCs for the 9 RNAs shown by the light dots in FIG. 7B with the highest p values.

FIG. 9 shows a comparison of 11 Machine Learning (ML) algorithms.

FIG. 10 shows general workflow that leads to the identification of the biomarker subsets that are described herein.

FIG. 11 shows data for the three best performing ML algorithms.

FIG. 12A shows a specific 6 plasma cell free RNA group that happens to consist of mRNA that are products of genes located on the number 21 chromosome.

FIG. 12B shows a specific 6 plasma cell free RNA group that consist of 5 small noncoding RNAs produced by genes located on a chromosome other than the number 21, and 1 mRNA that is a product of a gene located on the number 21 chromosome.

FIG. 12C show a specific 11 plasma cell free RNA group that consists of the 11 unique RNAs identified with C5.0.

FIG. 13 shows the sequence for the mRNA of ATP50 (SEQ ID NO: 3249).

FIGS. 14-1 to 14-4 shows the sequence for the mRNA of DOP1B (SEQ ID NO: 3250).

FIGS. 15-1 to 15-5 shows the sequence for the mRNA of NCAM2 (SEQ ID NO: 3251).

FIG. 16 shows the sequence for the mRNA of UBASH3A (SEQ ID NO: 3252).

FIG. 17 shows the sequence for the mRNA of CHODL (SEQ ID NO: 3253).

FIGS. 18-1 to 18-2 shows the sequence for the mRNA of PKNOX1 (SEQ ID NO: 3254).

FIGS. 19-1 to 19-2 shows the sequence for the mRNA of CYYR1 (SEQ ID NO: 3255).

FIGS. 20-1 to 20-2 shows the sequence for the mRNA of GART (SEQ ID NO: 3256).

FIGS. 21-1 to 21-2 shows the sequence for the mRNA of RASGRP4 (SEQ ID NO: 3257).

FIGS. 22-1 to 22-2 shows the sequence for the mRNA of FAM20A (SEQ ID NO: 3258).

FIGS. 23-1 to 23-4 shows the sequence for the mRNA of NEK9 (SEQ ID NO: 3259).

FIGS. 24-1 to 24-3 shows the sequence for the mRNA of ABCC1 (SEQ ID NO: 3260).

FIGS. 25-1 to 25-2 shows the sequence for the mRNA of SORBS2 (SEQ ID NO: 3261).

FIG. 26 shows the sequence for the mRNA of TMPRSS2 (SEQ ID NO: 3262).

FIGS. 27-1 to 27-3 shows the sequence for the mRNA of DSCAM (SEQ ID NO: 3263).

FIGS. 28-1 to 28-2 shows the sequence for the mRNA of ERG (SEQ ID NO: 3264).

FIGS. 29-1 to 29-3 shows the sequence for the mRNA of ICOSLG (SEQ ID NO: 3265).

FIG. 30 shows the sequence for the mRNA of C21orf33 (SEQ ID NO: 3266).

FIGS. 31-1 to 31-5 shows the sequence for the mRNA of ADAMTSS (SEQ ID NO: 3267).

FIGS. 32-1 to 32-3 shows the sequence for the mRNA of CXADR (SEQ ID NO: 3268).

FIGS. 33-1 to 33-2 shows the sequence for the mRNA of PFKL (SEQ ID NO: 3269).

FIGS. 34-1 to 34-2 shows the sequence for the mRNA of SLC19A1 (SEQ ID NO: 3270).

FIGS. 35-1 to 35-3 shows the sequence for the mRNA of PRDM15 (SEQ ID NO: 3271).

FIGS. 36-1 to 36-2 shows the sequence for the mRNA of COL6A1 (SEQ ID NO: 3272).

FIGS. 37-1 to 37-2 shows the sequence for the mRNA of ABCG1 (SEQ ID NO: 3273).

### DETAILED DESCRIPTION OF ILLUSTRATIVE EMBODIMENTS

Provided herein are methods for determining whether a subject has trisomy 21. In one embodiment, a method may include screening a fetus for trisomy 21. The method may include measuring a plurality of trisomy 21 biomarkers in a biological sample obtained from a first pregnant female, wherein the plurality of trisomy 21 biomarkers is chosen from any combination of the nucleic acids or a complement thereof. In one embodiment, the fetus of the first pregnant female is at least 6 weeks post-implantation, or at least 7 weeks, or at least 8 weeks, or at least 9 weeks, or at least 10 weeks, or at least 12 weeks through the end of pregnancy. The pregnant female may also have a pregnancy that is less than 32 weeks, less than 24 weeks, or less than 18 weeks.

The method may also include identifying the fetus as having trisomy 21 if expression of the plurality of biomarkers is altered to a statistically significant degree in the biological sample (e.g., first biological sample) compared to a second biological sample from a second pregnant female carrying a fetus not having trisomy 21. The method may also include identifying the fetus as not having trisomy 21 if expression of the plurality of biomarkers is not altered to a statistically significant degree in the first biological sample compared to a second biological sample from a second pregnant female carrying a fetus not having trisomy 21. In one embodiment, expression of a trisomy 21 biomarker is altered to a statistically significant degree if it is outside the 95% confidence interval for that trisomy 21 biomarker. In one embodiment, the method may further include recommending a genetic test chosen from amniocentesis, cordocentesis, and chorionic villus sampling or a combination thereof.

In one embodiment, the plurality of trisomy 21 biomarkers may include at least 6 trisomy 21 biomarkers, wherein the pregnant mother having at least 6 biomarkers whose expression is altered to a statistically significant degree to identify the fetus as having trisomy 21. In one embodiment, the plurality of trisomy 21 biomarkers includes at least 11 trisomy 21 biomarkers, and wherein the pregnant mother having their expression not altered to a statistically significant degree to identify the fetus as having trisomy 21. The plurality of trisomy 21 biomarkers may include at least 6 biomarkers, at least 10 biomarkers, at least 11 biomarkers, at least 24 biomarkers, at least 25 biomarkers, at least 27 biomarkers, at least 30 biomarkers, at least 40 biomarkers, at least 43 biomarkers, at least 45 biomarkers, at least 50 biomarkers and at least 54 biomarkers.

In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the first pregnant female carrying a fetus with trisomy 21 compared to the second pregnant female carrying a fetus not having trisomy 21. In one embodiment, the trisomy 21 biomarkers may be selected from polynucleotides that are down-regulated in the first pregnant female carrying a fetus with trisomy 21 compared to the second pregnant female carrying a fetus not having trisomy 21.

In one embodiment, the method may further include obtaining the biological sample from the first pregnant female. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

In one embodiment, a method may include detecting trisomy 21 in a fetus. The method may include detecting trisomy 21 biomarkers in a biological sample to yield an expression level of each detected trisomy 21 biomarker in a biomarker combination. In one embodiment, the biological sample includes plasma from a pregnant female. In one embodiment, the fetus of the first pregnant female is at least 6 weeks post-implantation. The method may also include comparing the expression level of each detected trisomy 21 biomarker in a combination of biomarkers to the expression level of the trisomy 21 biomarker in pregnant females carrying a fetus without trisomy 21. In one embodiment, an expression level of a detected trisomy 21 biomarker that is outside the 95% confidence interval for that trisomy 21 biomarker indicates the expression level of the trisomy 21 biomarker is altered. In another embodiment, the expression level of the detected trisomy 21 biomarker is determined by application of a machine learning algorithms that analyzes patterns and performs machine ranking. In one embodiment, at least 6 or 10 trisomy 21 biomarkers are detected. In one embodiment, a fetus carried by the pregnant female is identified as carrying a fetus having trisomy 21 when at least 6 biomarkers are outside the 95% confidence interval. In one embodiment, the method may further include recommending a genetic test chosen from amniocentesis, cordocentesis, or chorionic villus sampling. In one embodiment, the pregnant female and the pregnant females used to establish the 95% confidence interval for each trisomy 21 biomarker may be matched with respect to a co-variable such as gestational stage or ethnicity or a combination thereof.

In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the pregnant female carrying a fetus with trisomy 21 compared to the pregnant females carrying a fetus not having trisomy 21. In one embodiment, the trisomy 21 biomarkers may be selected from polynucleotides that are down-regulated in the pregnant female carrying a fetus with trisomy 21 compared to the pregnant females carrying a fetus not having trisomy 21.

In one embodiment, the method may further include obtaining the biological sample from the first pregnant female. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

In one embodiment, a method may include detecting trisomy 21 in a fetus. The method may include detecting trisomy 21 biomarkers in a biological sample from a pregnant female to yield a sample expression profile. In one embodiment, the biological sample includes plasma from a pregnant female. In one embodiment, the T21 biomarkers may be chosen from a sequence of (e.g., at least 5, 10, or 15 consecutive) nucleotides selected from any combination of nucleic acid biomarkers as defined herein, or a complement thereof. In one embodiment, the fetus of the first pregnant female is greater than 8 weeks post-implantation. The method may also include comparing the sample expression profile with a reference expression profile, wherein a difference between the sample expression profile and the reference expression profile is indicative of the presence or absence of trisomy 21 in the fetus. In one embodiment, the reference expression profile is from at least one second pregnant female carrying a fetus without trisomy 21, and a difference between the sample expression profile and the reference expression profile is indicative of the presence of trisomy 21. In one embodiment, the reference expression profile is from at least one second pregnant female carrying a fetus with trisomy 21, and a difference between the sample expression profile and the reference expression profile is indicative of the absence of trisomy 21. In one embodiment, the method may further include recommending a genetic test chosen from amniocentesis, cordocentesis, and chorionic villus sampling.

In one embodiment, the difference between the sample expression profile and the reference expression profile is statistically significant. In one embodiment, the sample expression profile includes at least 6 or 10 trisomy 21 biomarkers. In one embodiment, the trisomy 21 biomarkers may be selected from polynucleotides encoded by chromosome 21, or from polynucleotides encoded by any of chromosomes 1-20, 22 or X. In one embodiment, the trisomy 21 biomarkers may be selected from polynucleotides that are up-regulated in the first pregnant female carrying a fetus with trisomy 21 compared to the second pregnant female carrying a fetus not having trisomy 21. In one embodiment, the trisomy 21 biomarkers may be selected from polynucleotides that are down-regulated in the first pregnant female carrying a fetus with trisomy 21 compared to the second pregnant female carrying a fetus not having trisomy 21. In one embodiment, the first pregnant female with a fetus having trisomy 21 and the second pregnant female with a euploid fetus may be matched with respect to a co-variable such as gestational stage and ethnicity.

In one embodiment, the method may further include obtaining the biological sample from the first pregnant female whose fetus may have trisomy 21. The obtaining may include obtaining a blood sample. The blood sample may be processed to remove cells from the blood sample. The blood sample may be processed to obtain, and optionally isolate, cell-free plasma RNA. In one embodiment, the method may further include converting RNA polynucleotides present in the biological sample into cDNA molecules, and the measuring includes hybridization between a cDNA molecule and a complementary trisomy 21 biomarker. In one embodiment, the complementary trisomy 21 biomarker is in solution during the hybridization, and in one embodiment, the complementary trisomy 21 biomarker is immobilized on a solid support.

In one embodiment, an article includes a substrate and a plurality of different polynucleotides. In one embodiment, the polynucleotides are selected from any combination nucleic acids as described herein (e.g., defined groups), or a complement thereof. In one embodiment, the T21 biomarkers are selected from a sequence of at least 5, 10 or 15 consecutive nucleotides selected from any combination of the nucleic acid biomarkers, or a complement thereof. The polynucleotides are immobilized onto a surface of the substrate. In one embodiment, the polynucleotides are immobilized on the substrate surface to form a microarray. In one embodiment, at least 10 polynucleotides are immobilized on the substrate surface.

Also provided herein are kits. In one embodiment, a kit includes an article having a substrate, a plurality of different polynucleotides immobilized onto a surface of the substrate, and packaging materials and instructions for use. In one embodiment, the polynucleotides are selected from any combination of the defined groups of nucleic acid biomarkers, or a complement thereof. In one embodiment, the T21 biomarkers are selected from a sequence of at least 5, 10, or 15 consecutive nucleotides selected from any combination of the defined groups of the nucleic acid biomarkers, or a complement thereof. In one embodiment, the polynucleotides are immobilized on the substrate surface to form a microarray.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Provided herein are polynucleotides useful for determining whether a subject, or a subject's fetus, has trisomy 21 (T21), and methods for using the polynucleotides. The methods described herein, and other embodiments disclosed herein such as reagents and kits, are based in part on the surprising discovery of a plurality of molecular markers, the expression levels of which consistently differentiate between healthy subjects and subjects with T21. The molecular markers are derived from coding regions whose altered expression in an affected subject, as measured from an easily obtained biological sample, is indicative of the subject, or the subject's fetus, having T21. As used herein, the term “polynucleotide” refers to a polymeric form of nucleotides of any length, either ribonucleotides or deoxynucleotides, and includes both double- and single-stranded DNA and RNA. A polynucleotide can be obtained directly from a natural source, or can be prepared with the aid of recombinant, enzymatic, or chemical techniques. A polynucleotide can be linear or circular in topology. The terms cDNA, oligonucleotide, probe, and nucleic acid are included within the definition of polynucleotide and these terms are used interchangeably. The term polynucleotide also includes peptide nucleic acids (Nielsen et al., 1991, Science. 254:1497-500), and other nucleic acid analogs and nucleic acid mimetics (see, e.g., McGall et al., U.S. Pat. No. 6,156,501).

In one embodiment, a method provided herein includes detecting one or more T21 biomarkers in a biological sample. As used herein, a “biological sample” refers to a sample of tissue or fluid obtained from a subject, including but not limited to, for example, whole blood, blood plasma, serum, lymph fluid, synovial fluid, cerebrospinal fluid, urine, and saliva. In one embodiment, a biological sample includes serum. In one embodiment the methods provided herein are directed to non-invasive methods of detecting T21, and in such an embodiment a biological sample may be a fluid. In one embodiment, a biological sample includes blood plasma. In one embodiment, a biological sample includes whole blood. As used herein, “subject” refers to a prenatal or postnatal human. A prenatal human includes a fetus. Unless indicated otherwise, as used herein the term “fetus” refers to a human during prenatal development from the time of first cell division until birth. The fetus may be at any age after implantation. For instance, the fetus may be at 2 weeks post-implantation (PI), 4 weeks PI, 6 weeks PI, 8 weeks PI, 10 weeks PI, 12 weeks PI, 14 weeks PI, 16 weeks PI, 18 weeks PI, 20 weeks PI, etc. In one embodiment, the fetus is between 6 weeks and 20 weeks PI, or between 7 weeks and 14 weeks PI, or 15-20 weeks PI. A postnatal human refers to an individual at any stage of development after birth, including a newborn, a child, an adolescent, or an adult, and includes a pregnant human mother. In one embodiment where the subject is a pregnant human mother, the mother does not have T21. In the embodiment where the subject is a pregnant human mother, a method provided herein allows one to determine if the fetus carried by the pregnant mother has T21.

As used herein, a “T21 biomarker” is a polynucleotide that is indicative of T21 in a subject. A T21 biomarker is indicative of T21 when the expression level or quantity of the biomarker is altered more often in a subject having T21 compared to a healthy subject, which expression level may be higher for a subject having T21 for certain biomarkers, lower for a subject having T21, or in some instances the biomarker may be higher or lower for the subject having T21. The change in the expression level from a standard (e.g., subject without T21) to a statistically significant degree for a combination of biomarkers, whether the change is upregulation or downregulation, can provide the indication of T21 in a subject. In some instances, the same biomarker can increase in one patent but decrease in another patient, but along with the other identified combination of biomarkers, the change itself for that biomarker provides an indication of the subject having T21. In some aspects, a panel or combination of biomarkers can be assessed for a change in expression, and when a certain percentage thereof change expression, whether upregulated or downregulated, the subject is identified as having T21. A T21 biomarker having an altered expression level or quantity is one that is expressed at a greater level (e.g., over-expressed, upregulated) or expressed at a lower level (e.g., under-expressed, downregulated) when compared to a healthy subject or compared against a standard (e.g., average of a plurality of expression profiles for the biomarkers in subjects without T21). Whether the expression level or quantity of a biomarker in a subject having T21 is altered, e.g., greater than or less than the expression level or quantity of the biomarker in a healthy subject or standard, is determined using routine statistical methods or machine learning techniques using pattern recognition and ranking.

It should be understood that the term “biomarker,” can, depending on the context, refer to the physical polynucleotide itself or to a graphical or numerical representation of the polynucleotide such as an amount of fluorescence present at a spot on a microarray, a band on a gel image, a numerical value, and the like. For example, the amount of fluorescence at a particular spot on a microarray may be referred to as a T21 biomarker when the fluorescence is linked to a specific polynucleotide. This graphical or numerical biomarker reflects the existence of the underlying expressed polynucleotide in the test sample, which gave rise to an expression level.

In one embodiment, the detecting of one or more T21 biomarkers in a biological sample yields an expression level of each detected biomarker. In one embodiment, the detecting of two or more T21 biomarkers in a biological sample yields a sample expression profile. An “expression level” is any physical representation of the amount of a selected T21 biomarker, as determined from one or more biological samples from a subject. A “sample expression profile” is any physical representation of the amounts of a set of two or more selected T21 biomarkers, as determined from one or more biological samples from a subject. The subject may be one known to have T21, known to have T21 of a particular type (for instance, 47XX+21, 47XY+21, or mosiac), known to be free of T21, or the status of T21 in the subject may be unknown. In one embodiment, a sample expression profile for a subject may include information from a single biological sample that has been analyzed for T21 biomarker expression levels. In one embodiment, a sample expression profile for a subject may include information from multiple types of biological samples that have been analyzed separately for T21 biomarker expression levels.

The terms “normal” and “healthy” are used herein interchangeably to refer to a subject or subjects who do not have a chromosomal abnormality associated with T21. A normal or healthy sample refers to a sample or samples obtained from a normal/healthy subject.

One skilled in the art will appreciate that more than one sample from a subject may be examined. The expression level and/or sample expression profile may be represented in visual graphical form, for example on paper or on a computer display, in a three dimensional form such as an array, and/or stored in a computer-readable medium. An expression level and/or sample expression profile may correspond to a particular status of T21 (e.g., presence or absence of T21) or type (e.g., 47XX+21, 47XY+21, or mosiac), and thus provide a template for comparison to a patient sample. A negative control expression level and/or a control expression profile, also referred to herein as a reference expression level and a reference expression profile and a standard expression profile, can be obtained by analyzing a biological sample from at least one healthy subject, or multiple samples obtained from a group of healthy subjects. A positive control expression level can be from one or more subjects identified as having comparable T21 in terms of type. When multiple samples from a group are used, the levels of expression of each detected T21 biomarker may be an average, consensus, or composite derived from the multiple samples. Similarly, comparable profiles can be obtained for age-matched and/or sex-matched subjects, and comparable profiles can be obtained for pregnant mothers at the same or similar stage of pregnancy. In one embodiment, expression levels and/or expression profiles can be obtained from a pregnant mother, and if the fetus is later determined to be healthy, such expression levels and/or expression profiles can be used as control expression levels and/or control expression profiles.

One skilled in the art will appreciate that multiple nontest factors may alter the marker level measured and may be mathematically adjusted by one of several well known and routine approaches. For example, the median level of each T21 biomarker may be determined at each gestational epoch in control women. If there is a statistically significant change with gestation, regression analysis of median on gestation weighted for the number of samples per epoch may be performed to determine the normal median curve that best fits the data. All results, both affected and unaffected pregnancies, may be expressed as multiples of the gestation-specific median (MoM) based on the fitted curve. In controls, potential co-variables may be examined, including maternal weight, smoking, prior preterm birth, diabetes or use of prophylactic progesterone and ethnicity, to see if they are significantly associated with the MoM. As the sample pool grows, it is likely other variables (such maternal medical diseases) may need to be considered. Plasma levels of fetal-placental derived sequences may decline on average with increased adiposity due to a fixed output being diluted into a greater volume of blood. If any co-variables are confirmed, the levels can be adjusted by, for instance, dividing the observed MoM by the expected median according to the co variable level found in unaffected pregnancies. Typically, the non-parametric Wilcoxon Rank Sum Test is used to select the subset of markers where there is a significant difference in the MoM distribution between affected and control pregnancies. As a large number of potential markers are to be tested, an extreme P-value of 0.005 may be used for an initial selection.

The risk of T21 may be modeled by the a priori risk of the disorder expressed as odds (a: b) multiplied by the likelihood ratio (LR) for the marker profile derived from multivariate Gaussian frequency distributions. All current aneuploidy and pre-eclampsia markers follow an approximately log Gaussian distribution over most of their range for both affected and unaffected pregnancies, and it is expected to be true for the T21 biomarkers disclosed herein. In some embodiments, the data may show the distribution is not Gaussian. [These Gaussian distributions are defined by the marker sequence means and standard deviations after log transformation. For a single marker, the LR is calculated by the ratio of the heights of the two overlapping distributions at the specific level. For extreme results that fall beyond the point where the data fits a Gaussian distribution, it is standard practice to use the LR at the end of the acceptable range. The method is the same for more than one marker except that the heights of multivariate log Gaussian distributions are used. These are defined, in addition to means and standard deviations, by the correlation coefficients between markers within affected and unaffected pregnancies. In some embodiments, machine learning algorithms may be used for analysis, which can include pattern recognition and ranking.

The method of numerical integration may be used to model the best combination of markers from the initial subset. This involves division of each marker operating range into up to 100 equal units, calculation of the volumes under the affected and unaffected multivariate Gaussian curves risk as well as the risk in the mid-point of the volume. This determines the distribution of risks in affected and unaffected pregnancies. These distributions will be calculated for all marker combinations and the sensitivity compared for a fixed specificity.

A second approach may be considered based on the well-known fact that a strong association does not guarantee effective discrimination between affected and unaffected. Nor does a high AUC guarantee good prediction of actual risk. Hence, model calibration via reclassification can be useful in order to accept only those markers least likely to have been identified at random. Prognostic models may be built for predictive accuracy after confirmed T21 with only non-T21 biomarker variables (age, race, maternal weight, gestation age, maternal comorbidities, etc.) and then build prognostic models to include T21 biomarkers. Dimensionality of the models may be reduced by translating the RNA marker contributions into a few components or composite scores. Principal components analysis may be used to derive the principal components of the T21 biomarkers factors. For instance, leading components that explain more than 85 percent of the total variation in genetic predictors may be retained and included in a prognostic model. Models that are more complicated (more predictors) may appear to have better predictive performance even if that is not the case. Therefore, the model performance may be quantified with respect to calibration and discrimination. Calibration may be examined by comparing the observed with the expected frequencies while the discriminatory accuracy may be assessed using the receiver operating characteristic (ROC) curve estimation for survival data. The true positive fraction or sensitivity and the false positive fraction (1−specificity) may be discussed using the derived prognostic models. For instance, the discriminatory accuracy may be compared between the models with and without validated genetic markers using the area under a ROC curve (AUC). Other validation techniques including cross-validation and bootstrap methods can also be carried to shed some insights about a model's adequacy. Alternatively, prognostic models can be constructed for T21 status (affected or unaffected) using logistic regression models. Modeling procedures may be similar to those previously described for routinely used Cox models.

In one embodiment, a T21 biomarker is RNA. In one embodiment, the RNA that is detected is cell-free, and is referred to herein as cell-free RNA. Cell-free RNA includes coding RNA (mRNA) and non-coding RNAs such as siRNA, miRNA, snoRNA, piRNA, exRNA, scaRNA, long ncRNAs and snRNA. In one embodiment, cell-free RNA is from whole blood, blood plasma, or serum, and is referred to herein as cell-free plasma (CFP) RNA. CFP RNA includes coding RNA (mRNA) and non-coding RNAs such as, but not limited to, siRNA, miRNA, snoRNA, and snRNA. For instance, when the sample is blood, the CFP RNA to be detected is present in the plasma portion of the blood. Thus, in one embodiment, a biological sample is processed to remove cells prior to the detecting. In one embodiment, a biological sample is processed to minimize cell lysis. In one embodiment, the CFP RNA that is detected may be mRNA, non-coding RNA, or the combination thereof. Optionally, the CFP RNA may be isolated.

RNA may be obtained from a biological sample using routine methods. In one embodiment, RNA is obtained using a process based on a phenol/guanidium isothiocyanate/glycerol phase separation. Such a process may result in large quantities of CFP nucleic acid with total RNA yields of 8-30 ug or more from only 2 mL of plasma and full range of RNAs including not only mRNA but also small noncoding RNAs such as miRNA and snoRNA. This amount is more than enough for both array and RNAseq technologies and the performance of numerous PCR reactions using a clinically practical, single patient sample.

The RNA isolation method described herein allows for the isolation of 8 micrograms to 30 micrograms of CFP RNA from a 2 mL sample, which is more than enough for both microaarray gene screening and PCR validation. The method may include obtaining 2 mL or more of sample from a subject, such as plasma, and following the steps as described in Example 1.

The analysis of samples of blood obtained from pregnant mothers who later gave birth to healthy infants or gave birth to infants with T21 has led to the discovery of T21 biomarkers. Examples of T21 biomarkers are described at SEQ ID NO:8-3,273. Different combinations of the T21 biomarkers listed at SEQ ID NO:8-3,273, or the complement thereof, allow the skilled person to predict whether the fetus carried by a pregnant mother has T21. Changes in the expression levels of these biomarker polynucleotides in a subject, as measured in a biological sample from the subject, thus may be used to indicate the presence, absence, or type or T21 in a subject, such as a fetus carried by a mother, or an infant, child, adolescent, or adult.

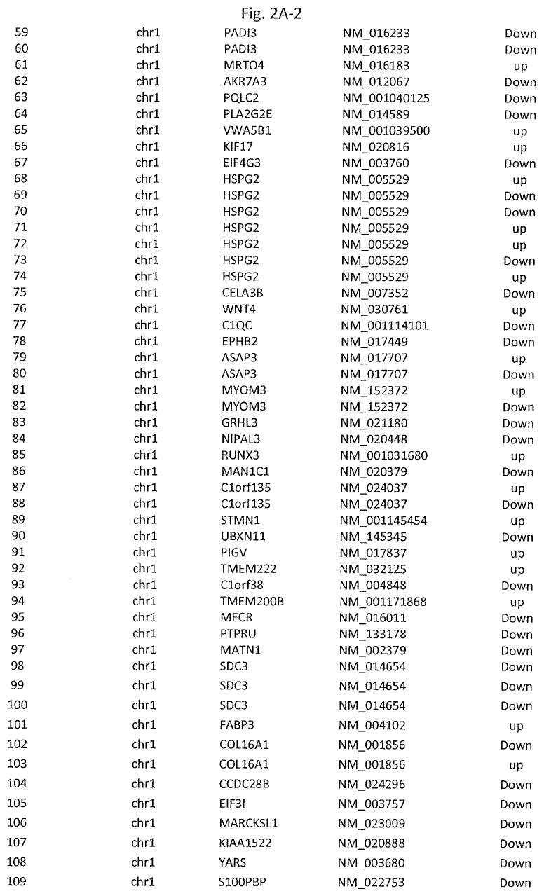

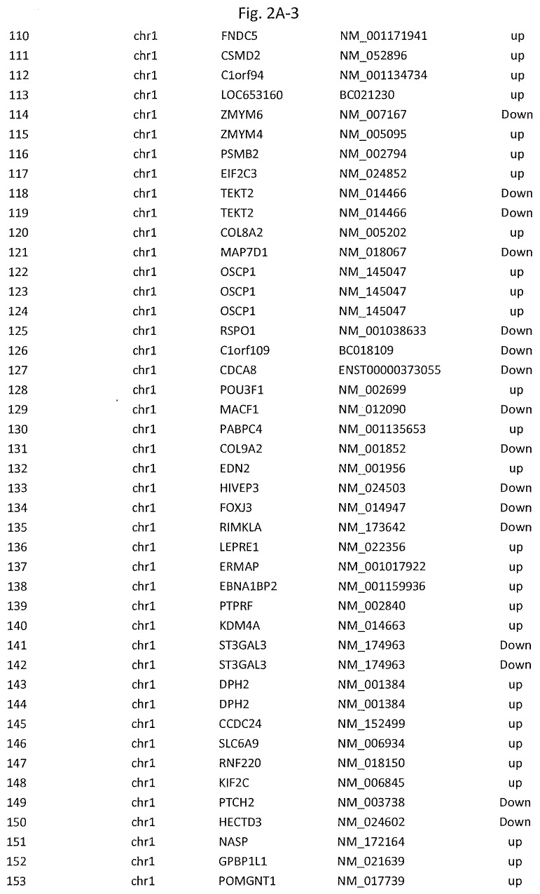

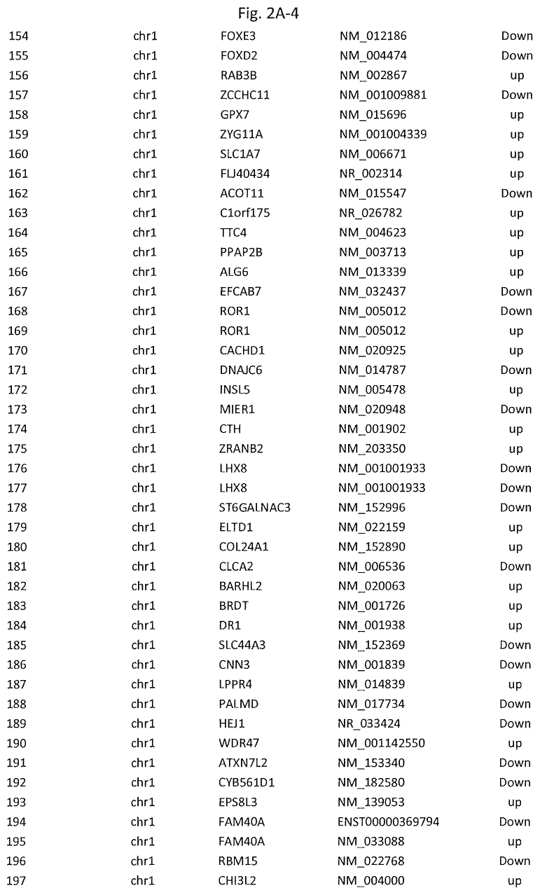

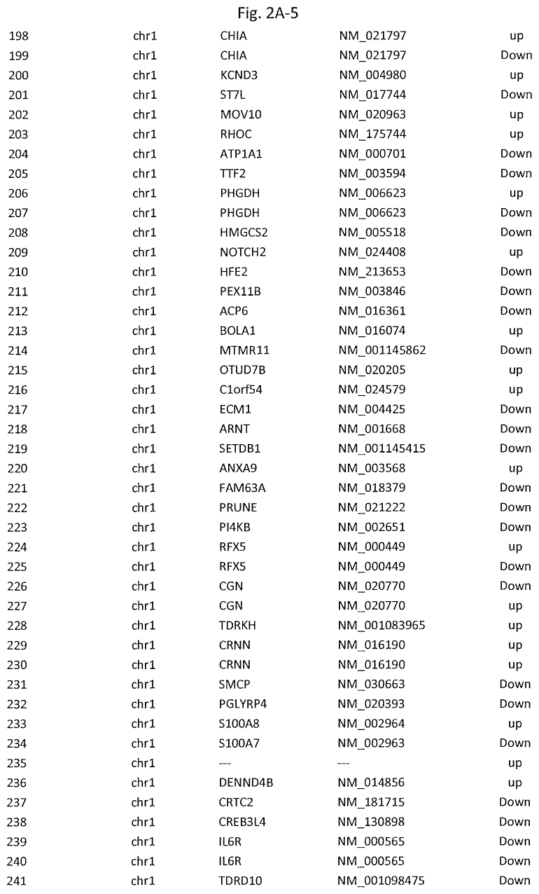

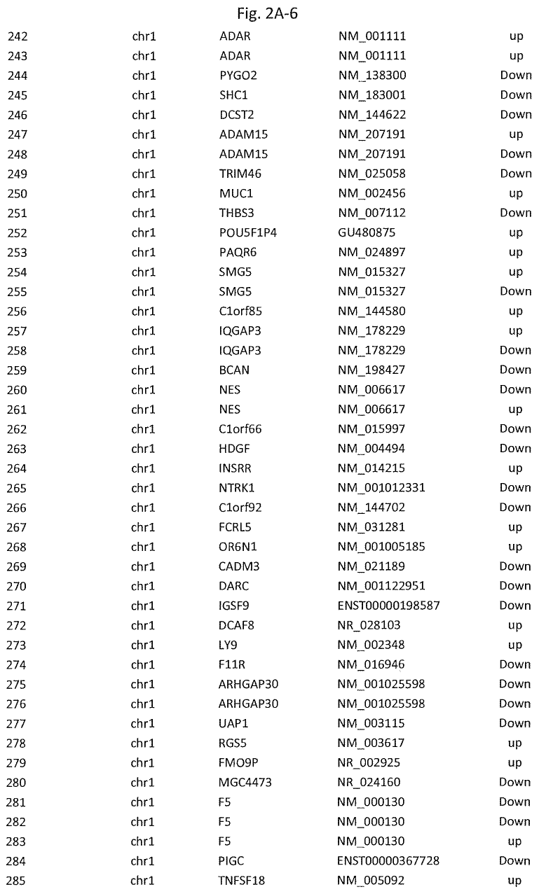

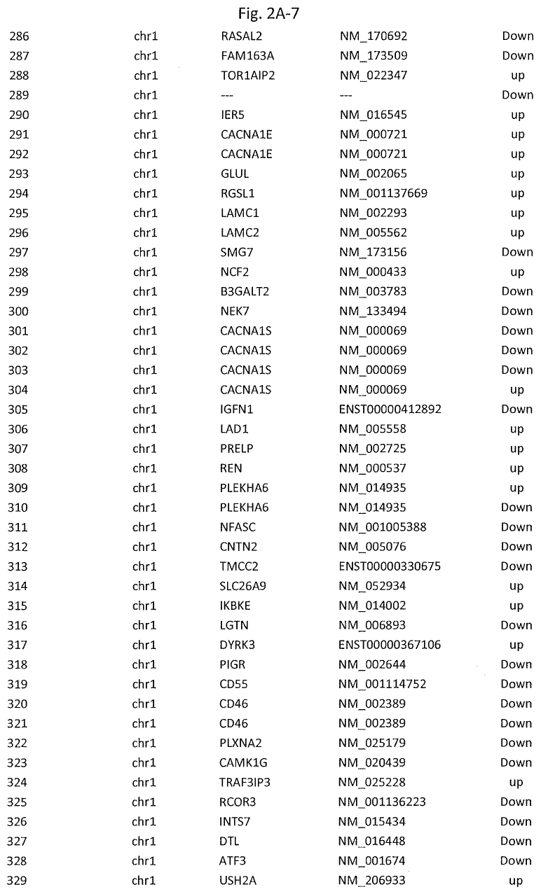

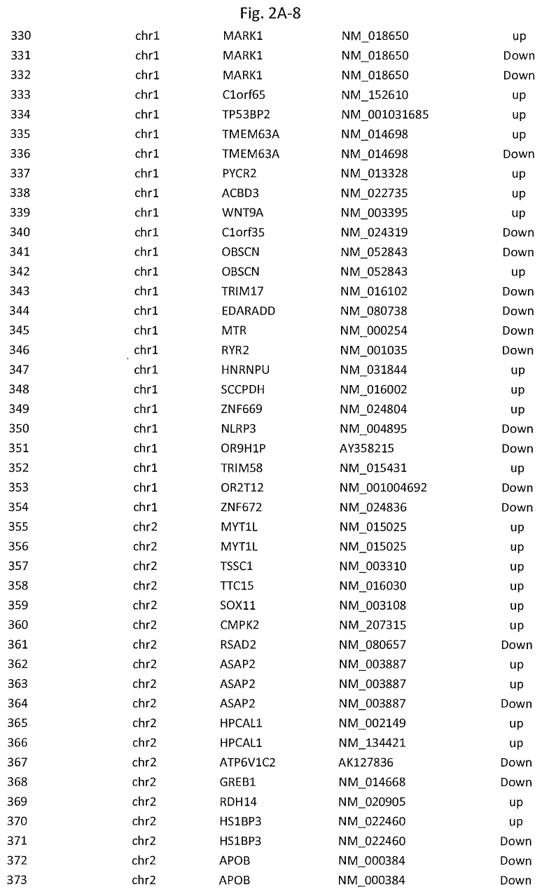

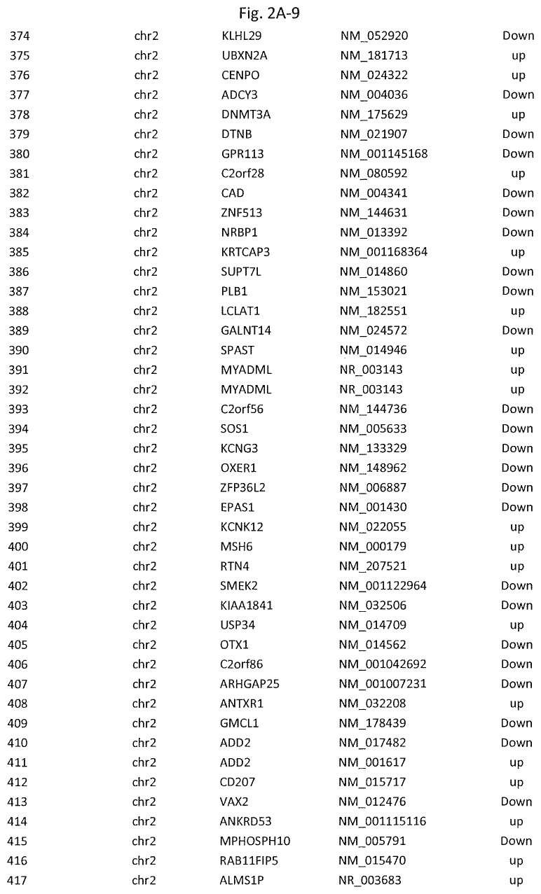

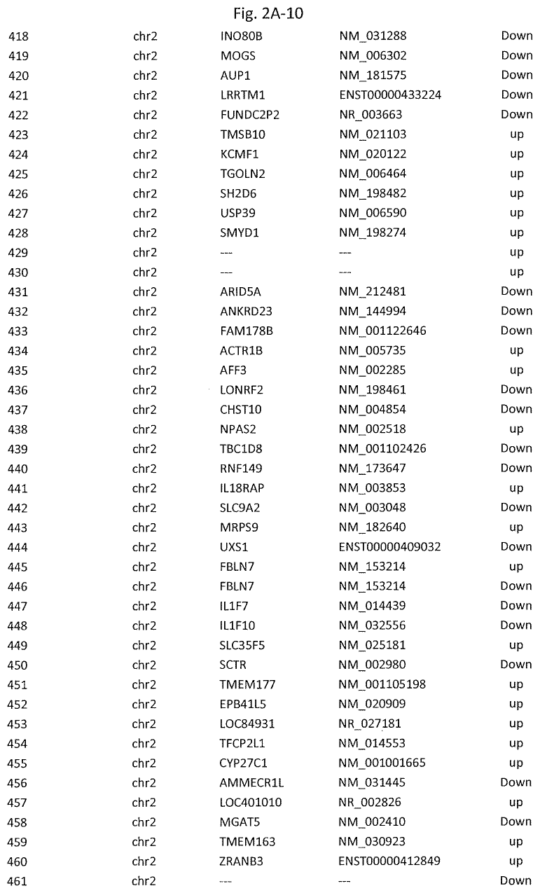

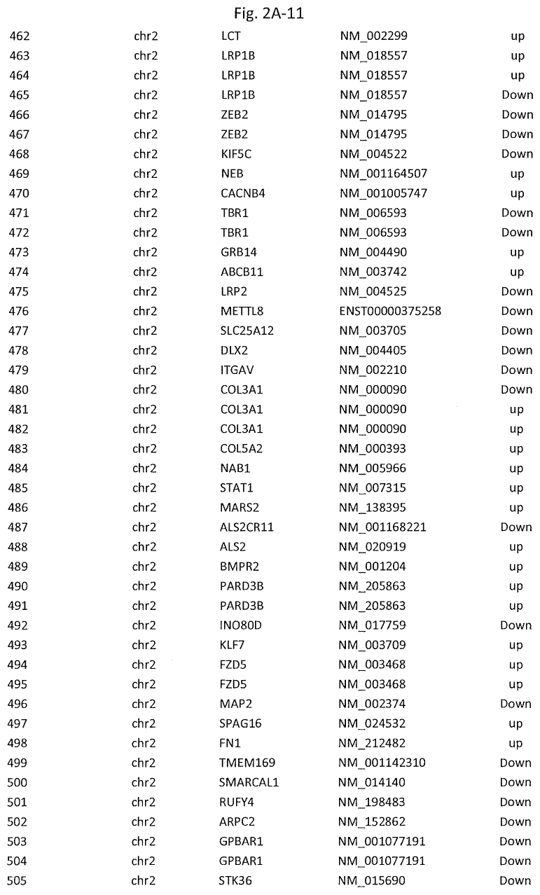

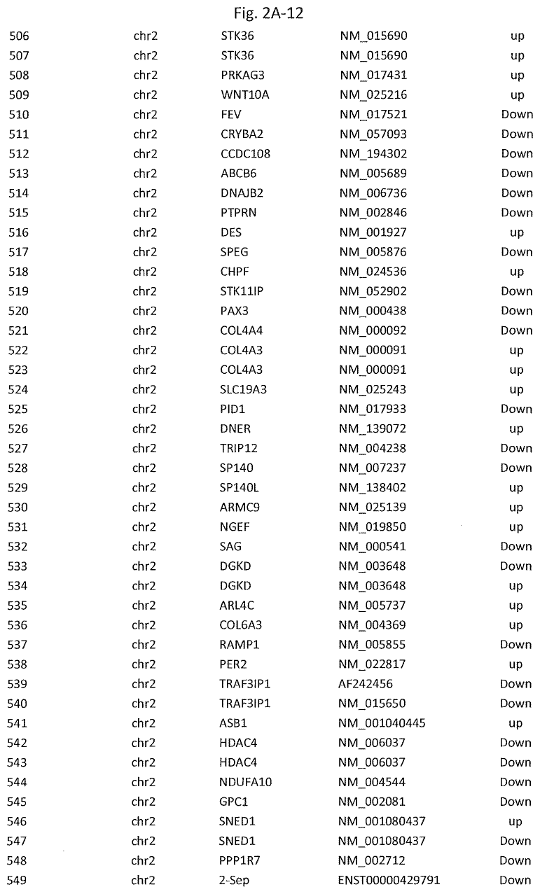

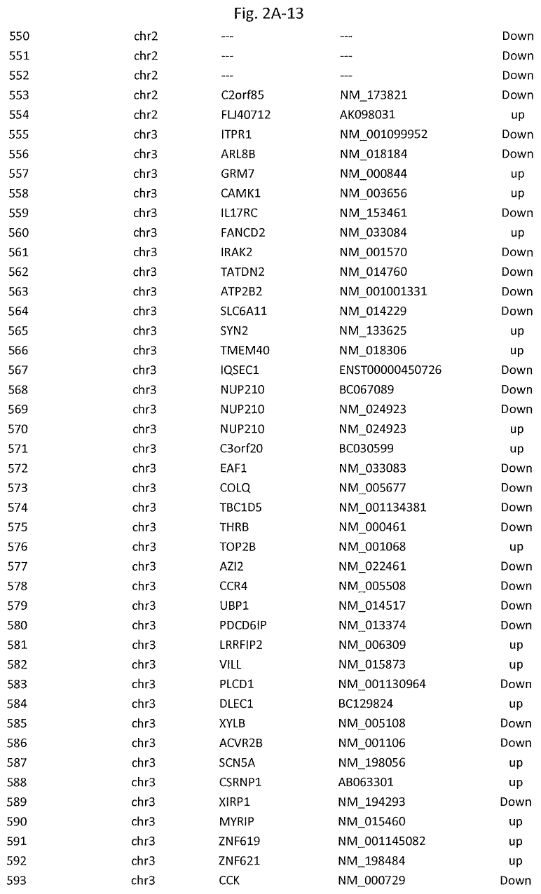

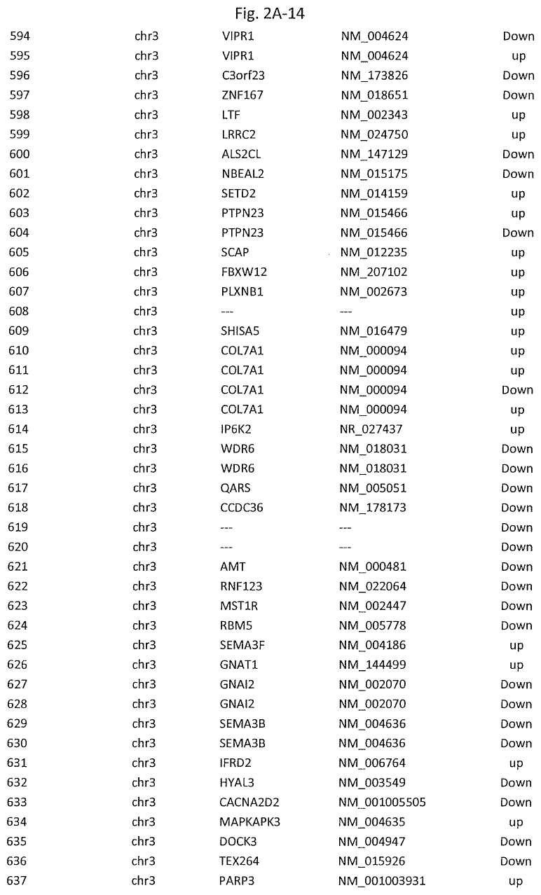

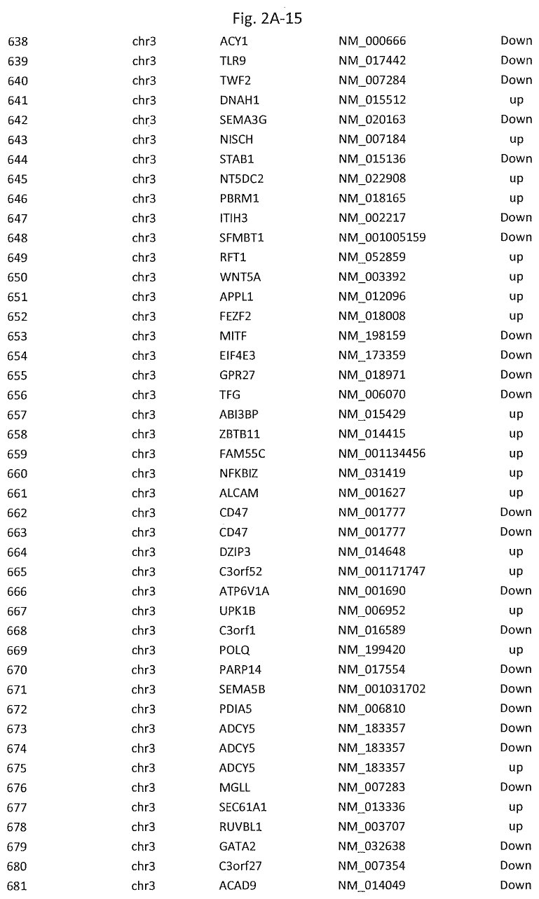

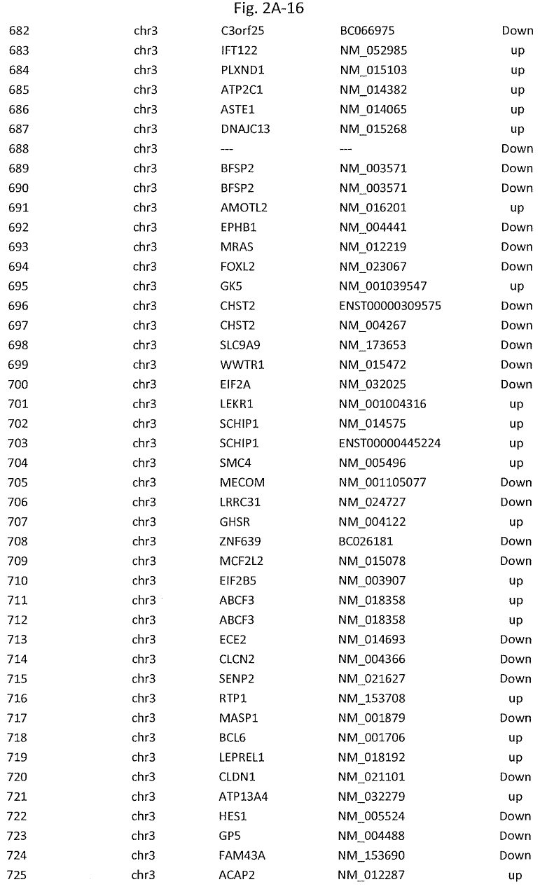

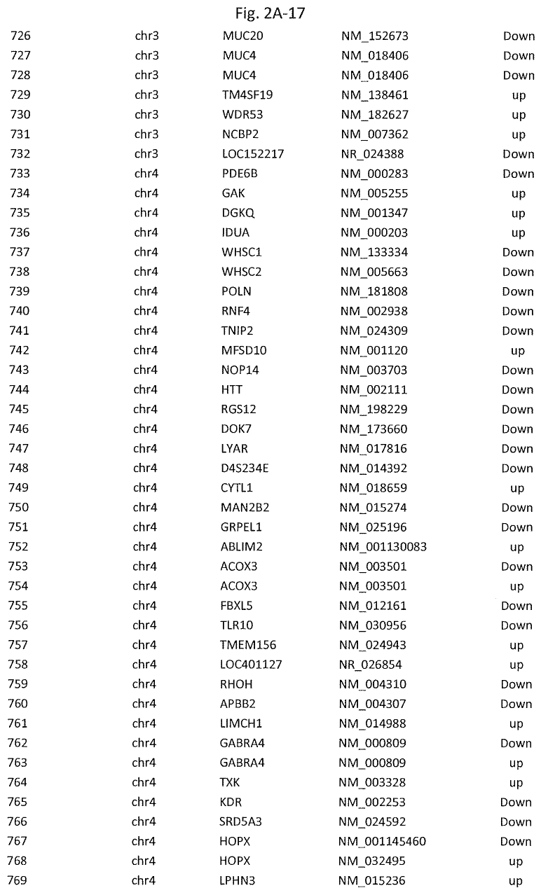

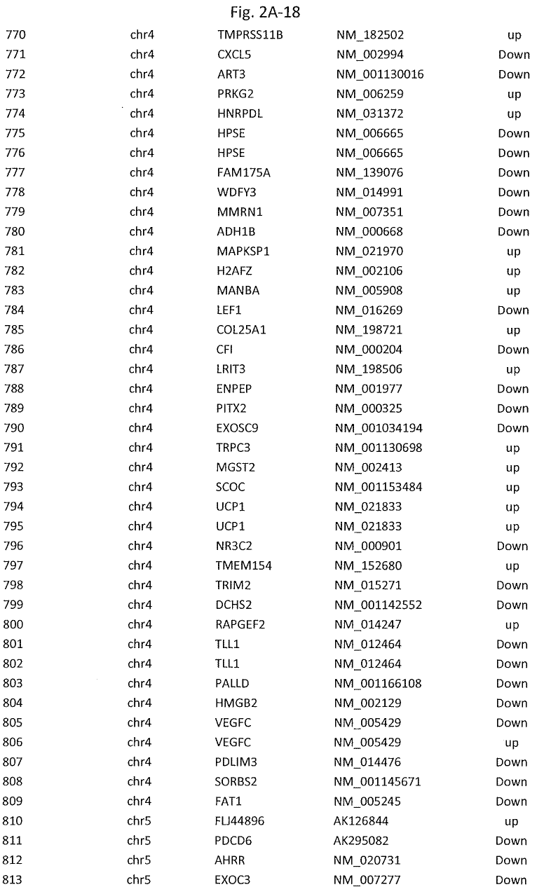

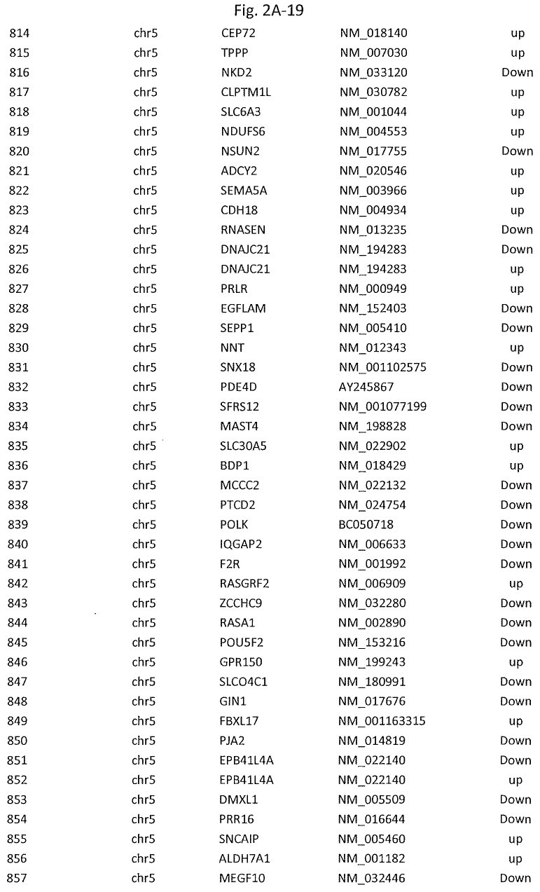

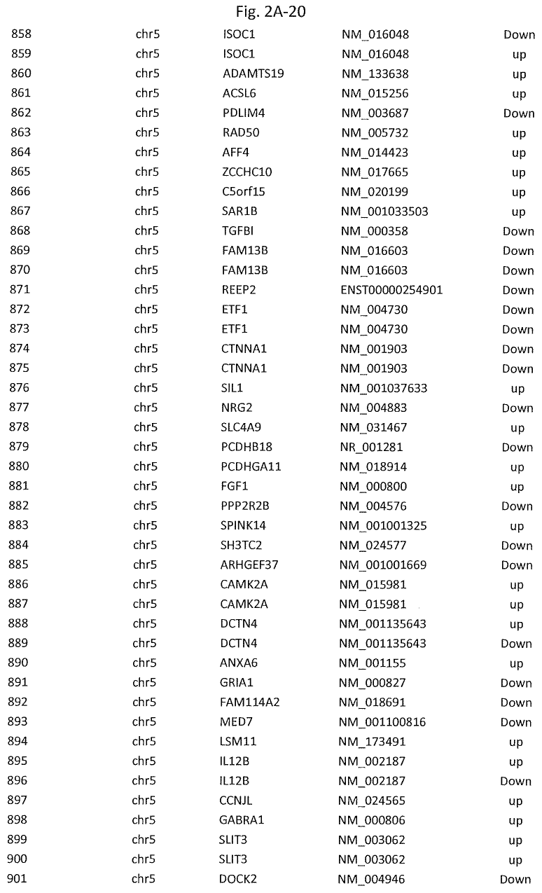

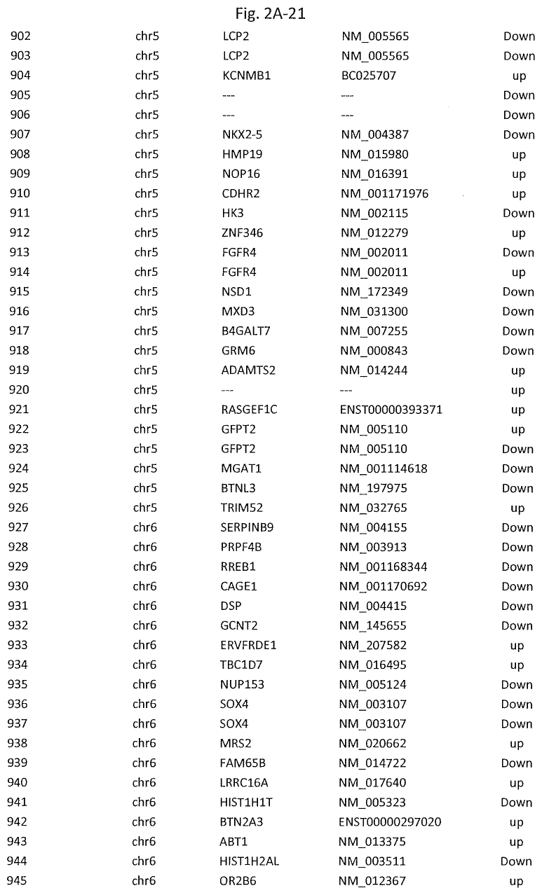

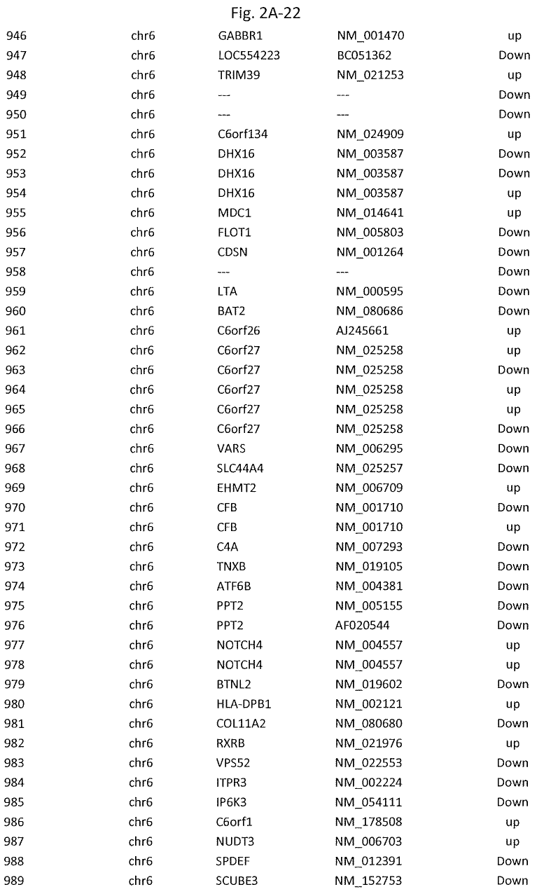

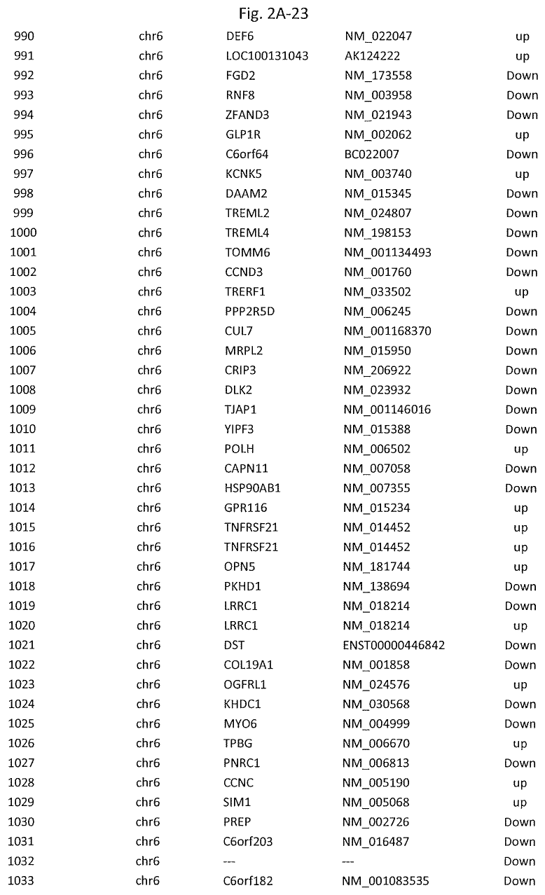

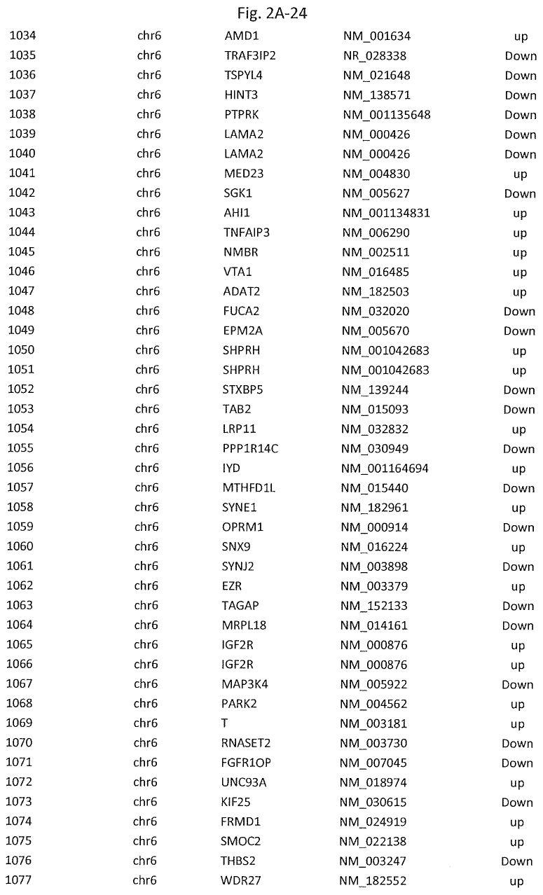

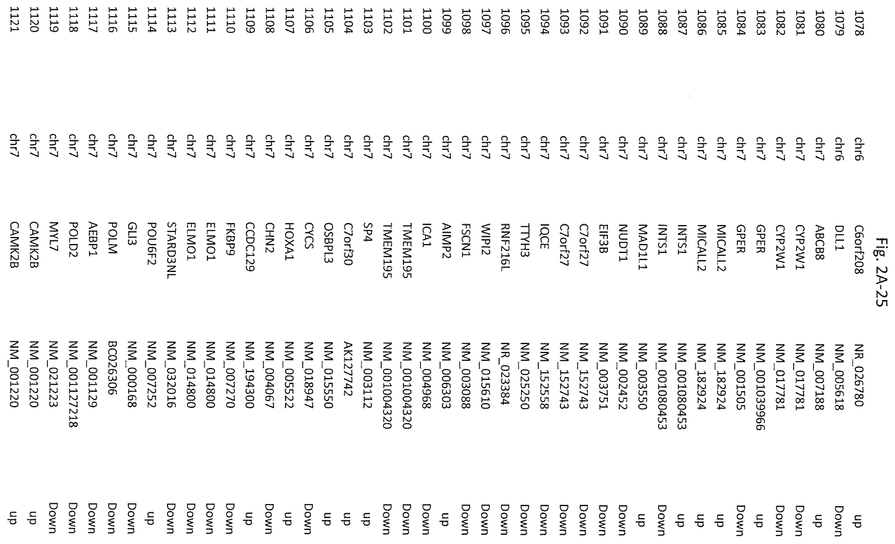

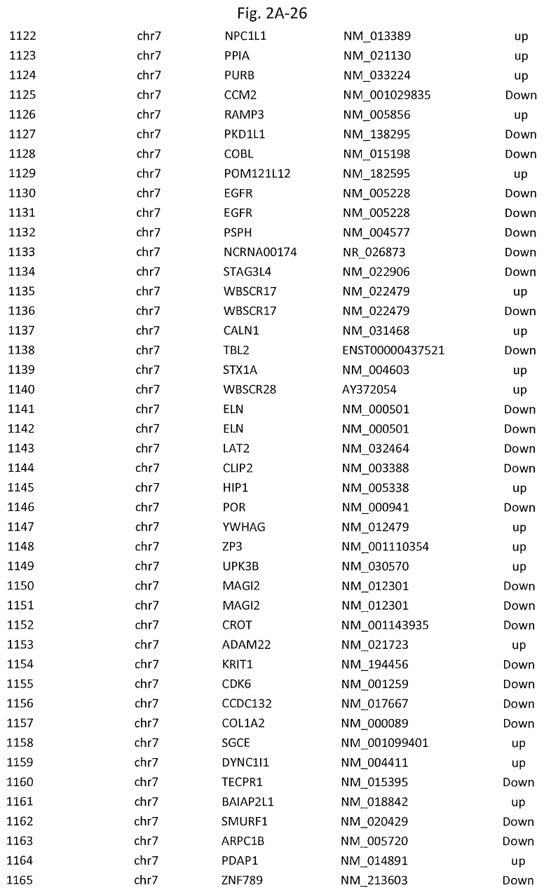

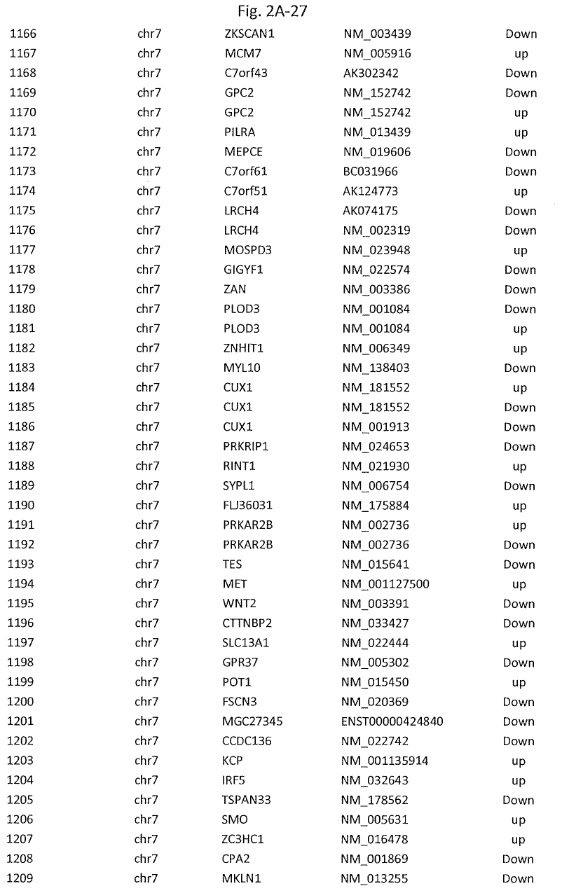

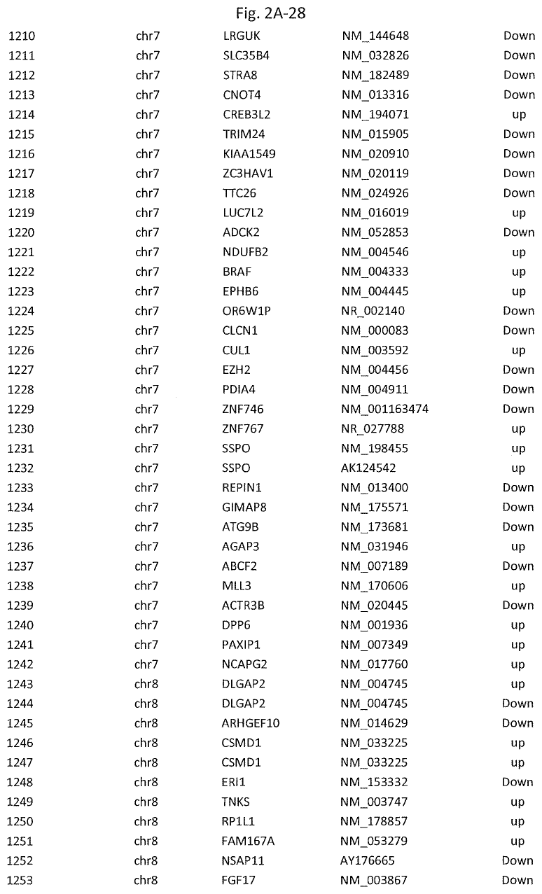

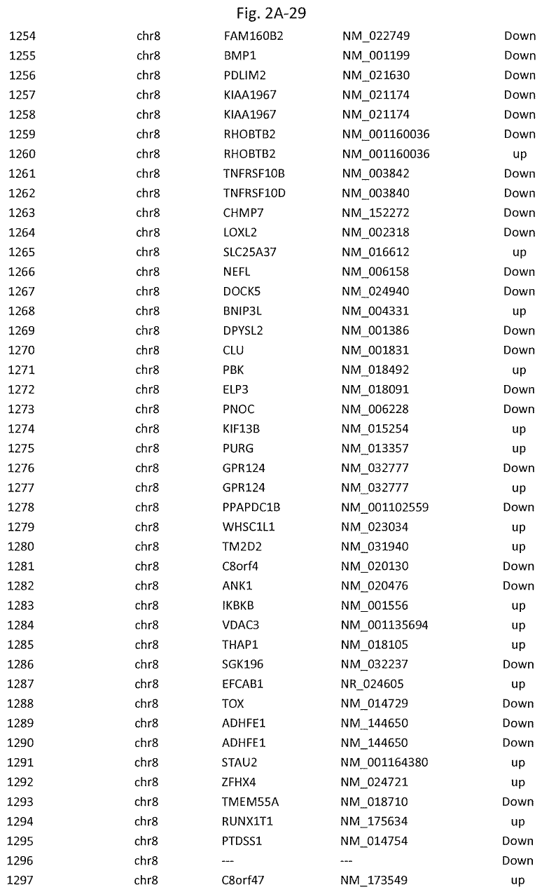

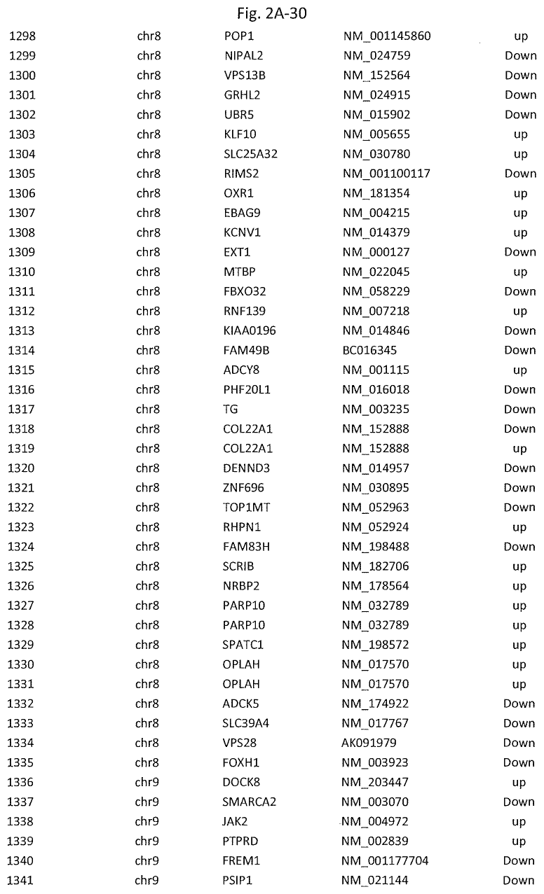

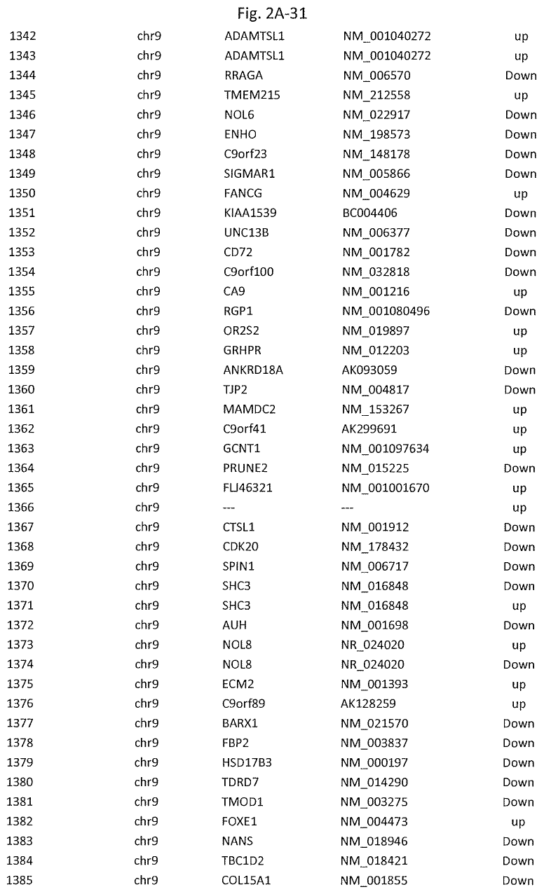

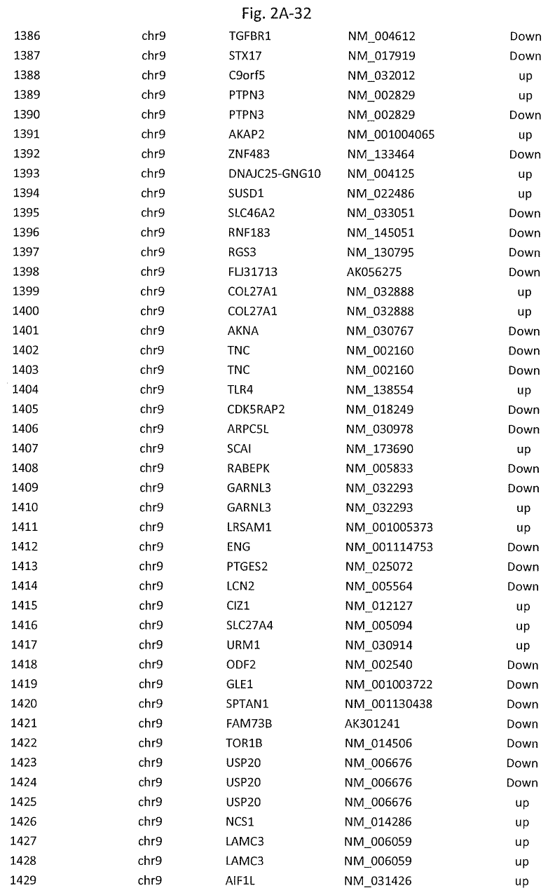

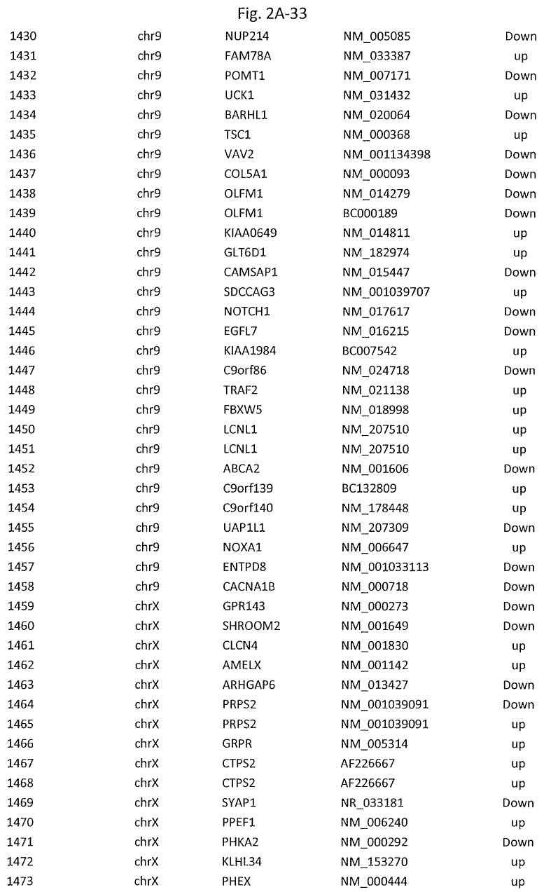

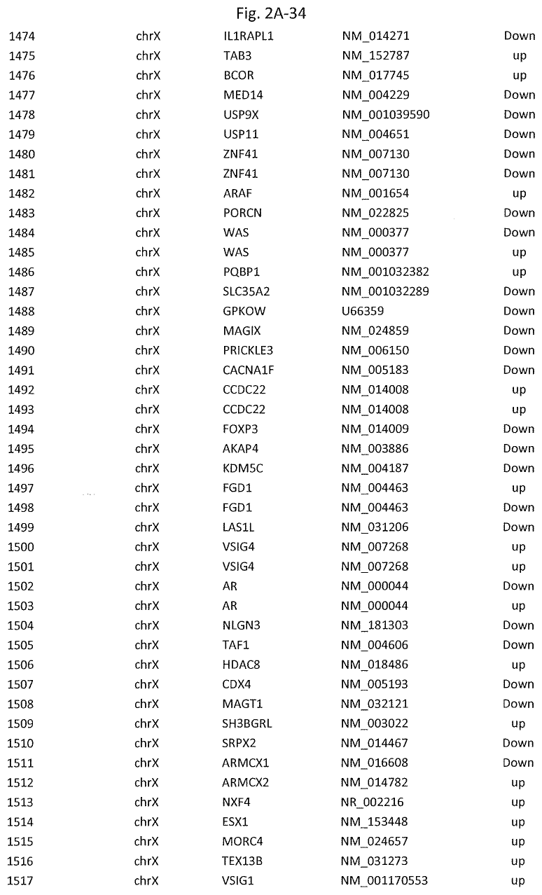

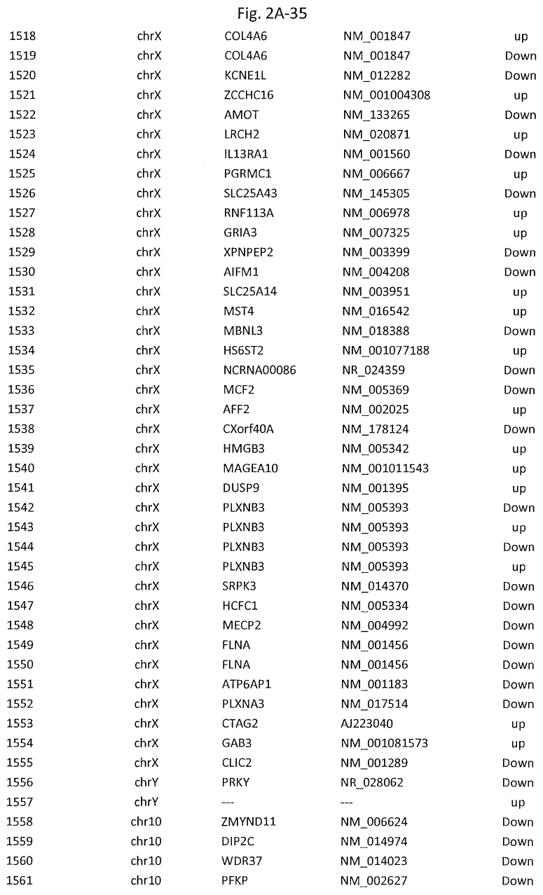

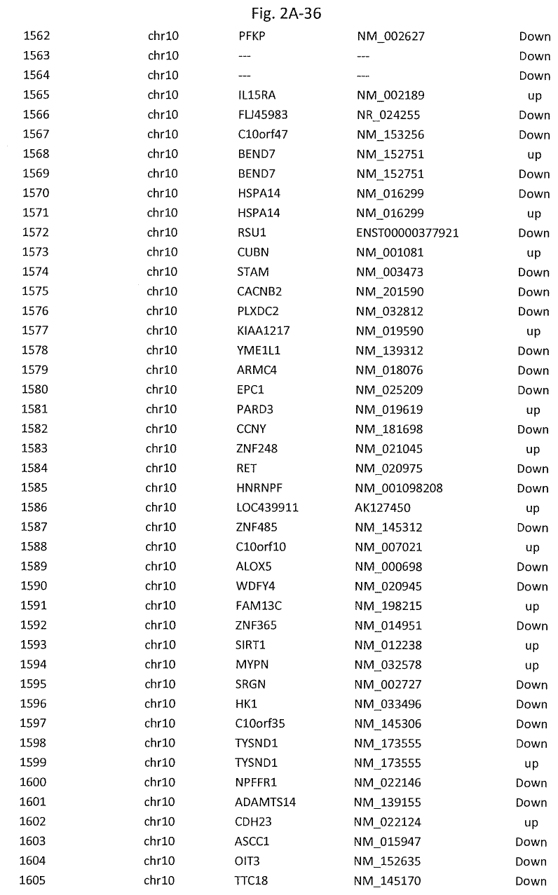

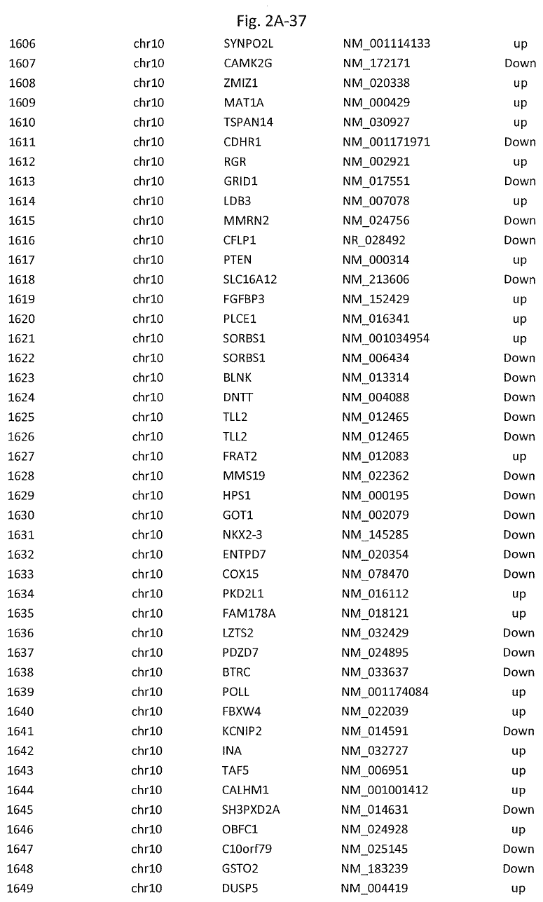

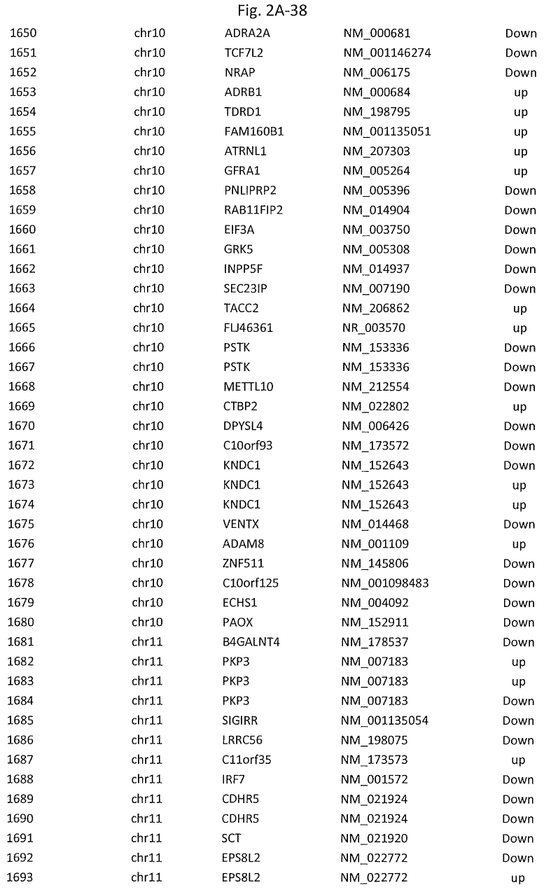

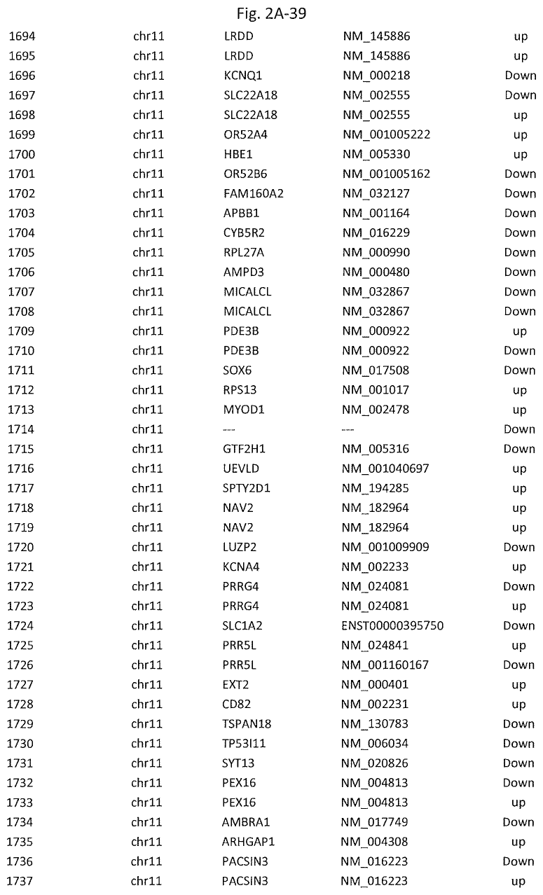

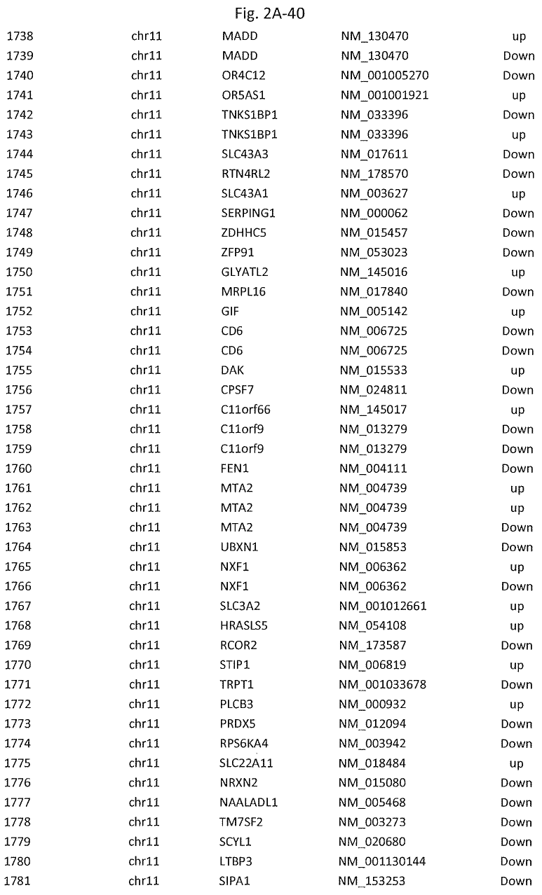

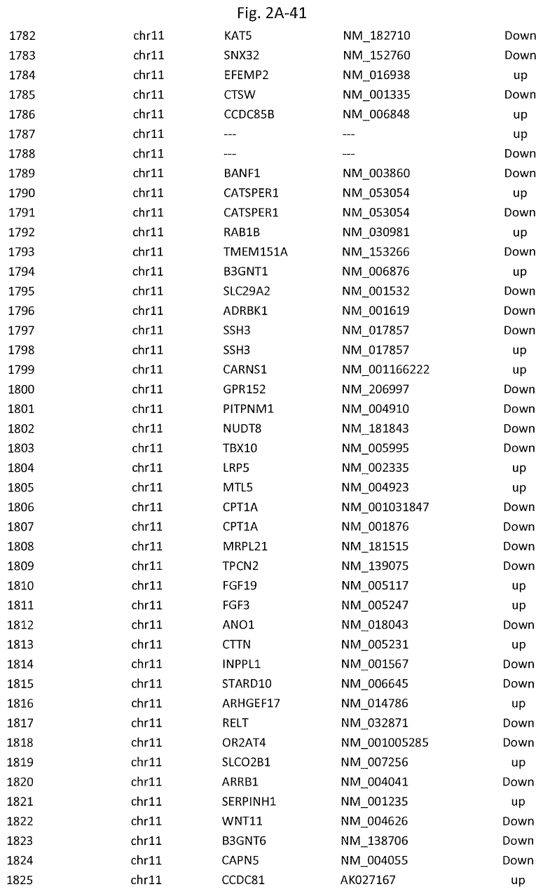

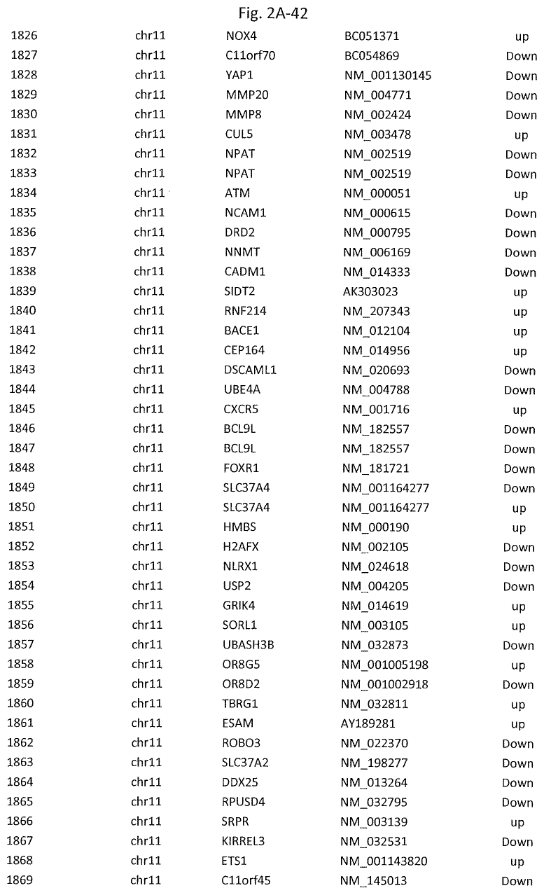

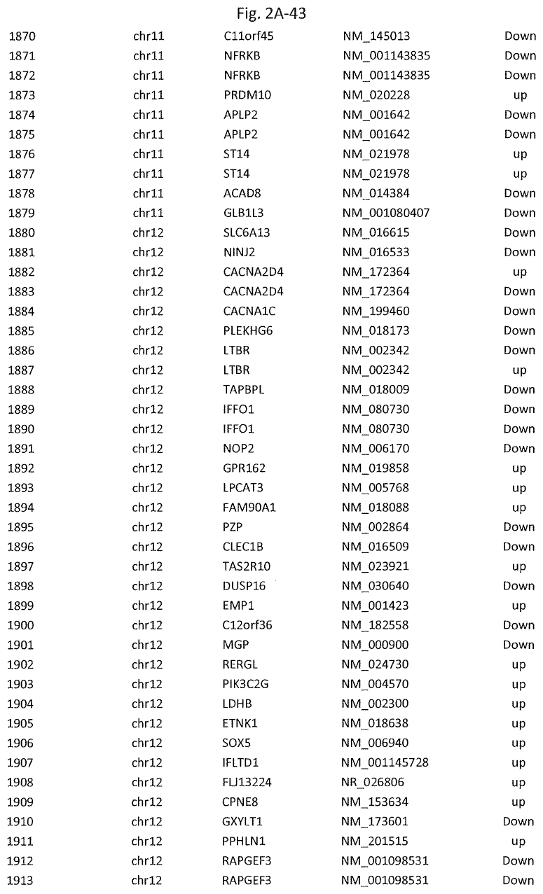

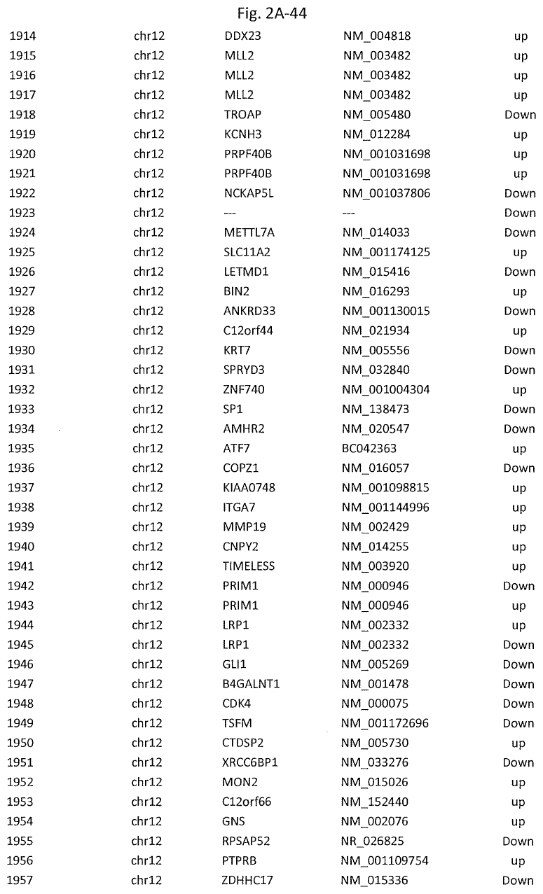

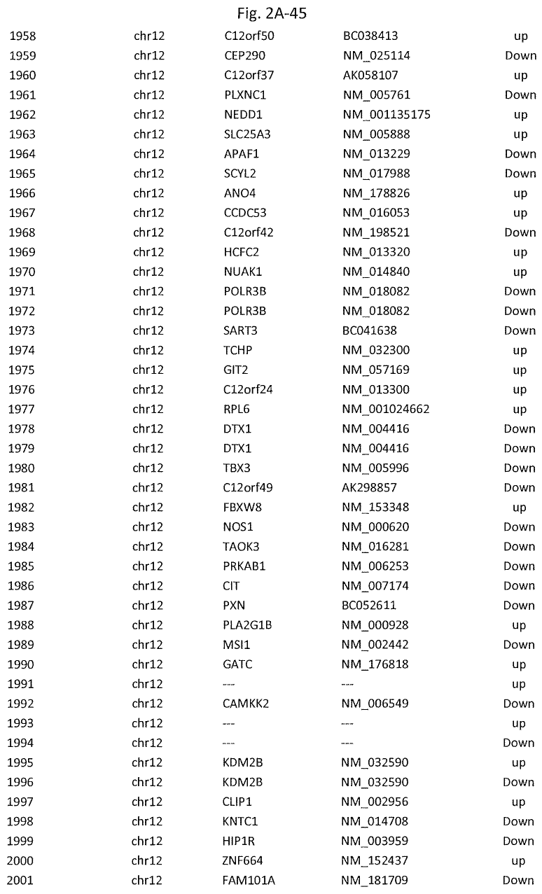

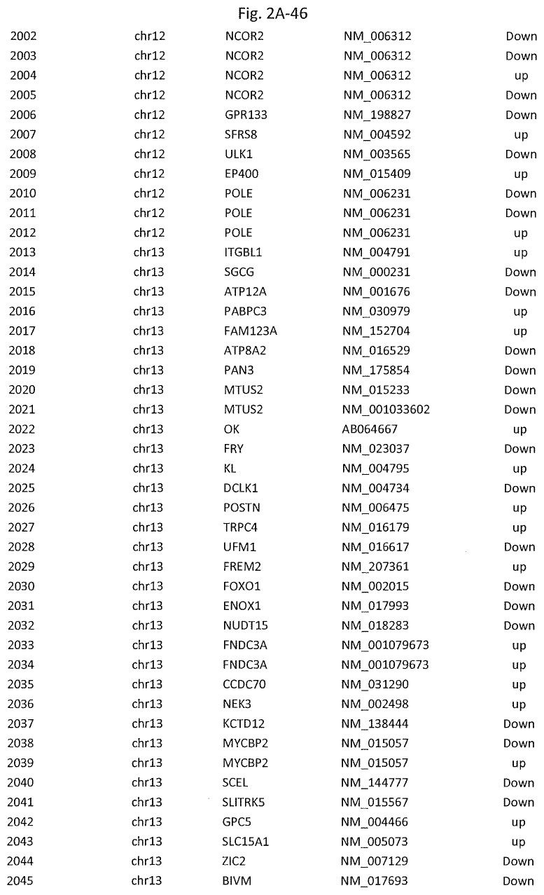

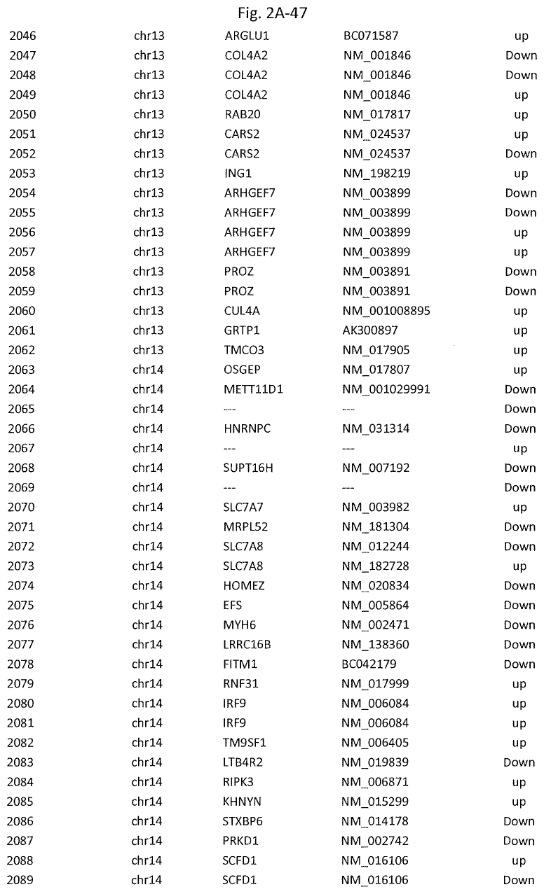

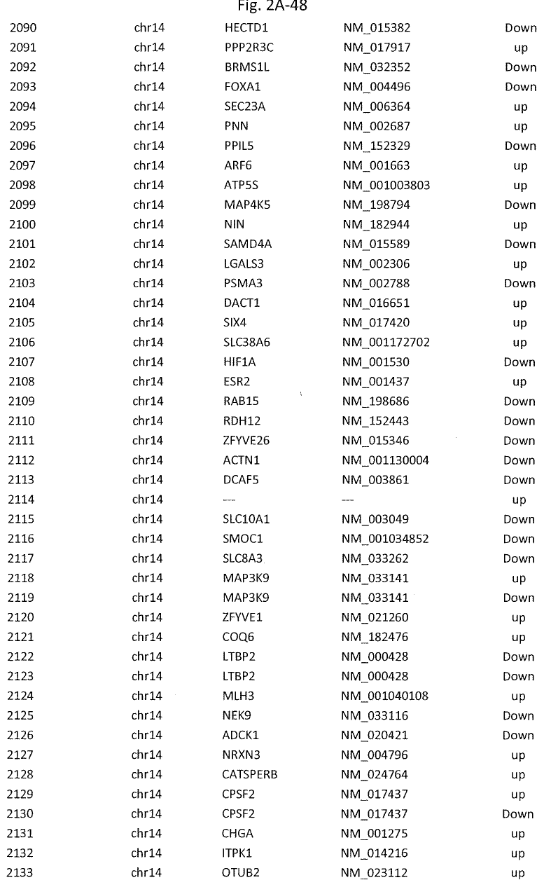

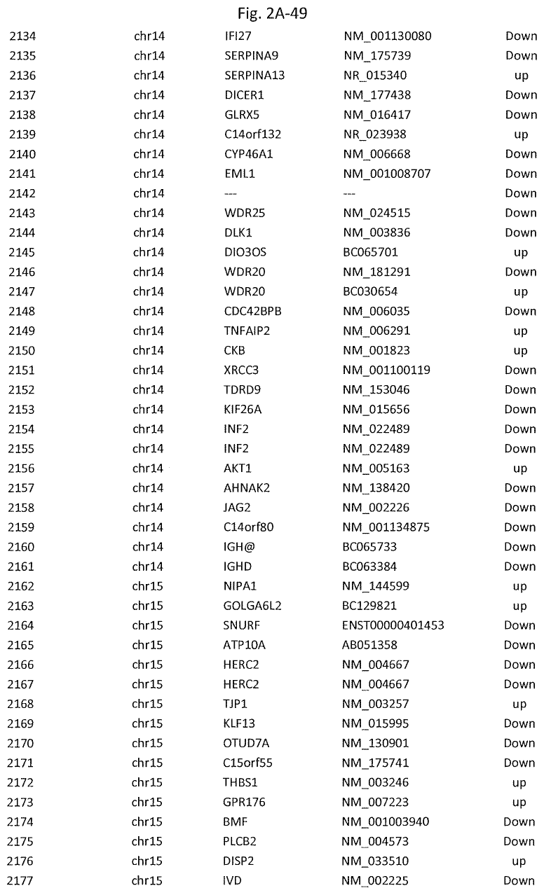

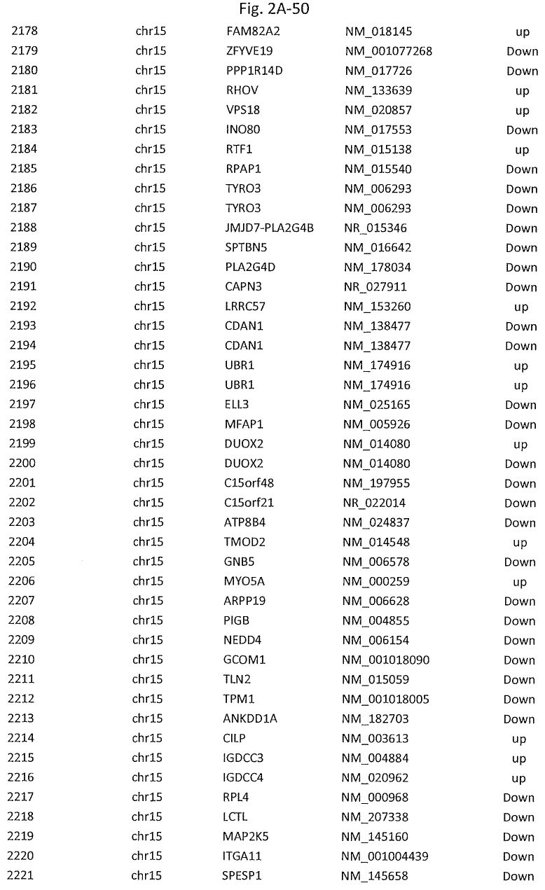

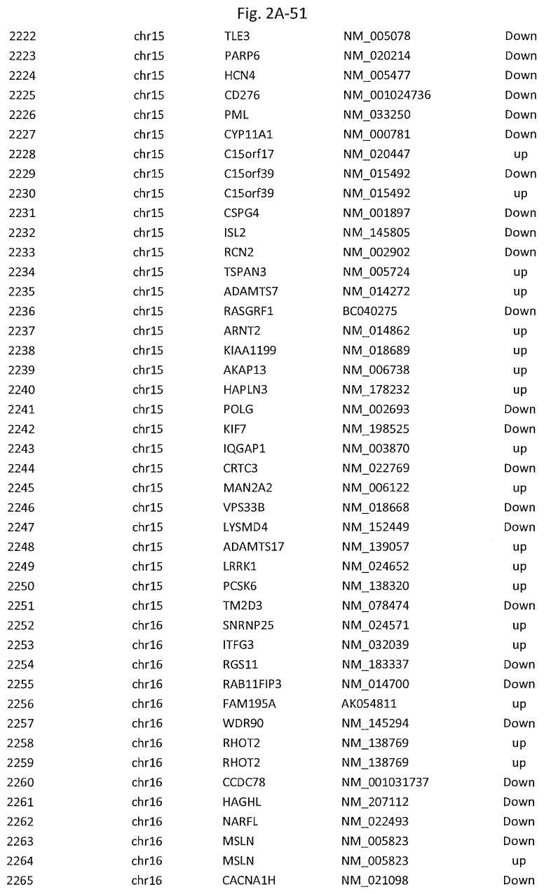

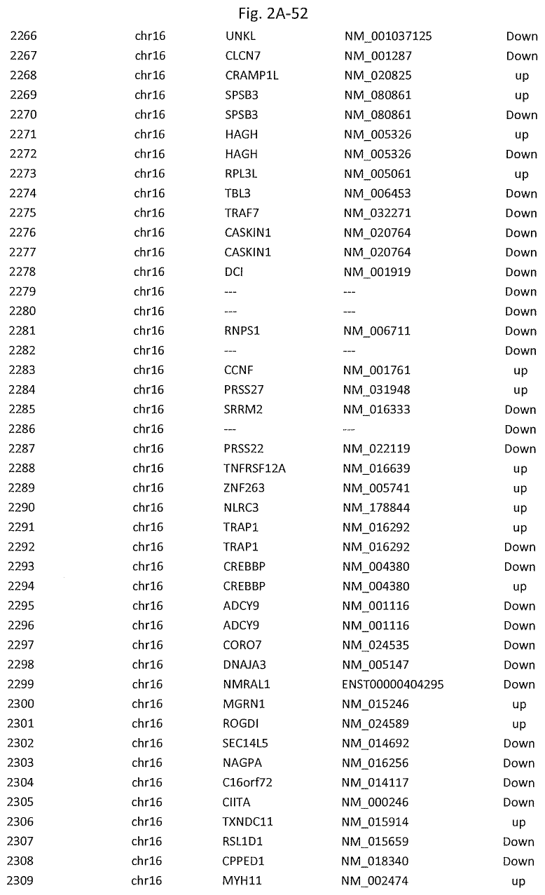

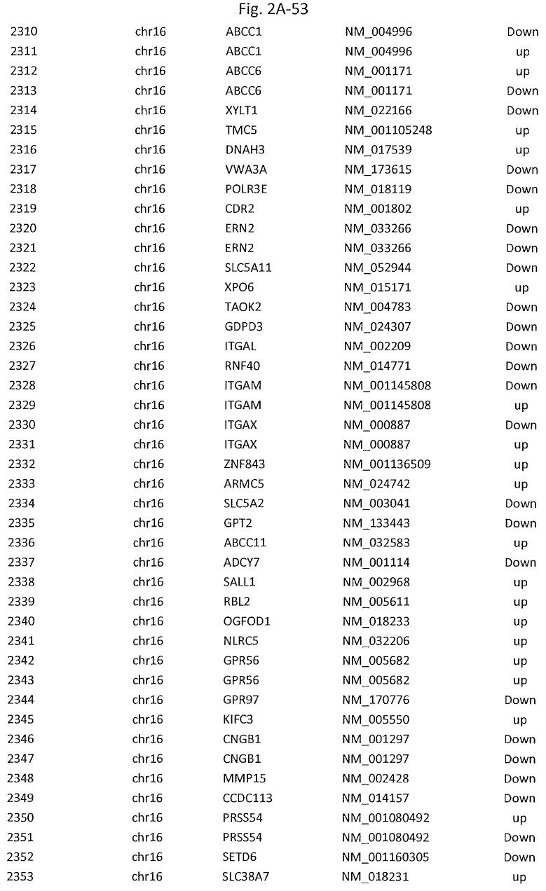

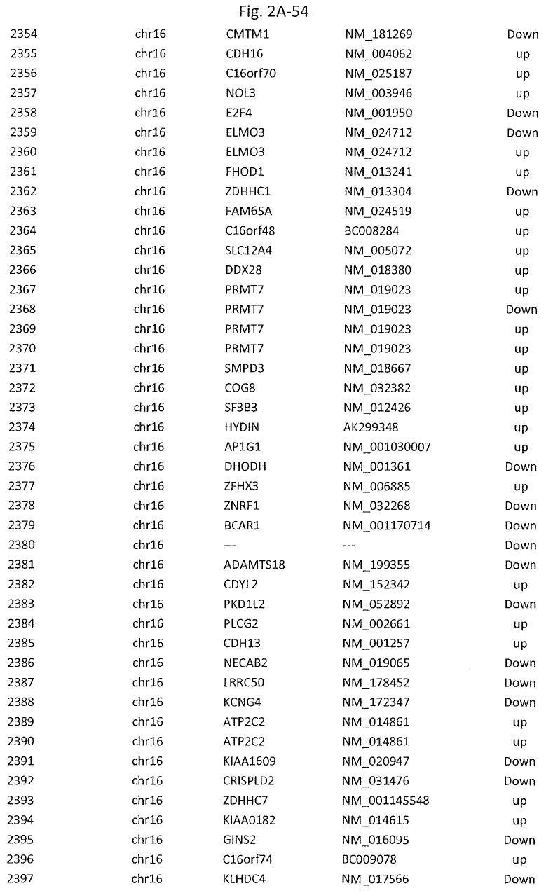

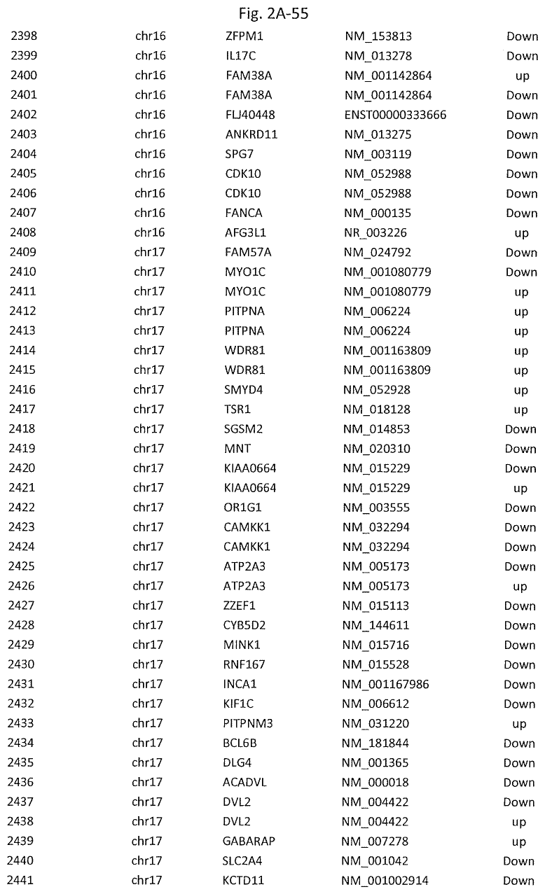

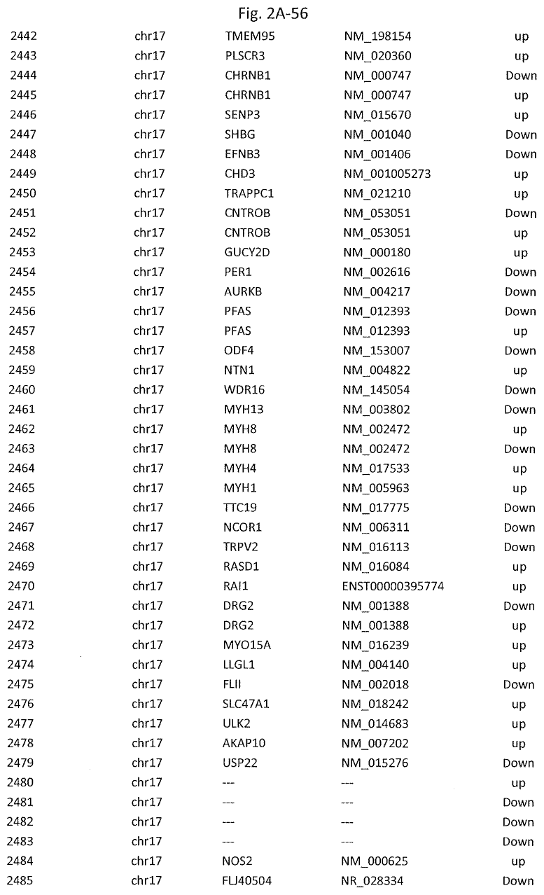

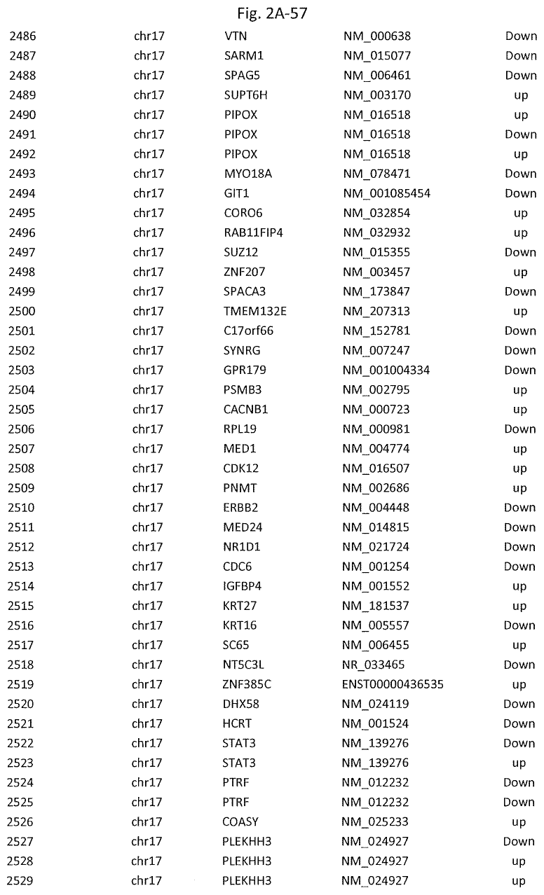

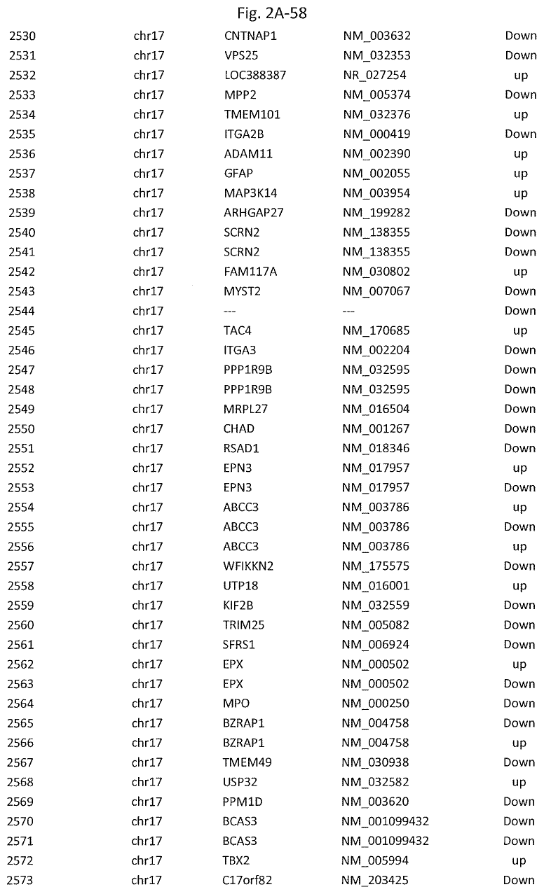

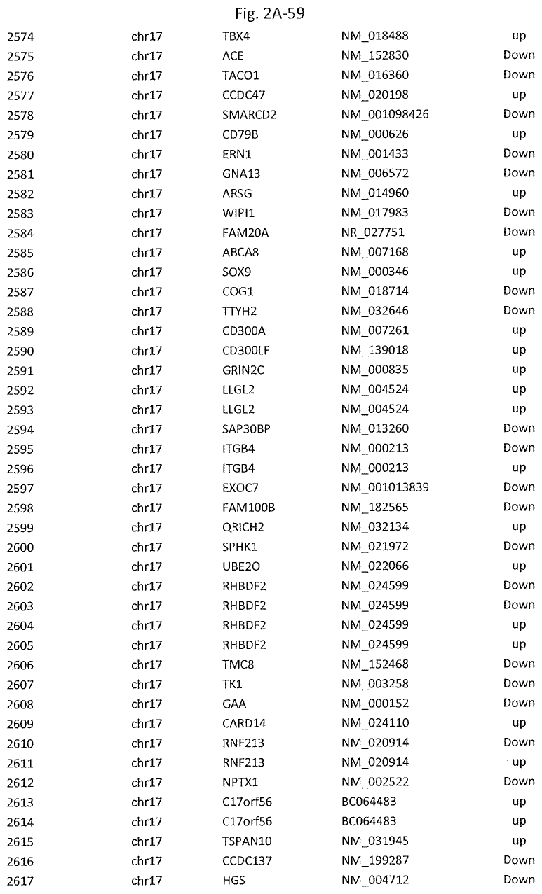

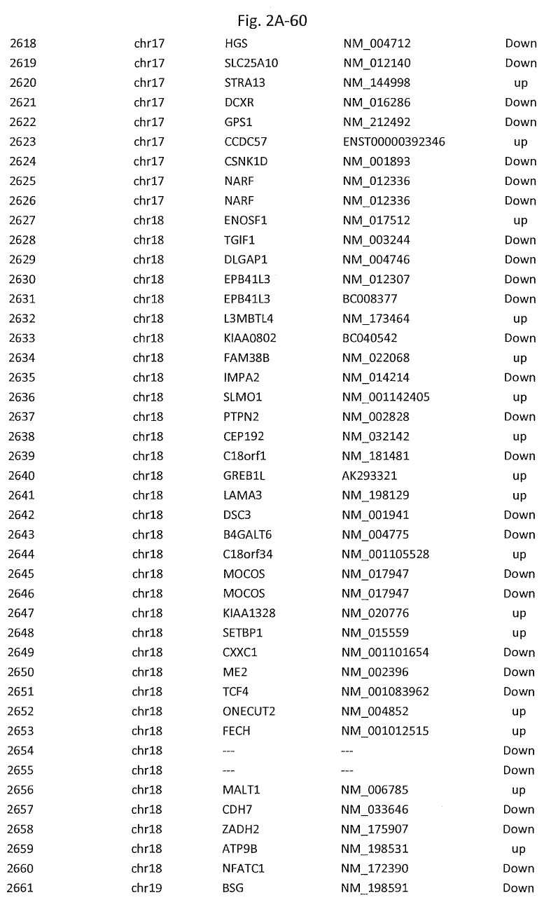

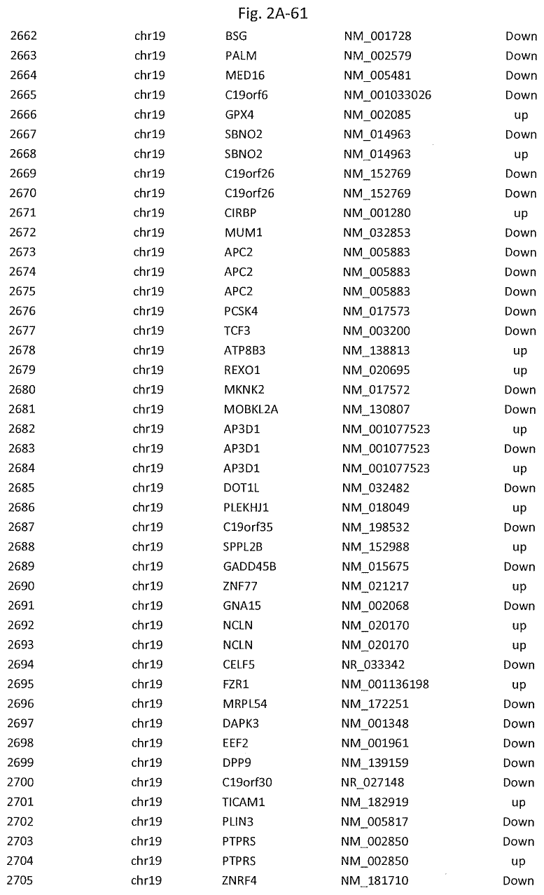

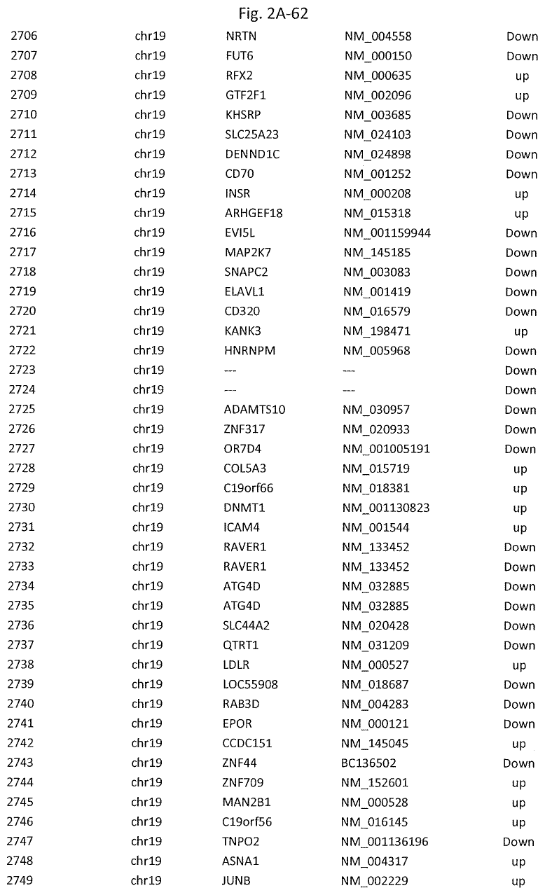

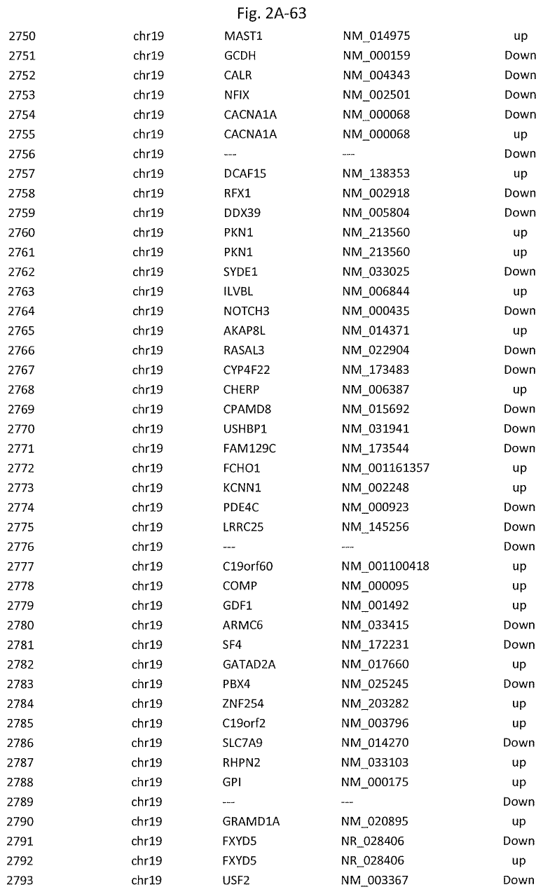

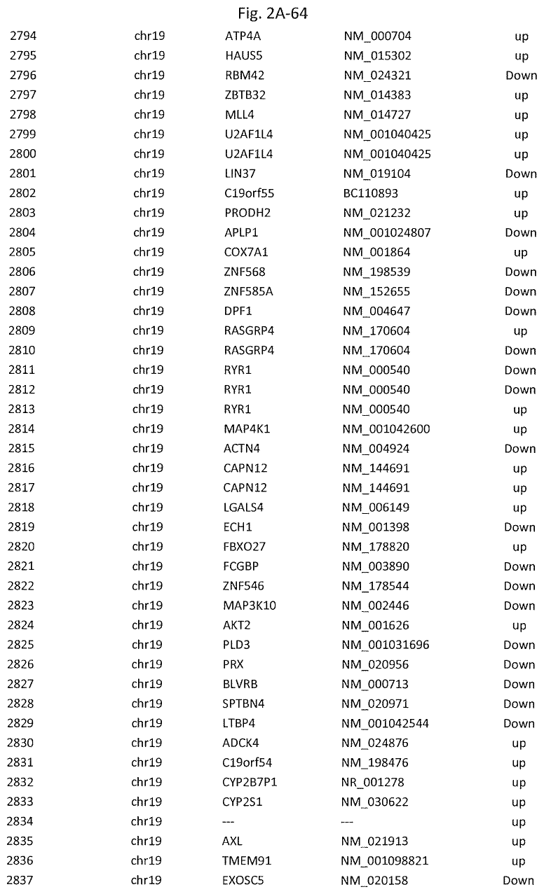

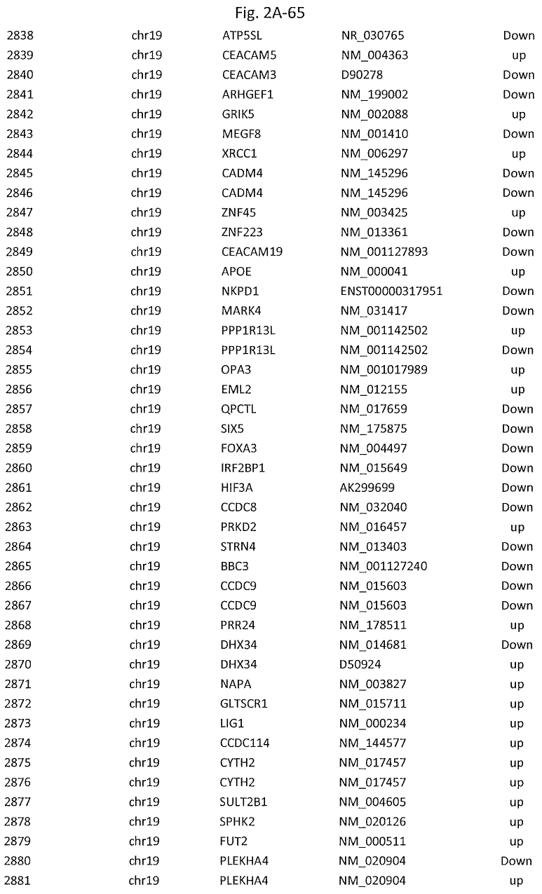

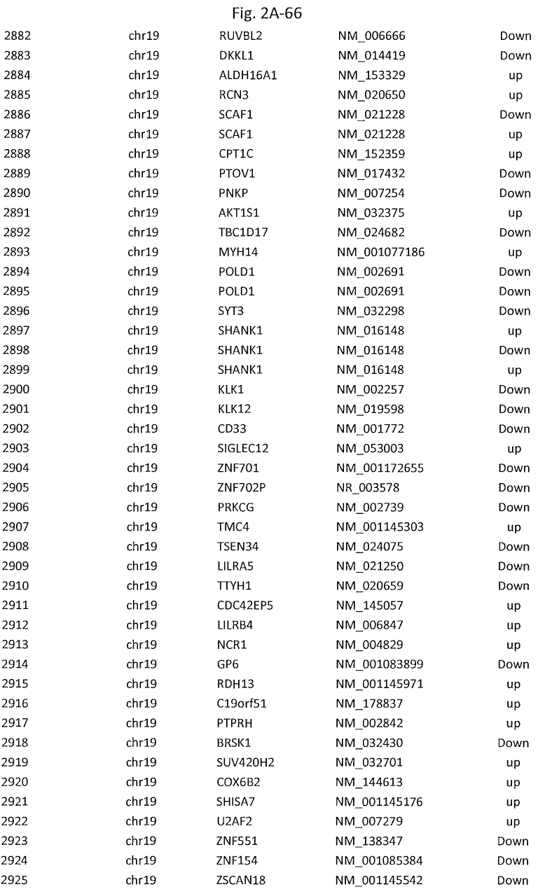

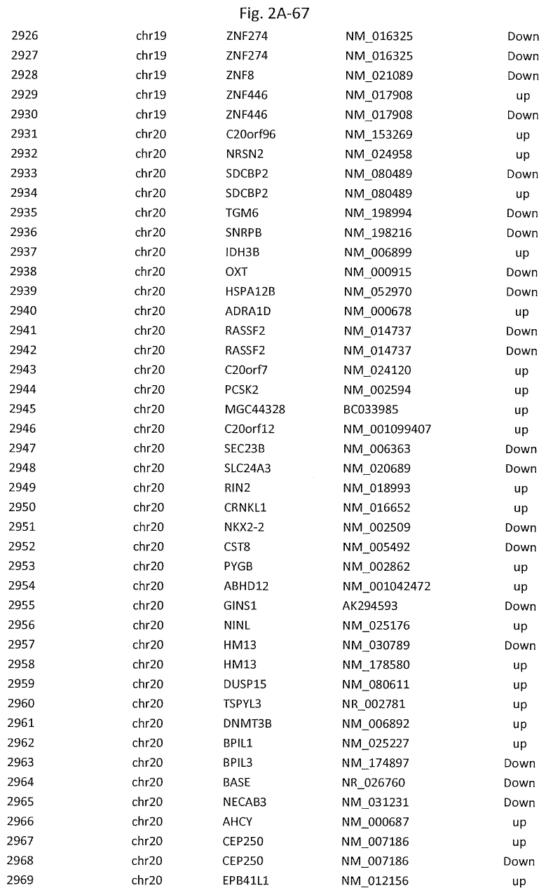

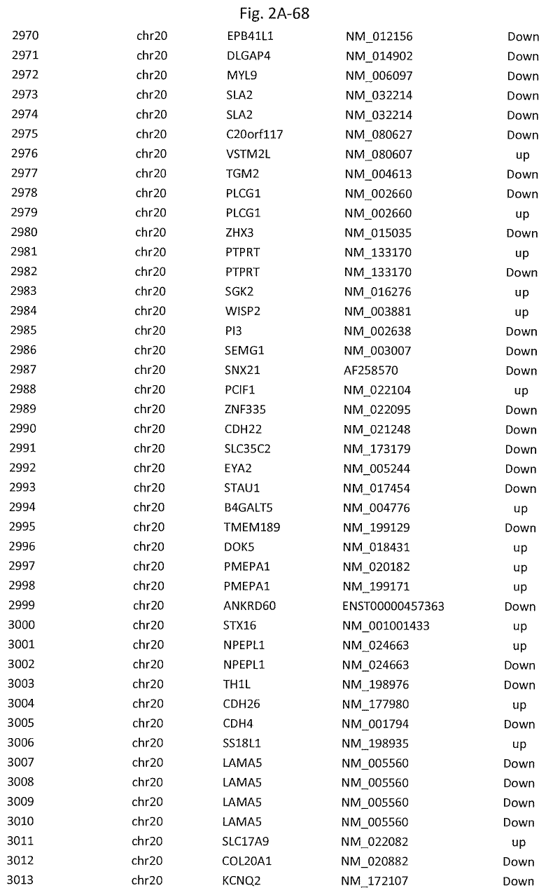

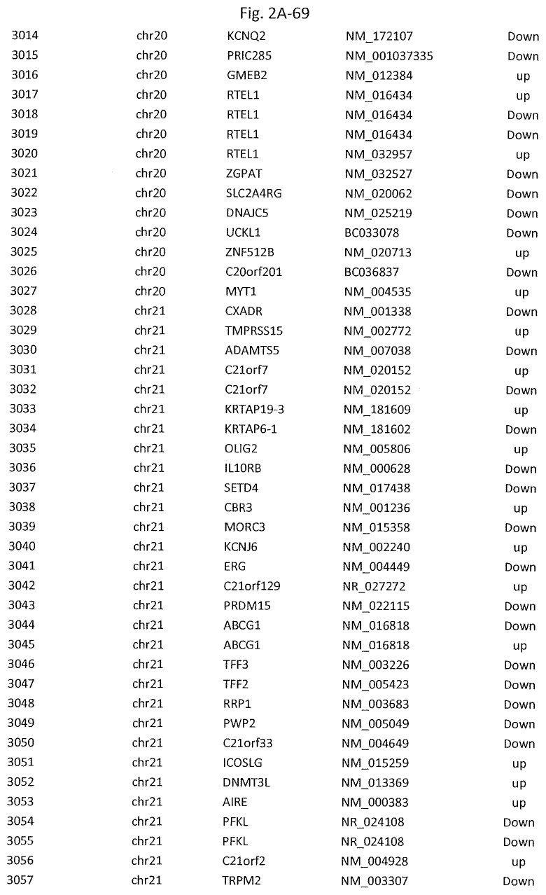

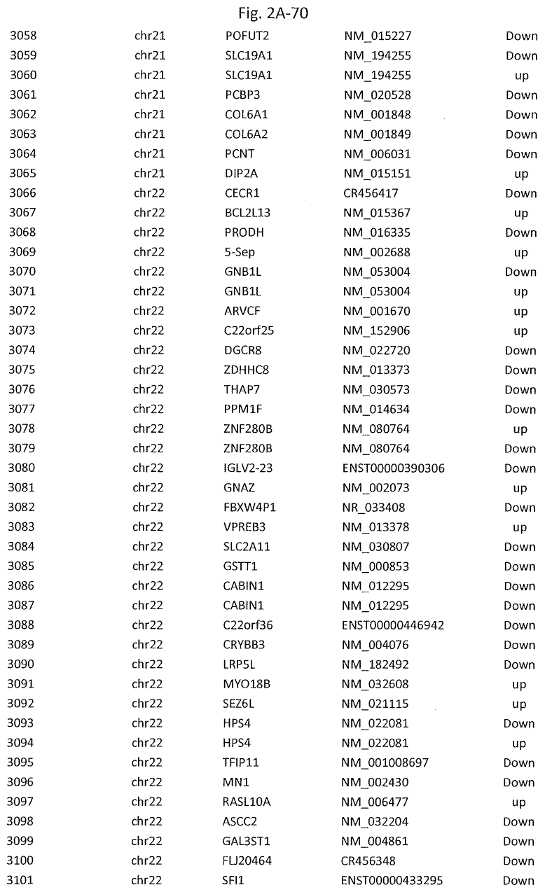

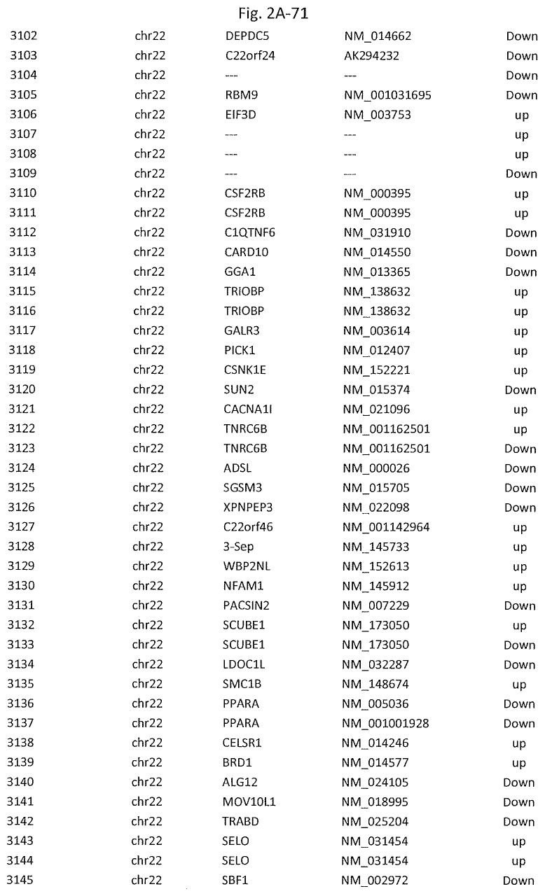

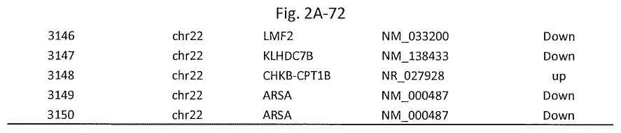

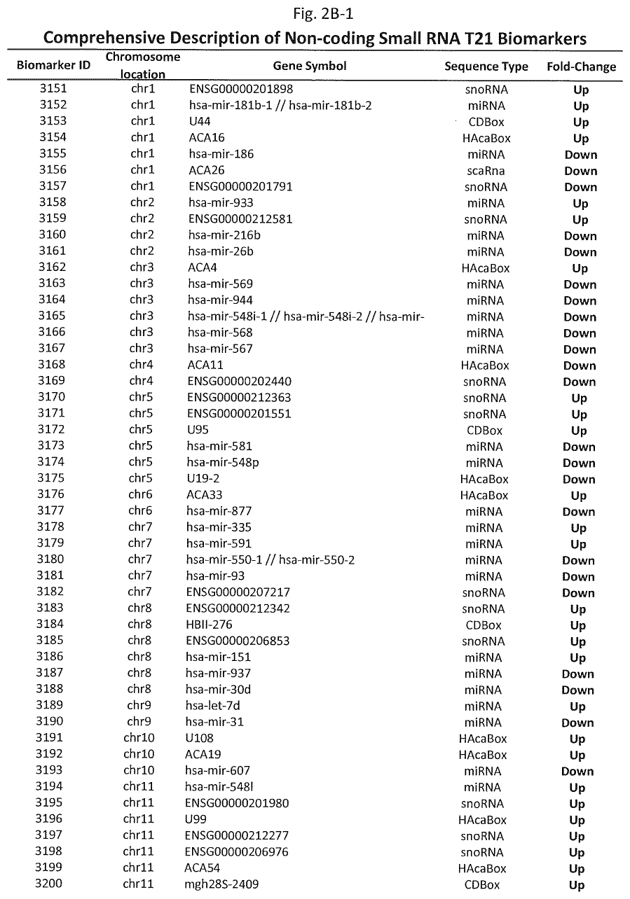

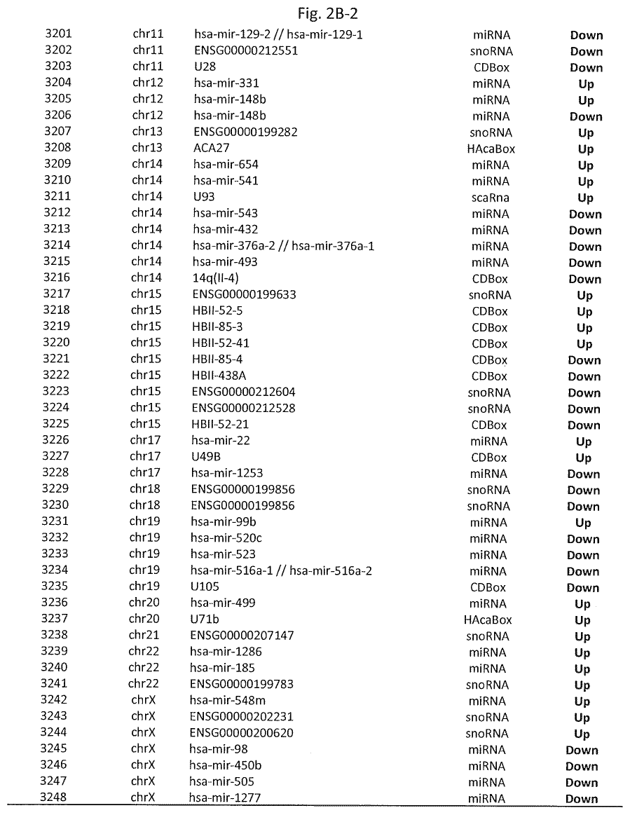

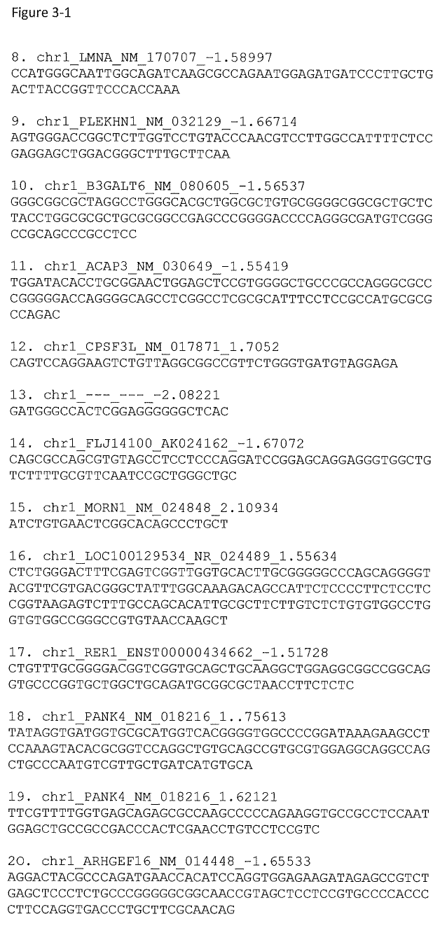

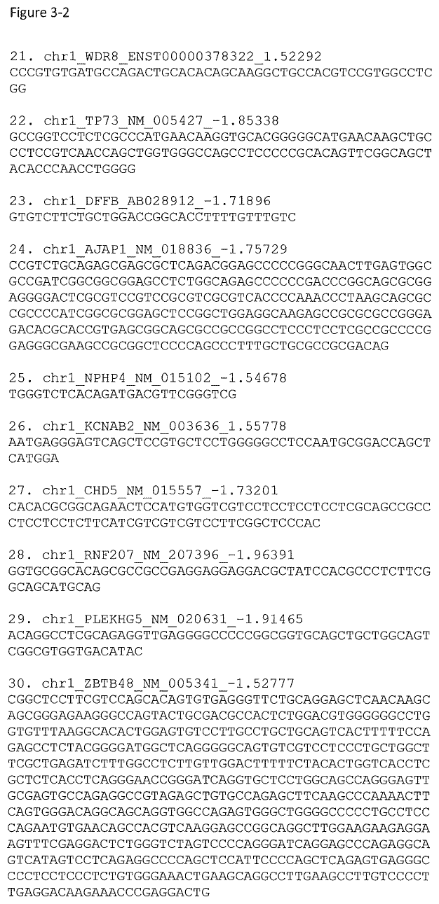

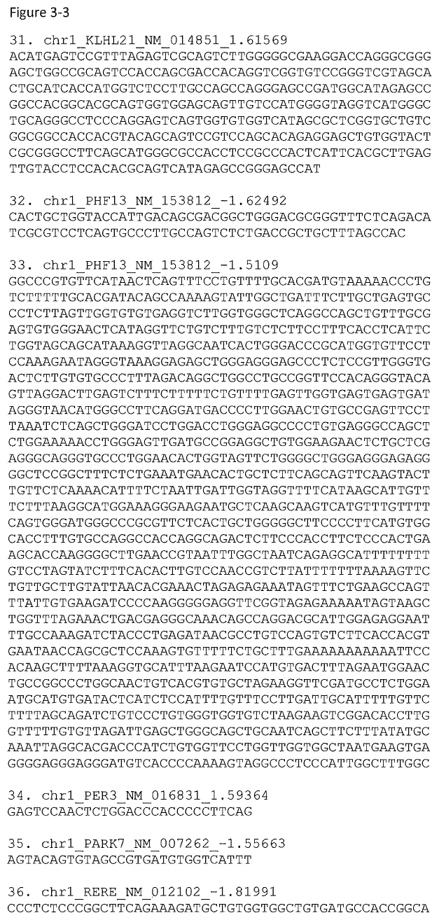

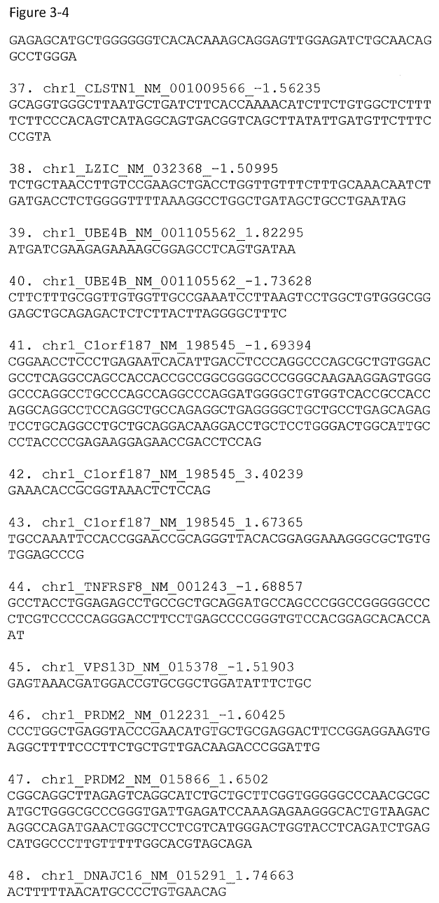

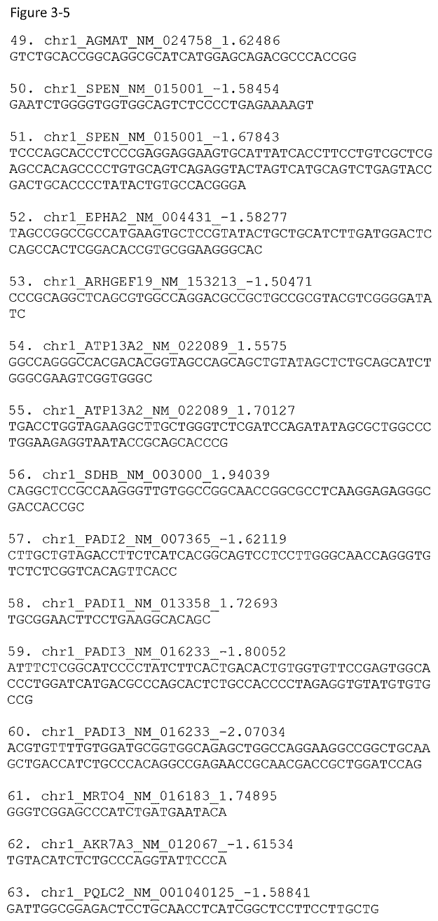

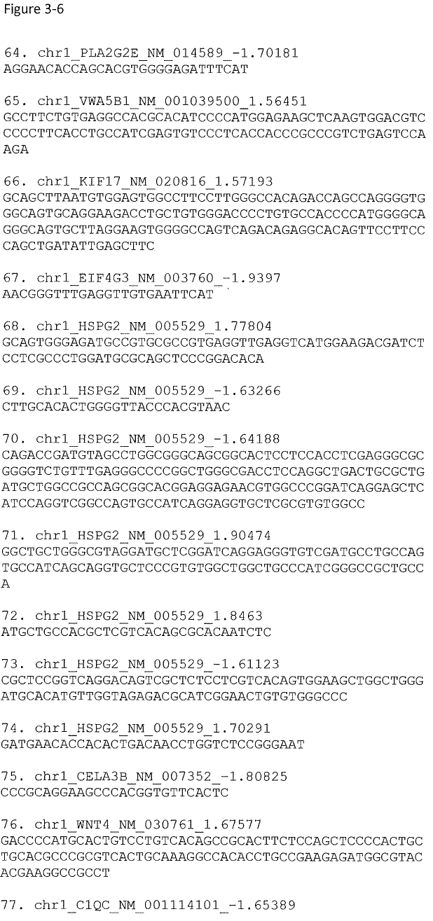

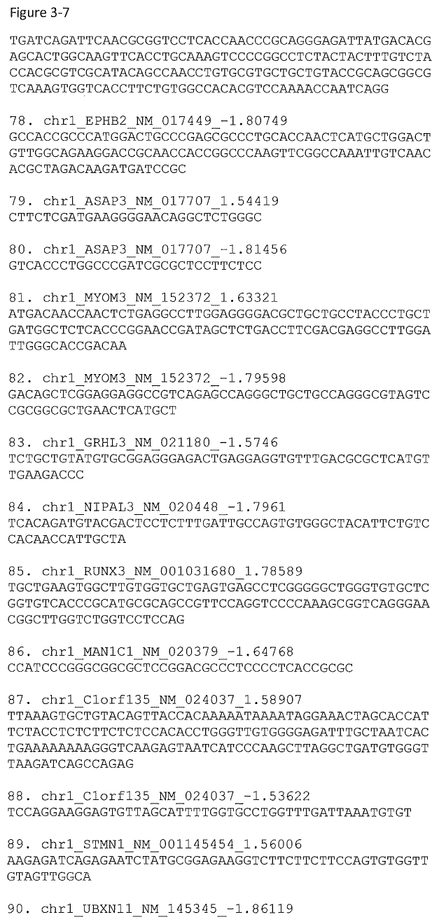

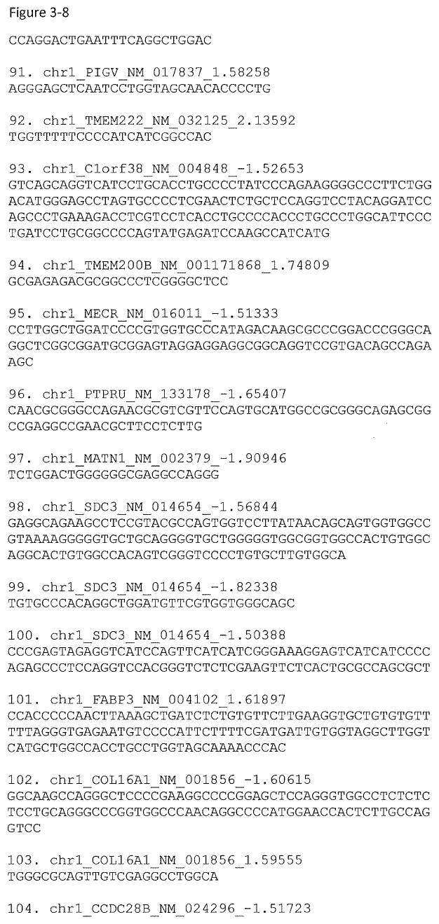

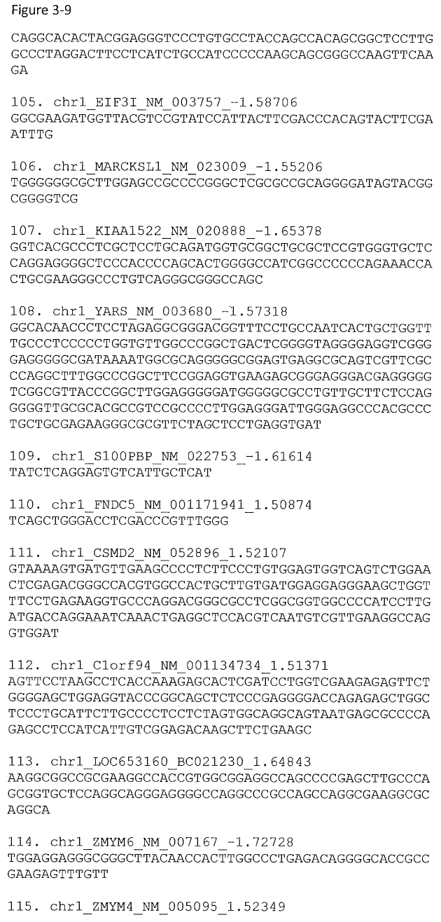

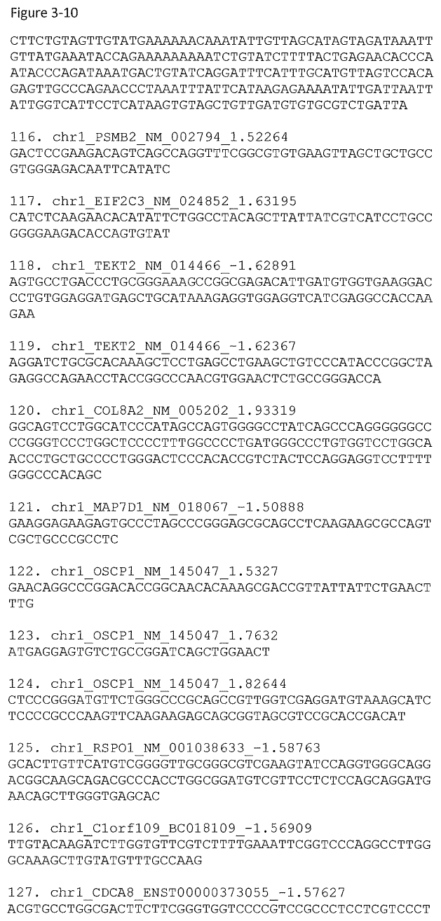

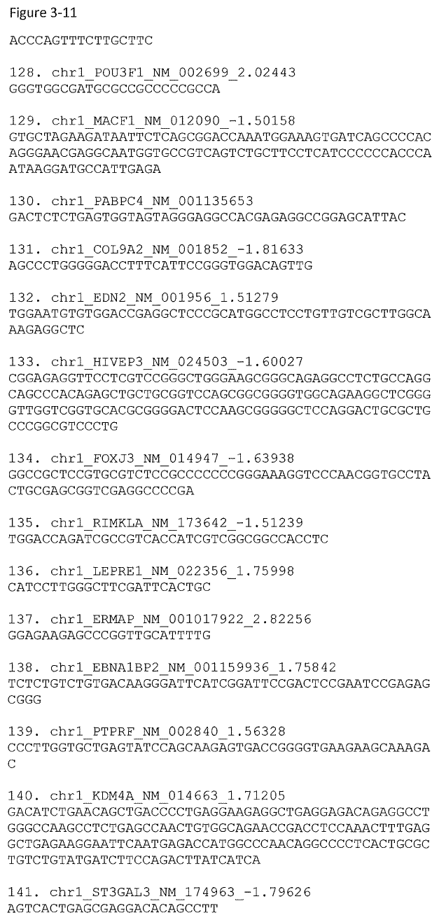

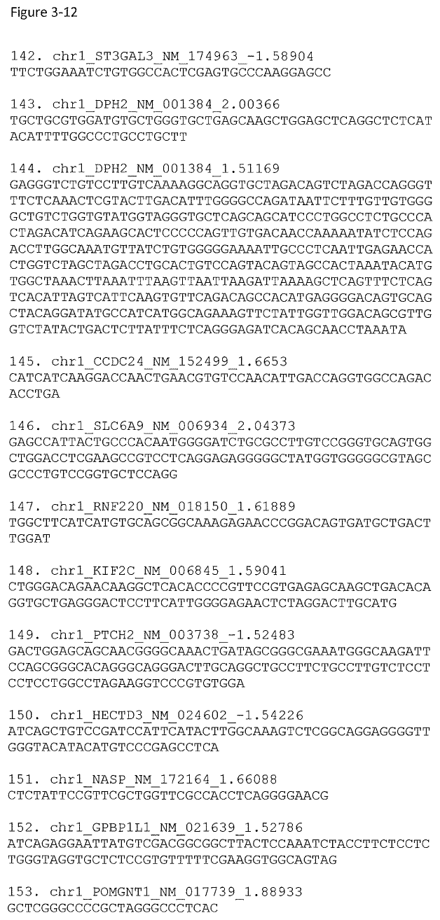

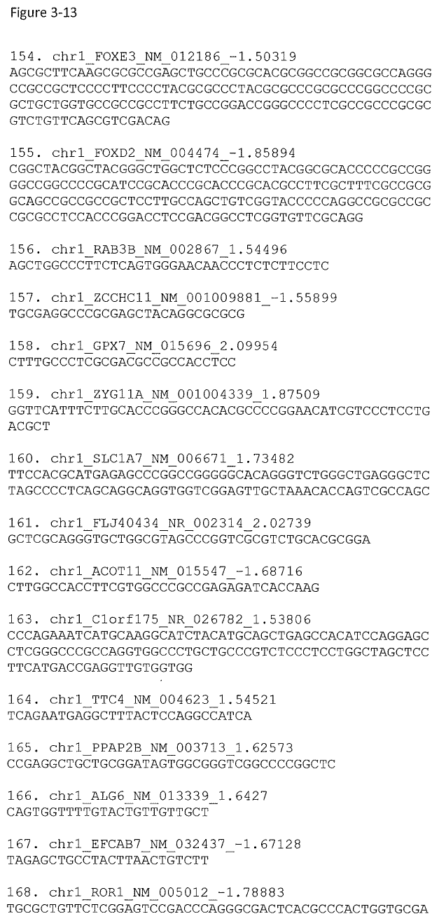

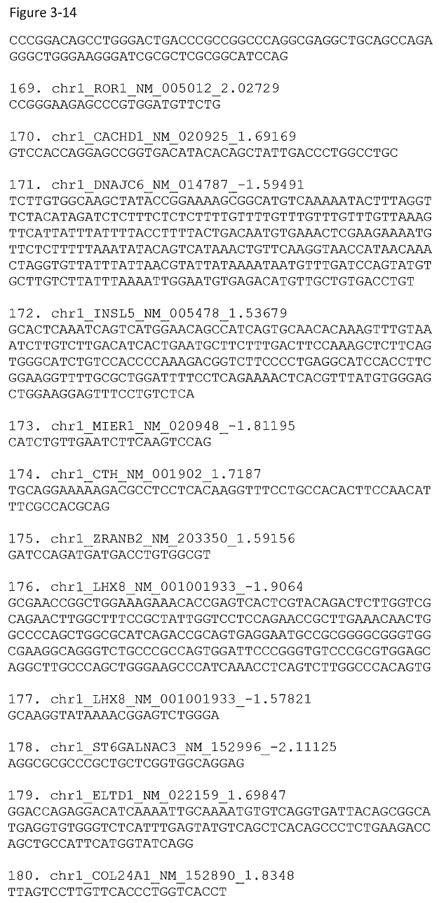

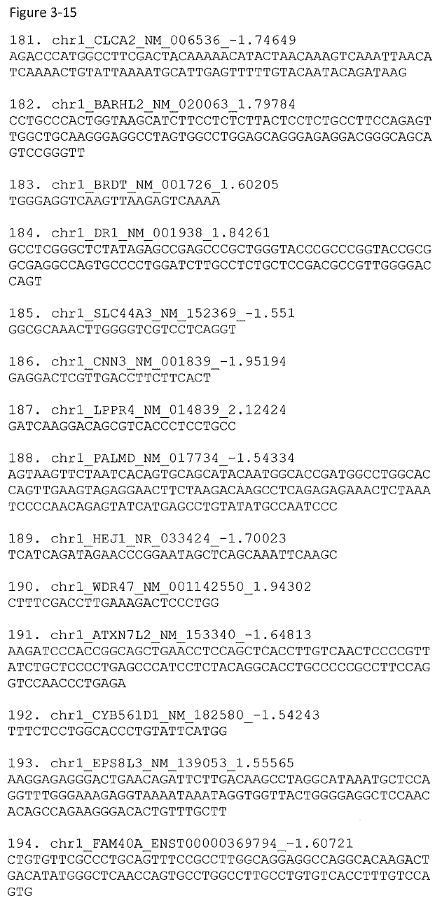

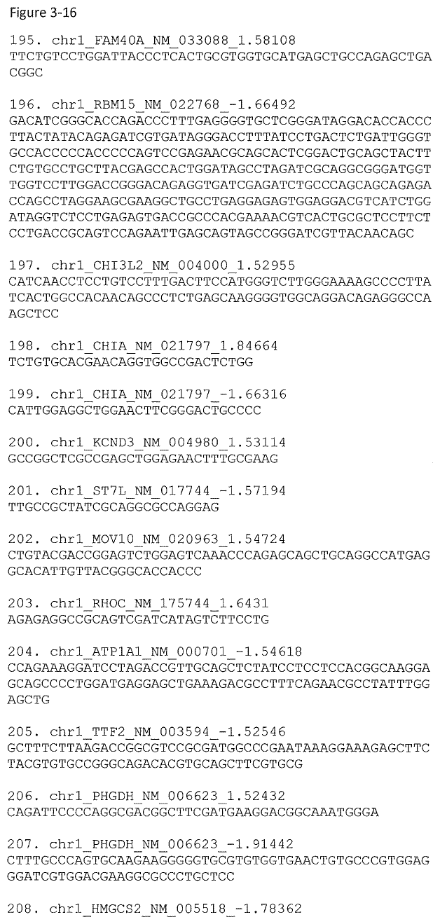

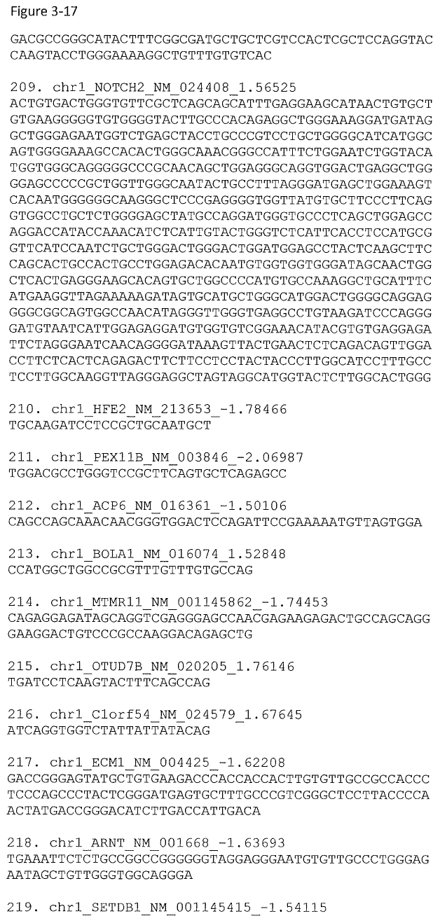

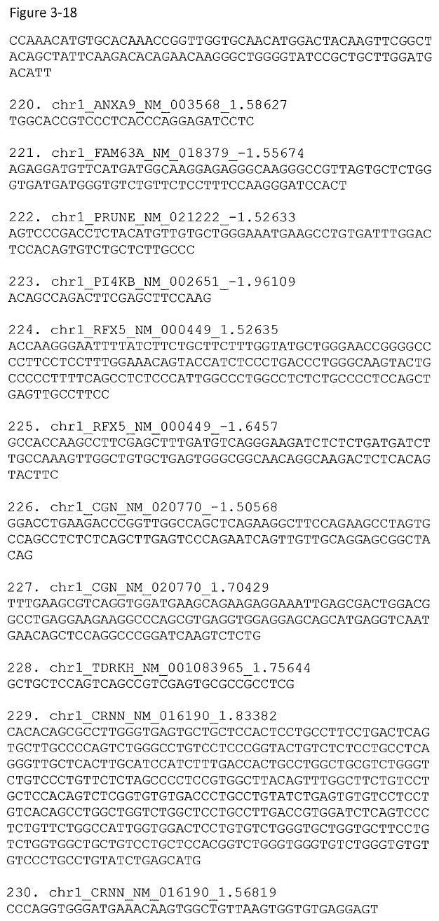

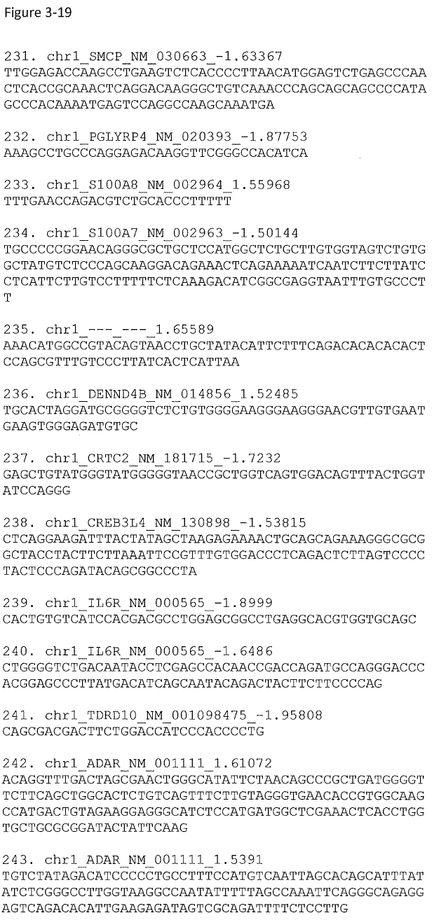

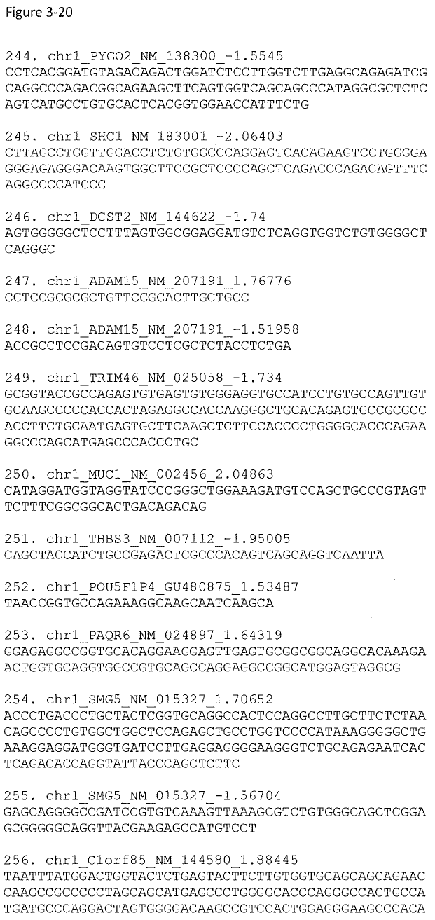

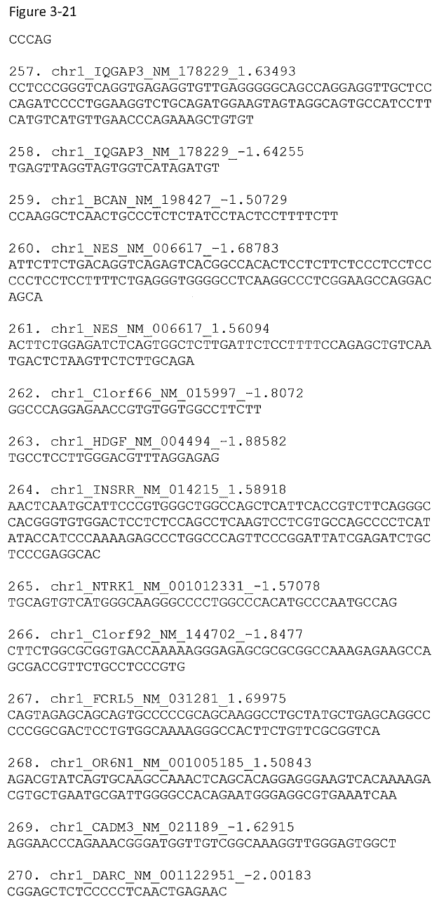

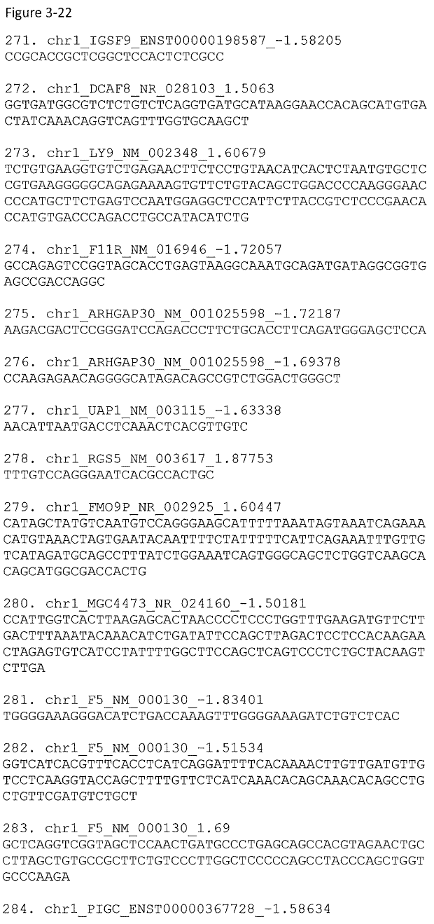

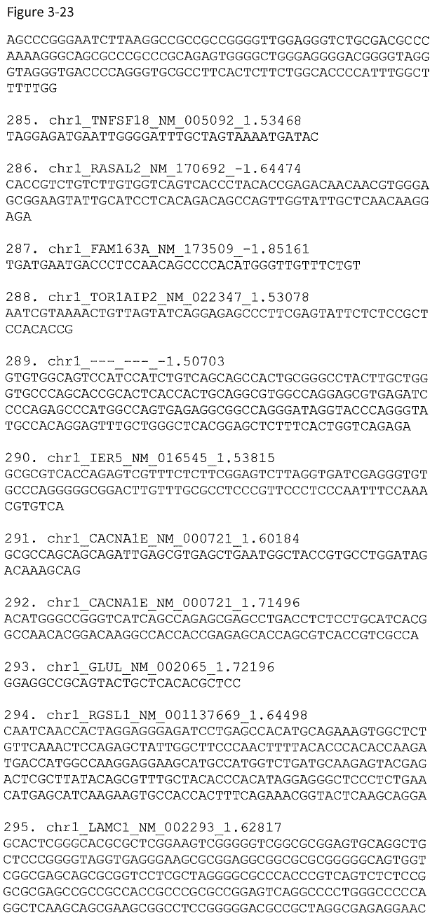

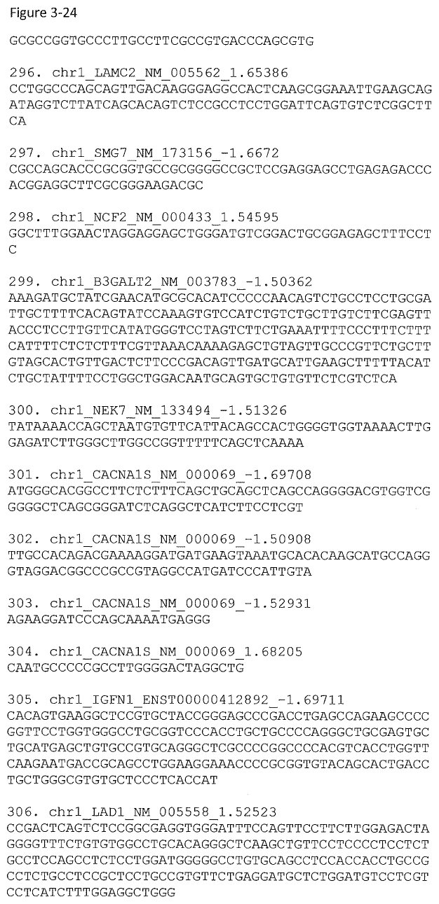

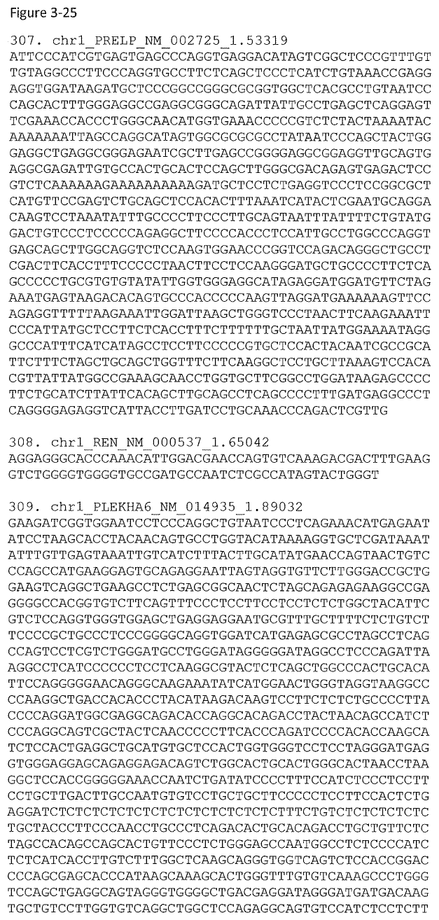

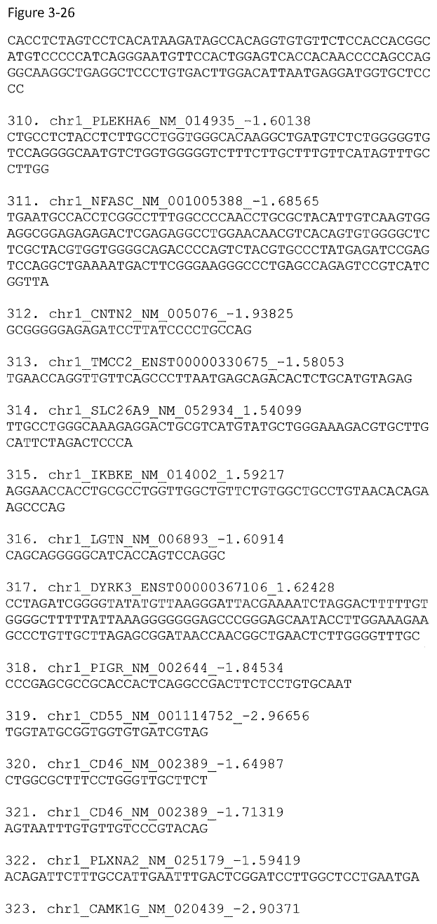

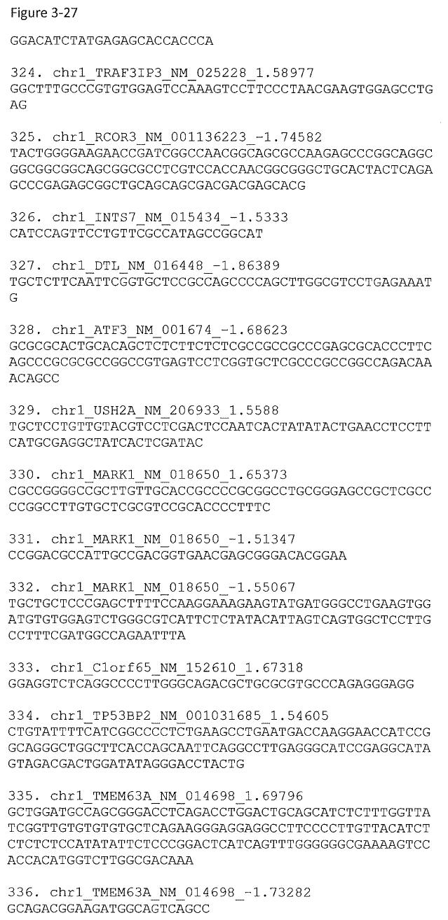

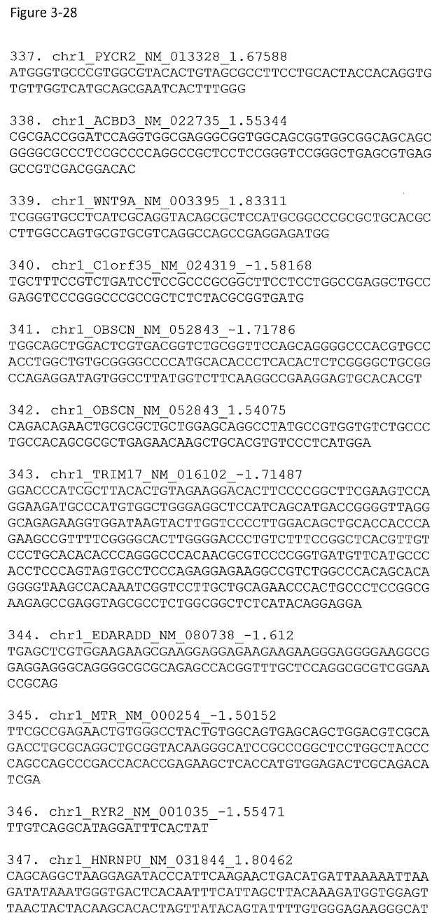

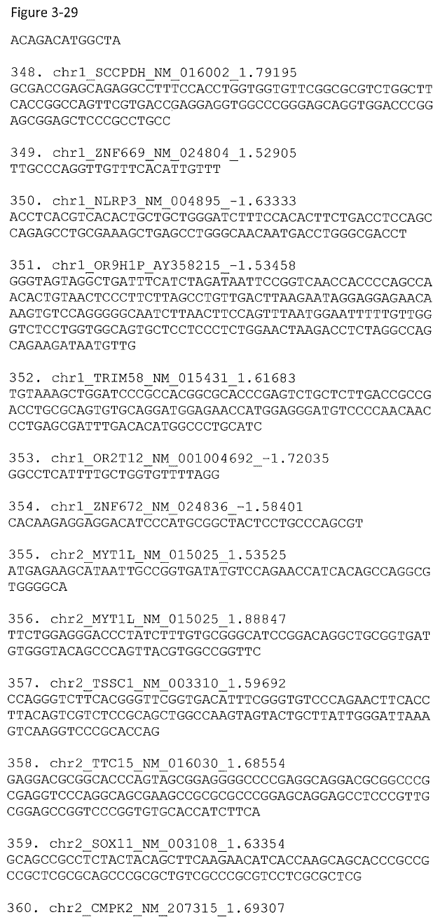

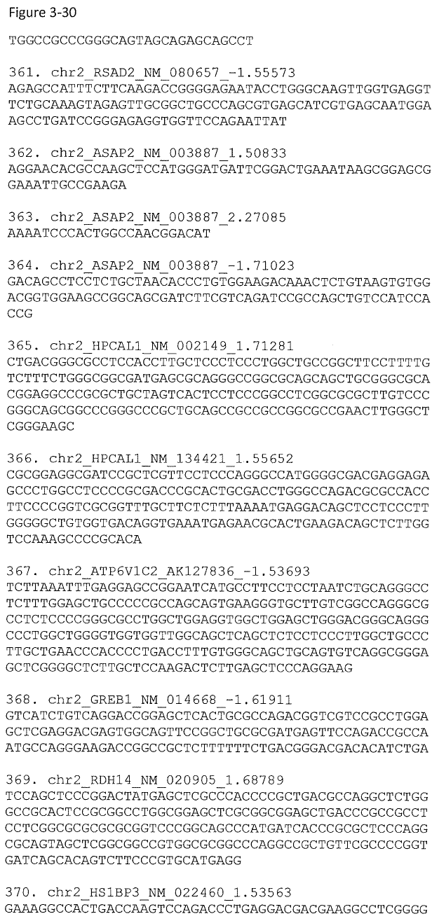

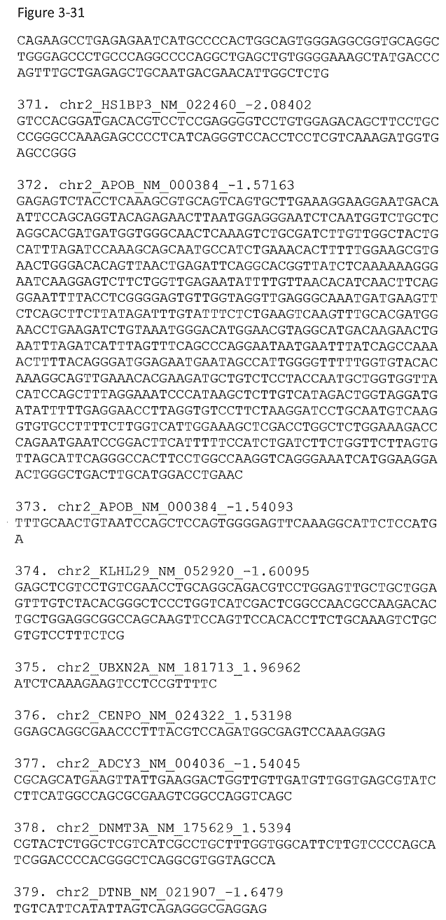

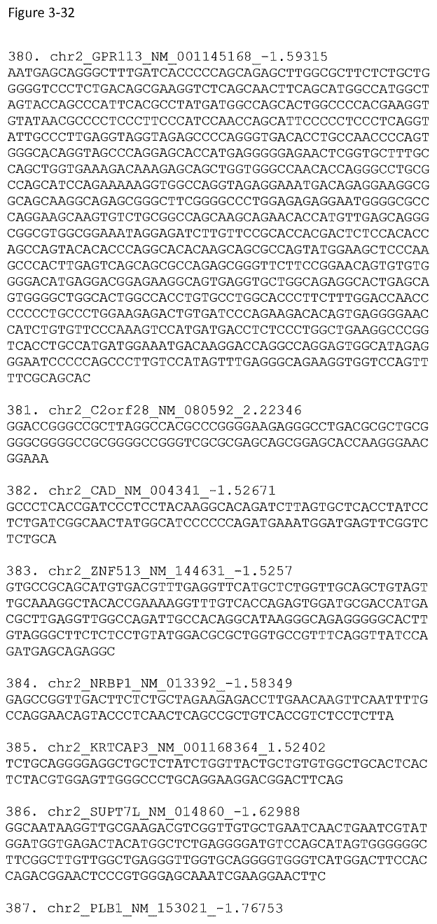

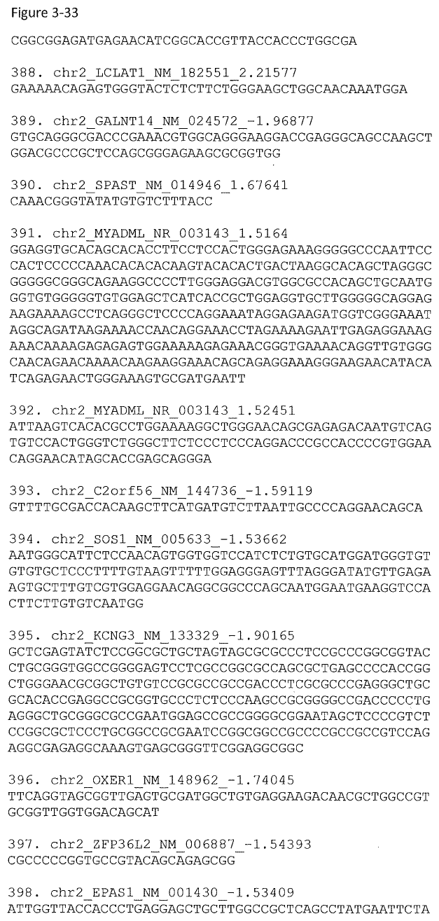

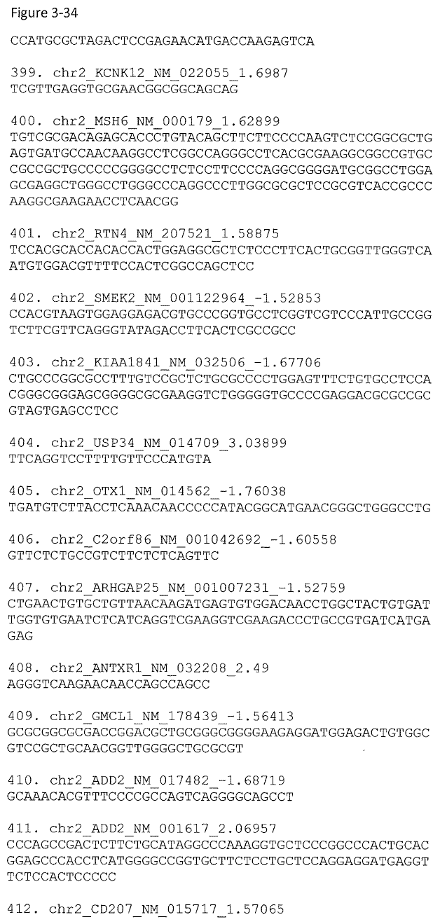

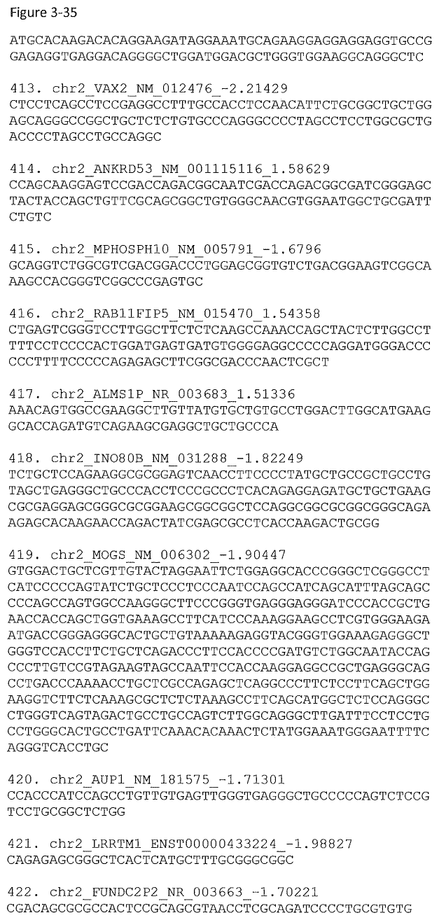

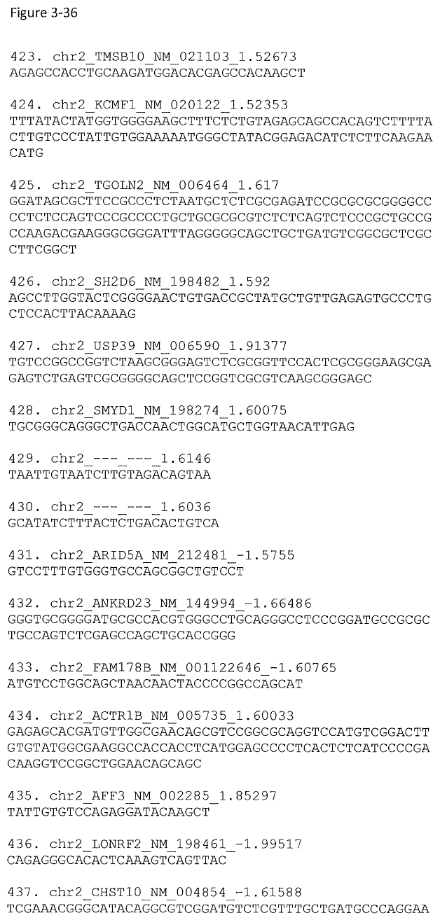

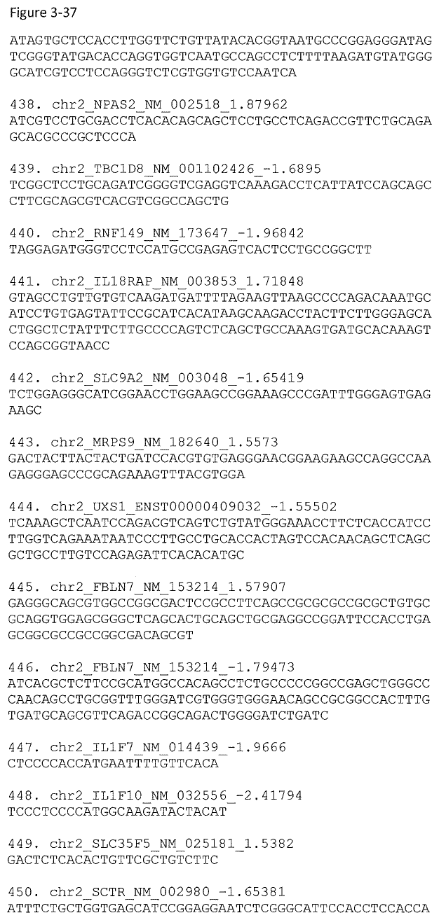

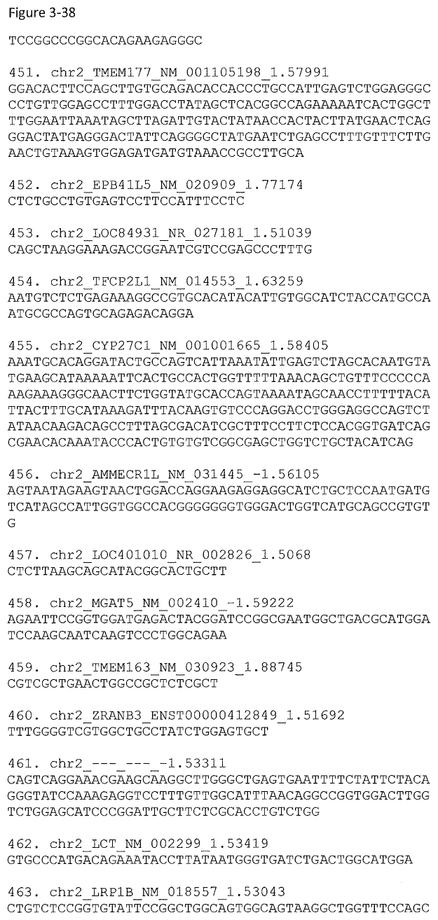

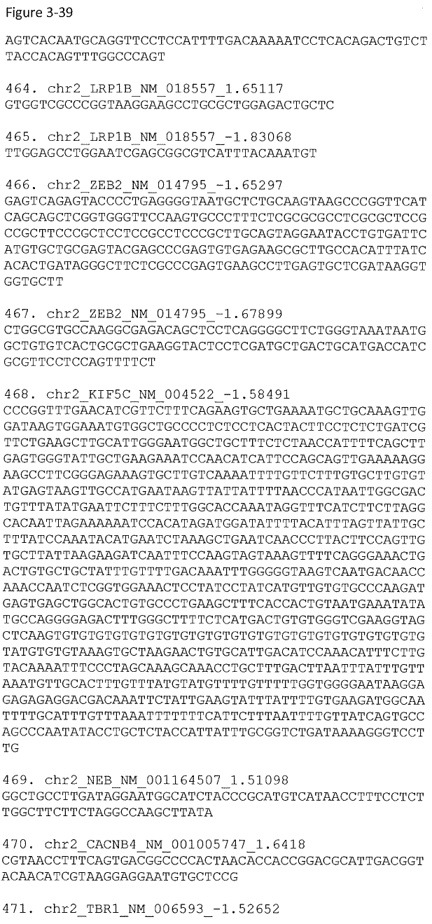

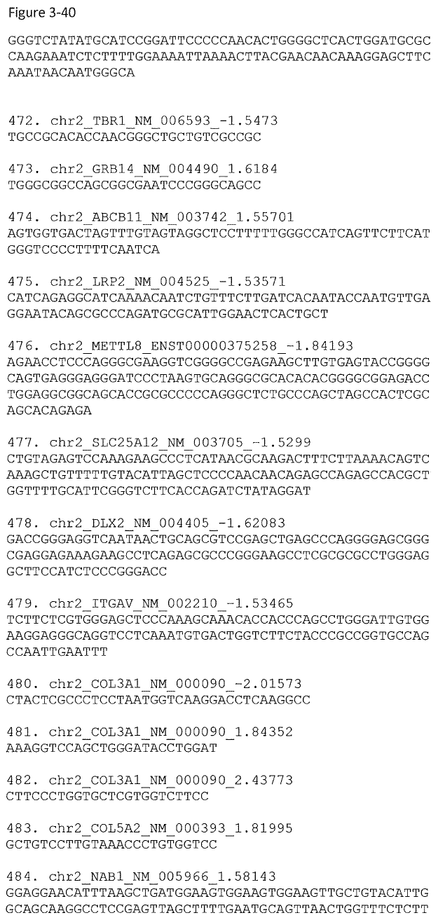

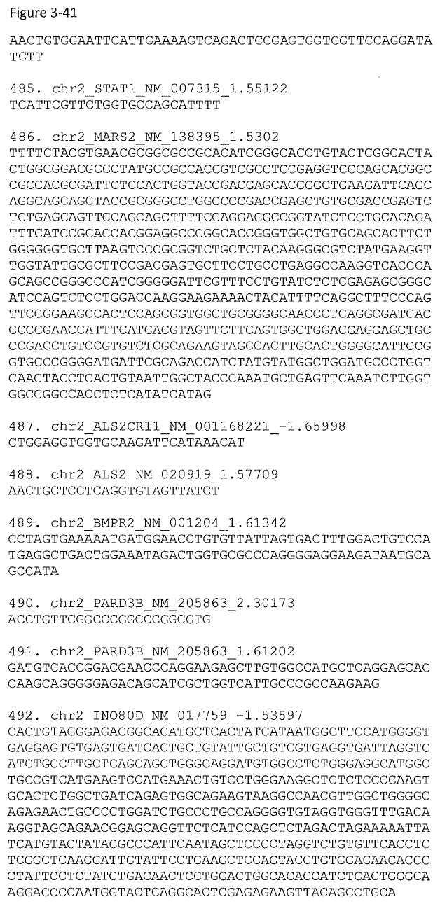

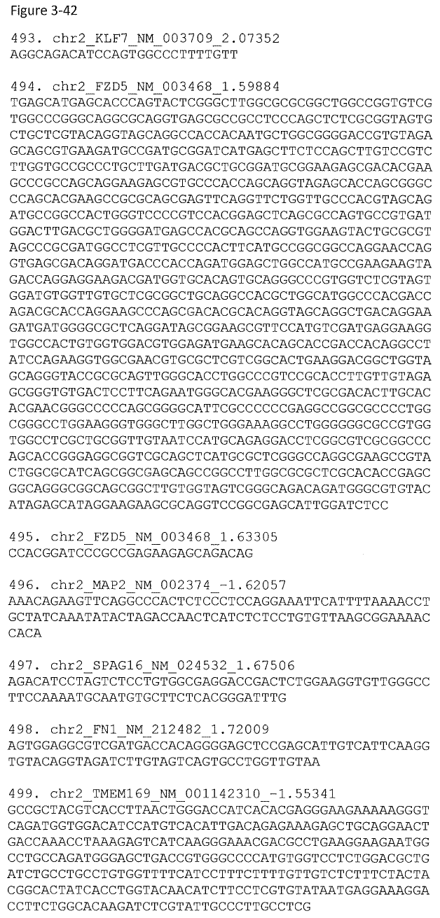

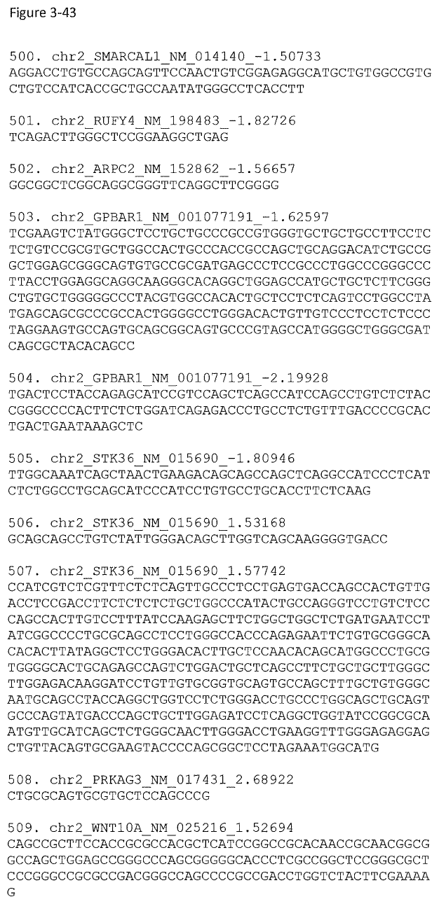

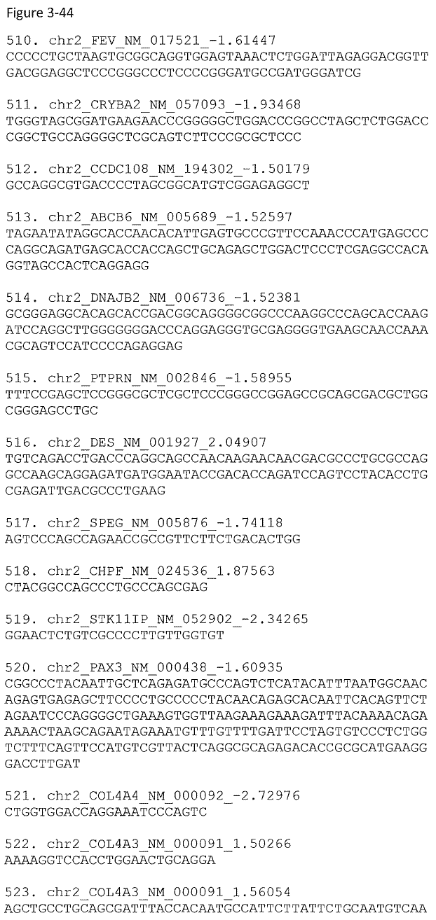

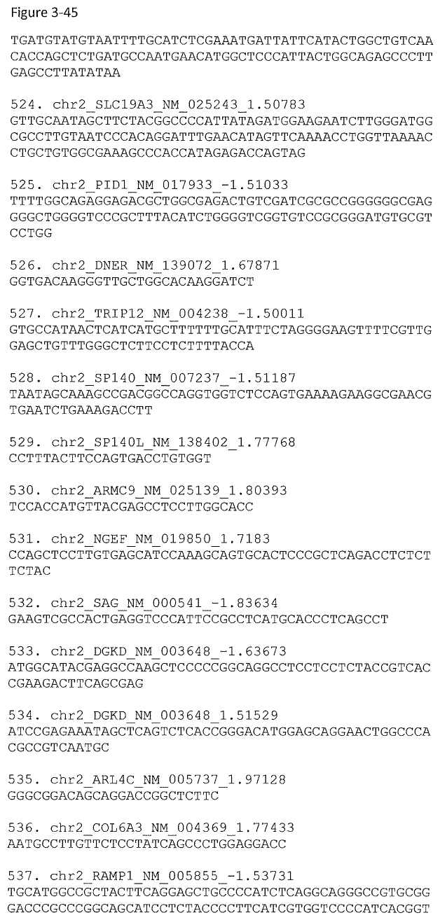

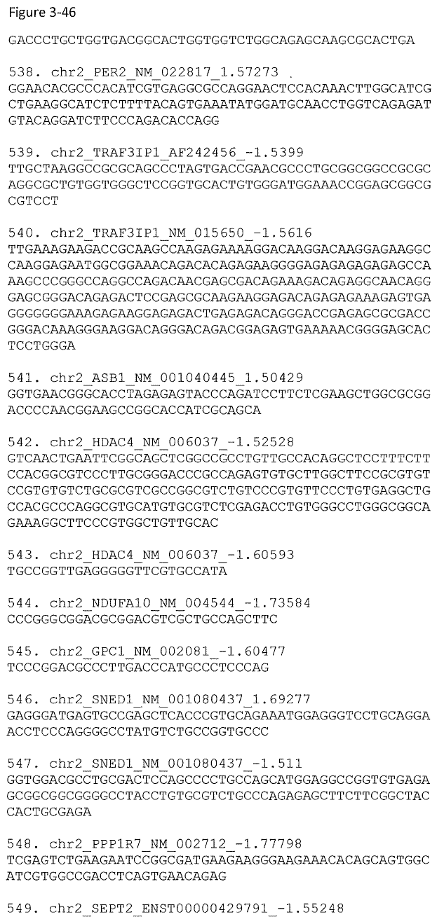

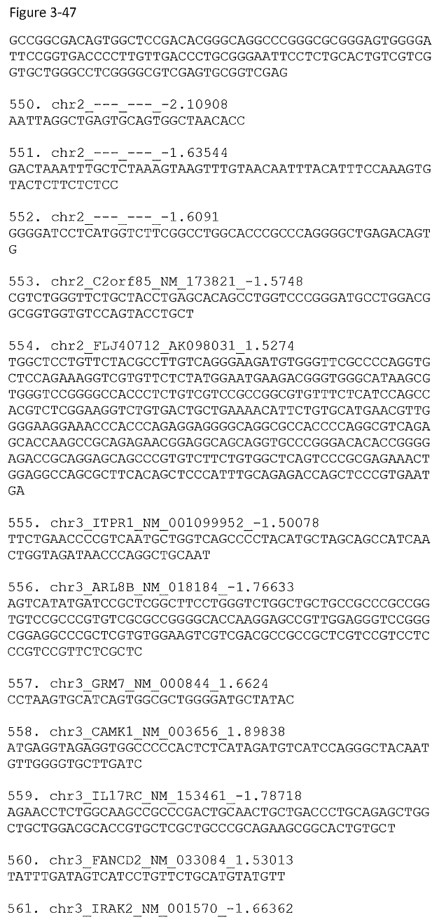

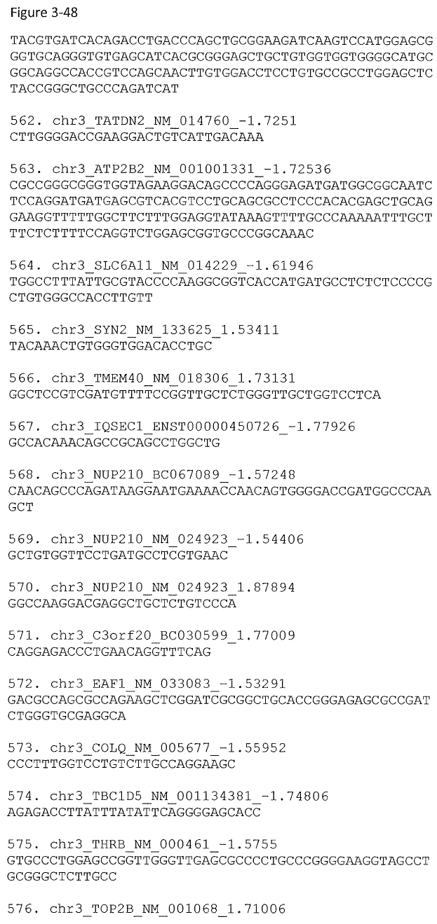

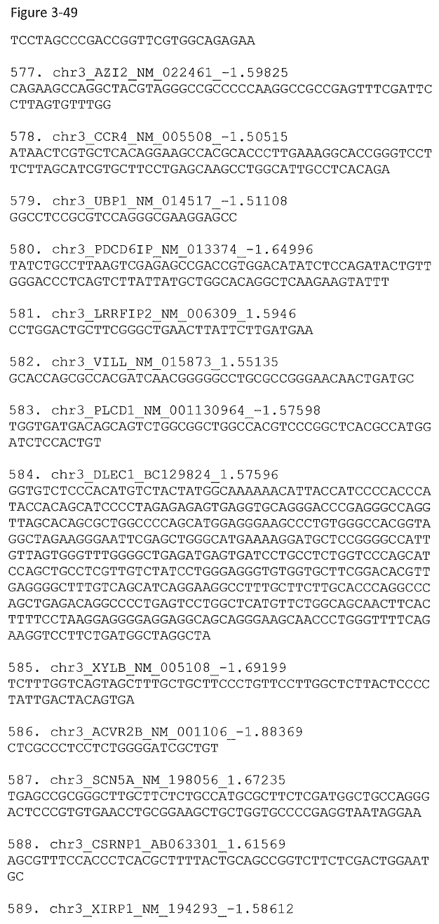

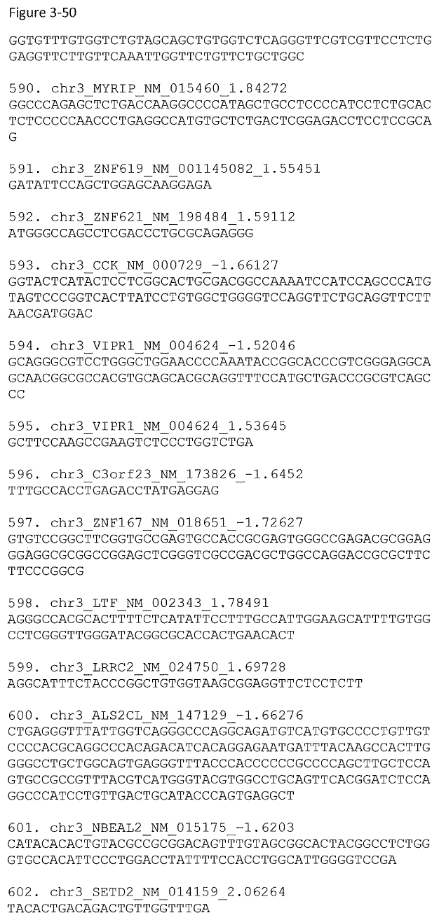

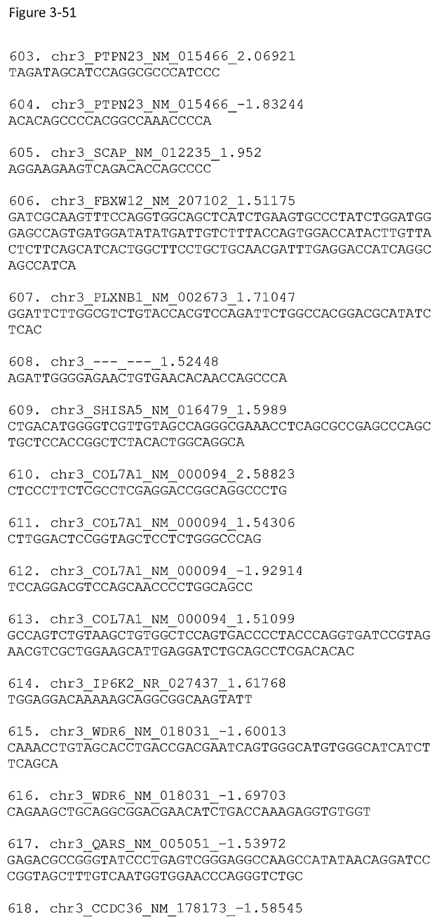

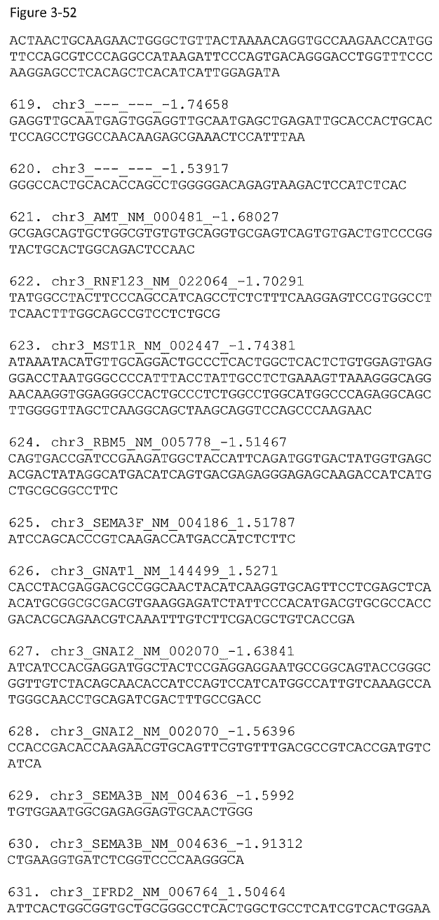

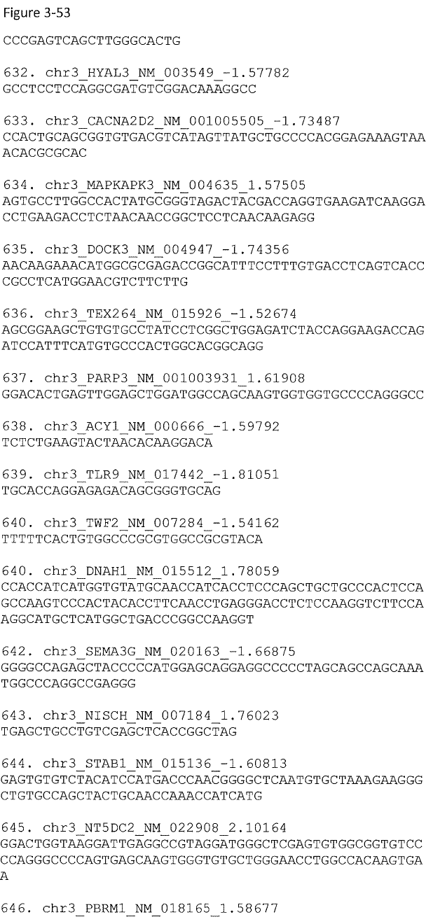

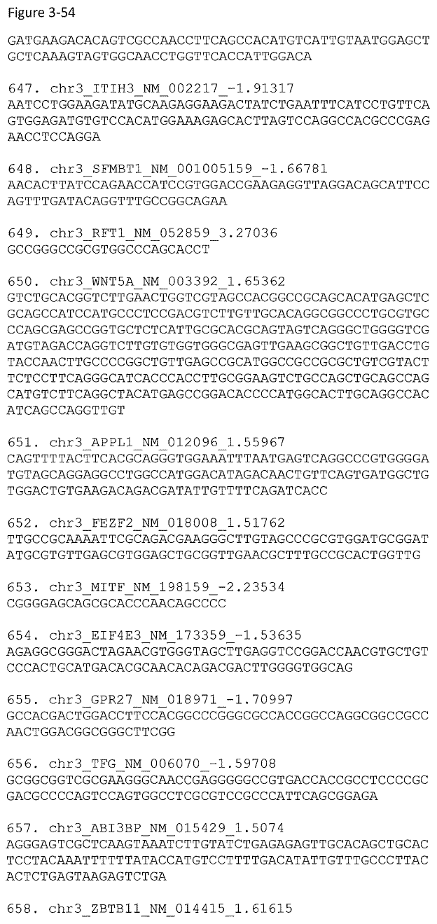

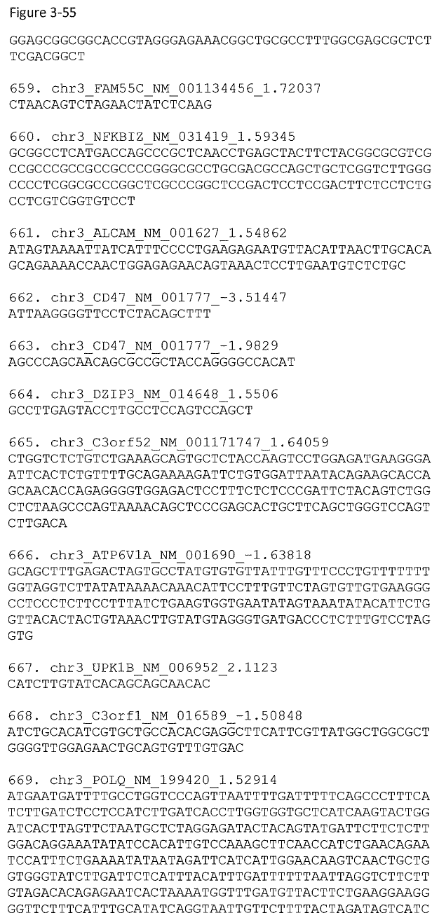

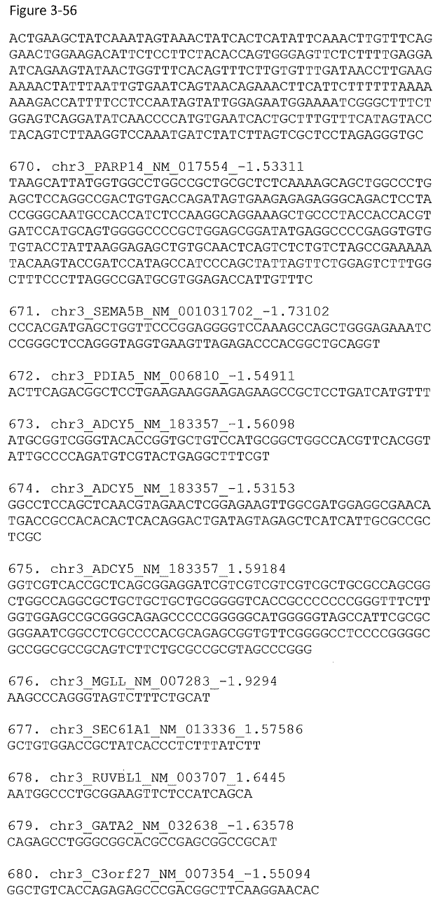

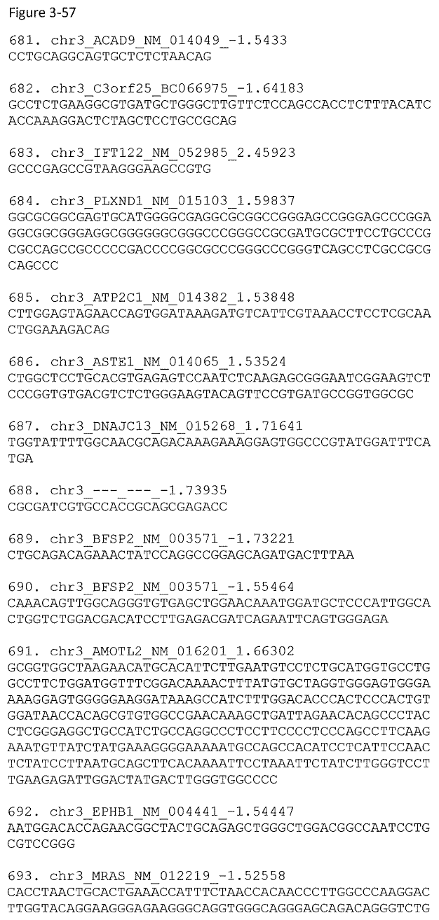

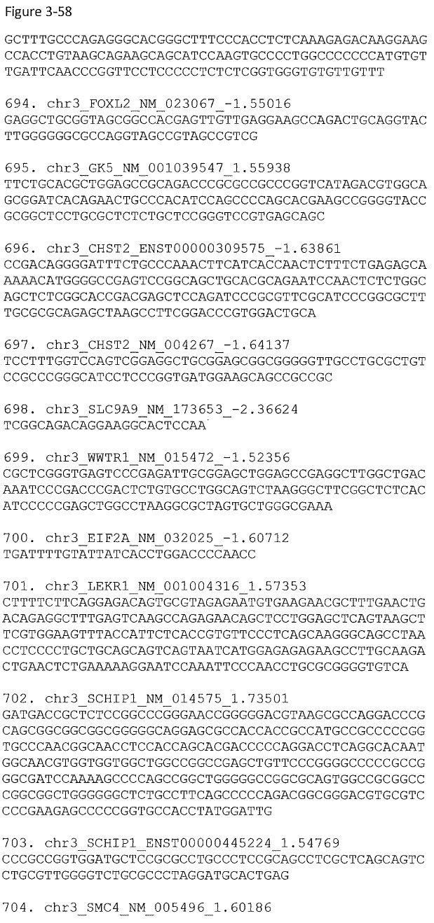

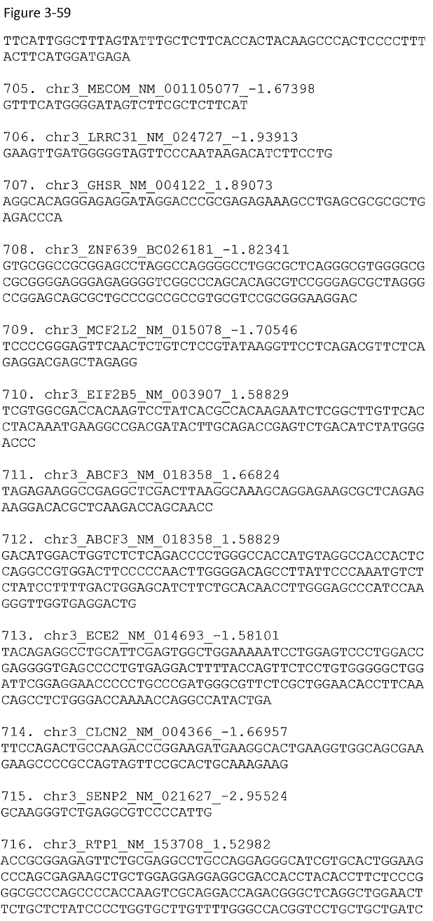

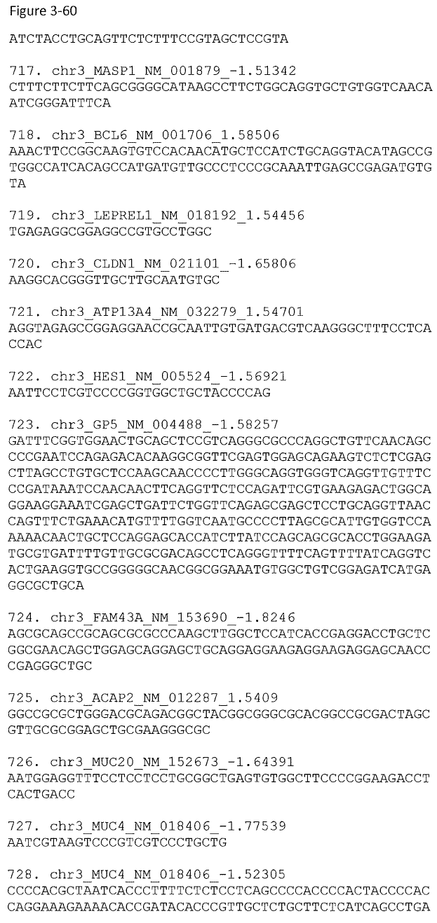

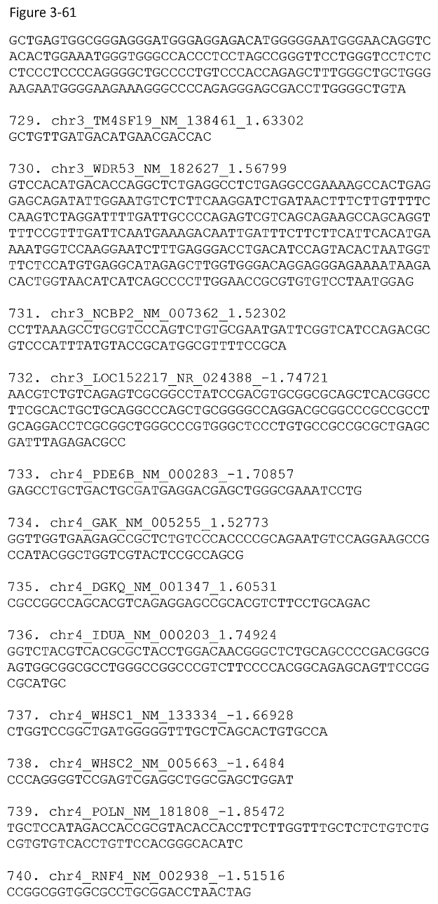

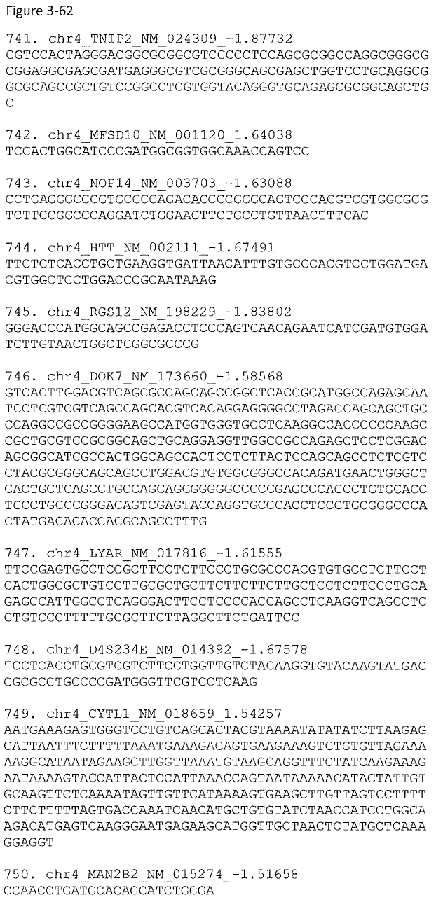

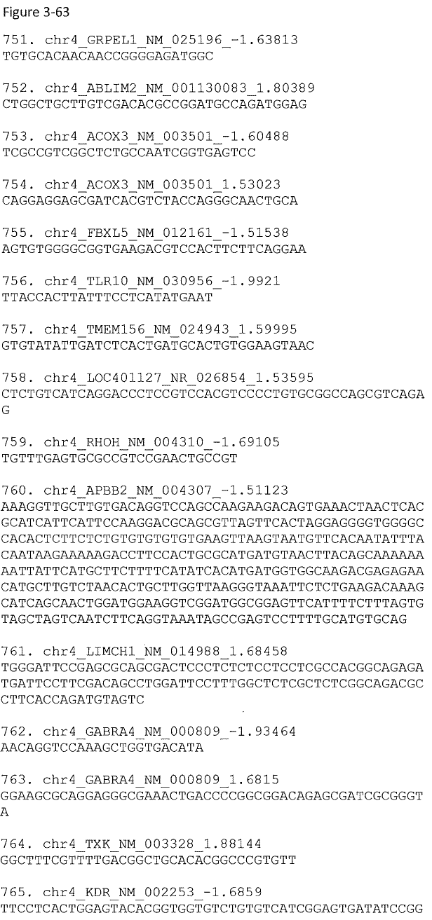

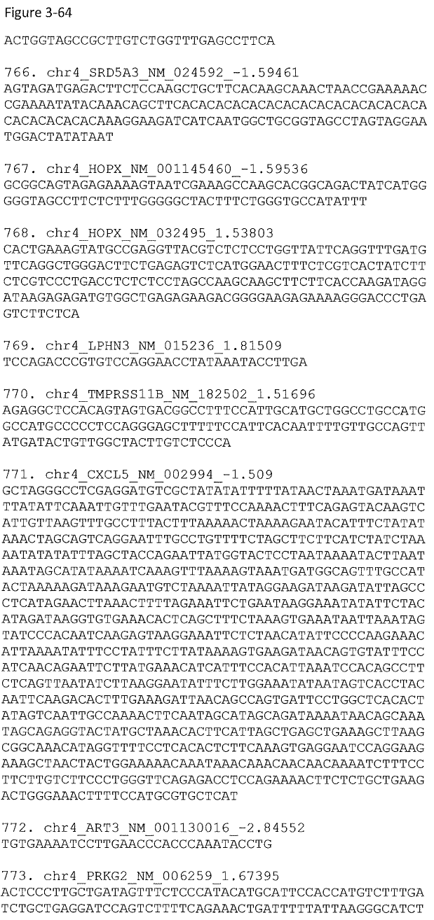

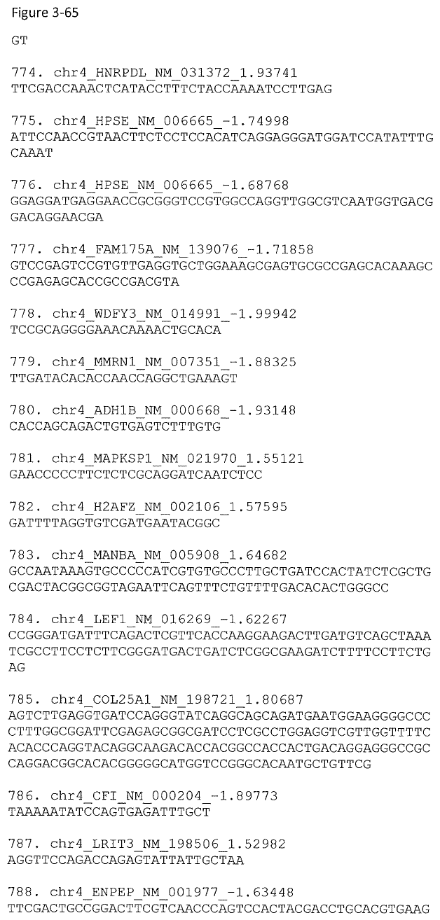

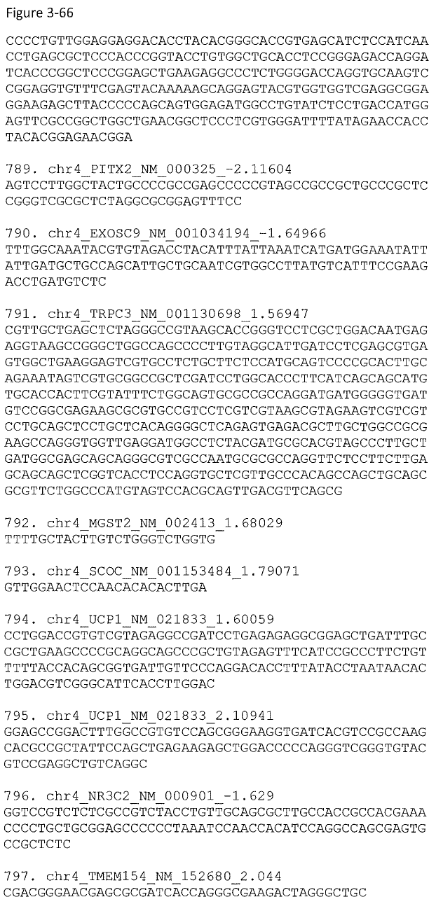

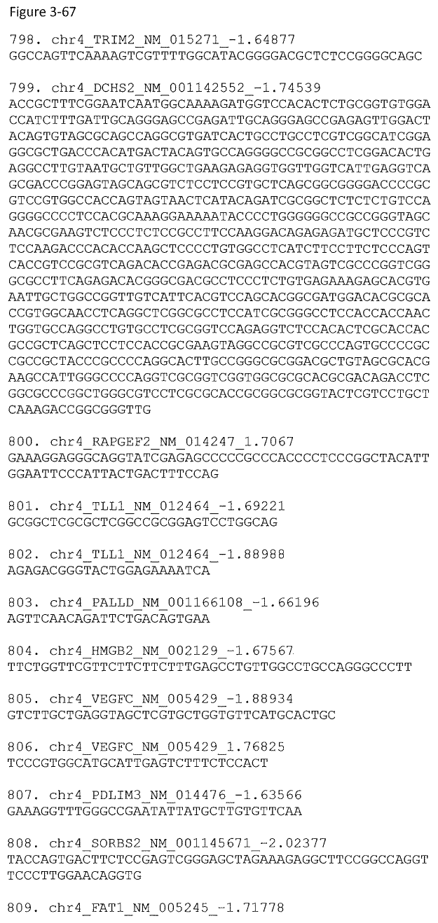

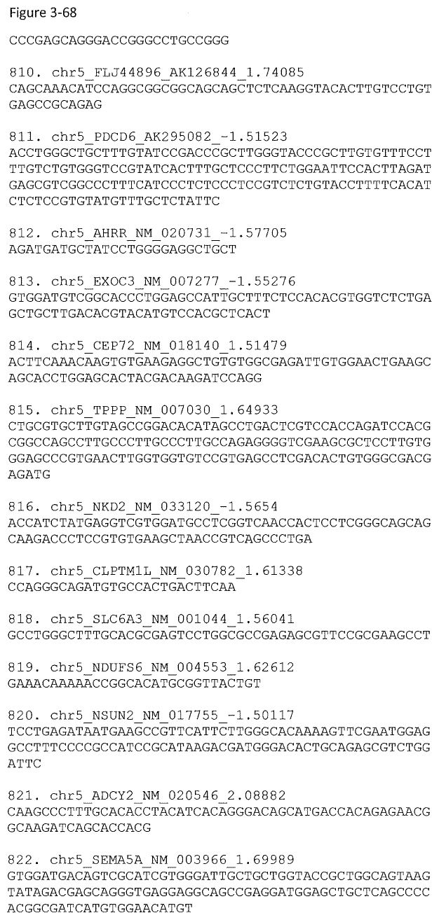

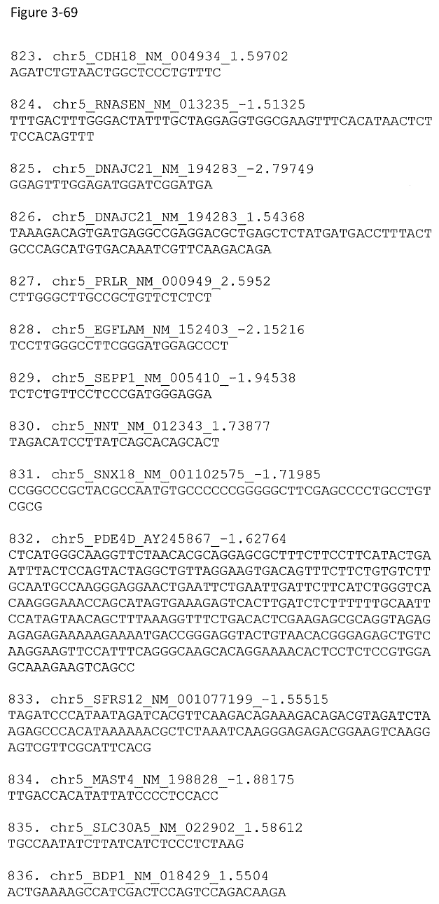

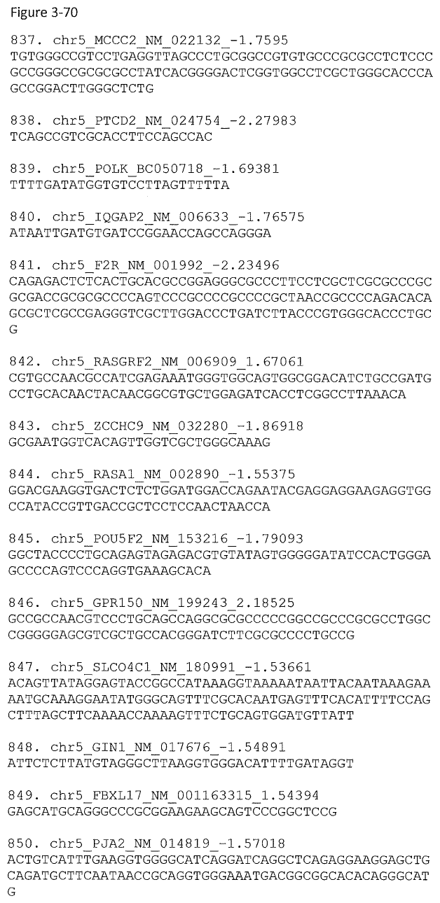

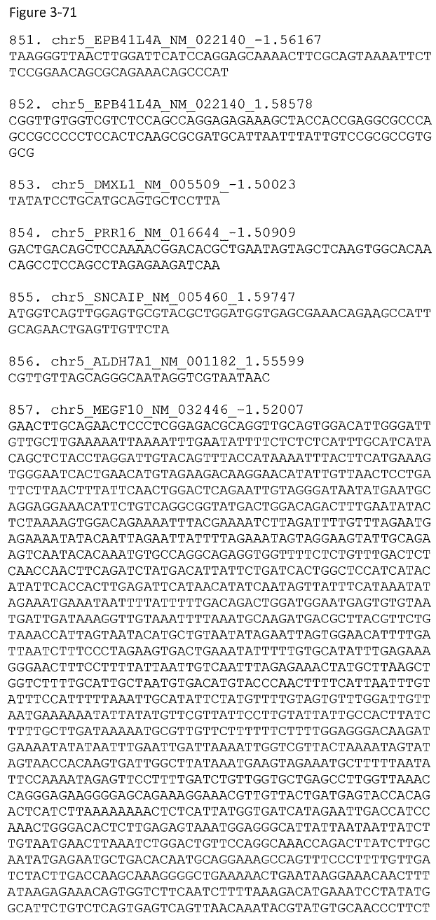

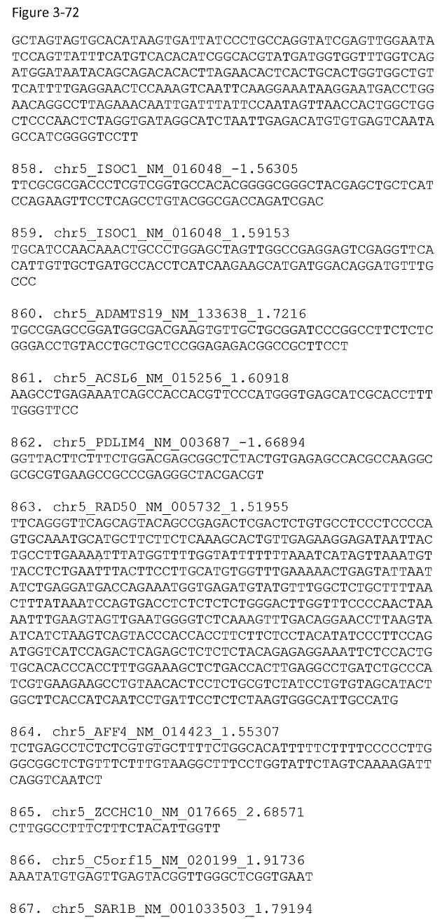

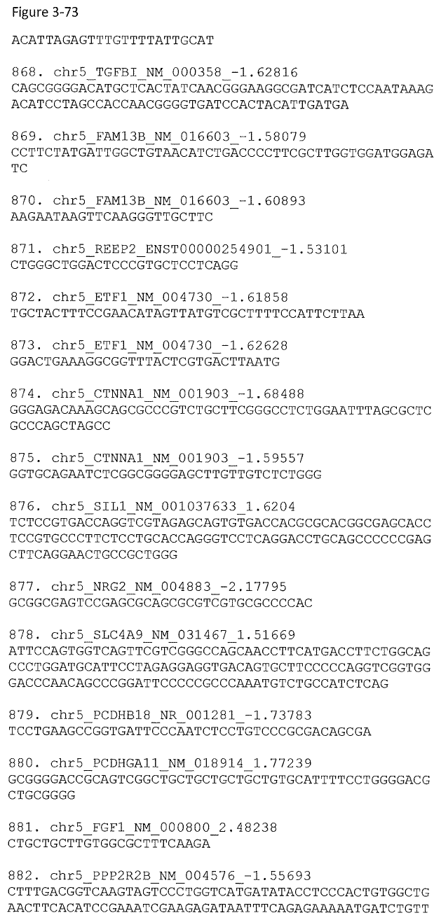

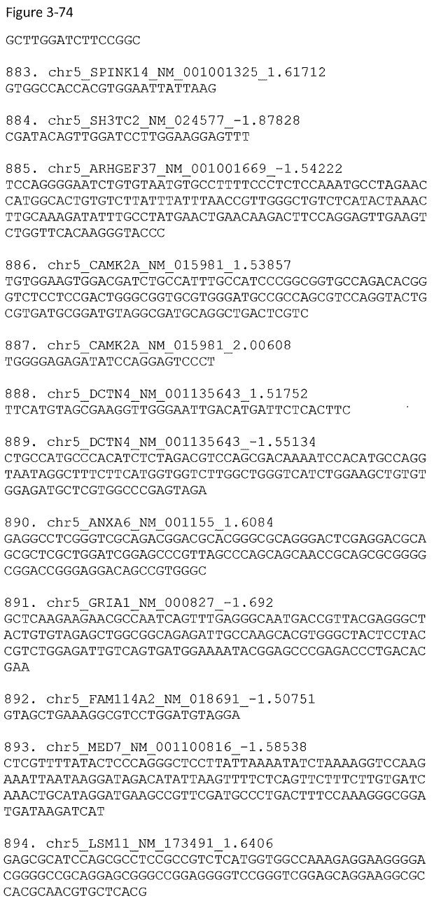

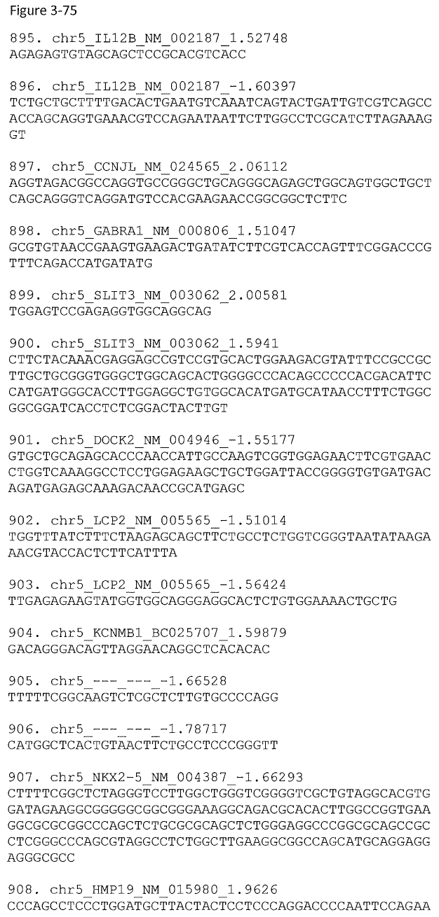

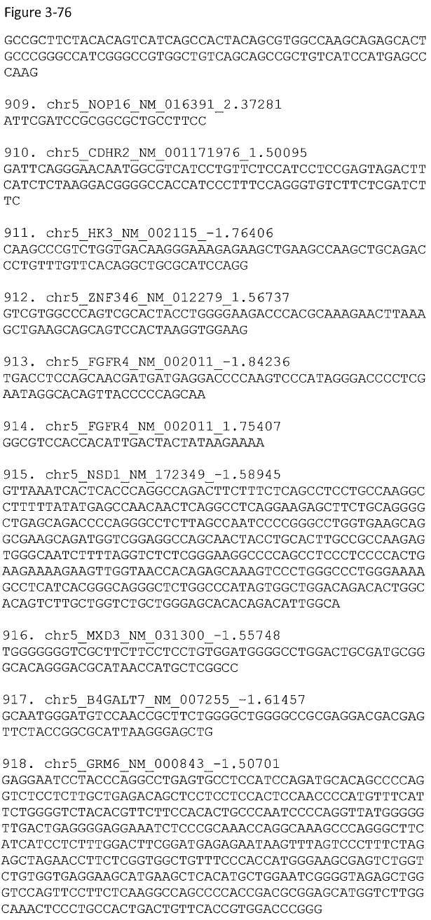

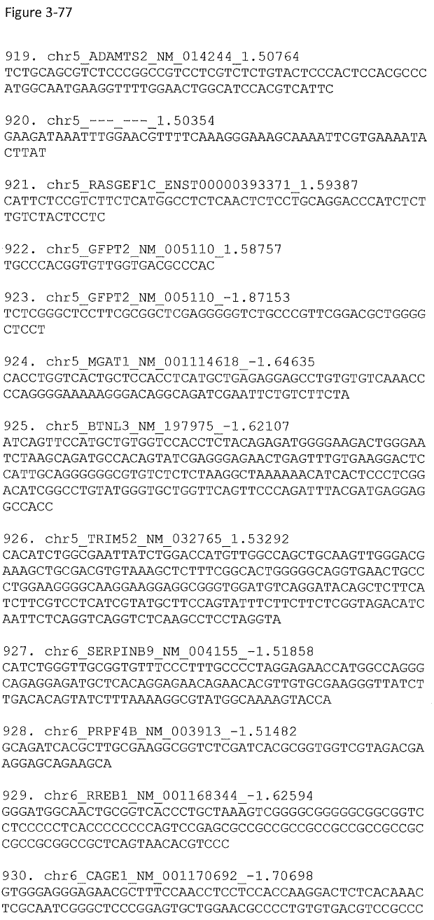

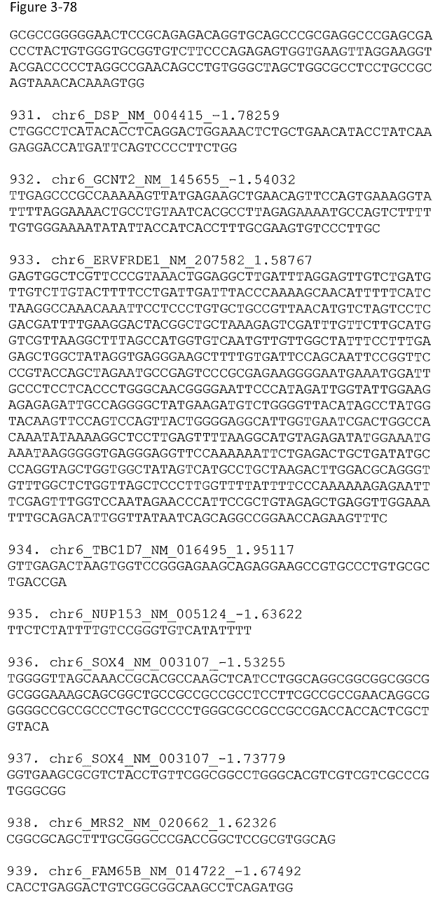

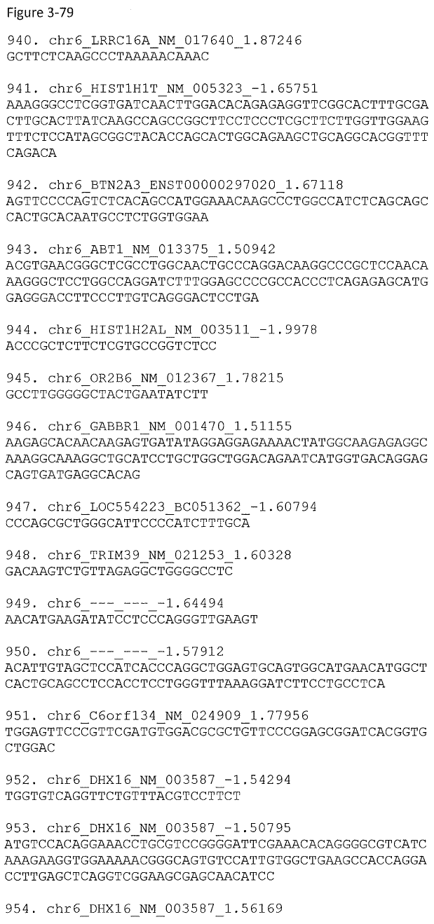

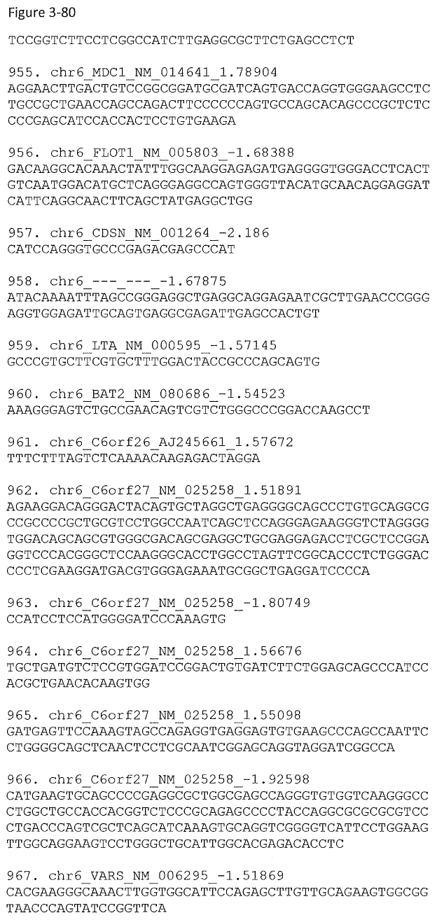

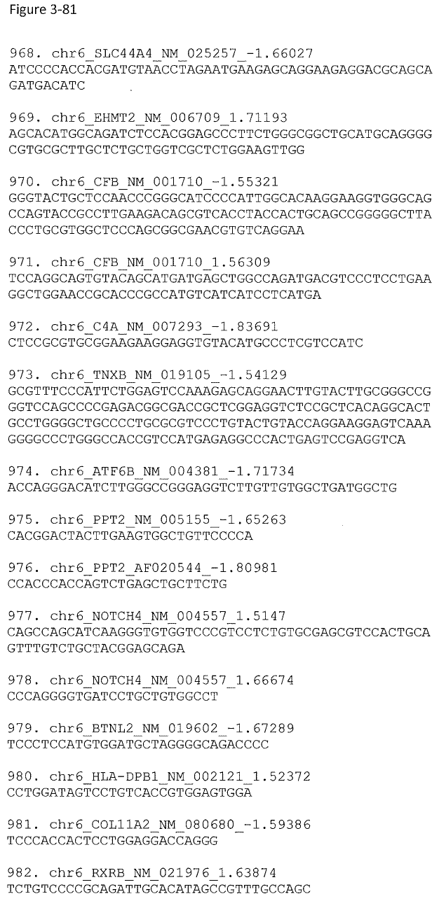

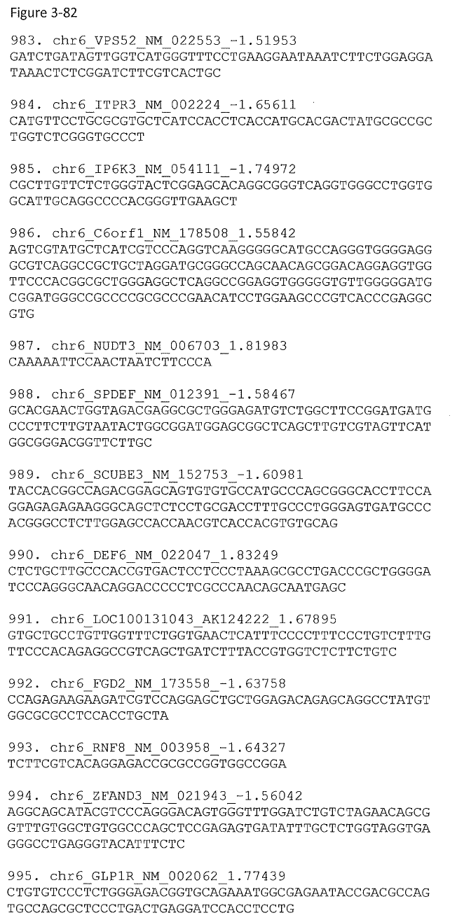

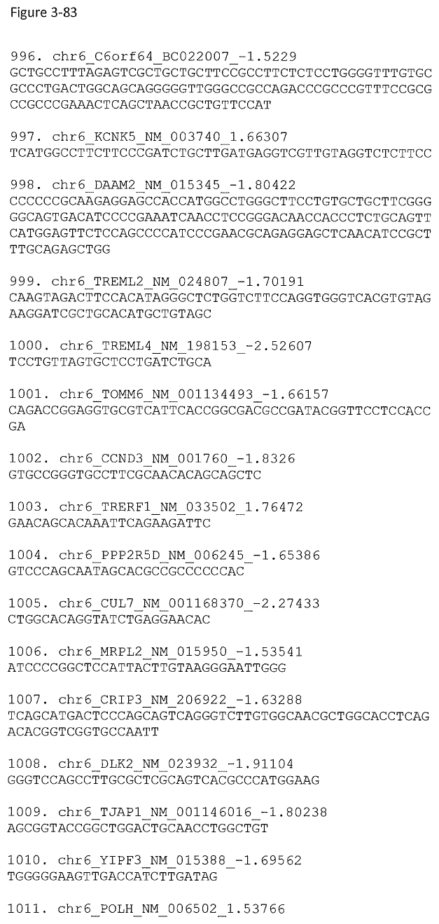

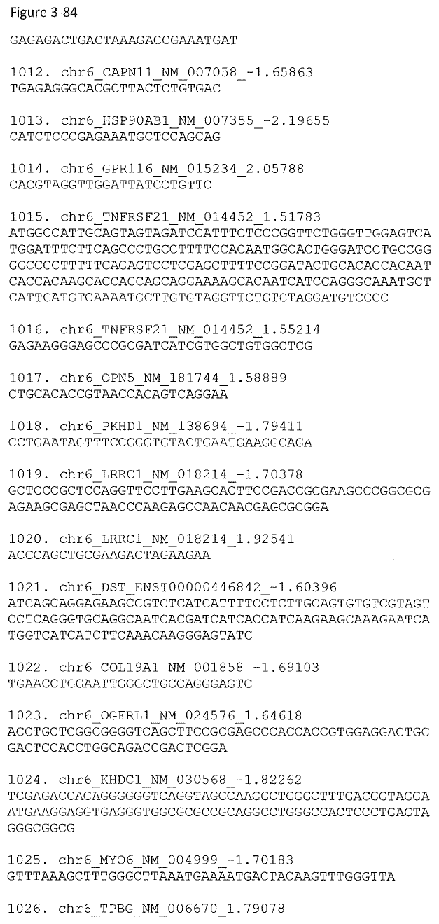

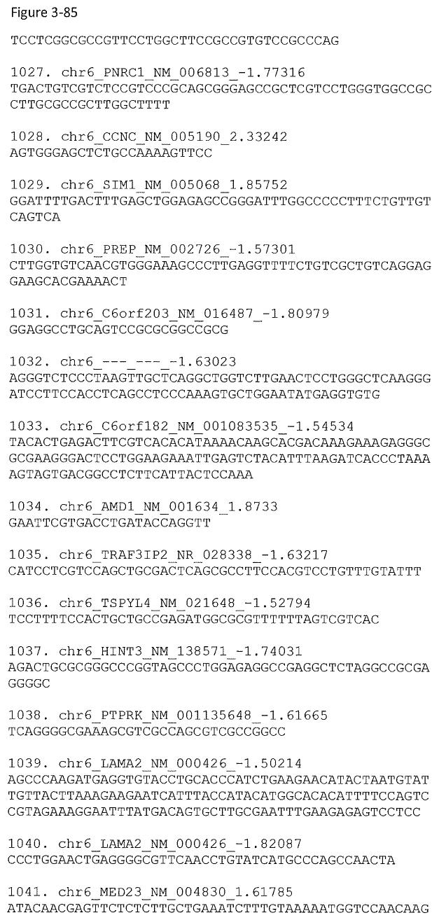

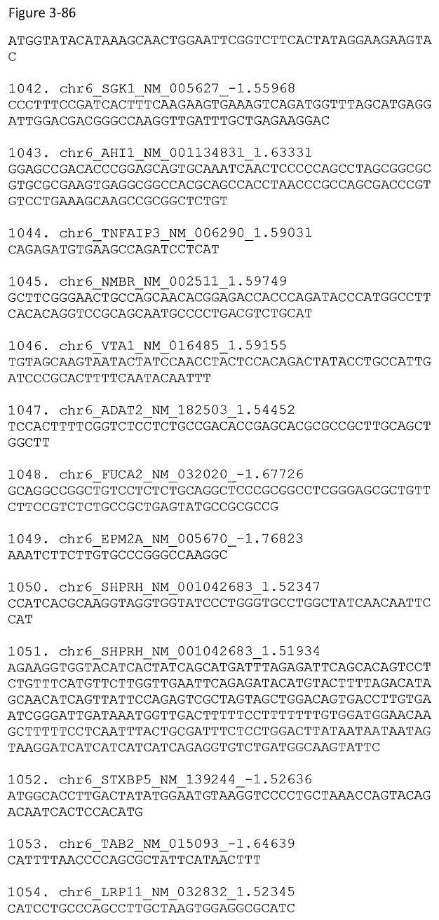

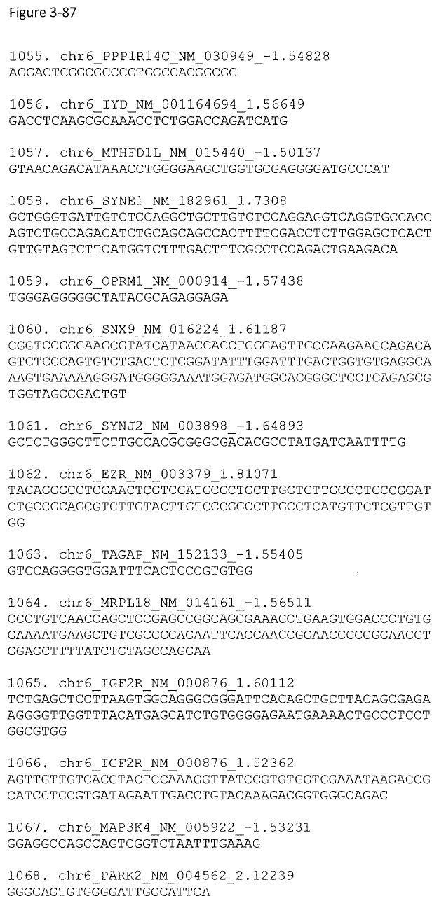

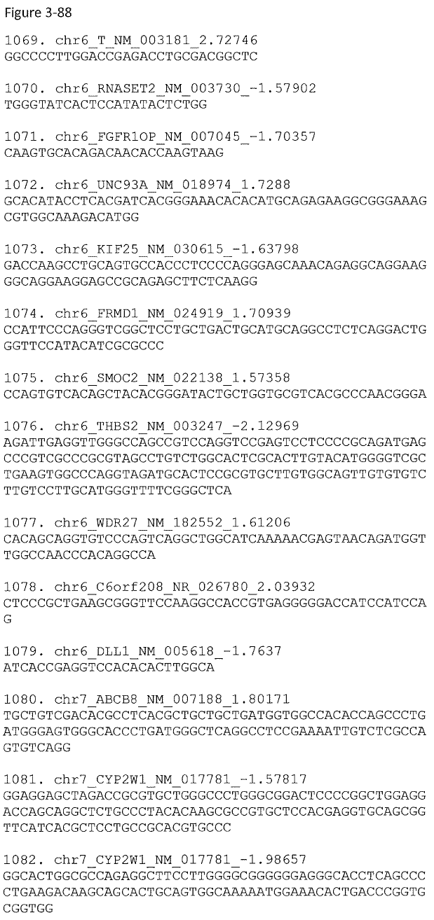

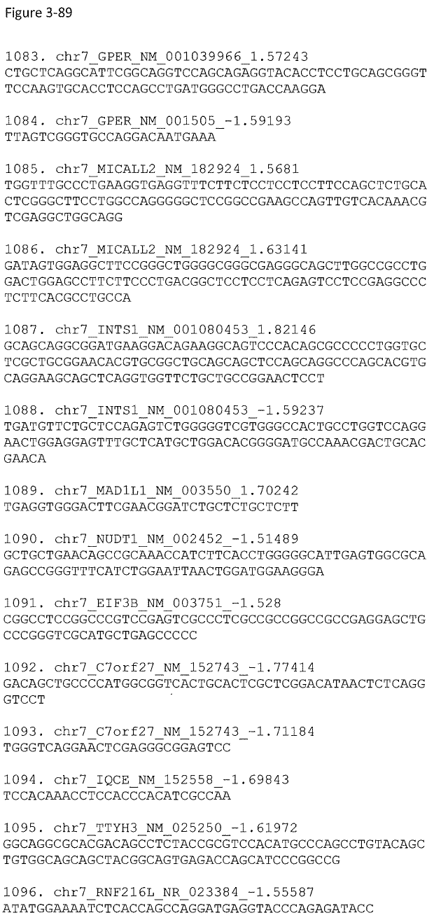

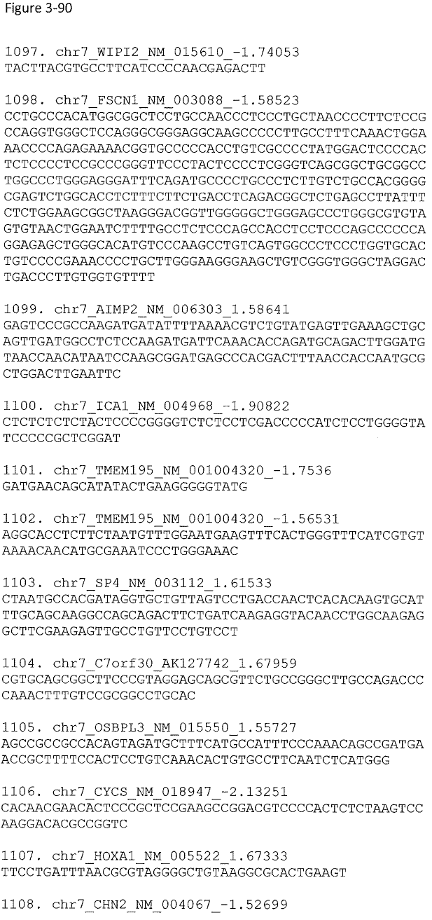

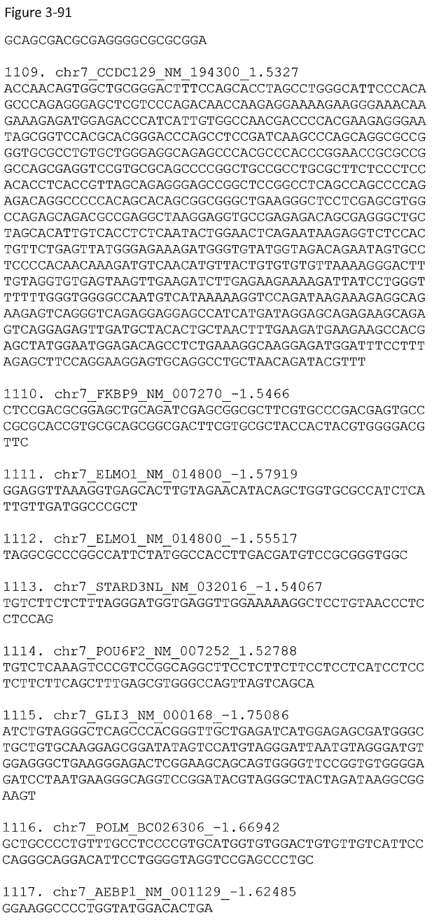

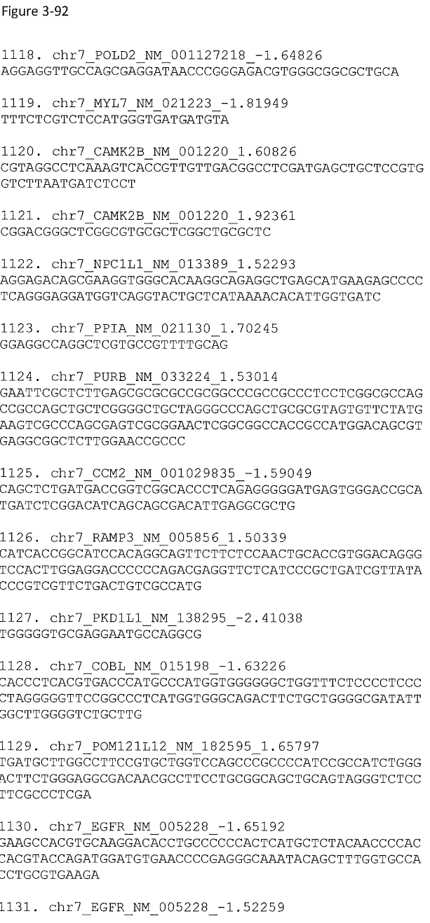

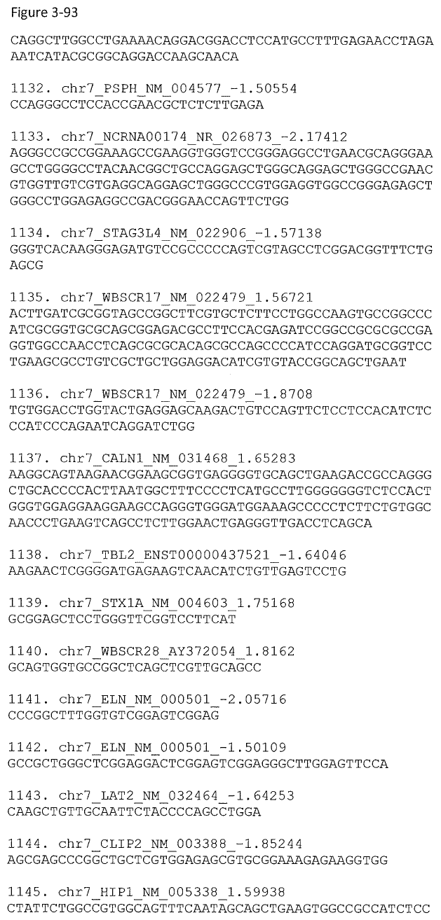

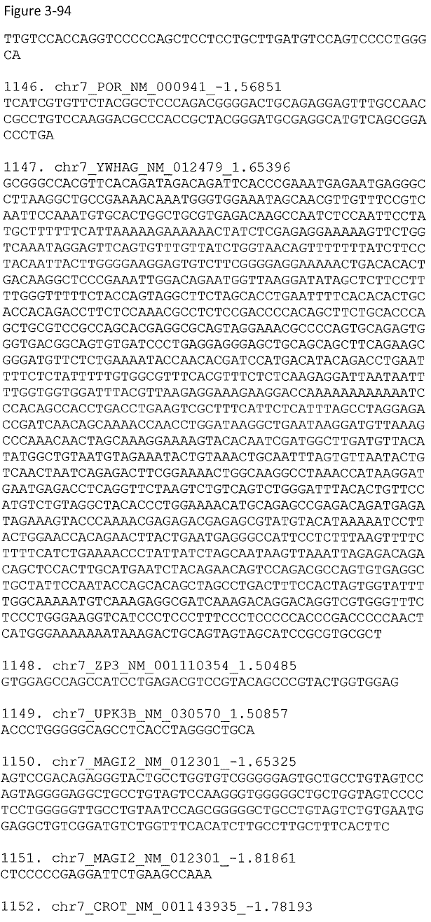

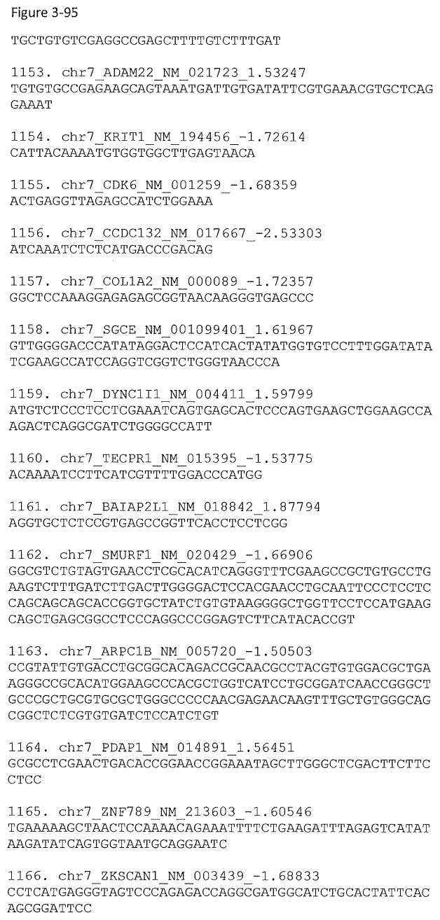

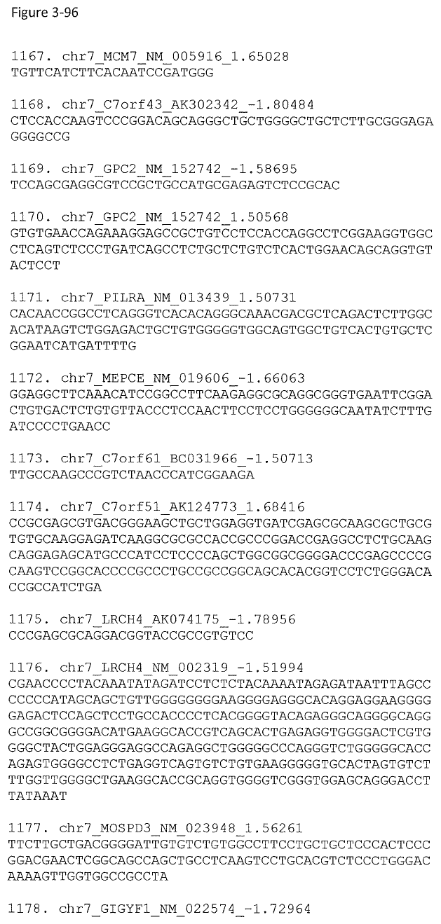

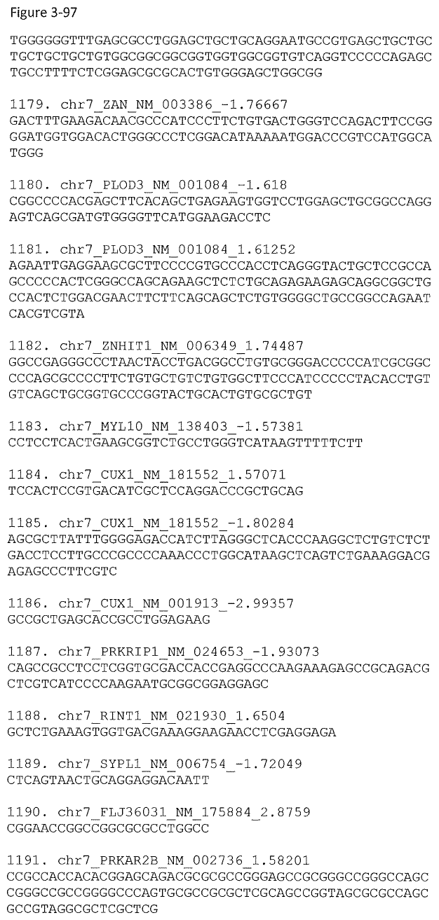

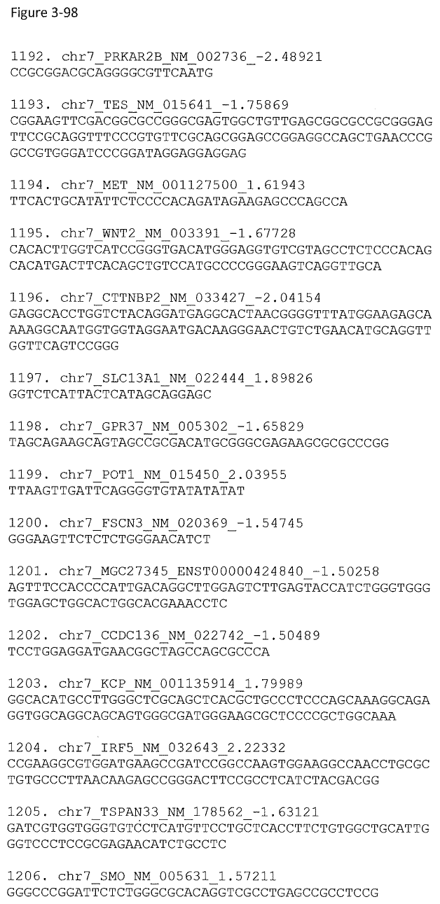

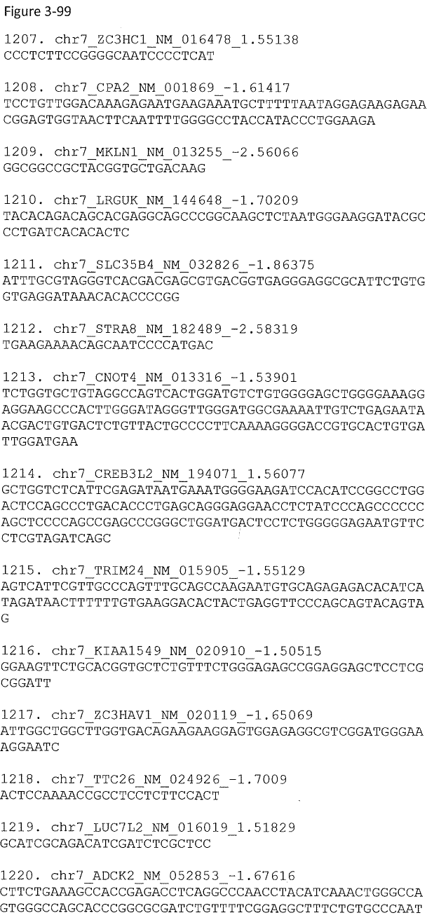

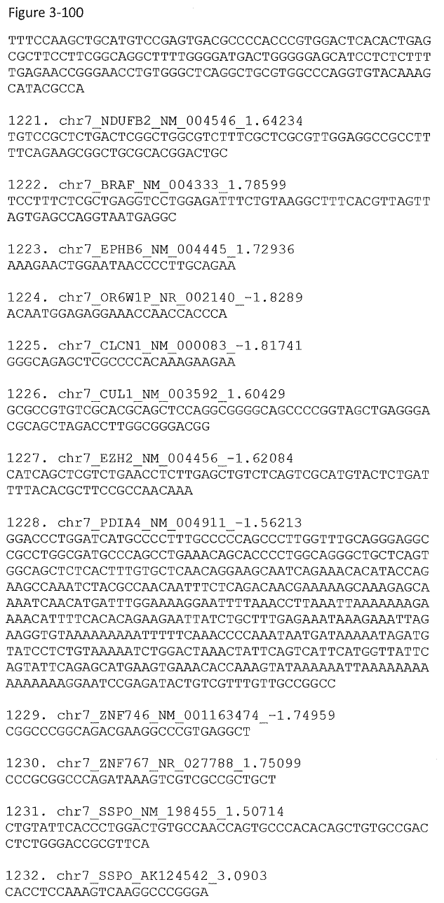

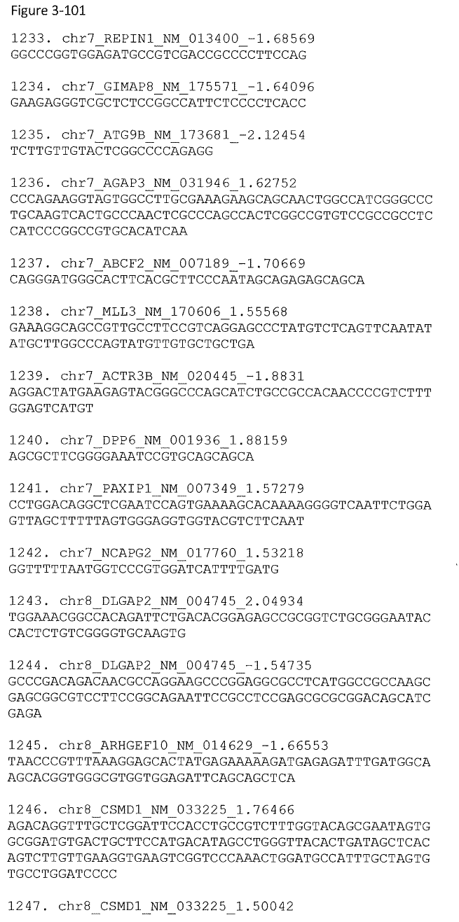

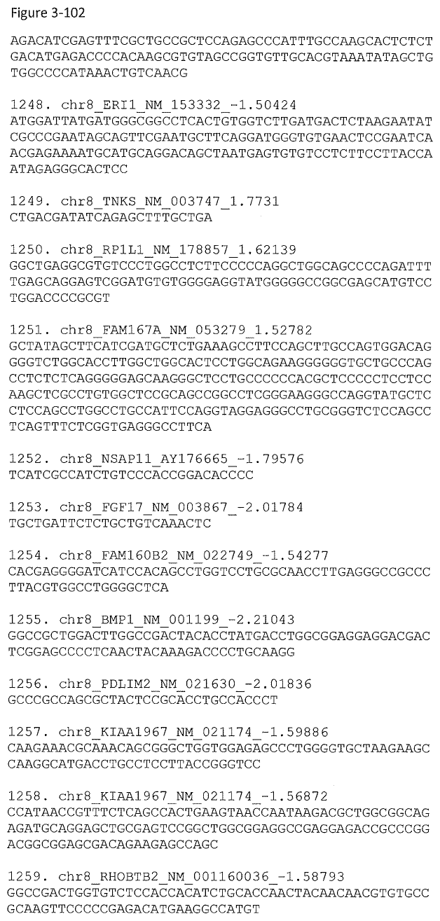

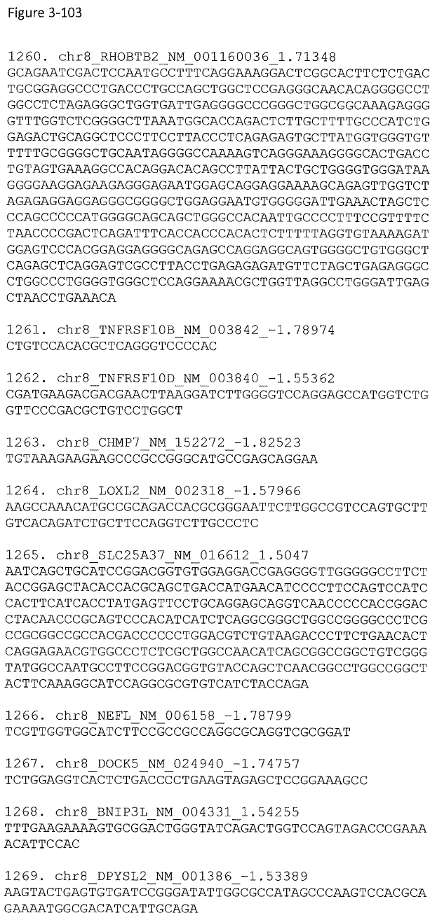

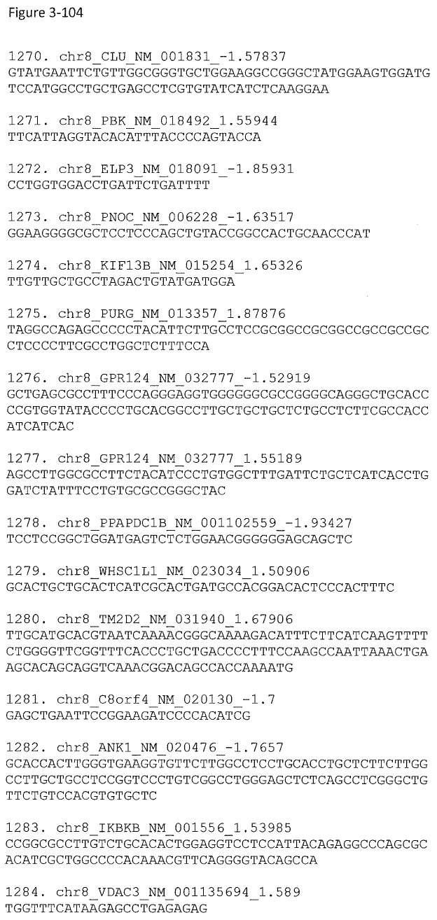

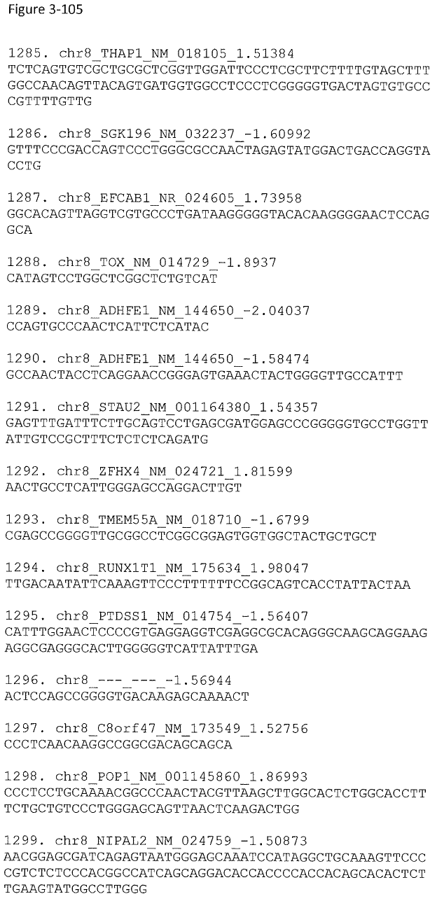

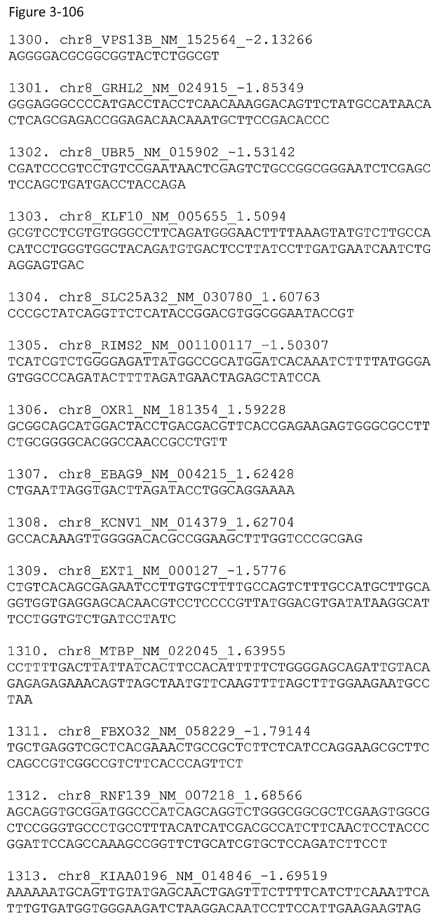

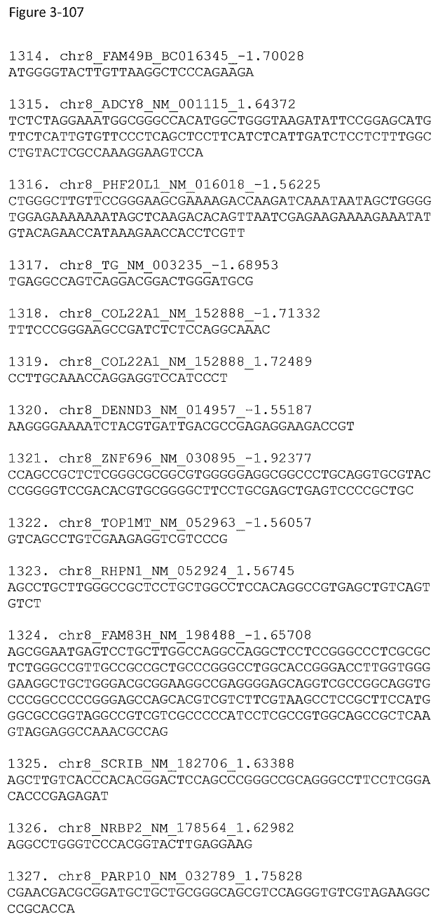

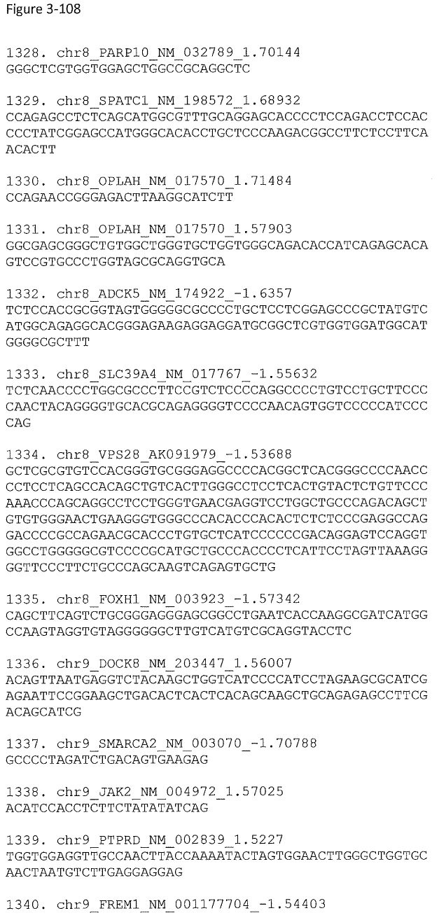

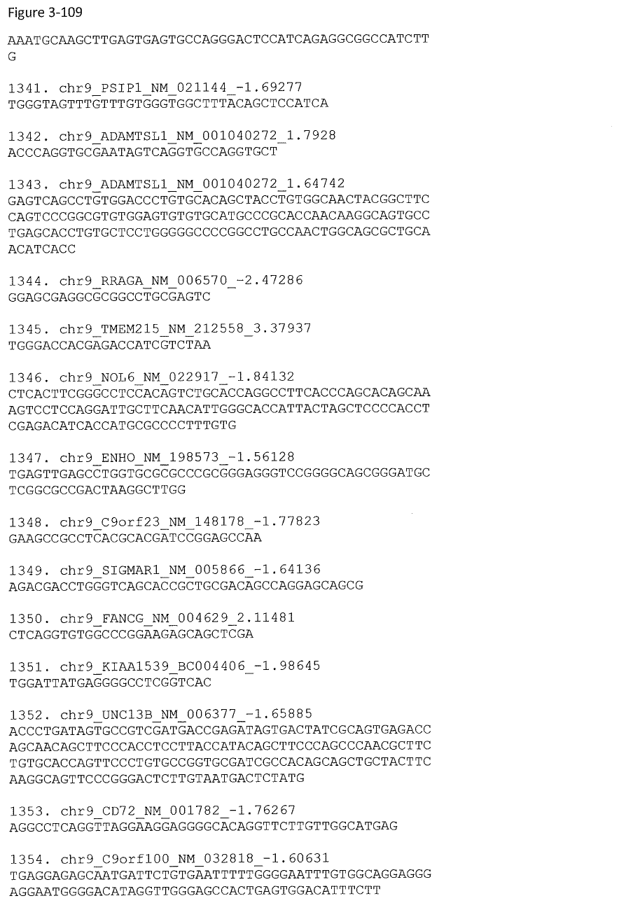

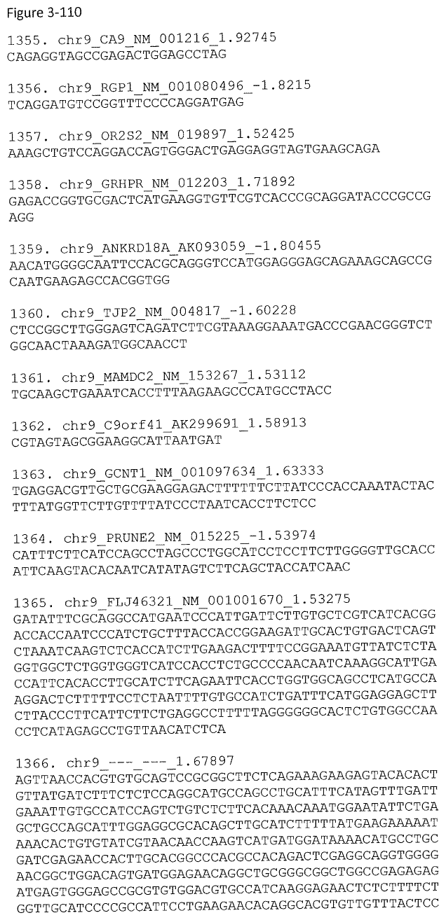

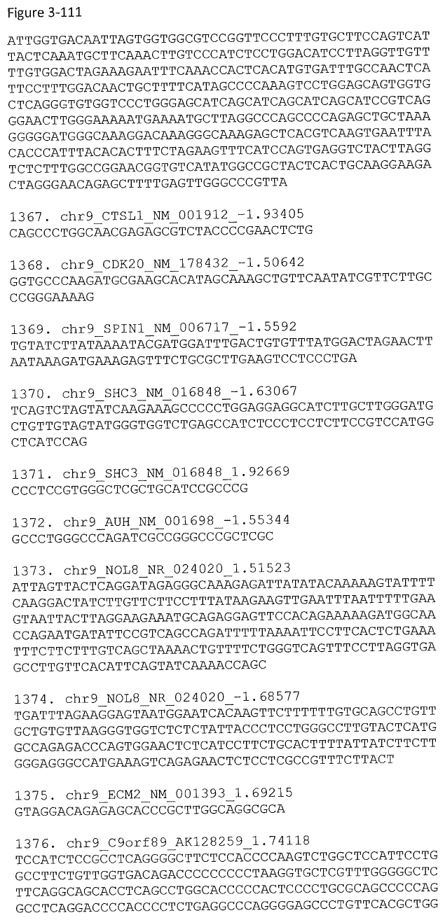

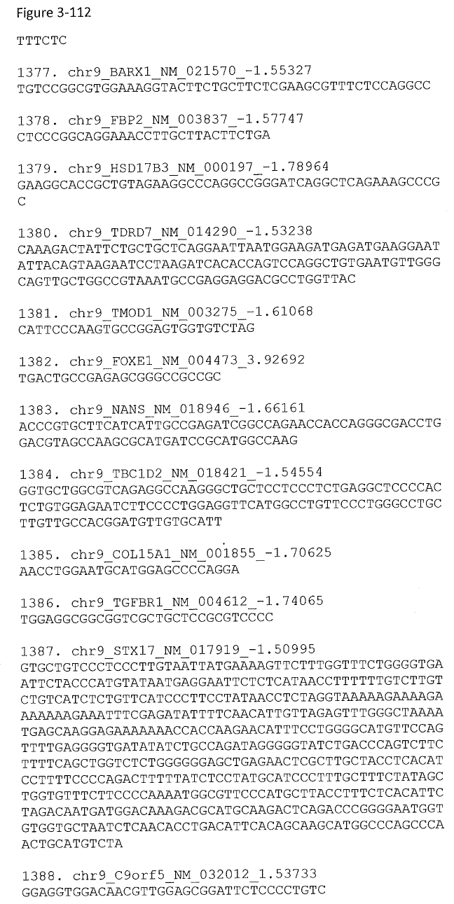

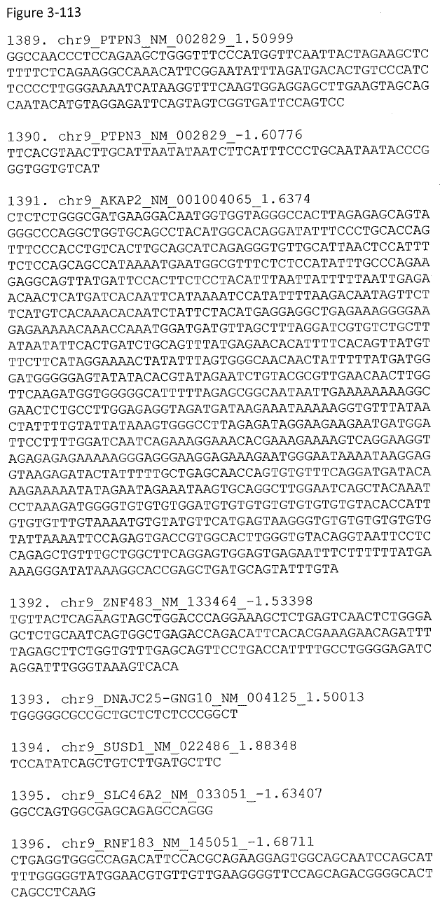

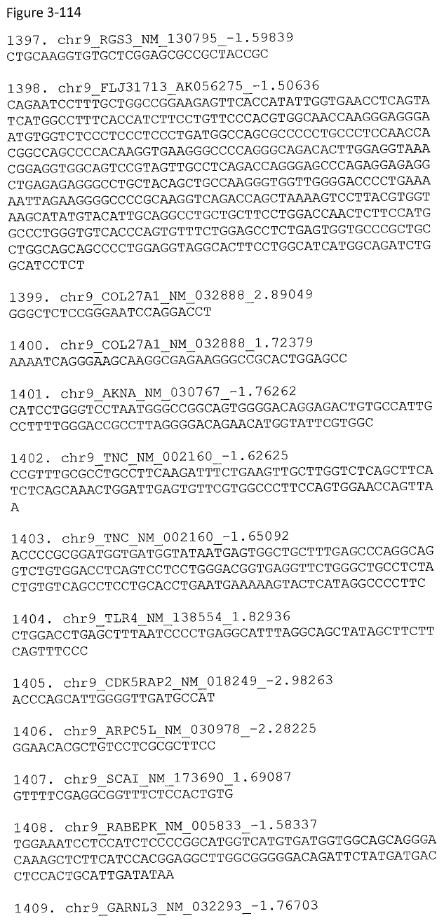

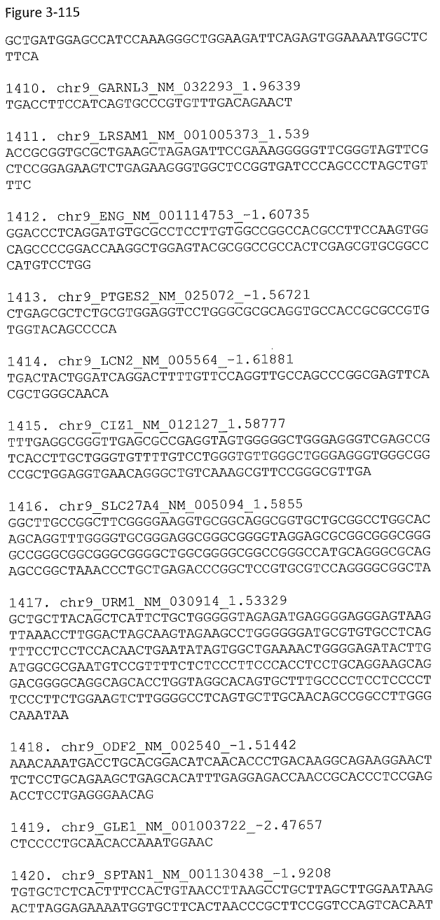

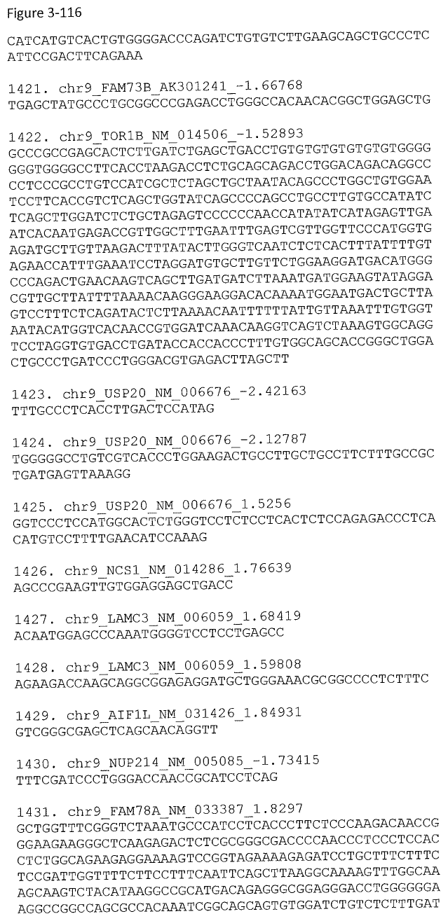

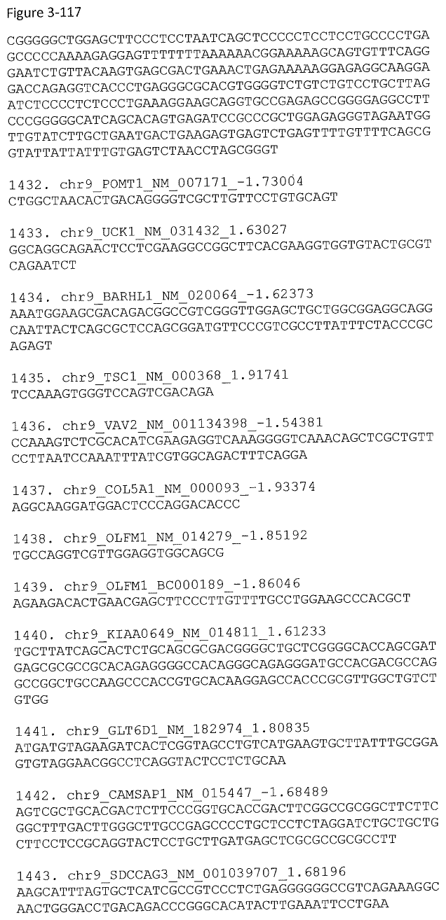

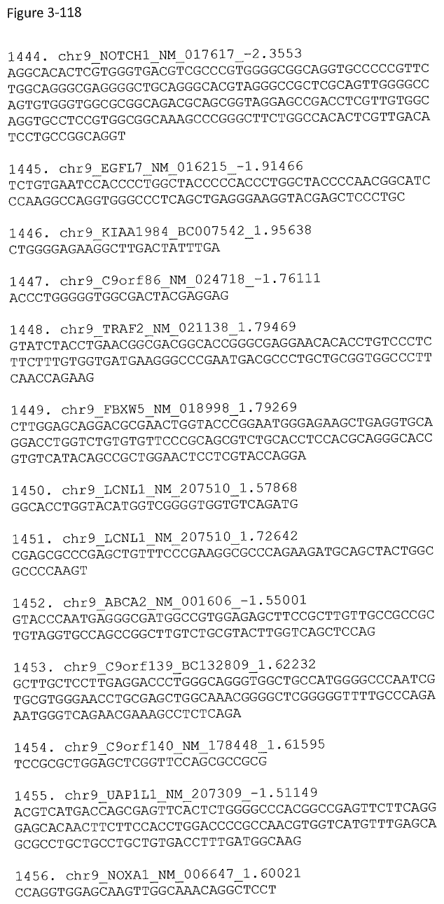

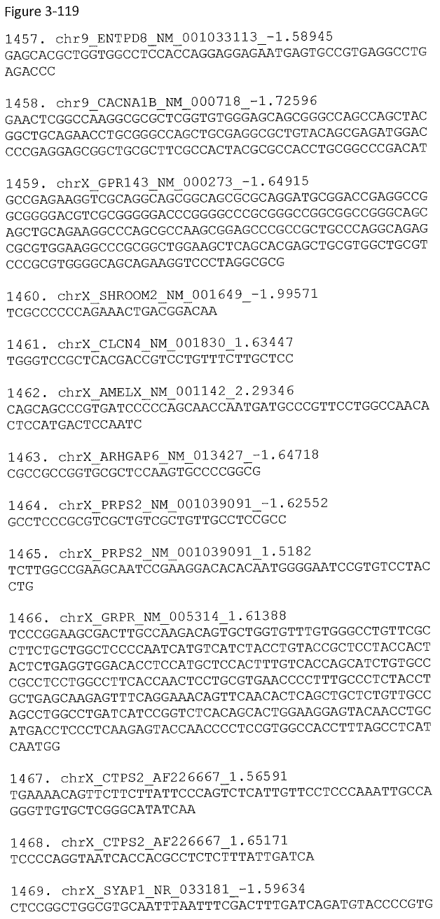

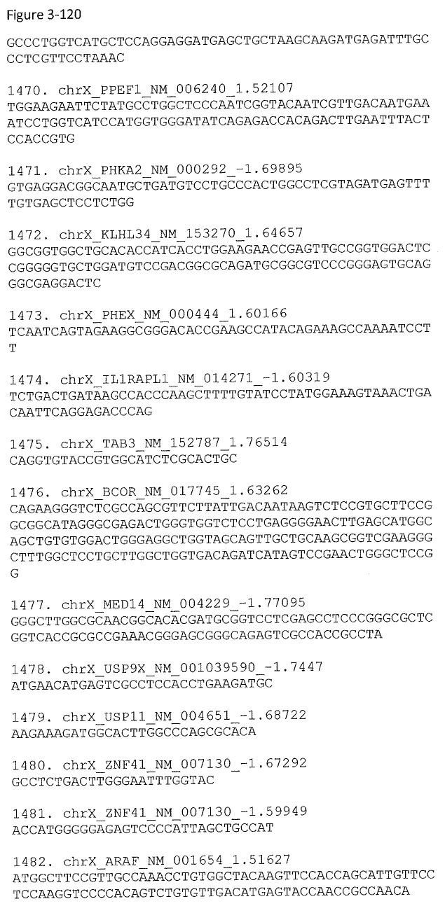

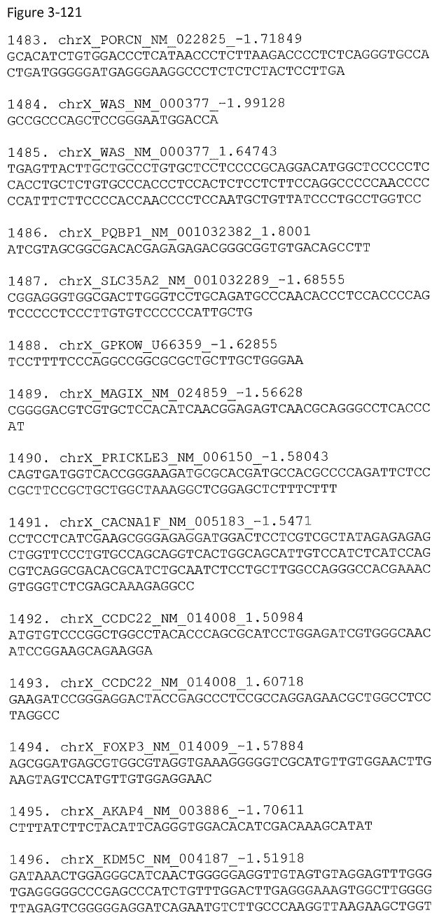

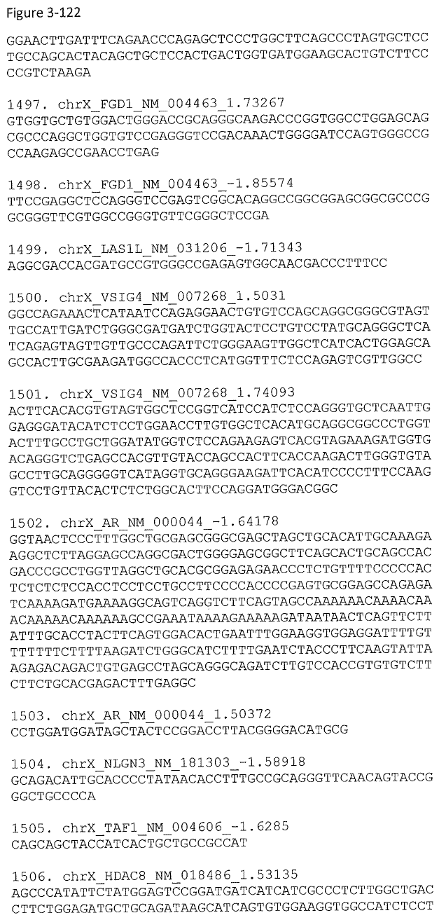

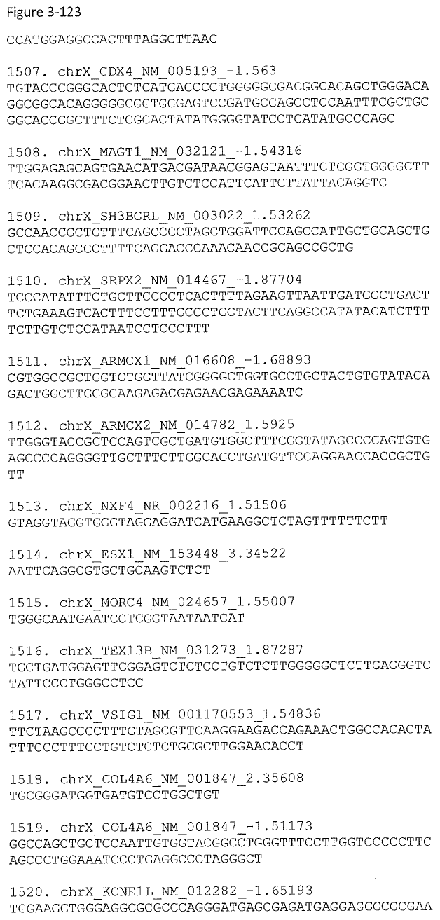

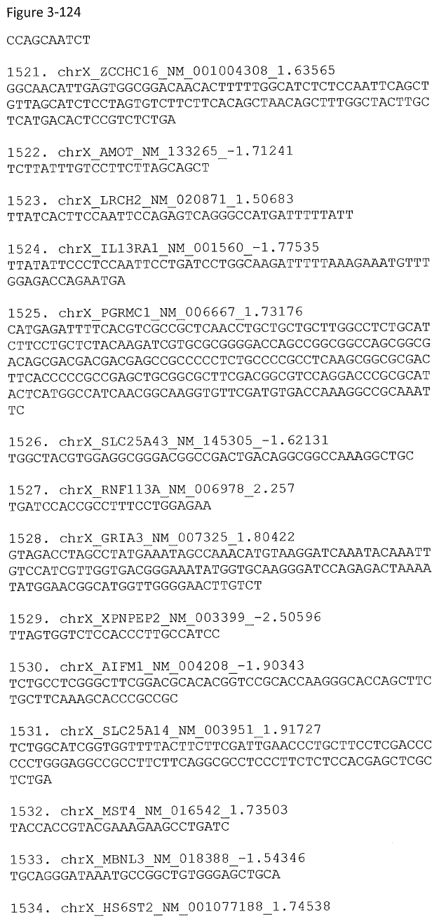

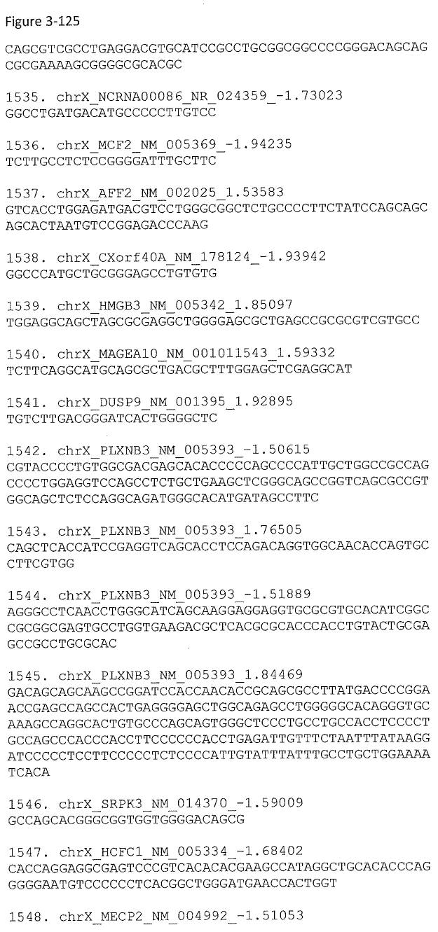

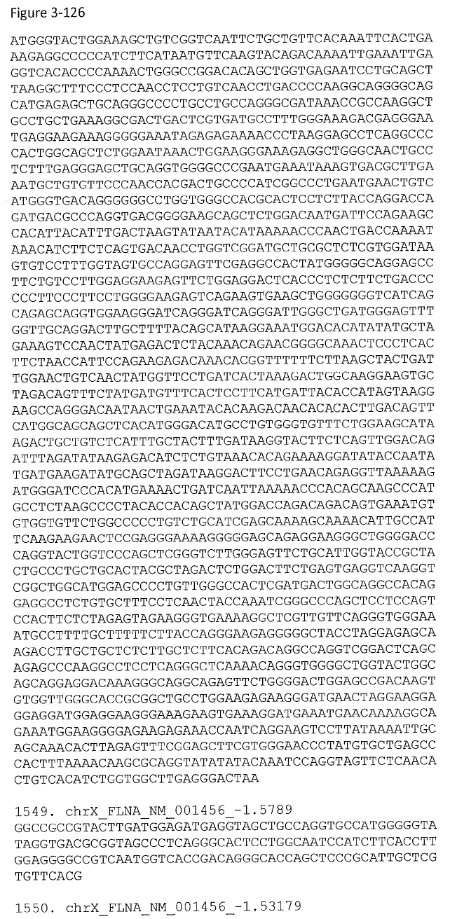

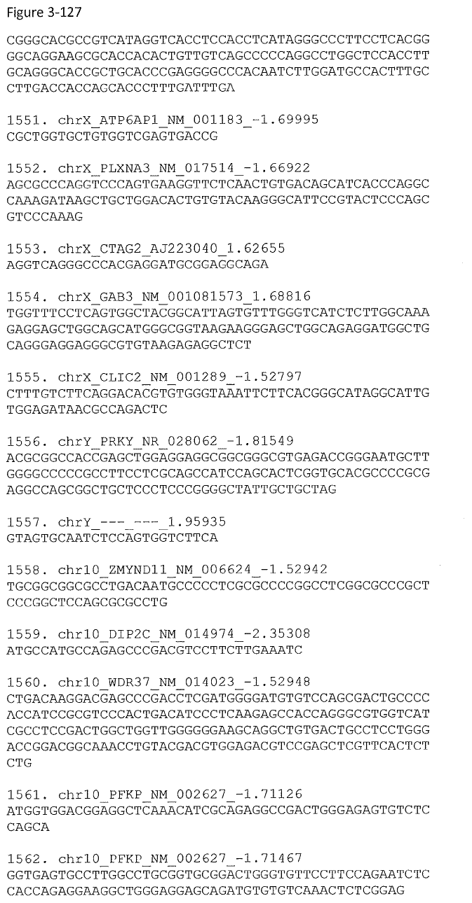

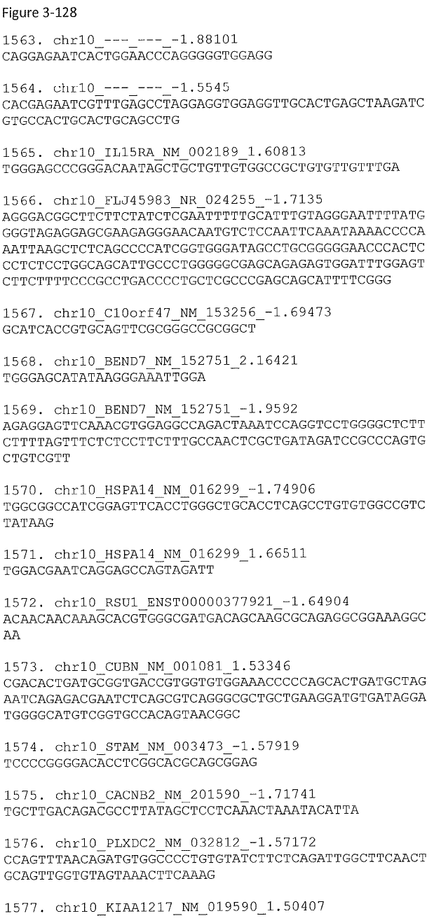

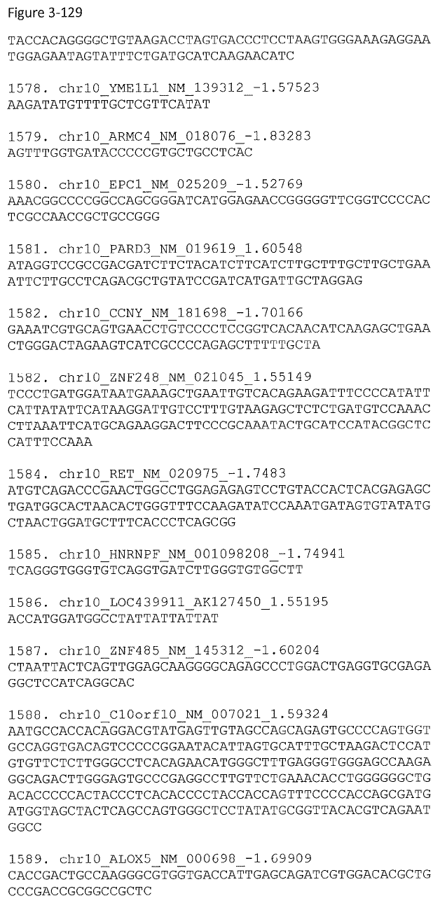

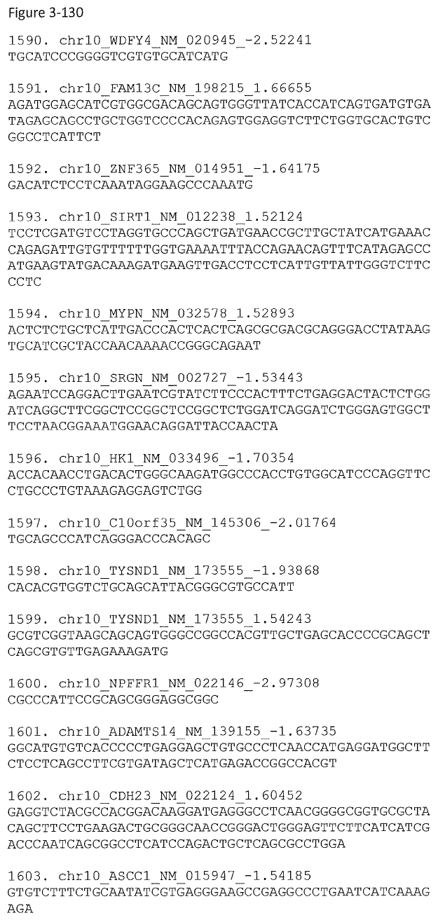

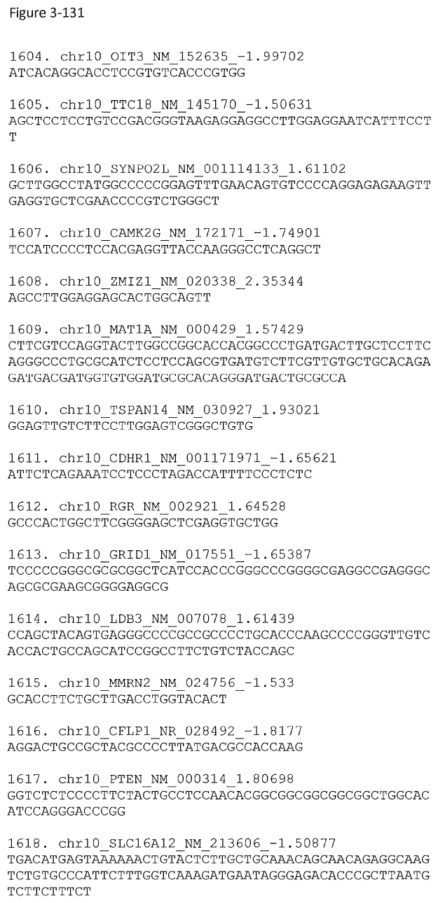

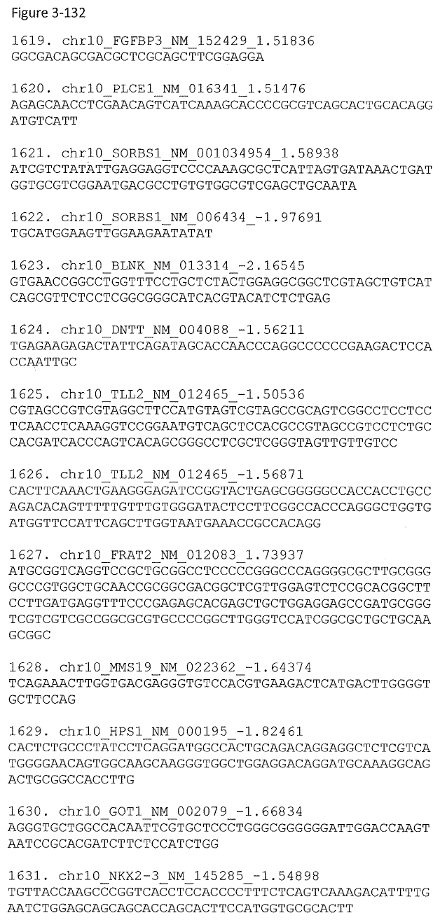

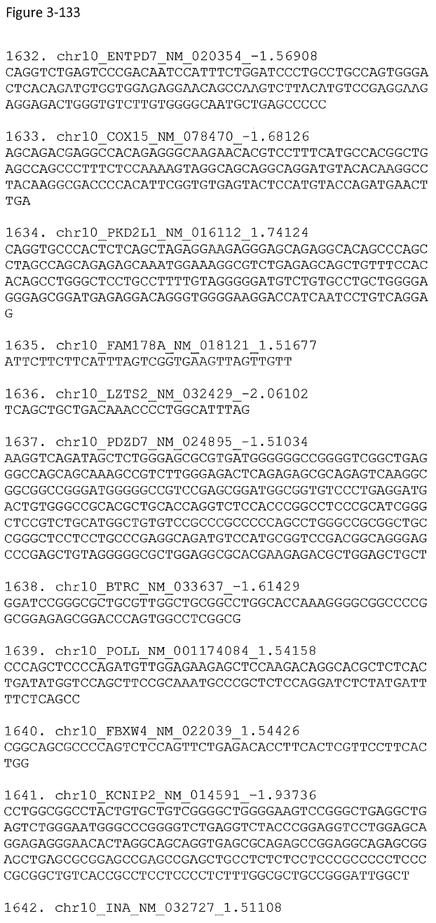

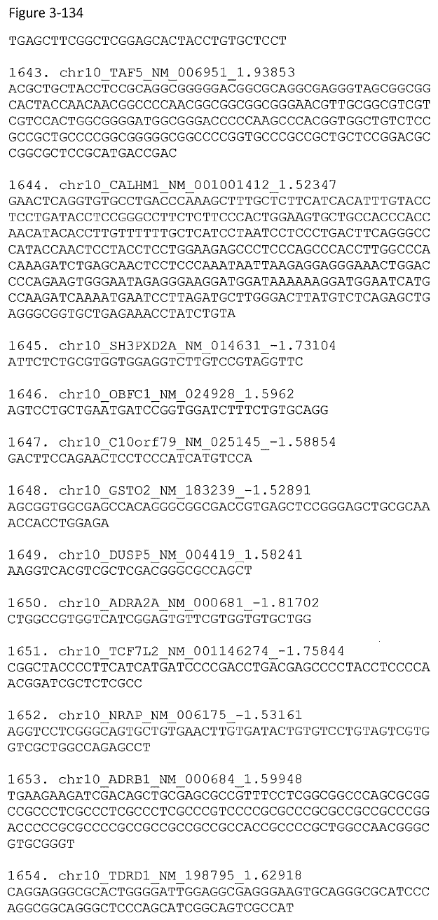

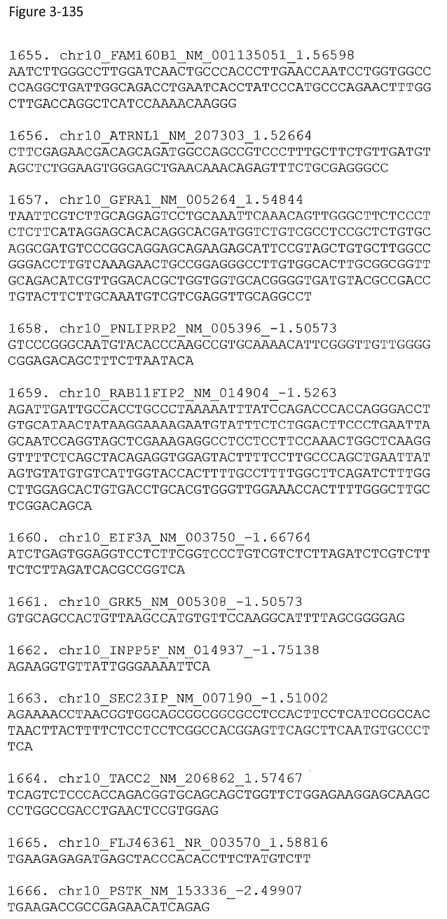

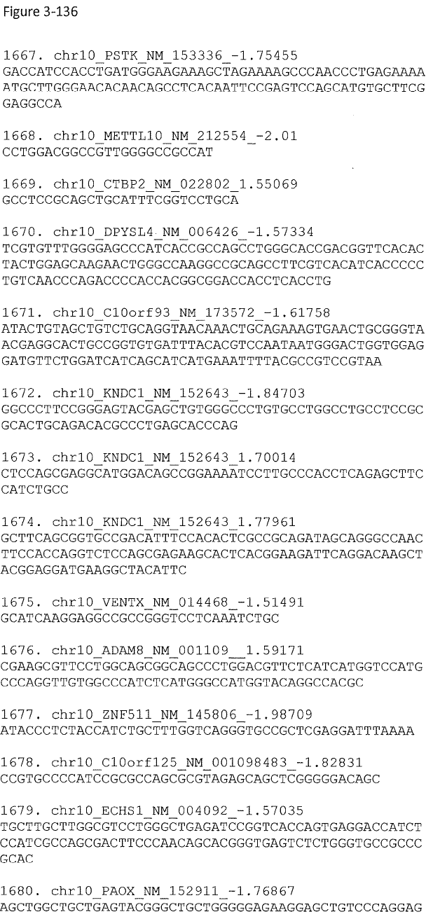

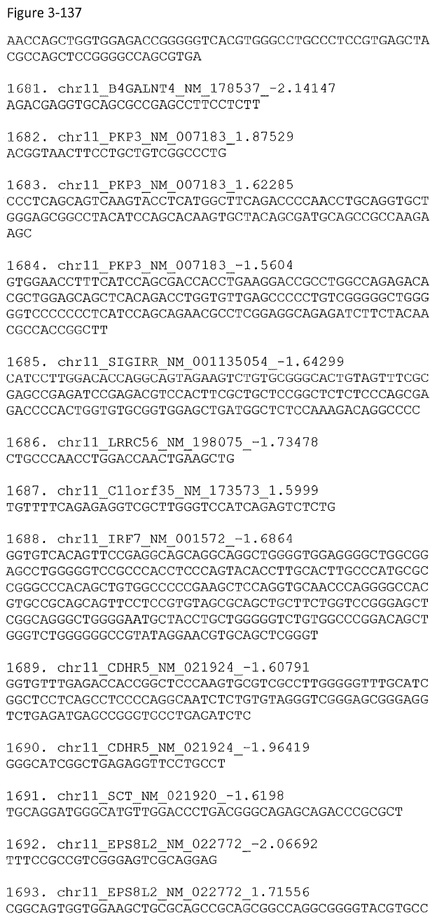

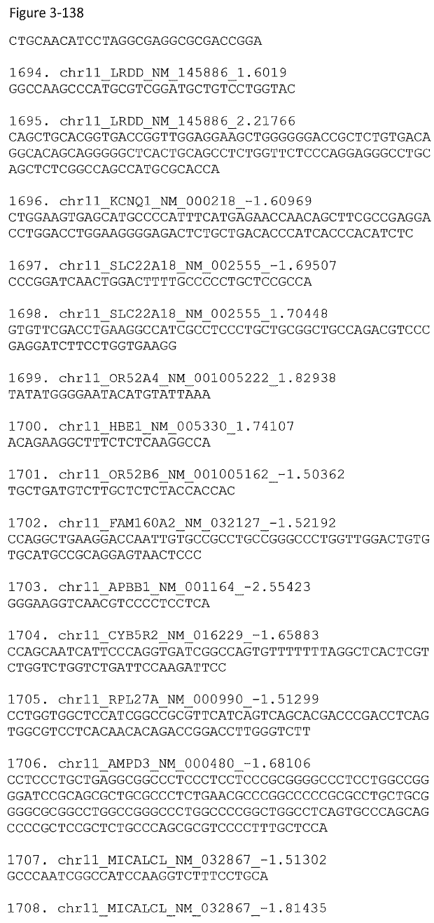

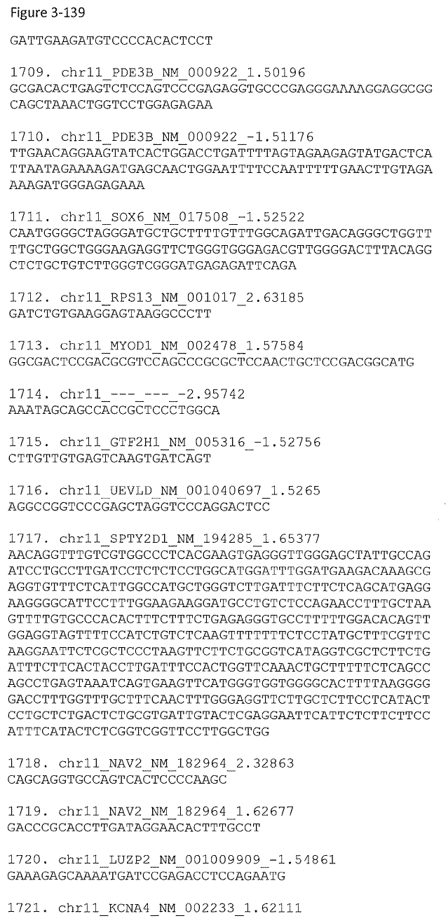

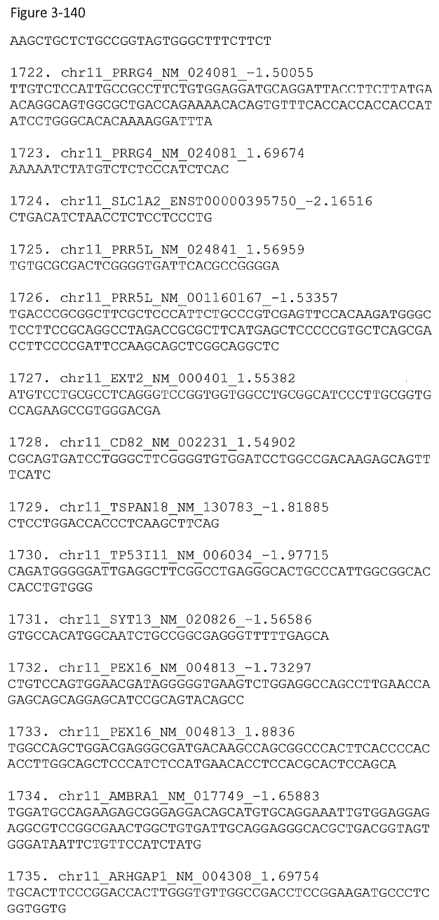

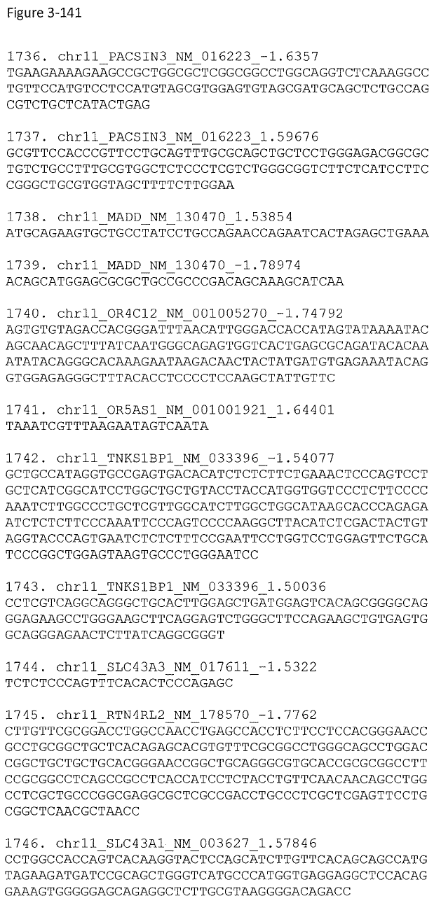

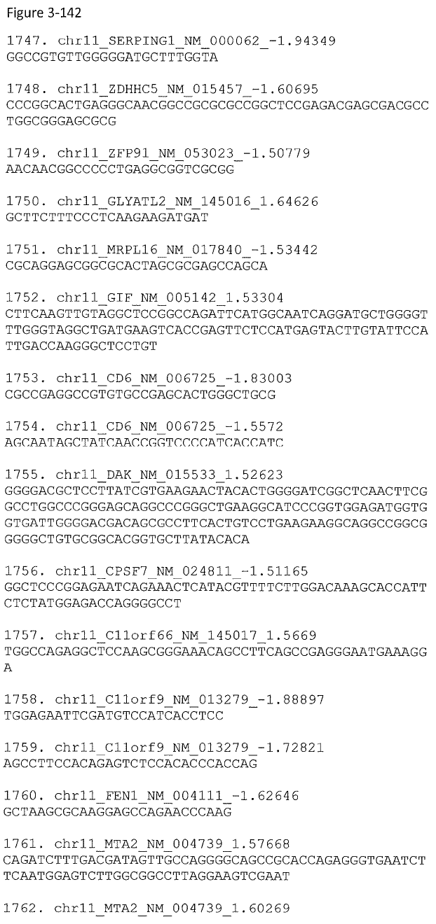

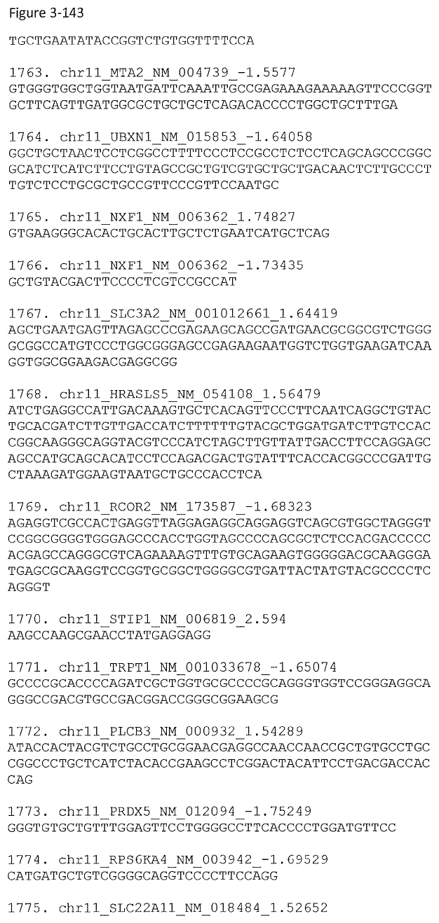

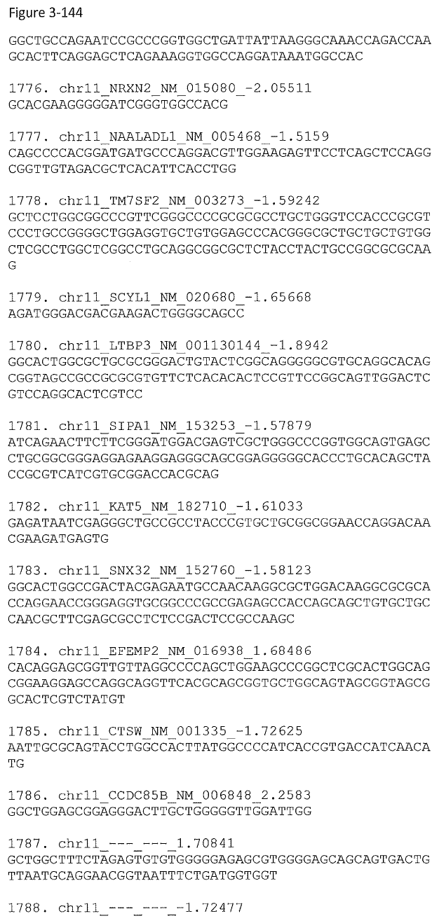

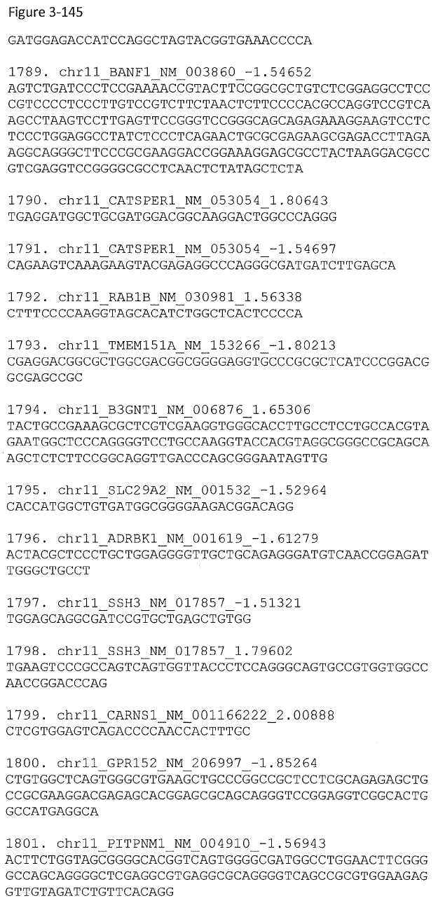

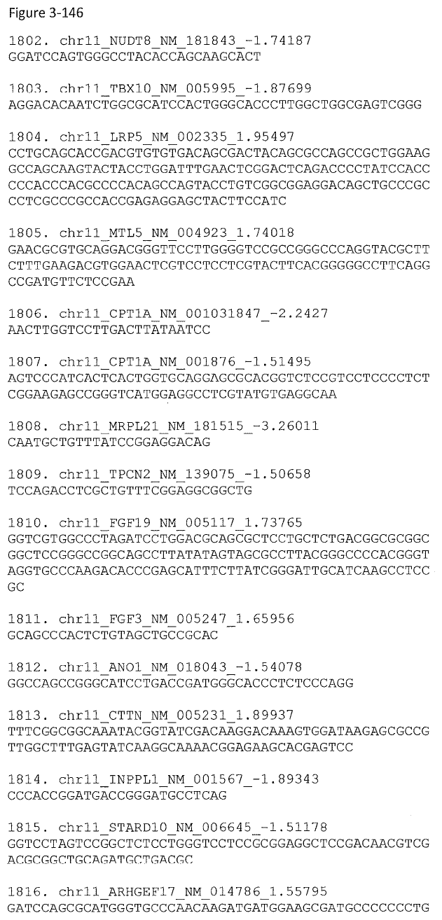

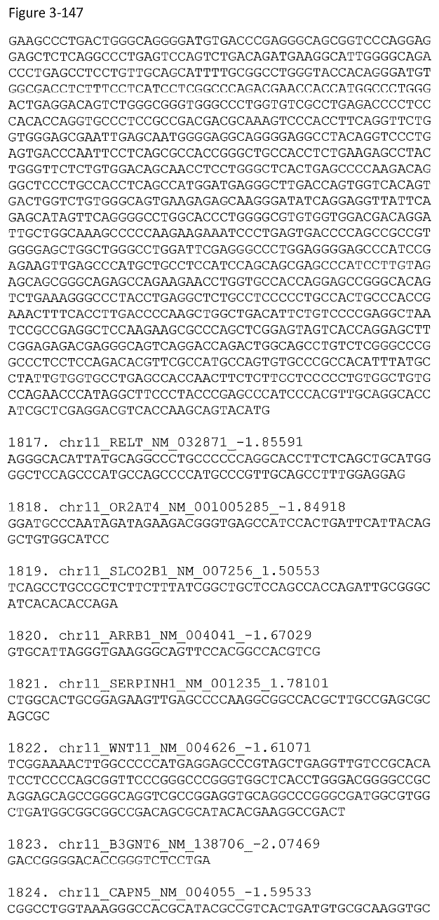

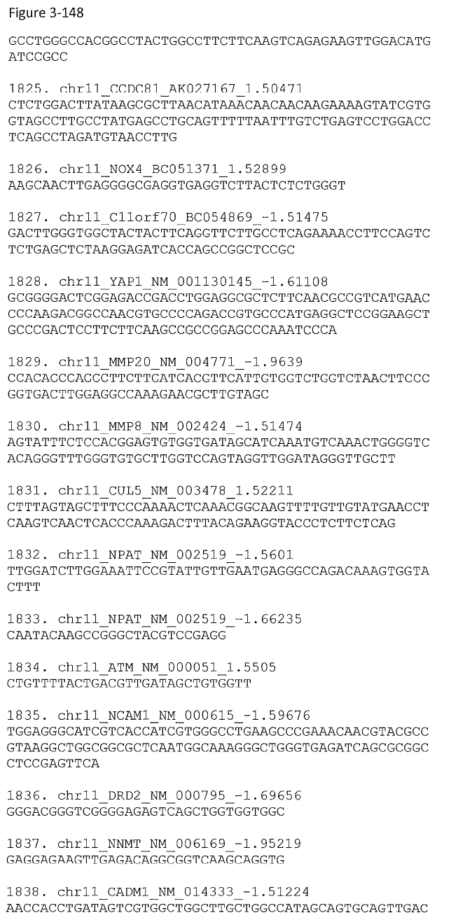

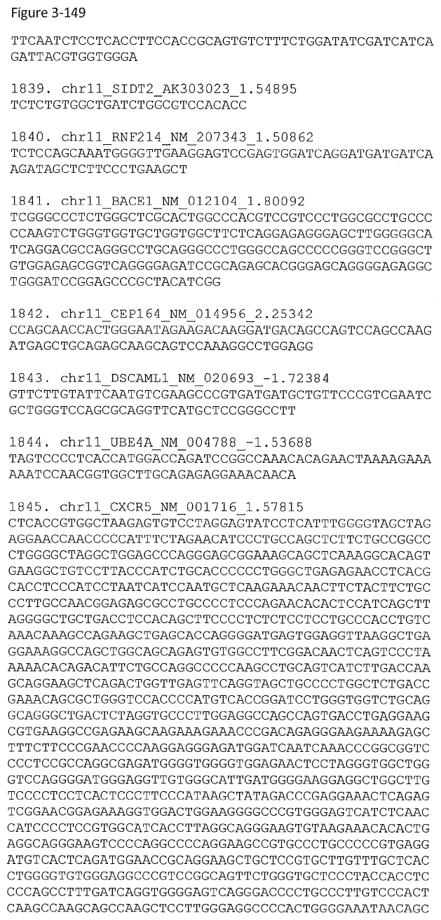

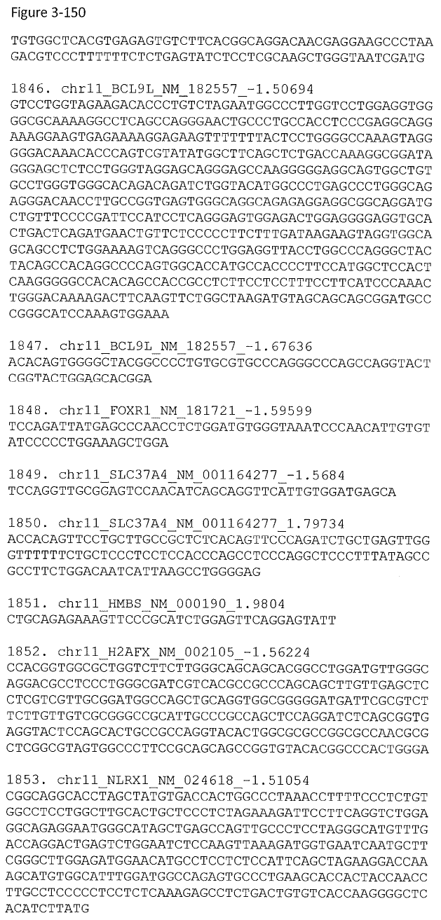

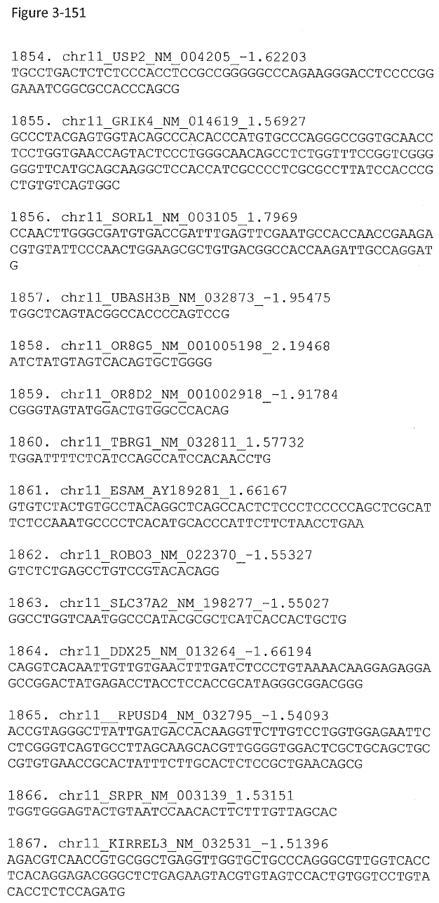

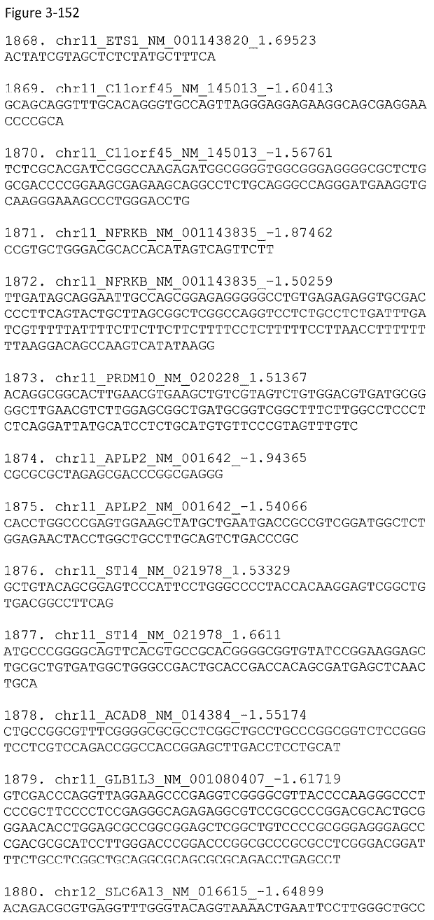

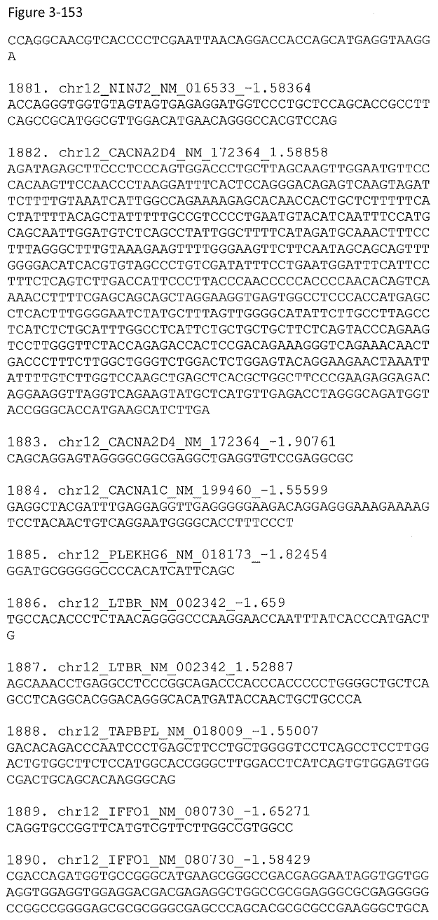

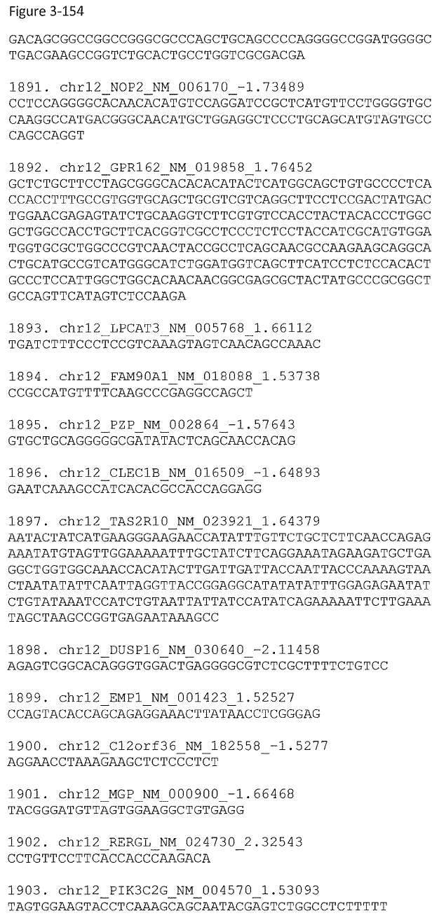

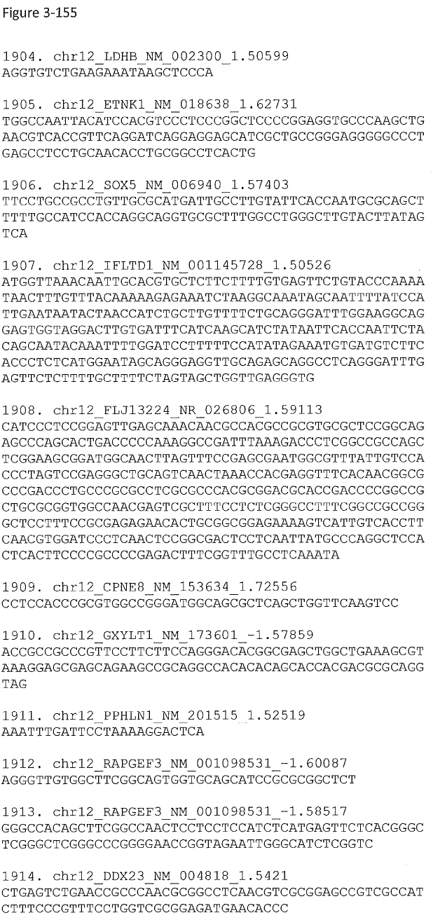

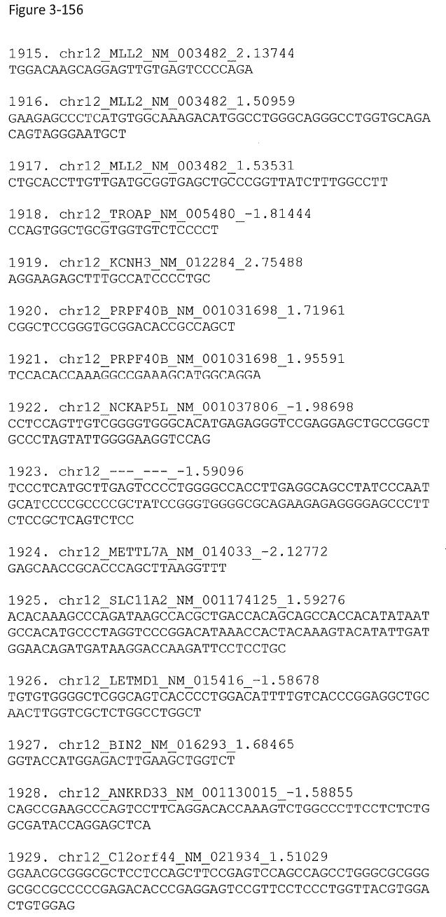

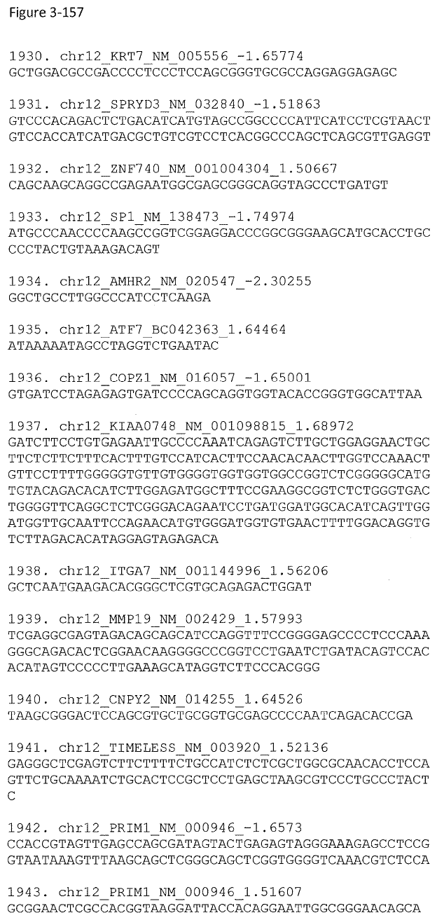

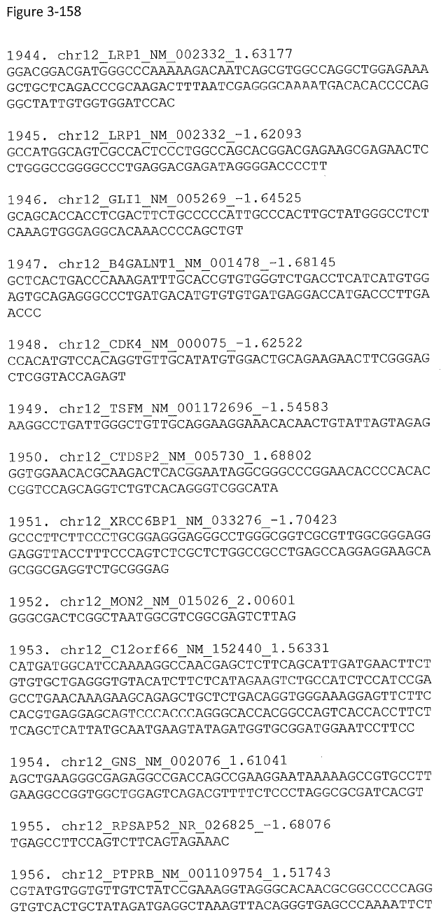

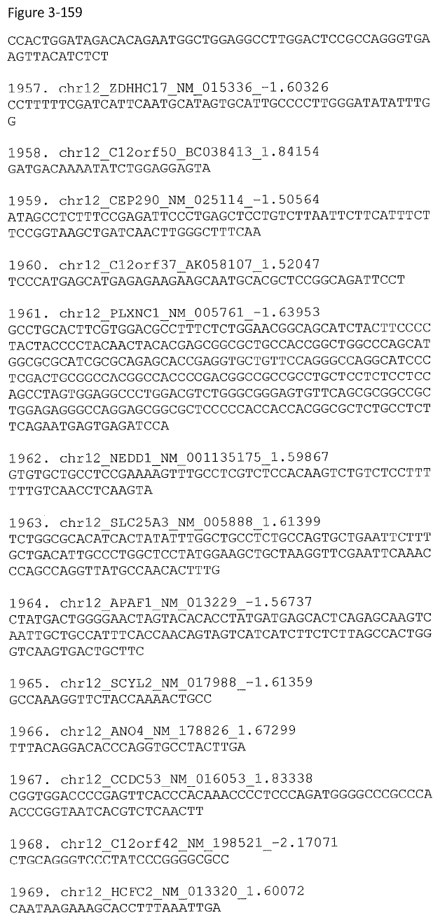

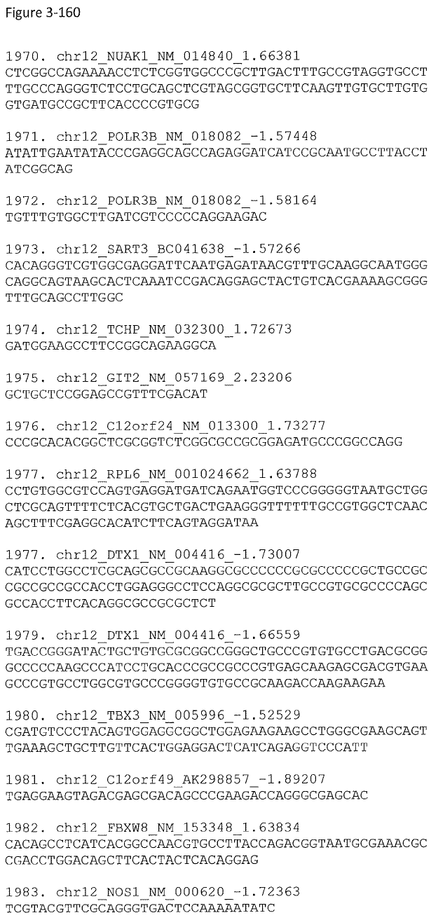

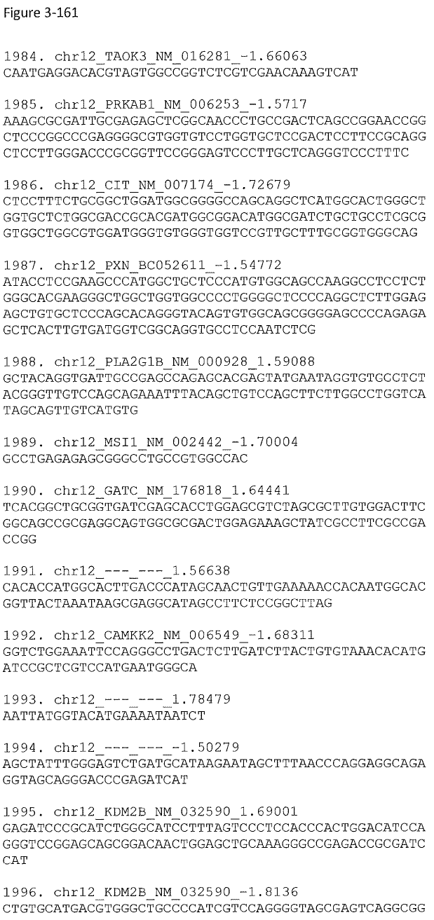

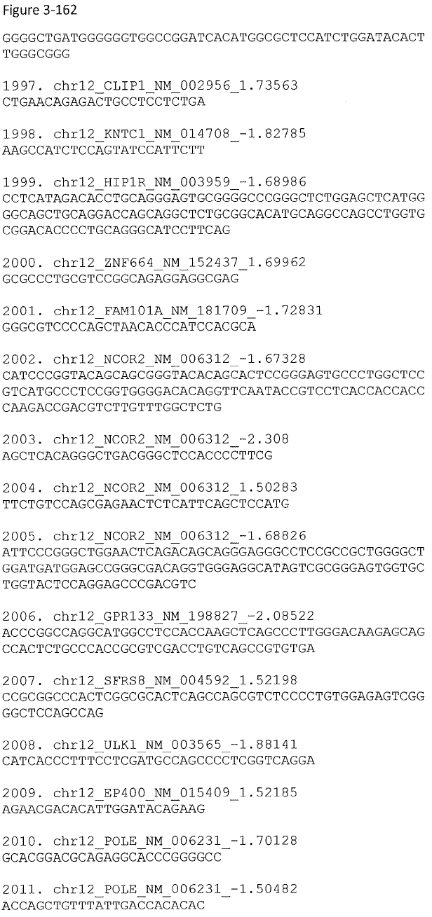

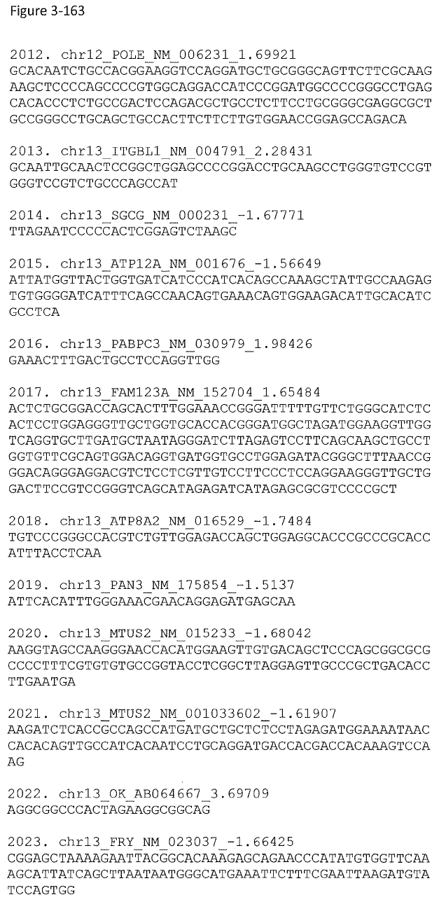

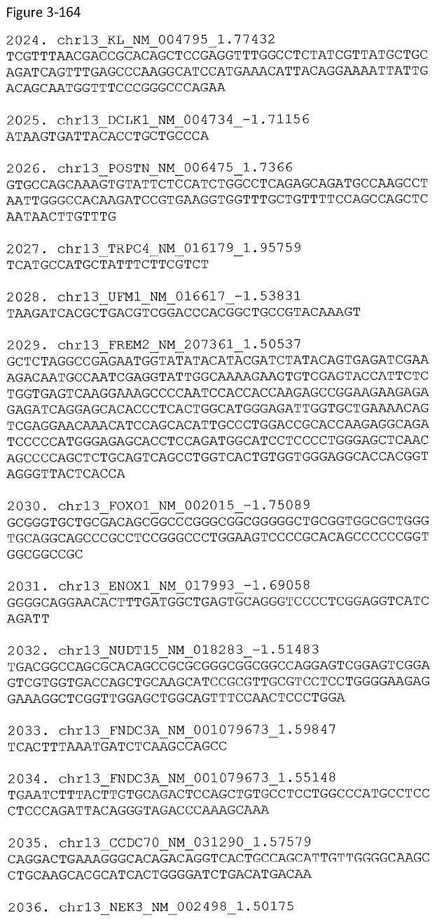

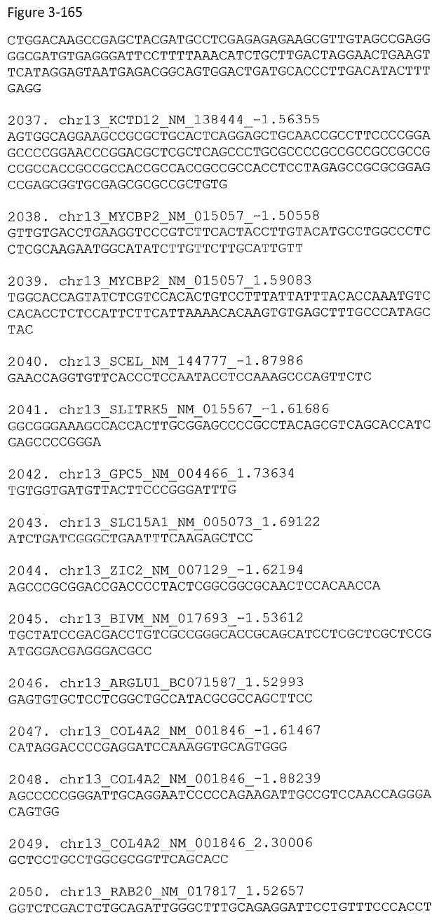

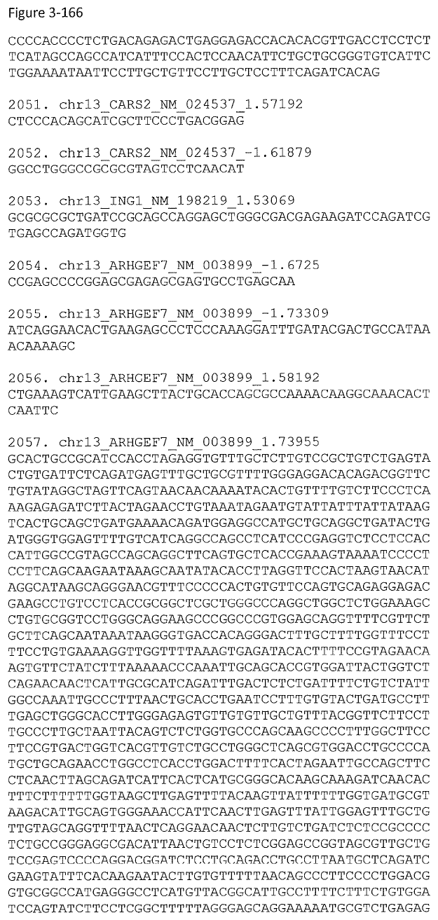

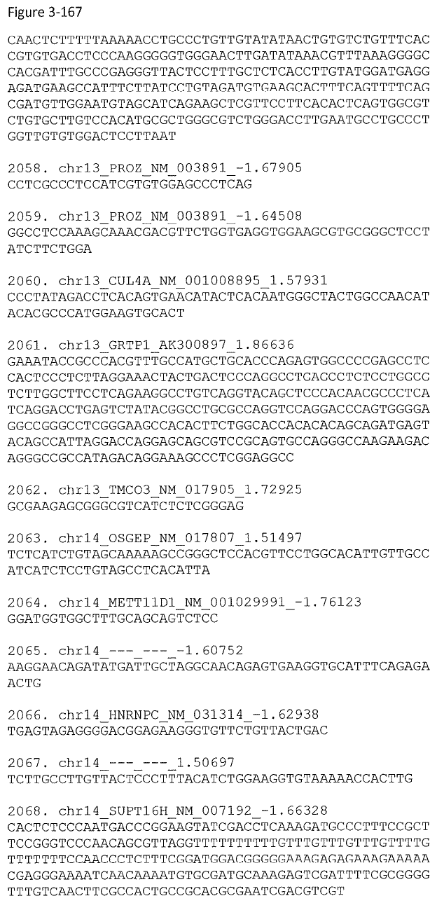

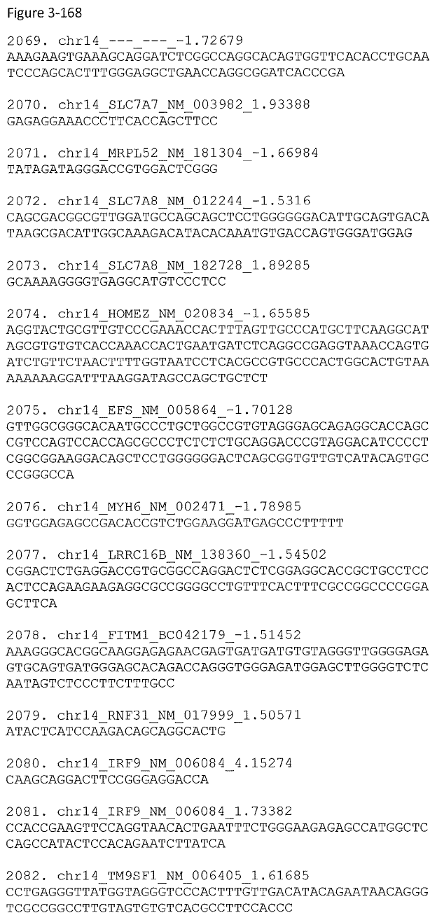

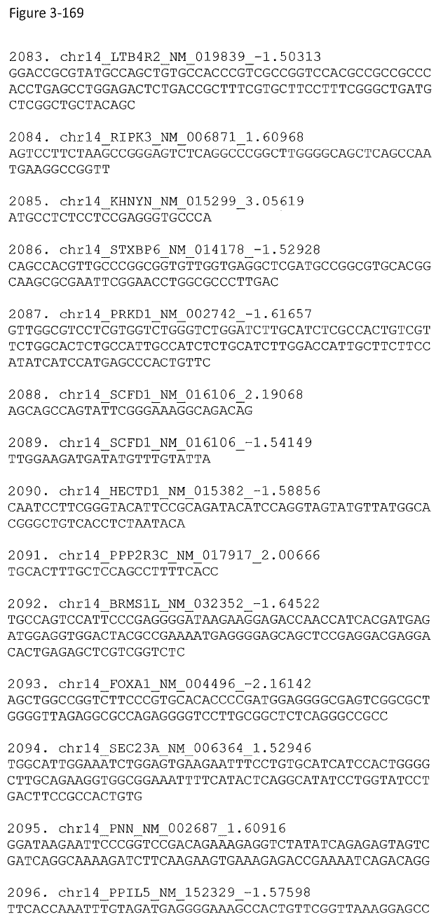

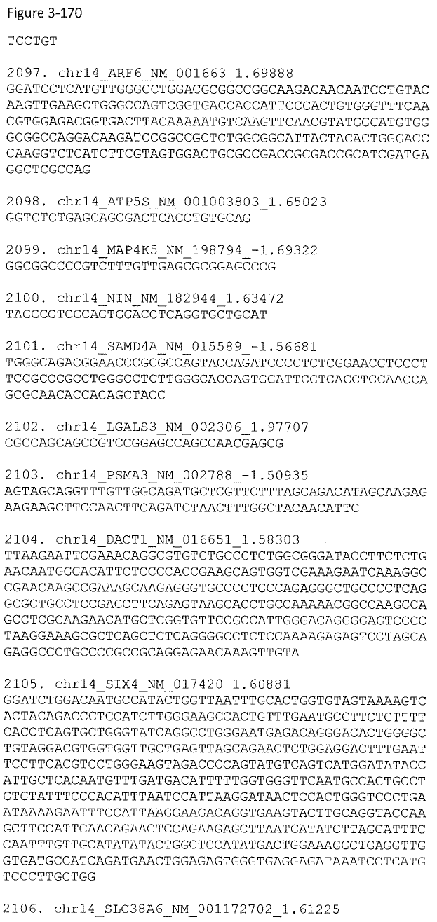

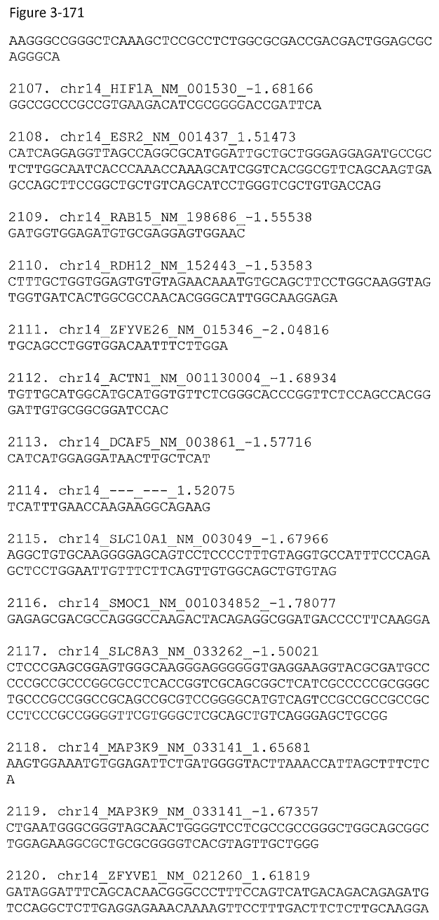

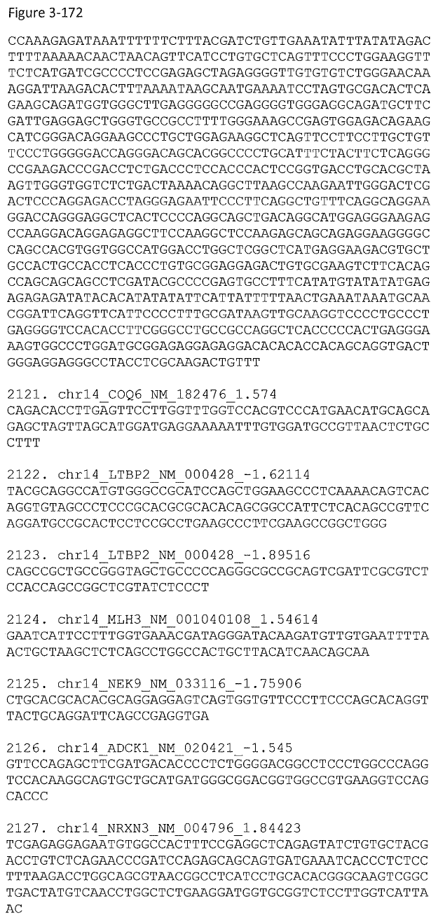

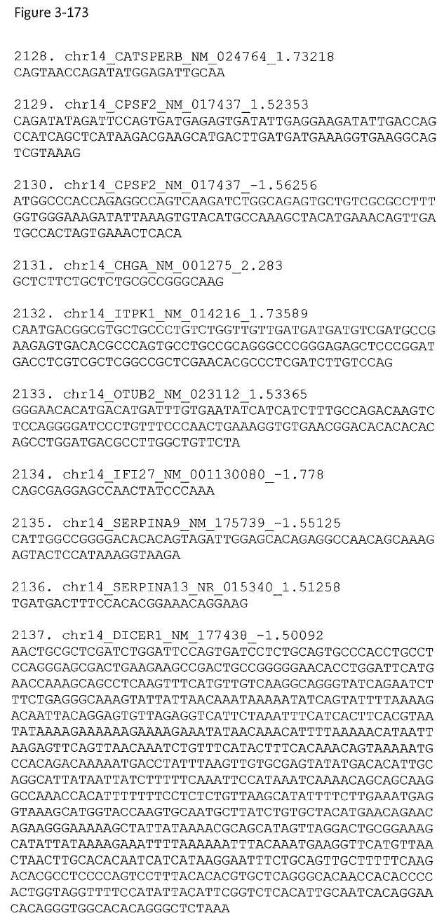

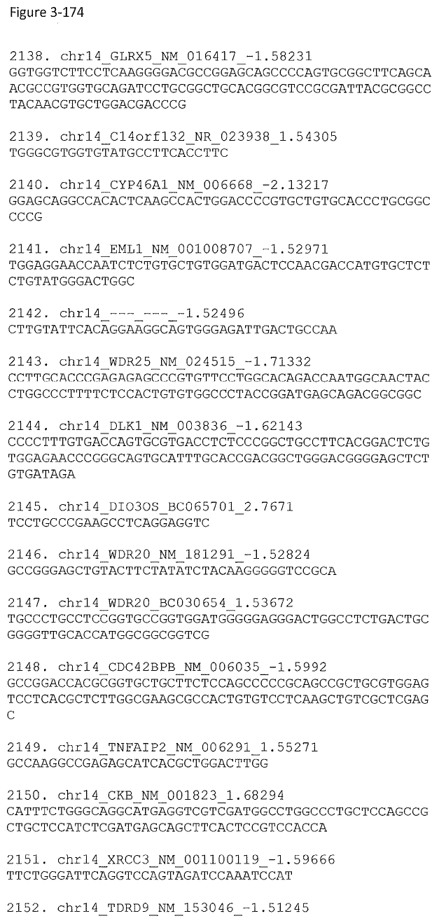

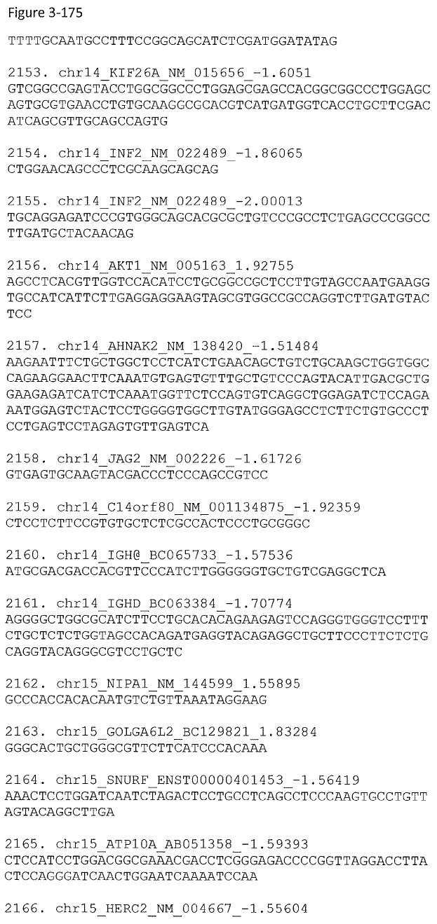

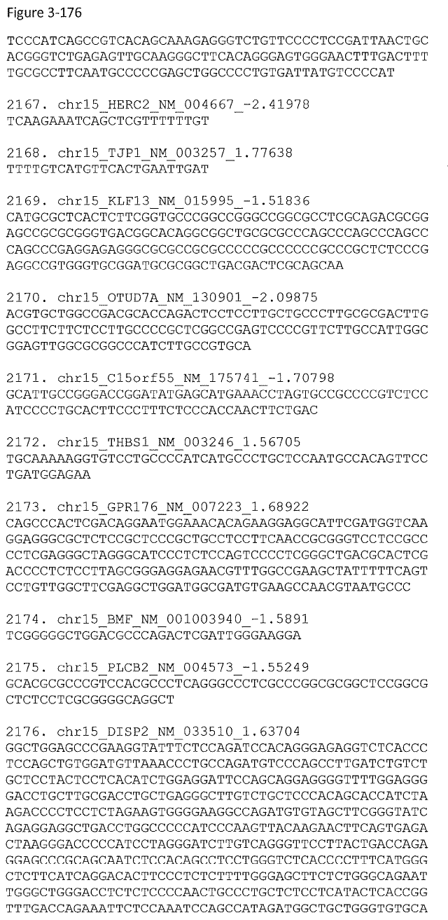

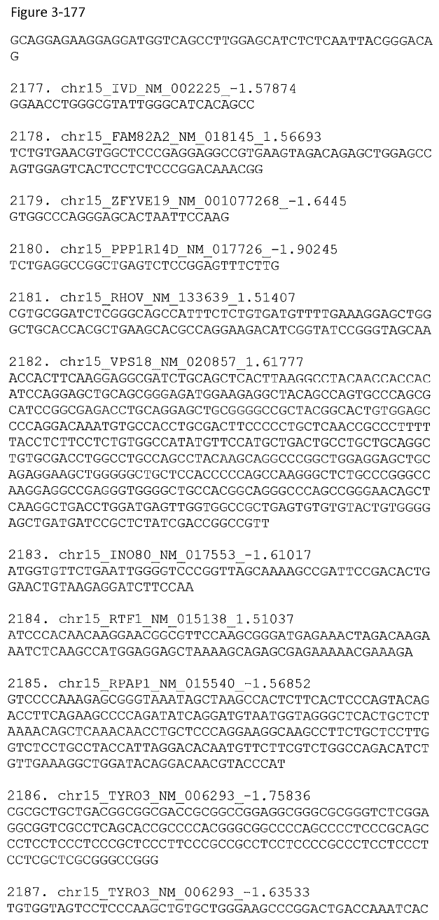

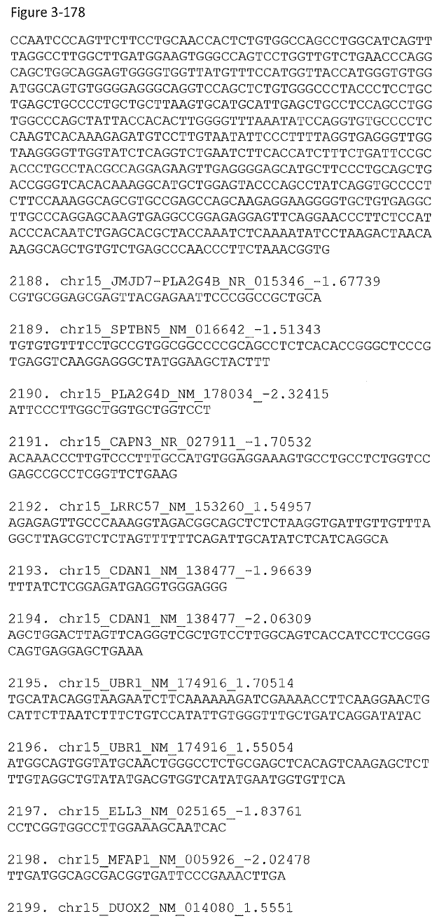

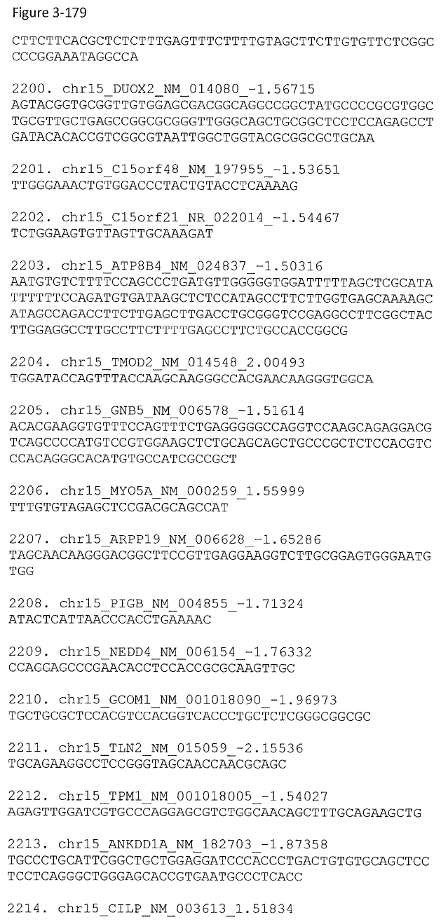

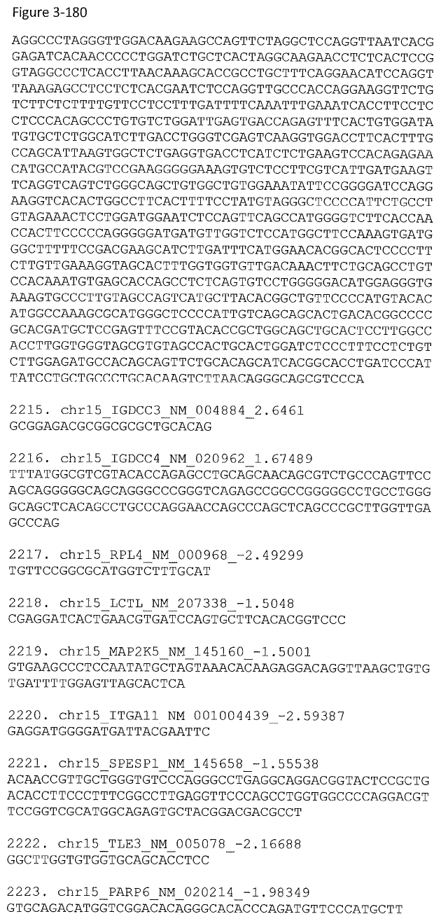

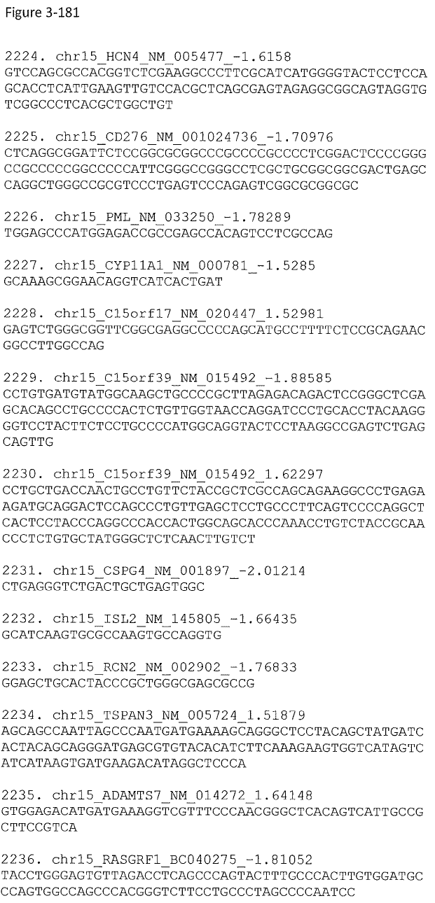

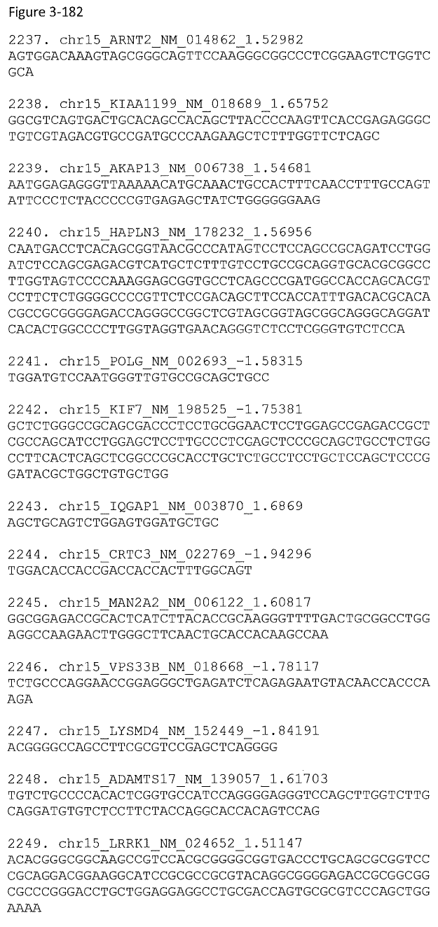

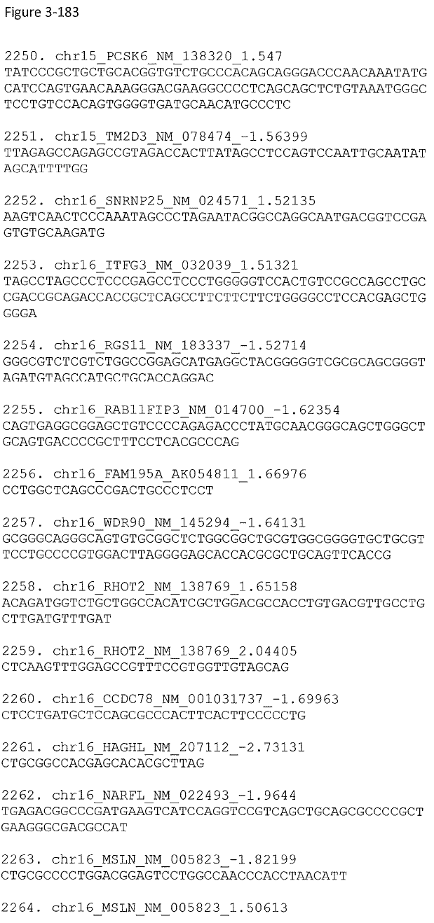

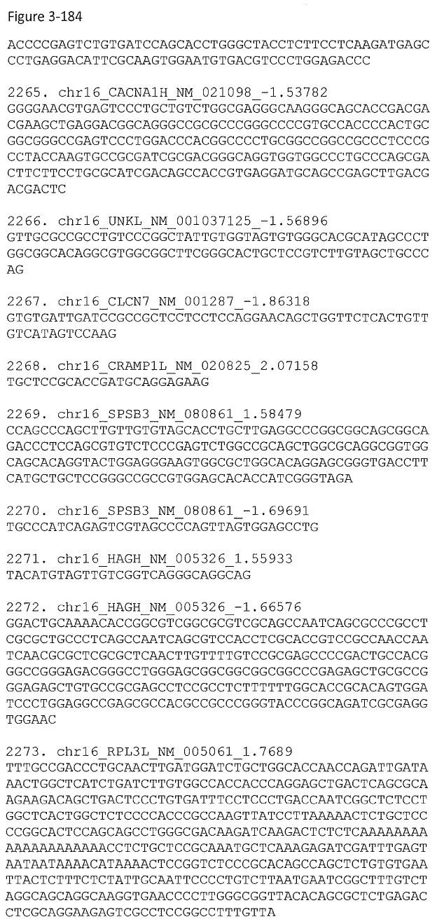

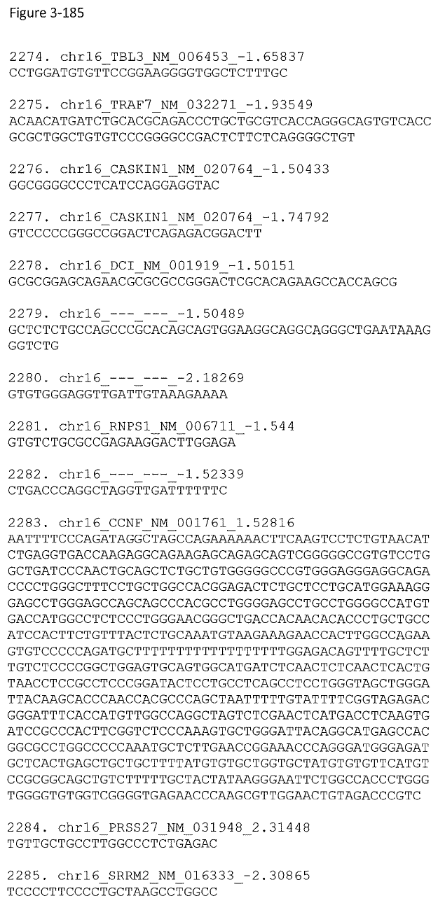

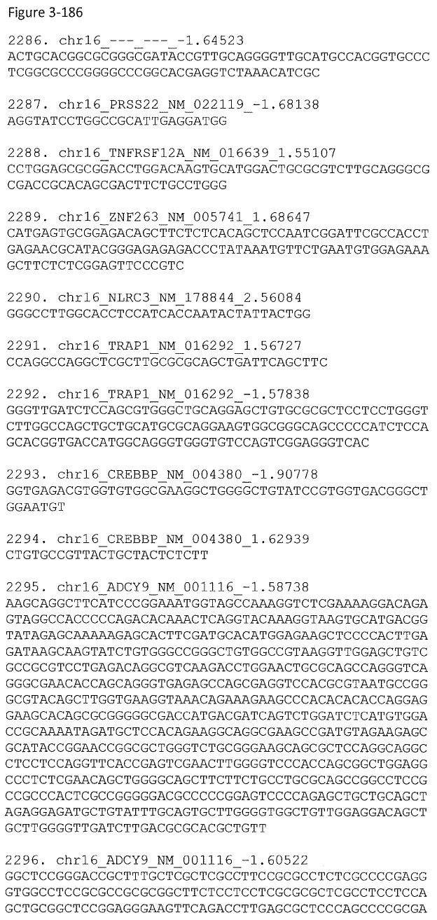

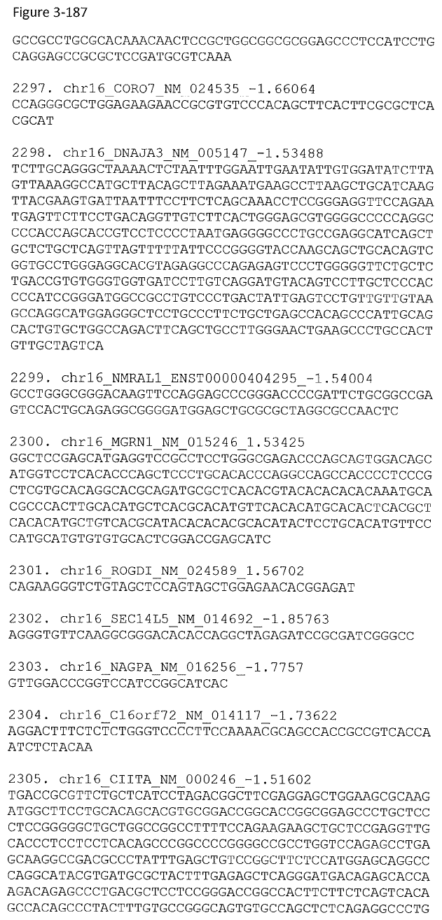

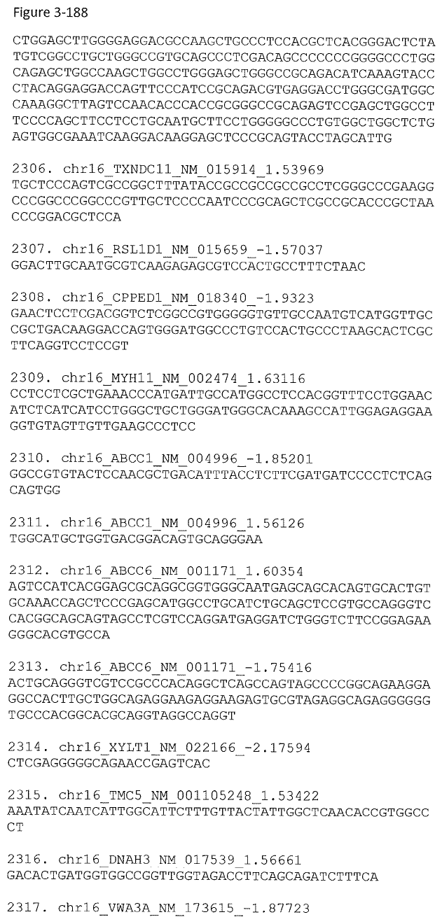

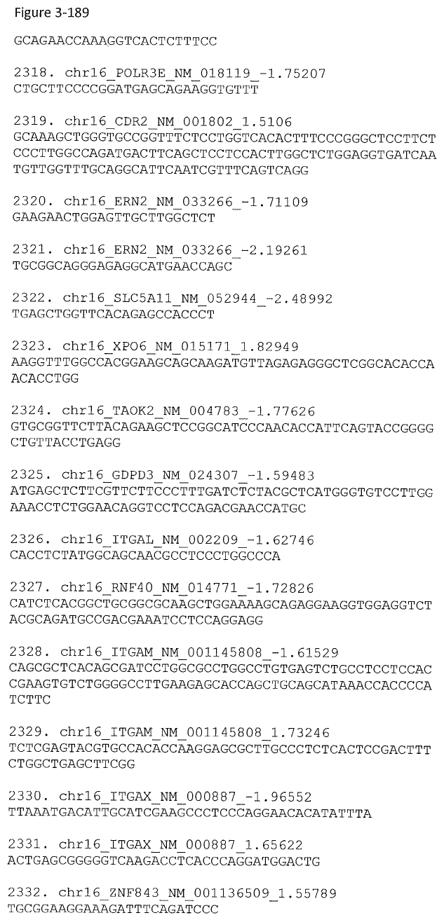

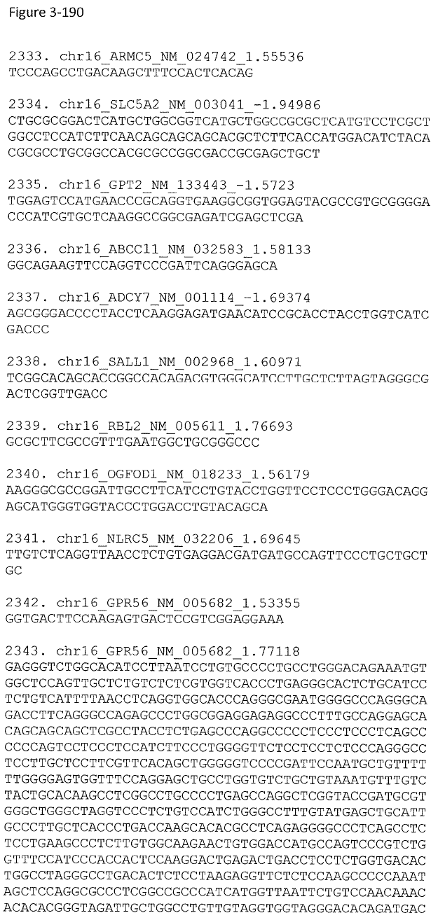

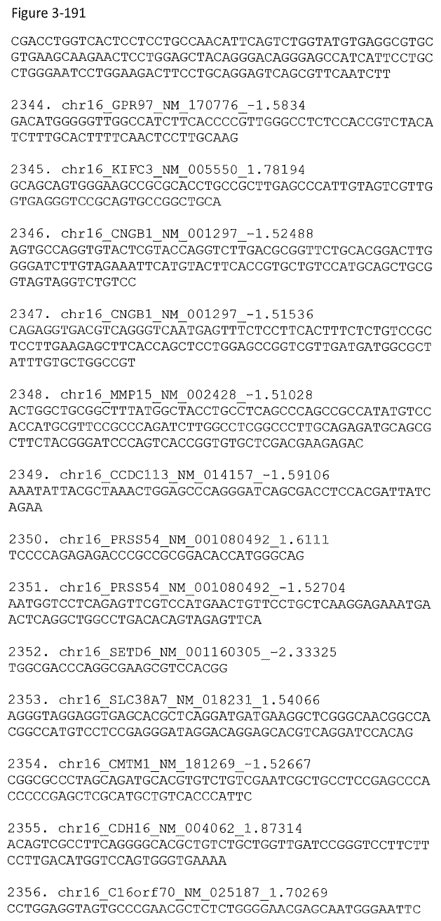

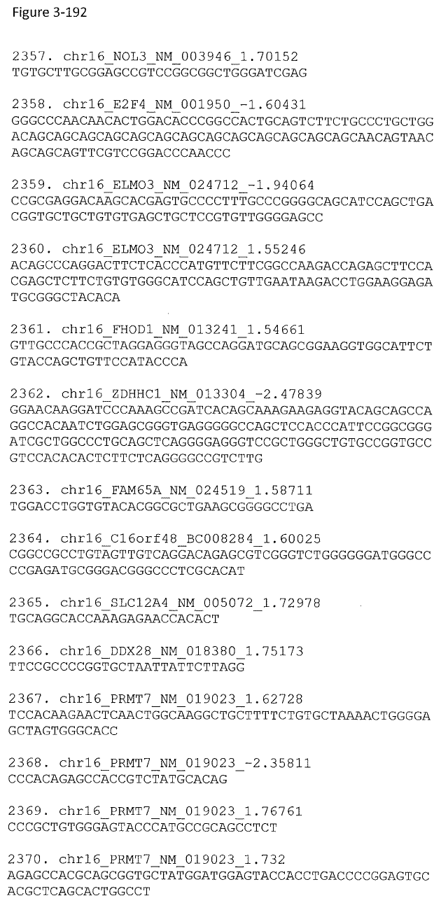

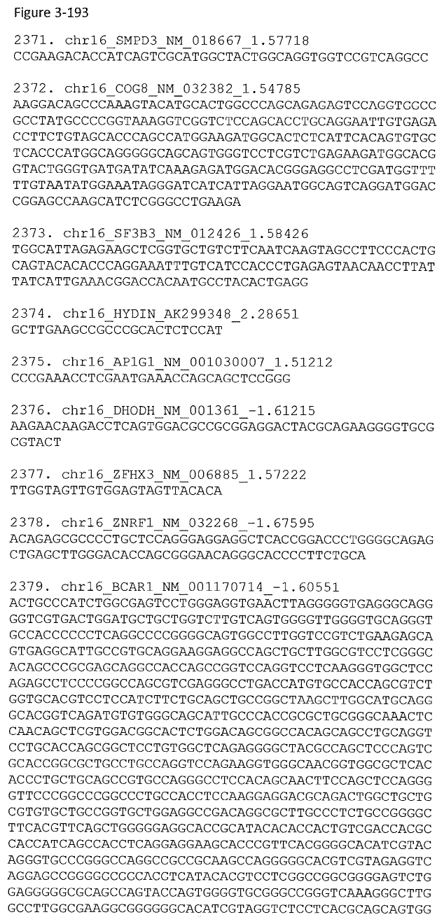

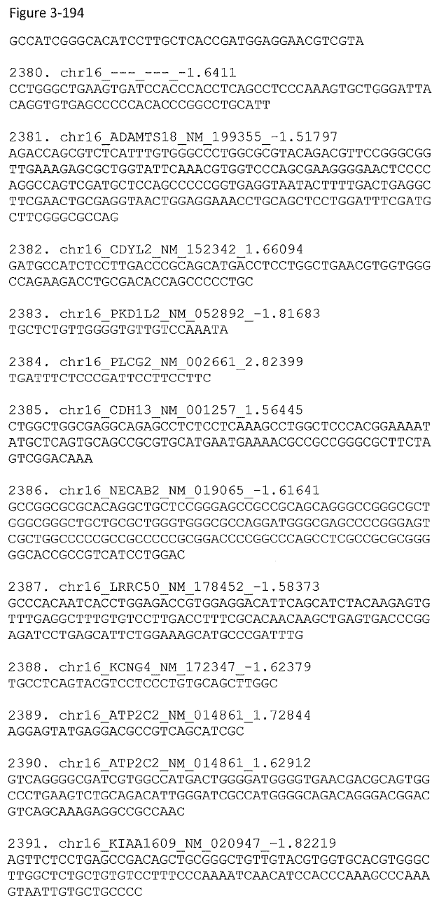

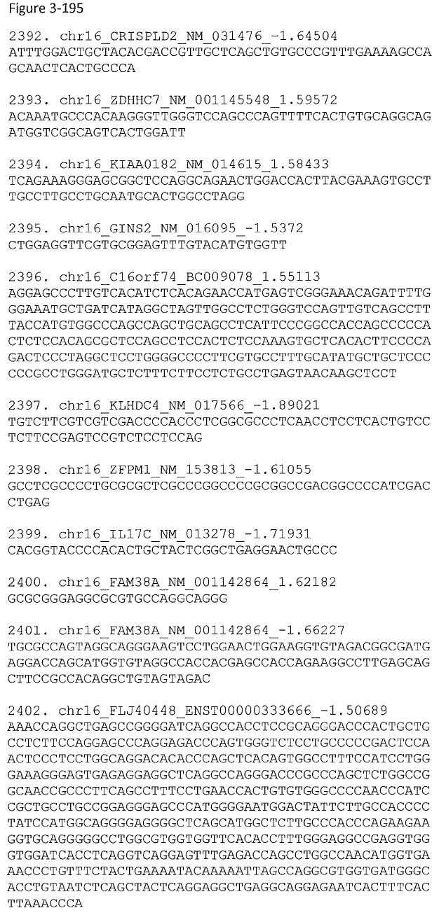

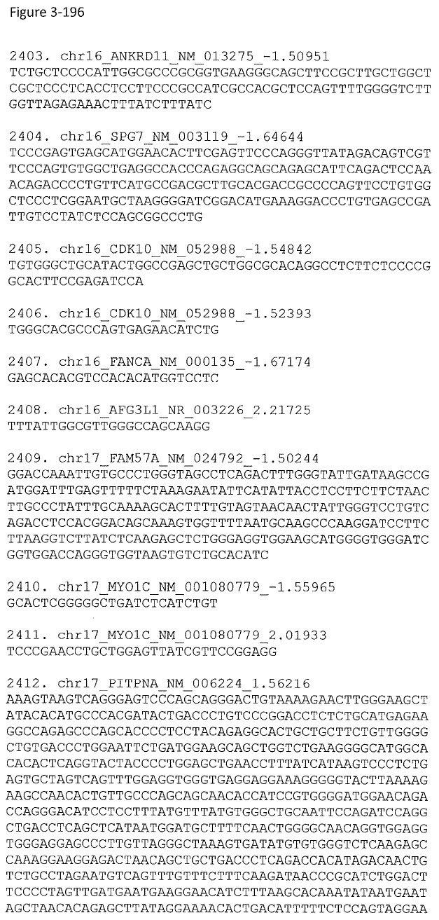

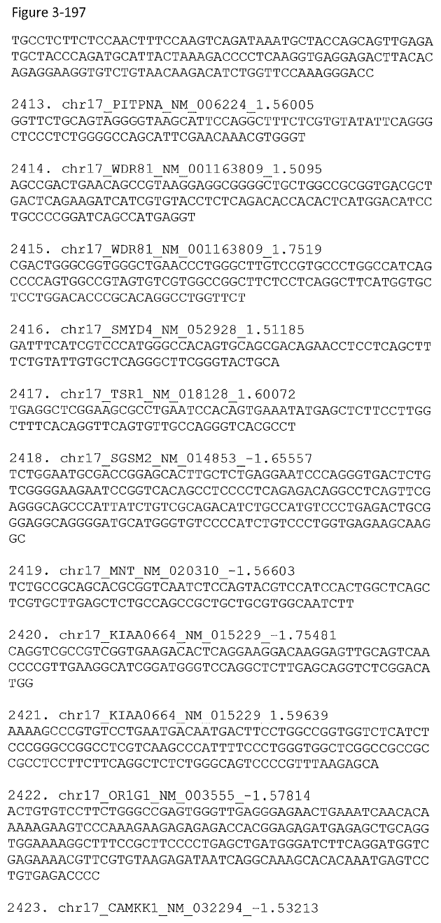

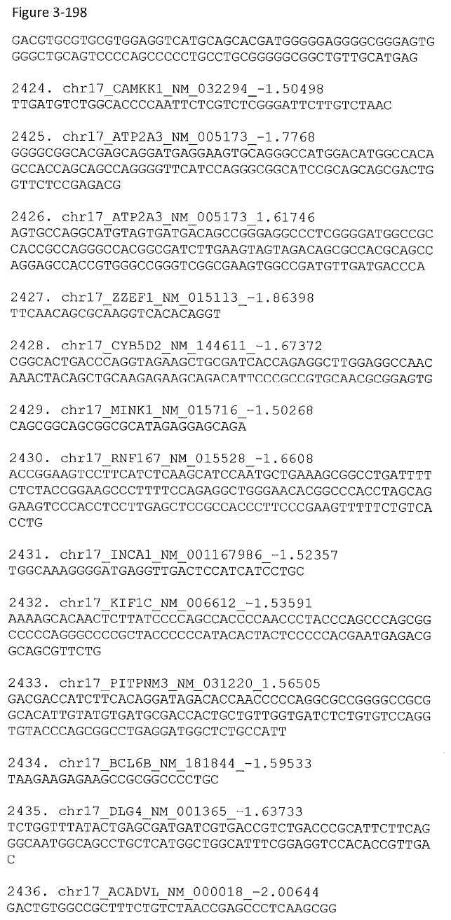

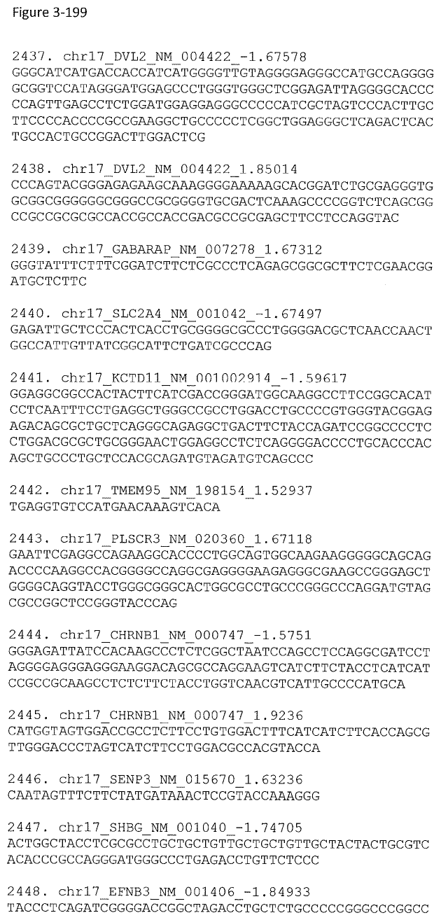

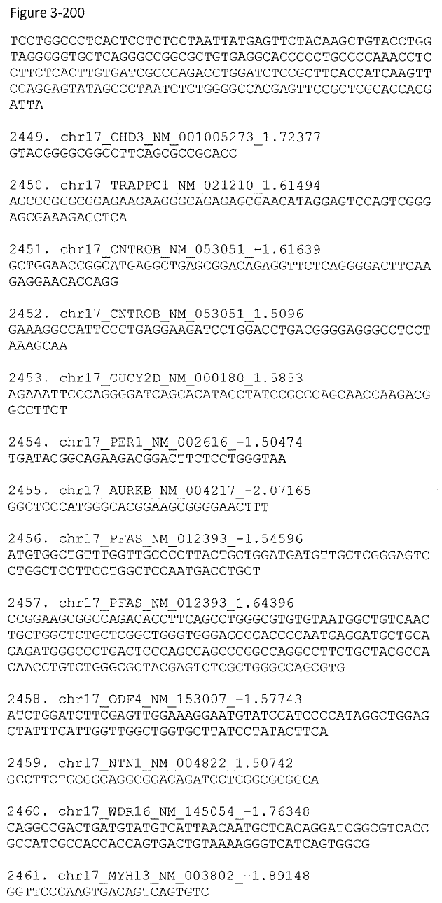

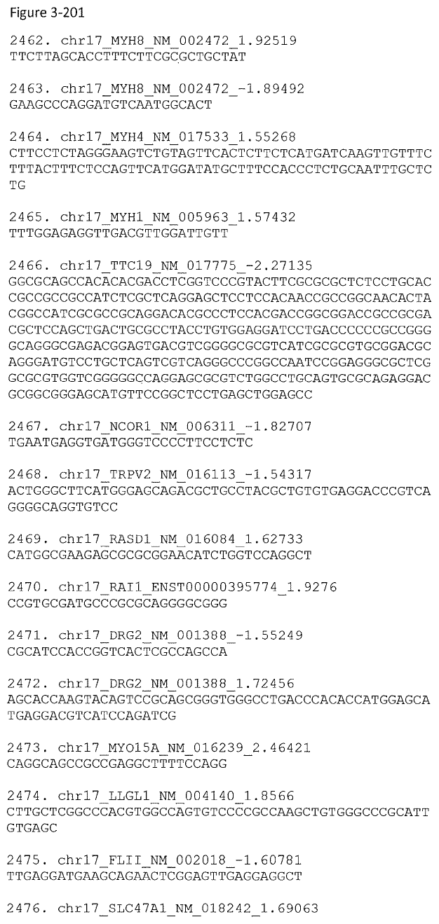

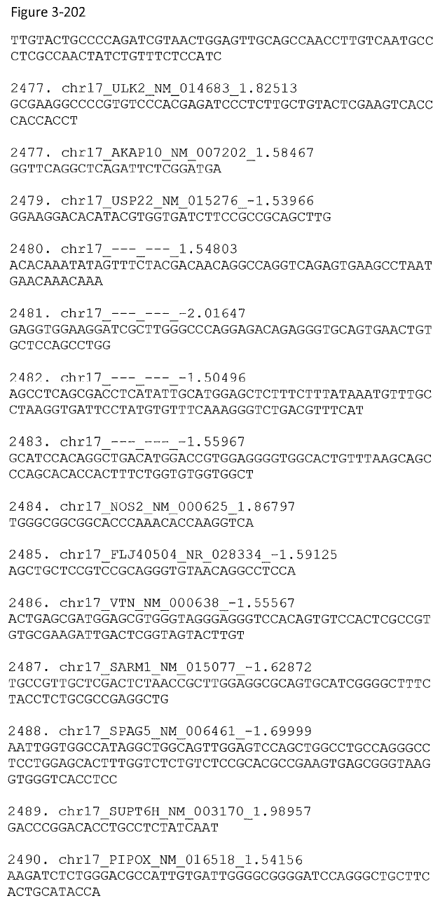

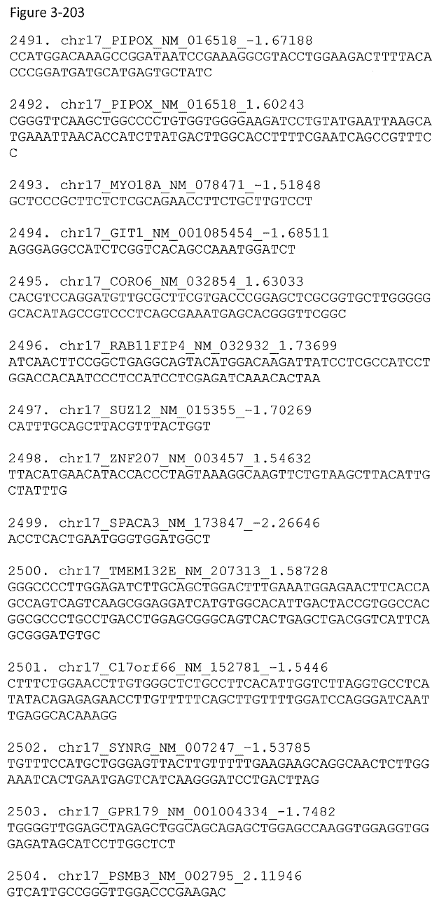

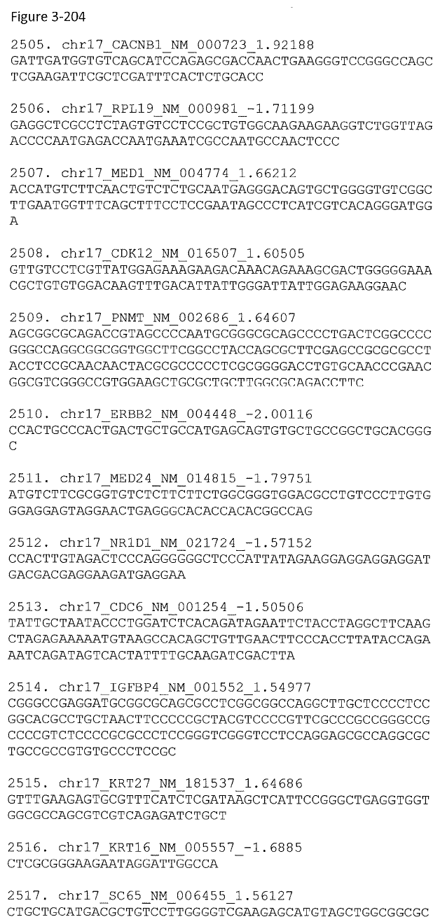

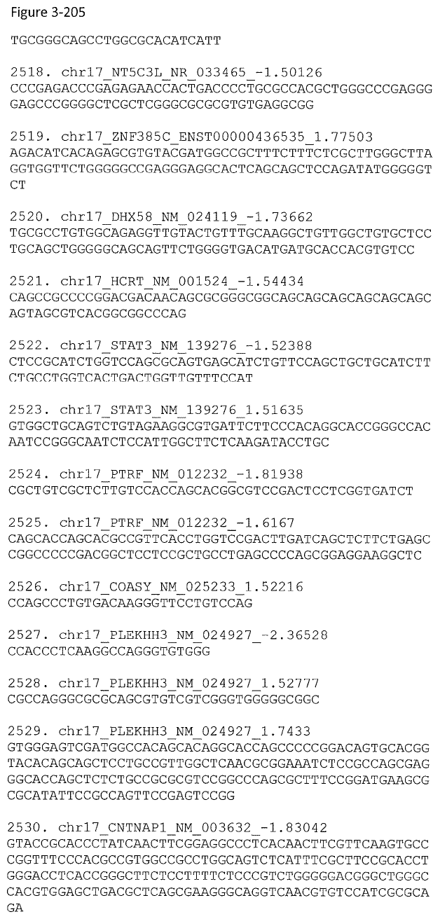

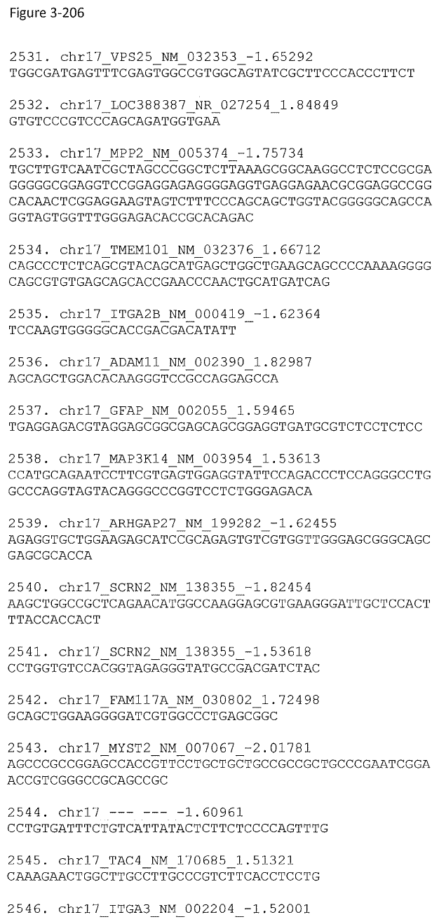

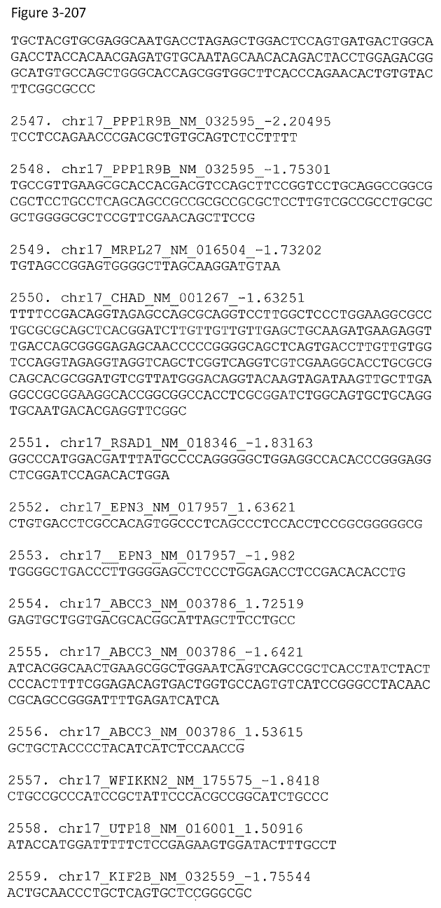

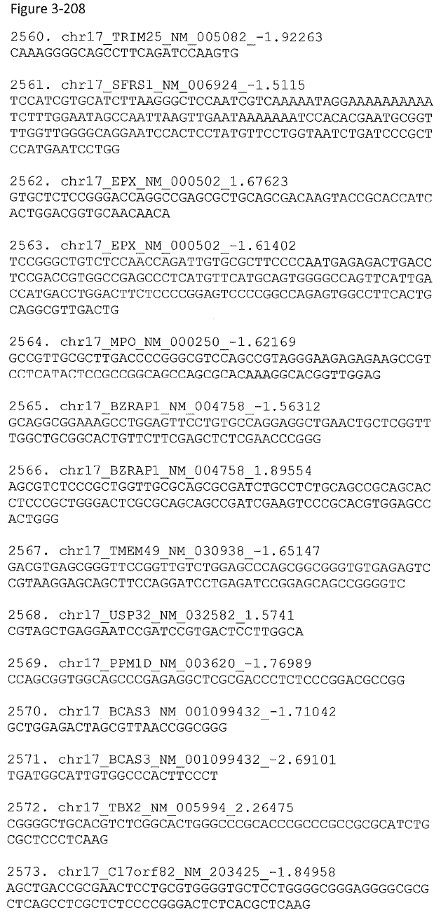

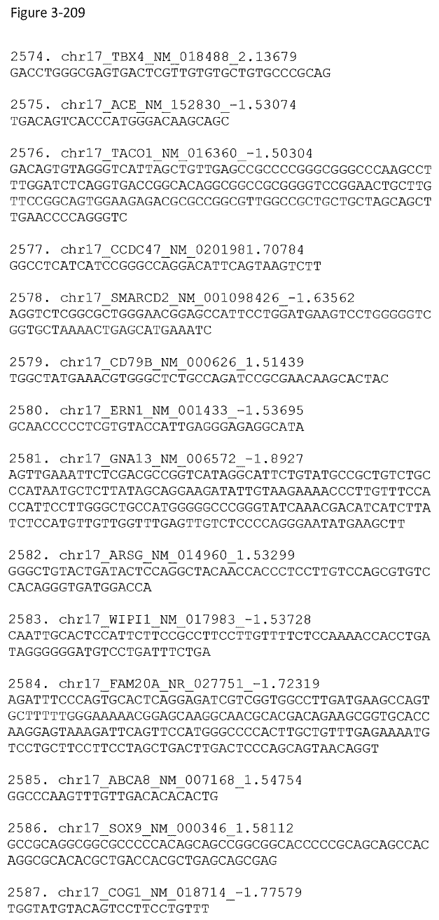

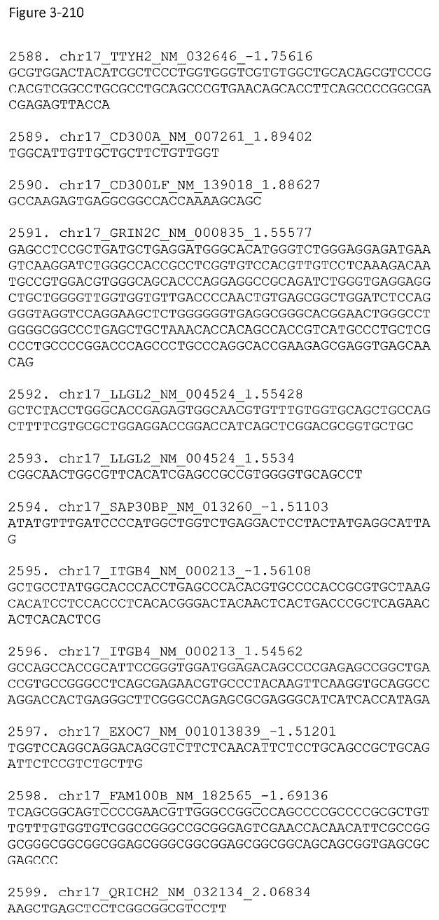

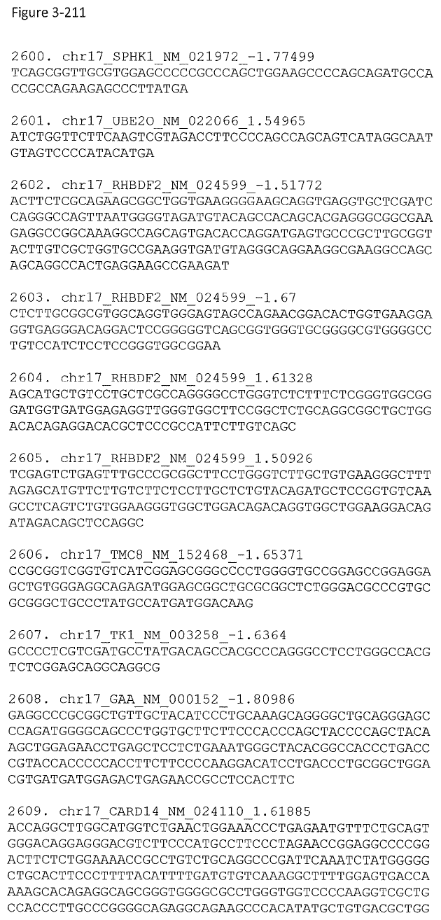

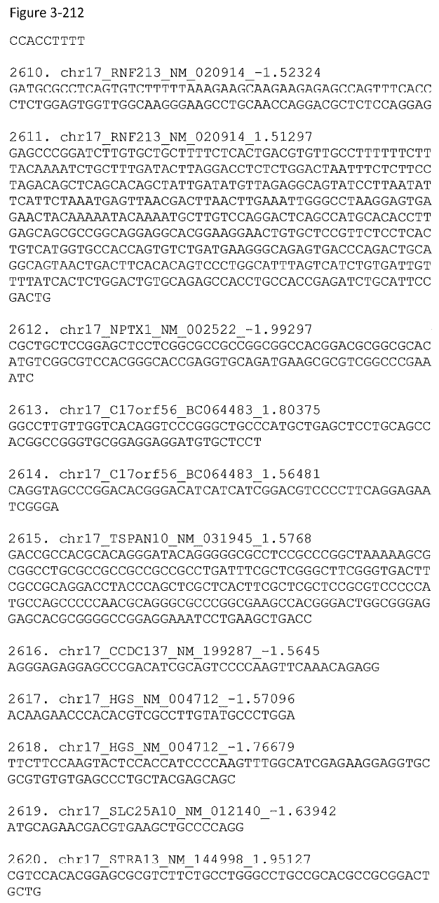

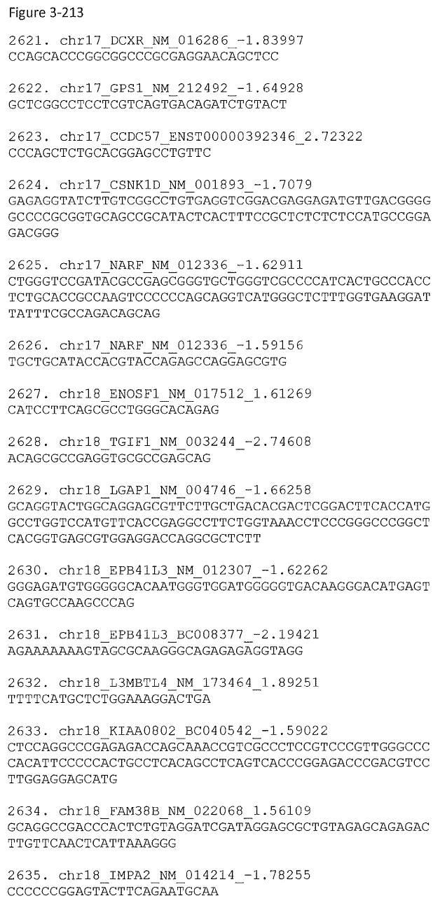

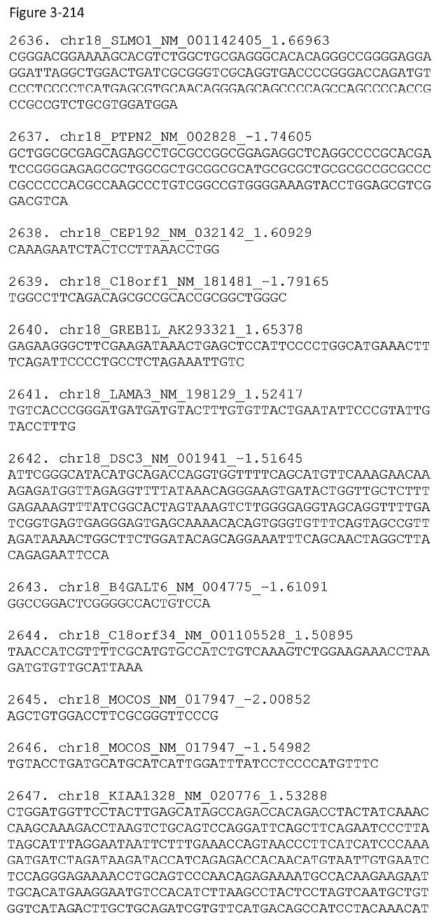

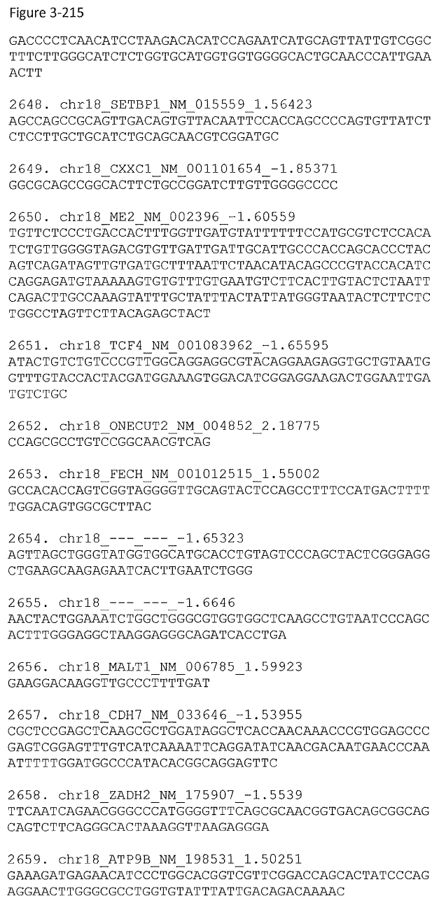

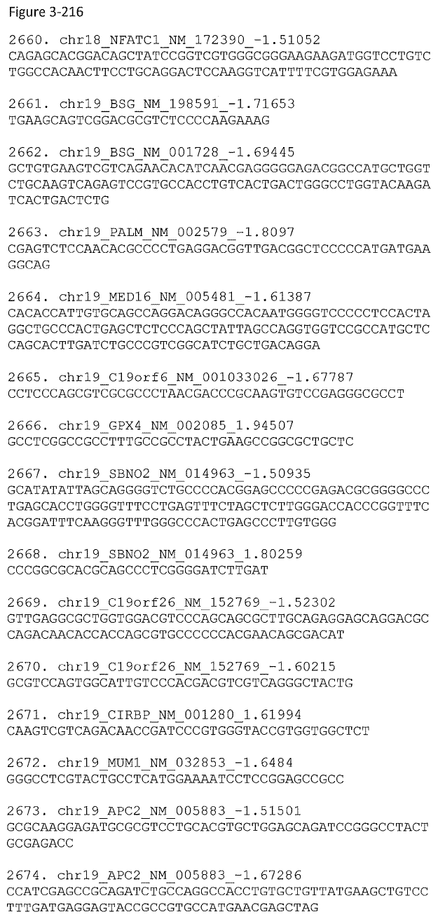

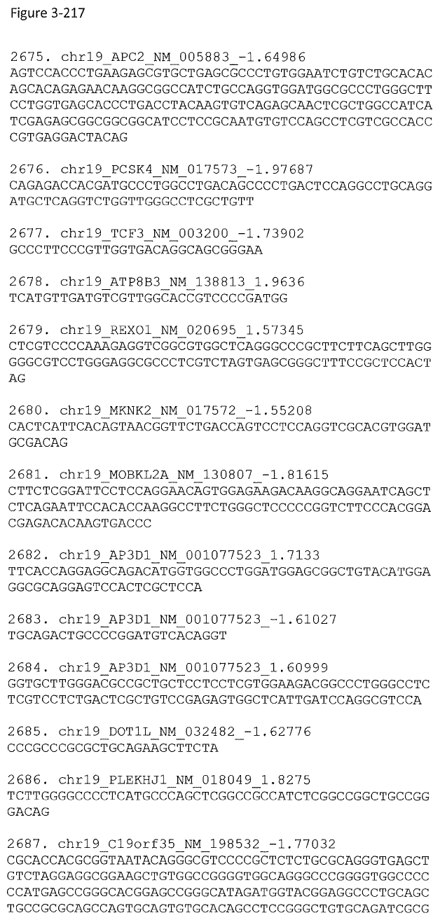

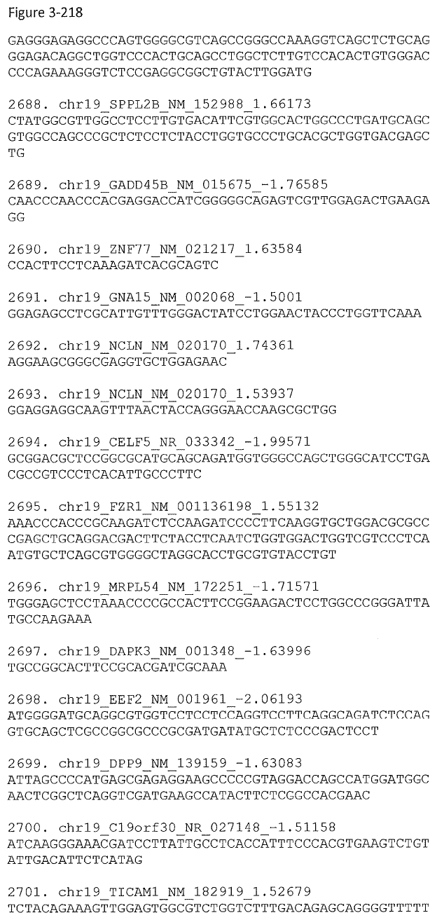

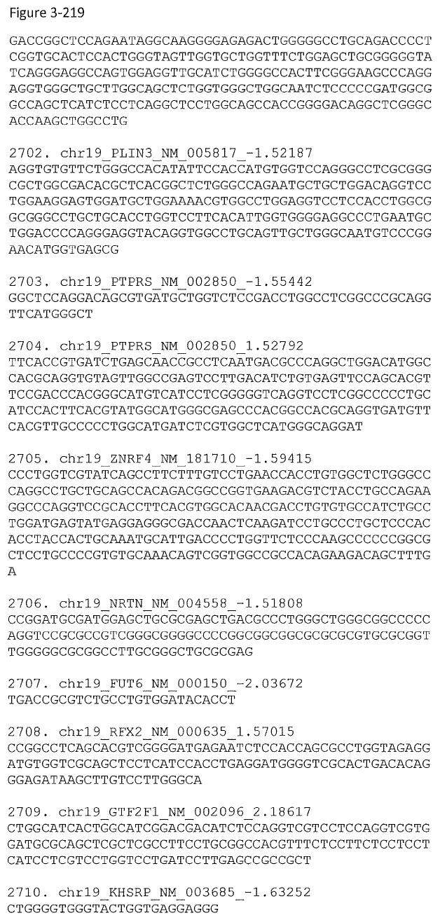

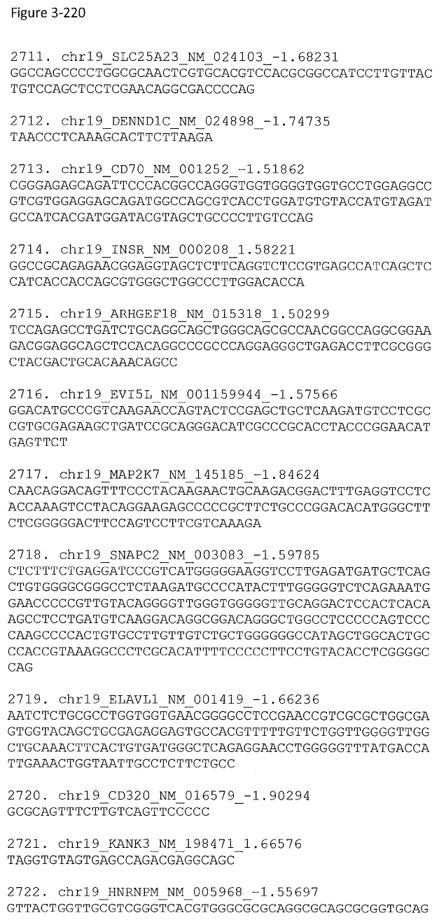

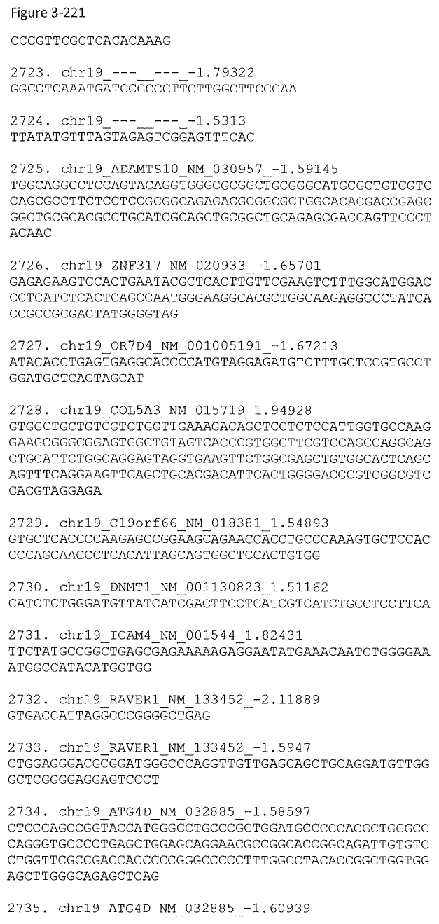

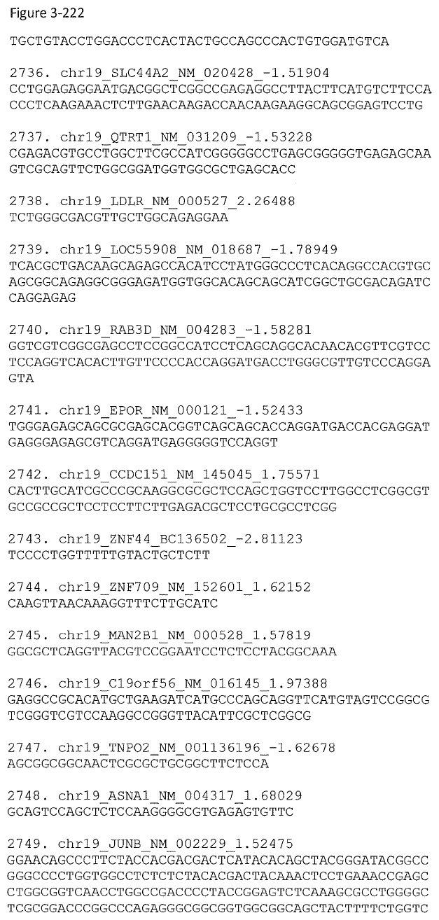

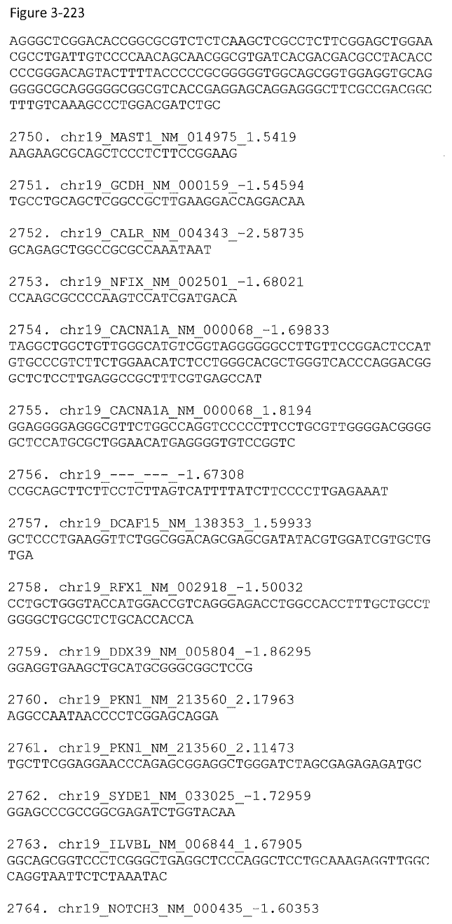

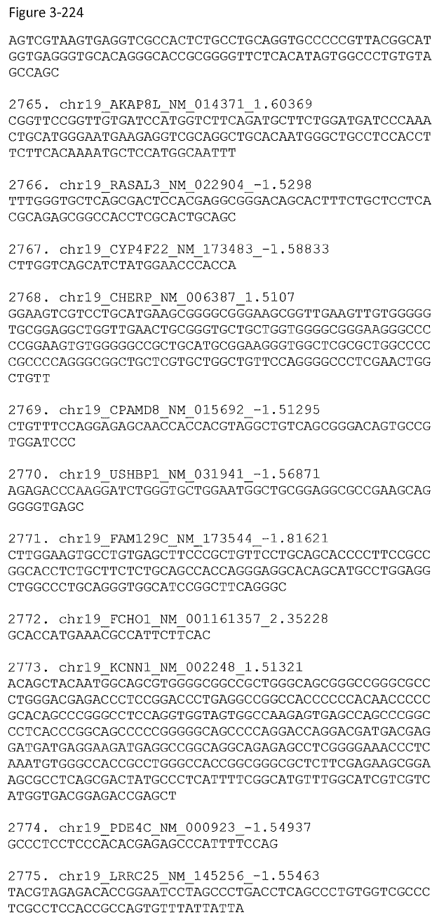

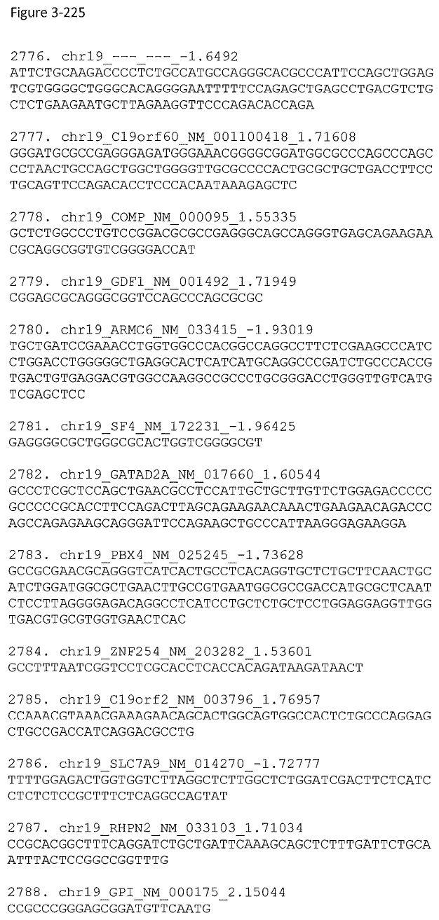

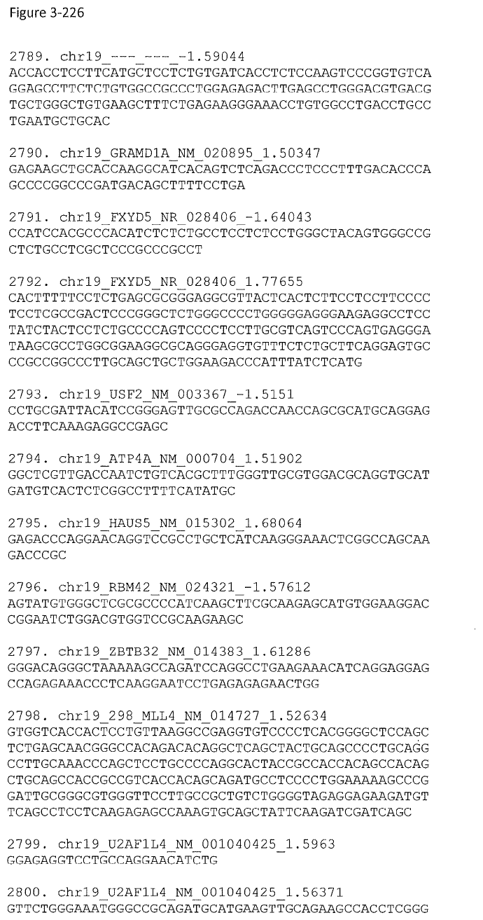

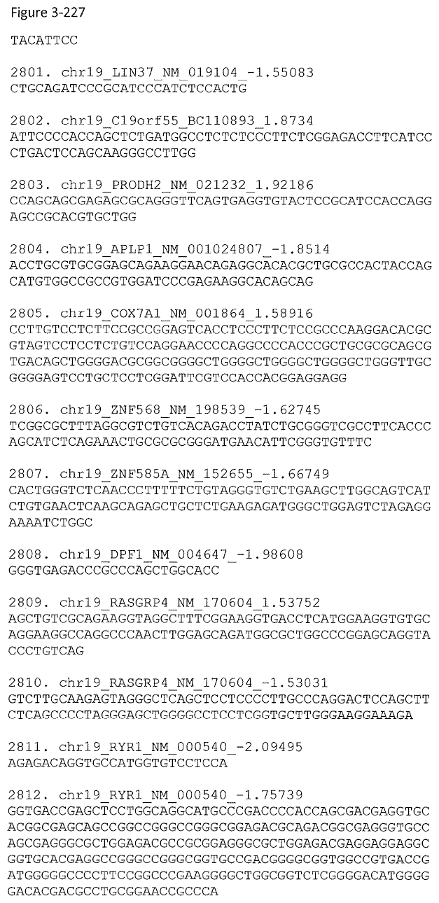

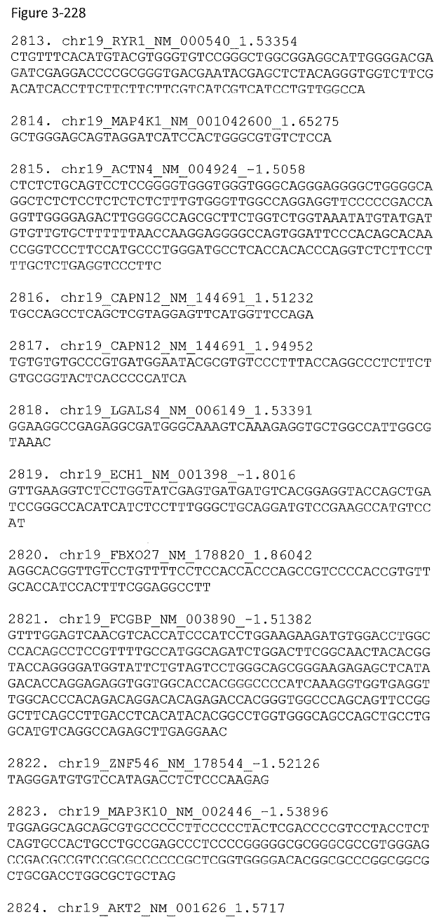

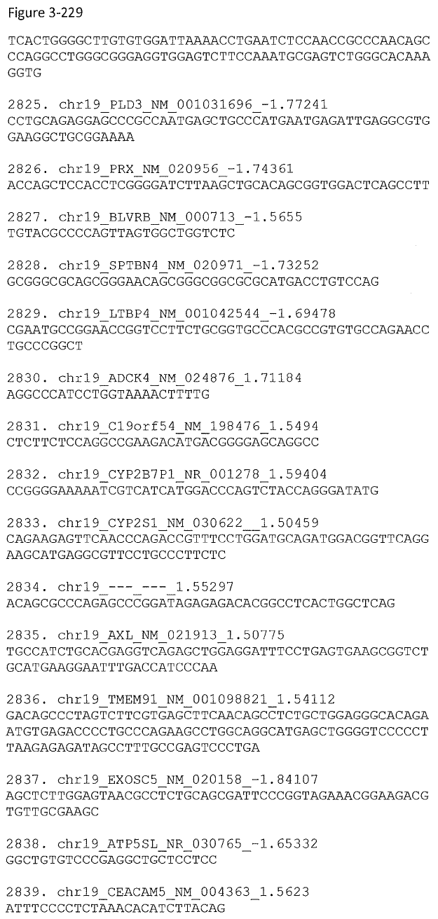

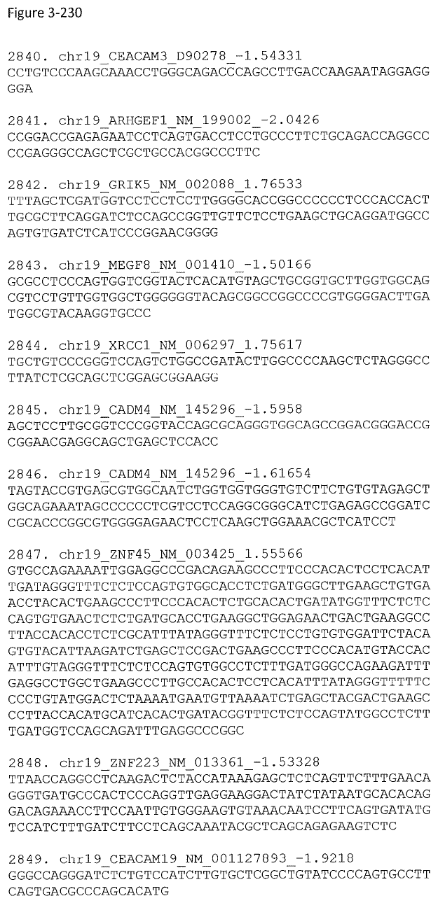

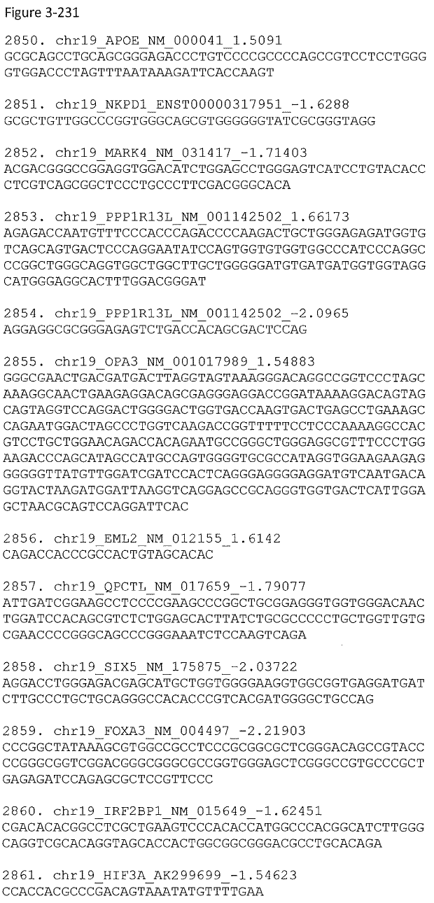

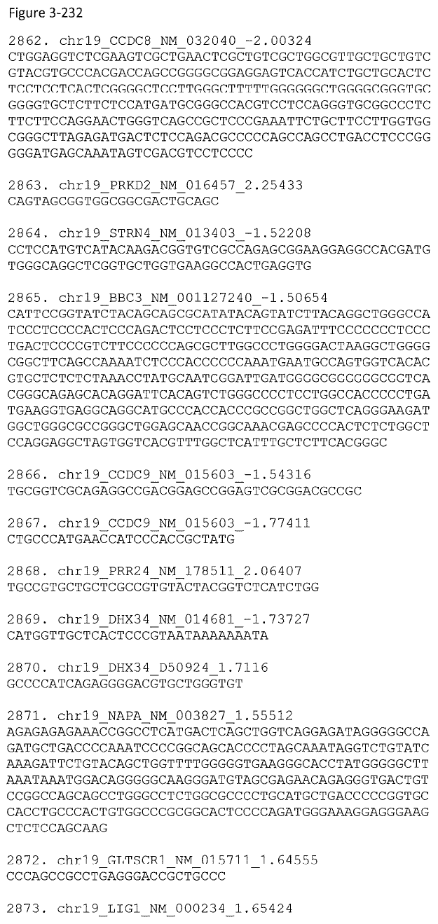

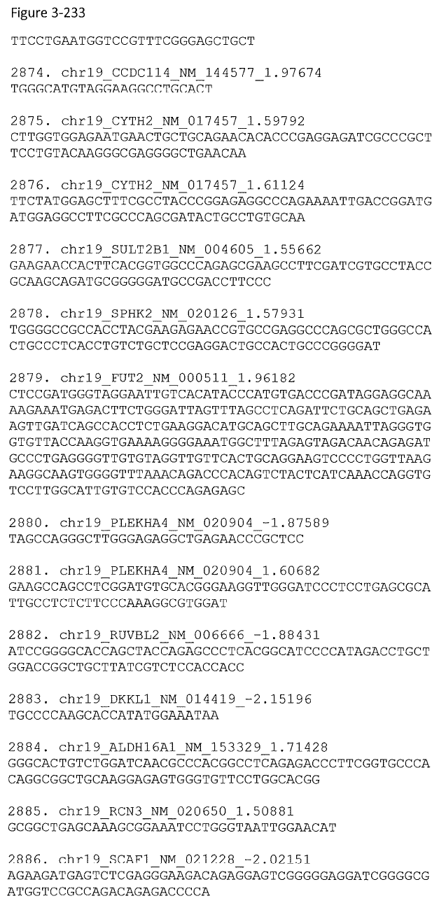

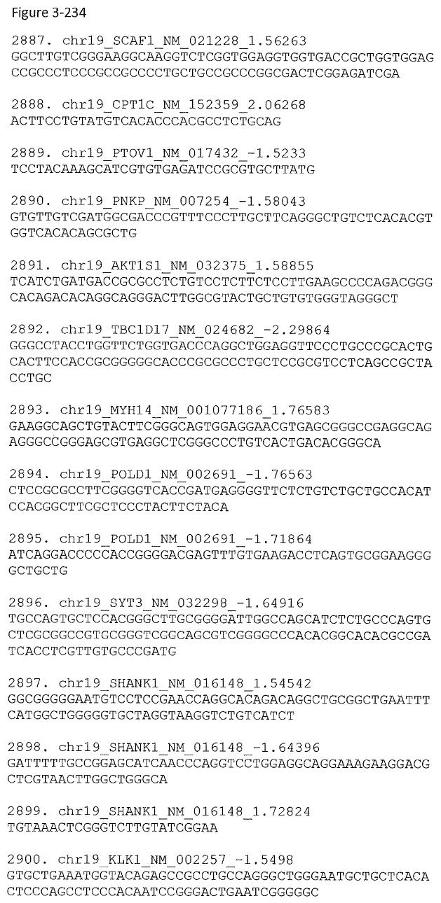

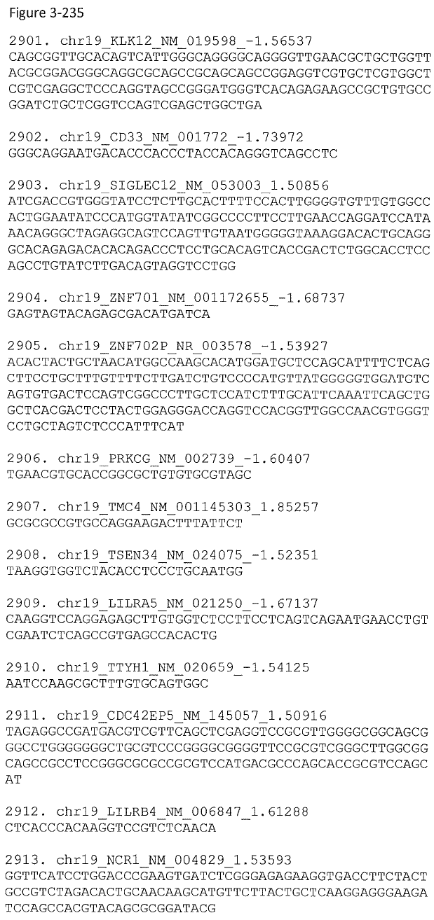

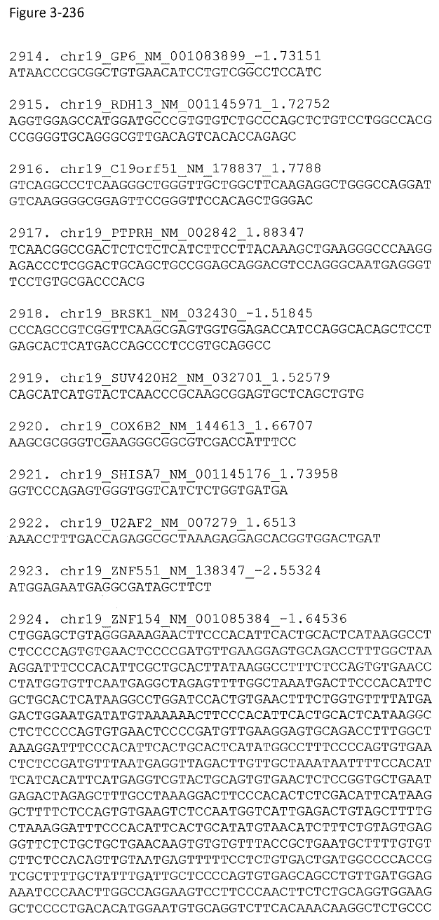

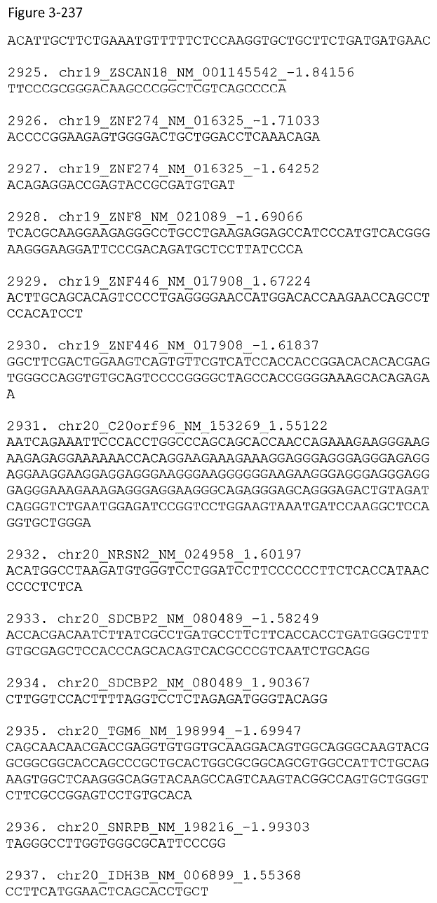

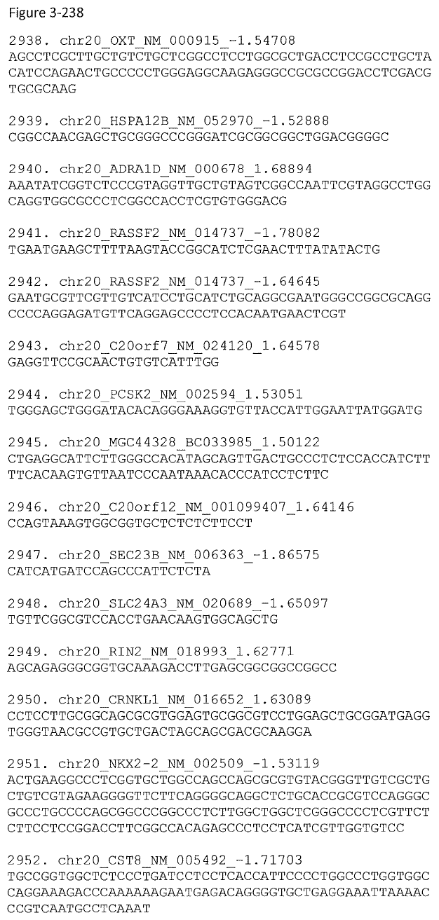

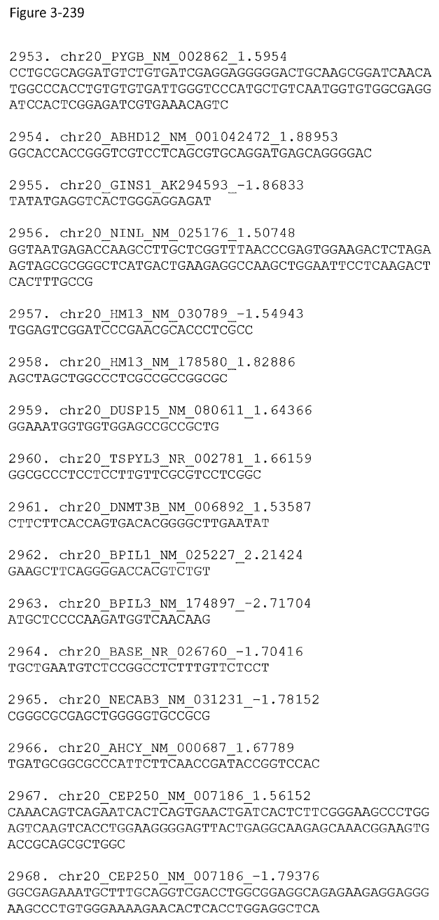

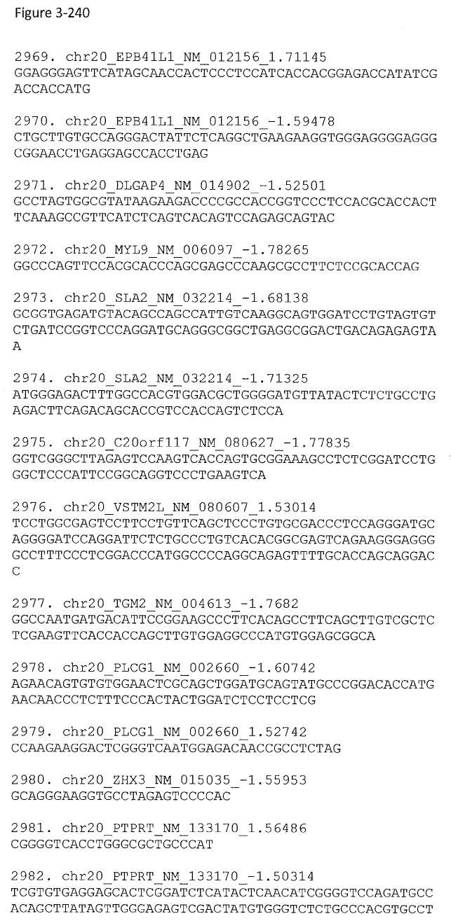

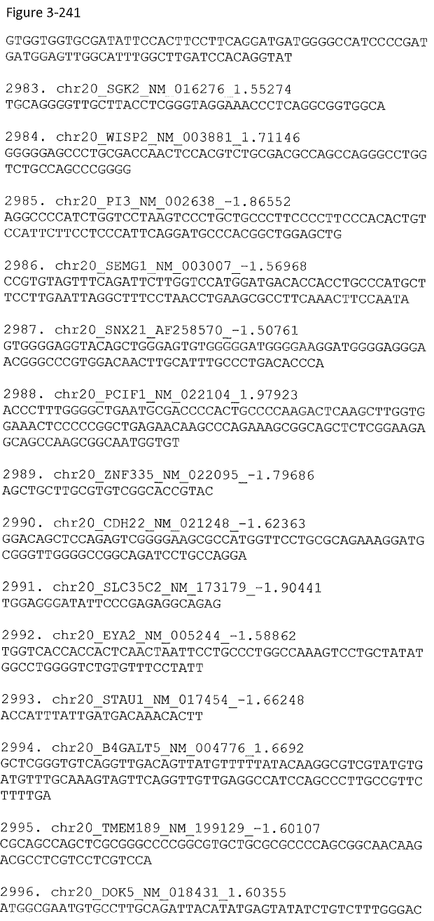

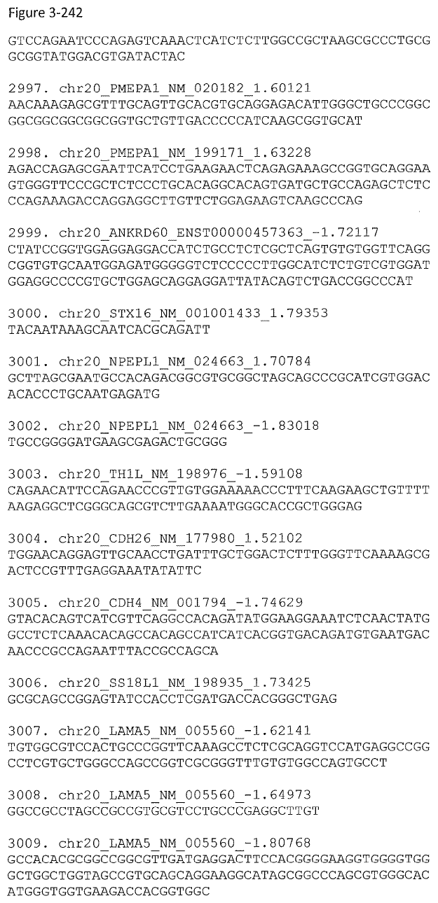

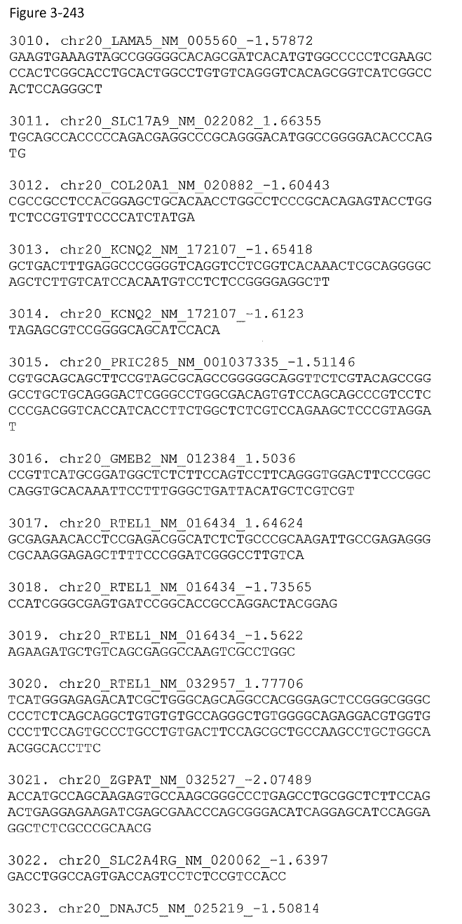

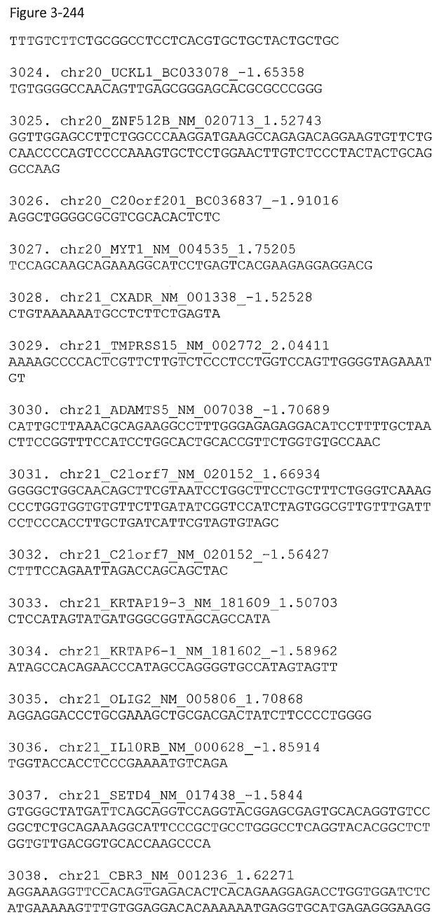

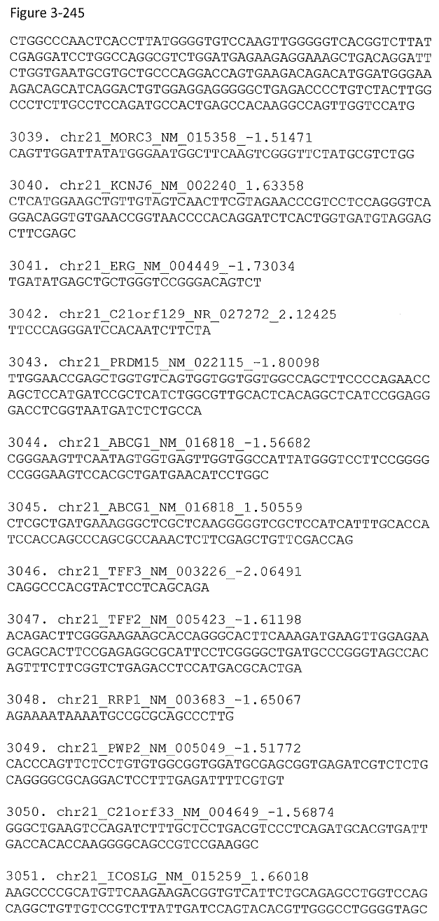

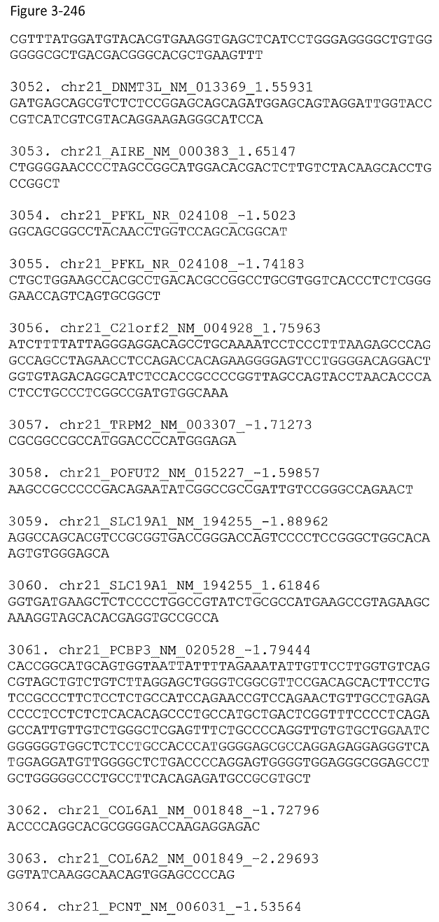

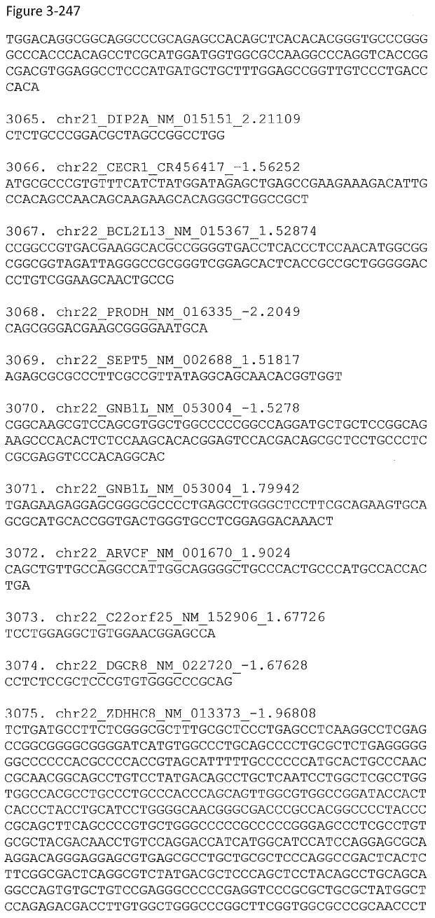

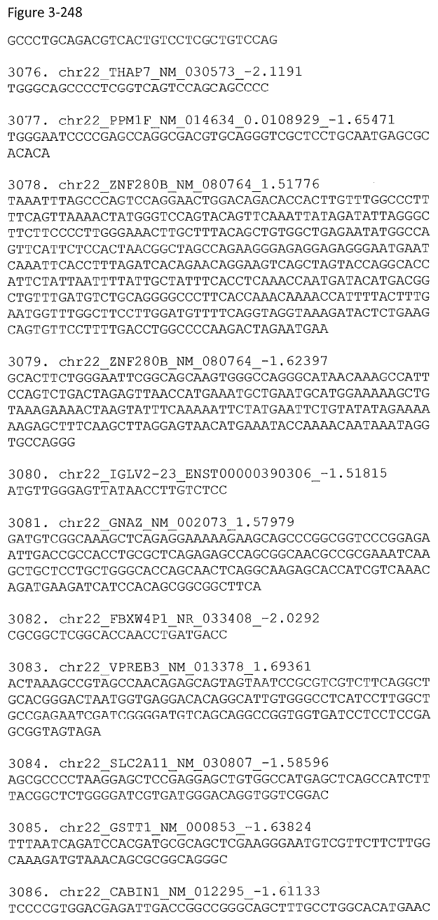

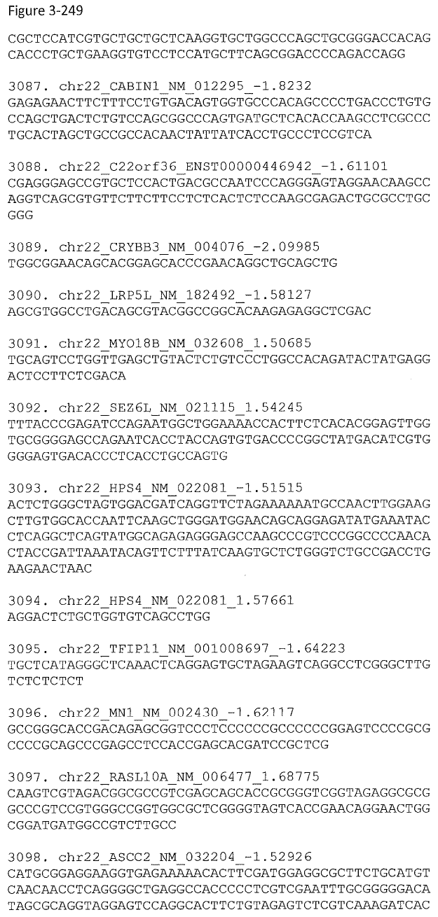

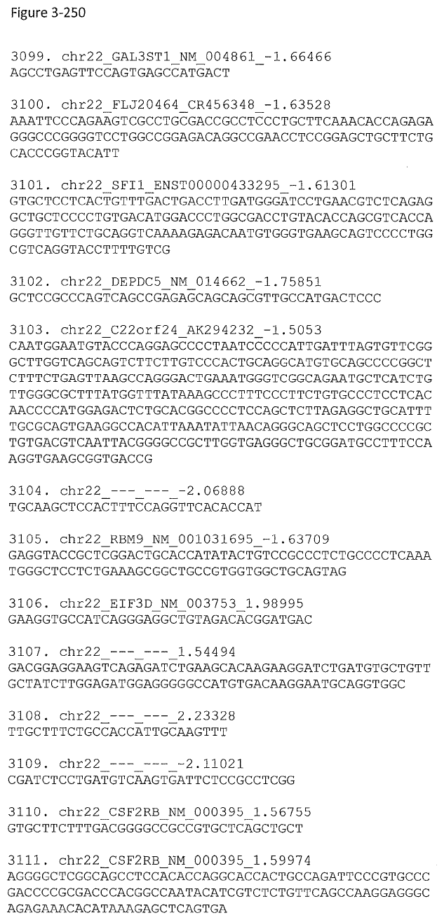

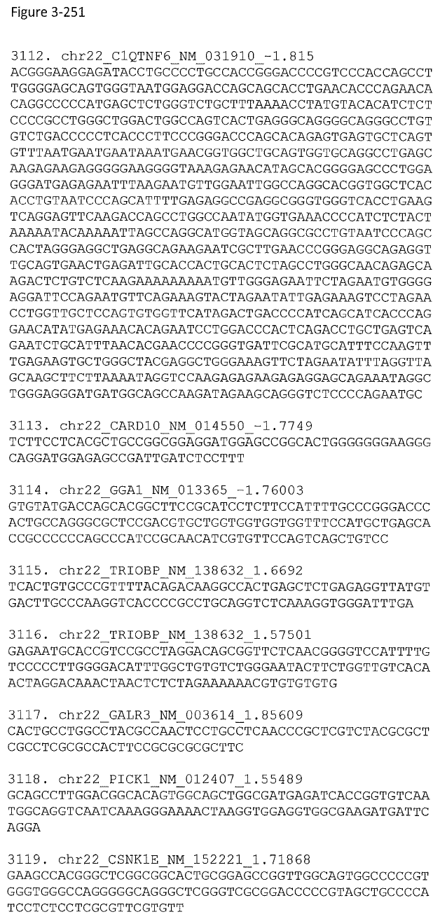

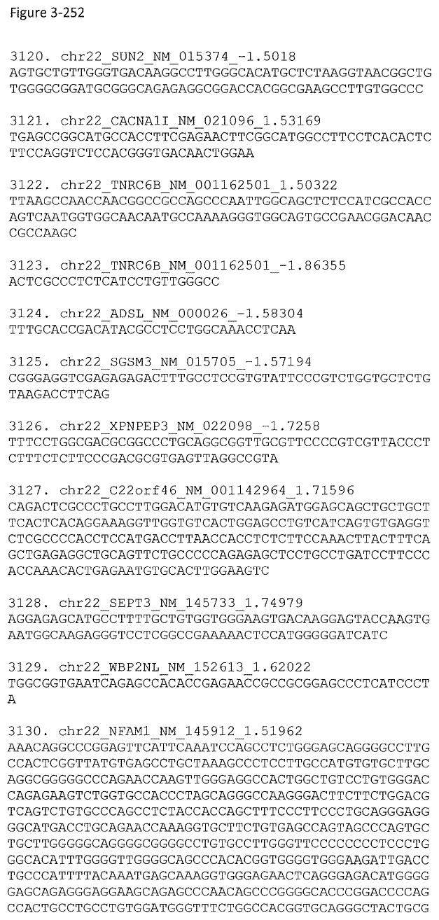

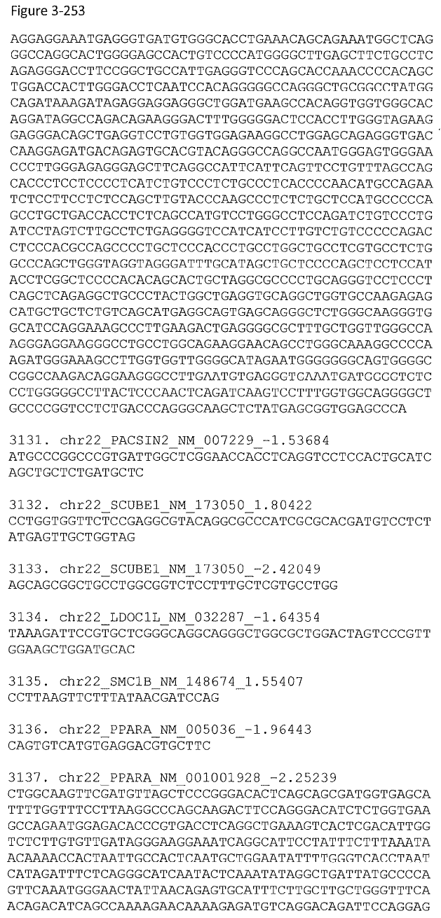

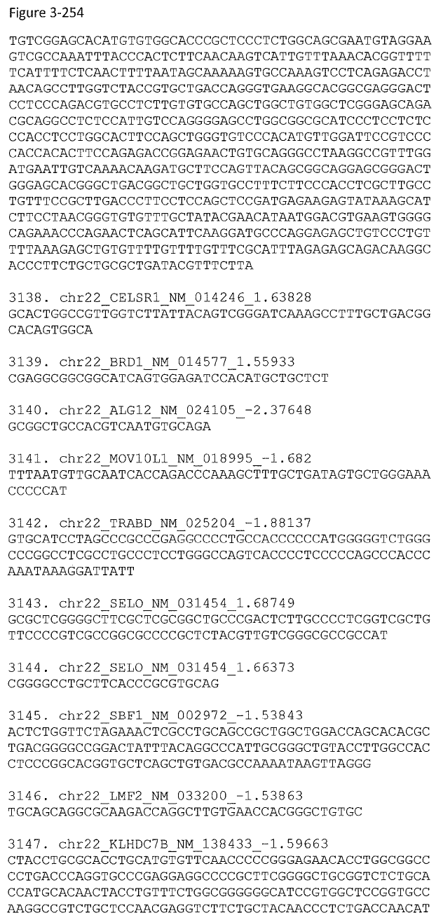

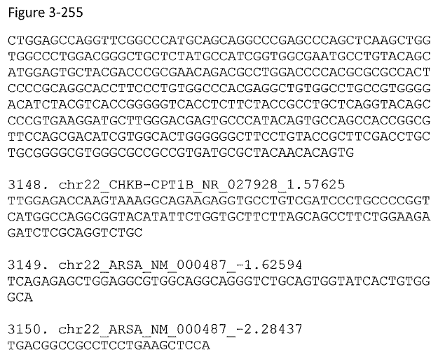

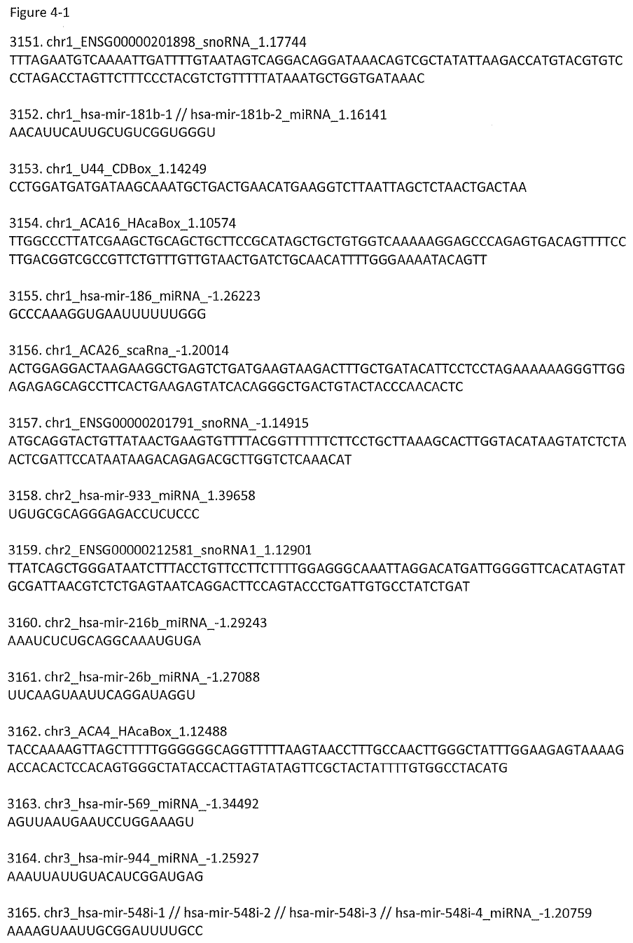

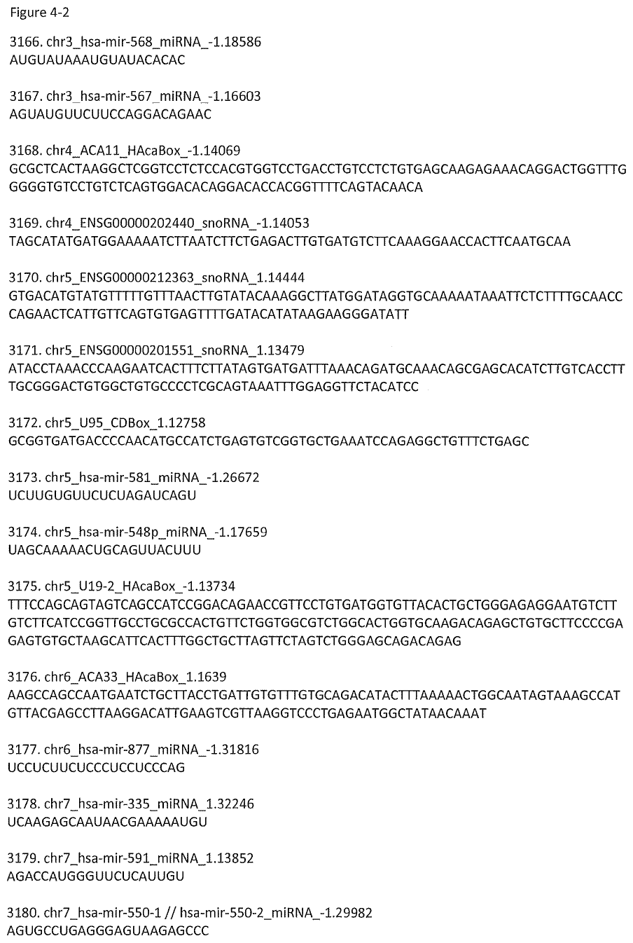

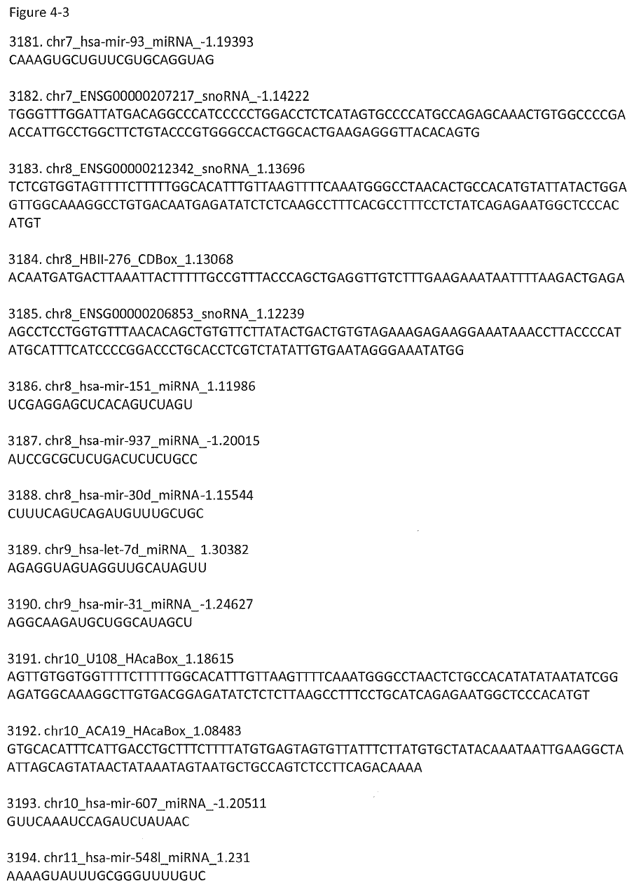

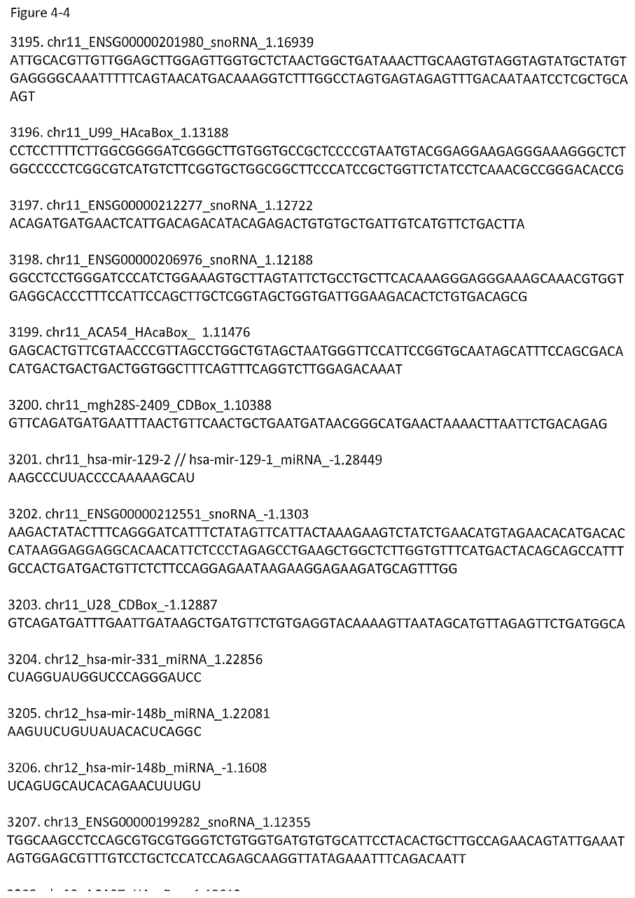

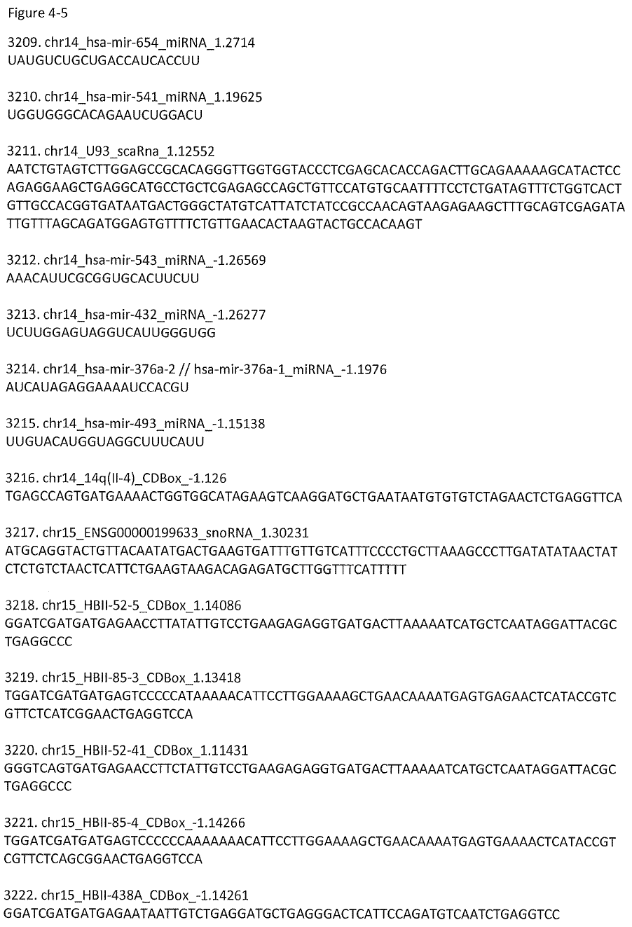

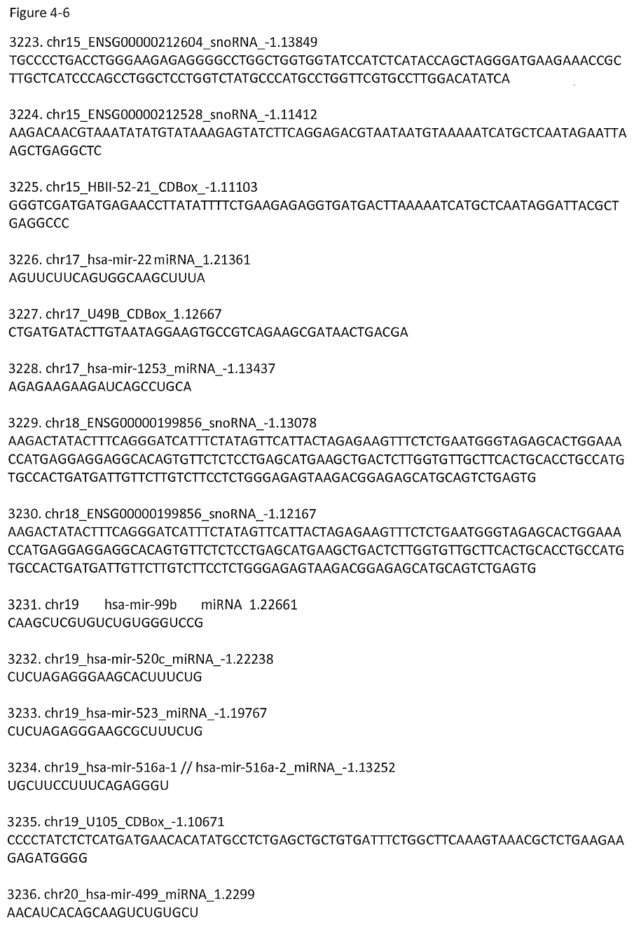

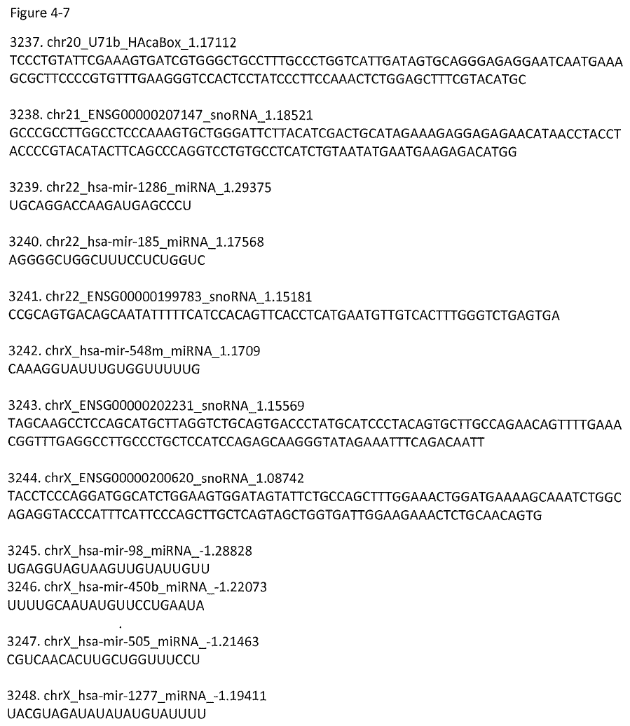

The panel of T21 biomarkers includes a subset encoded by chromosome 21 (e.g., SEQ ID NOs: 3,028-3,065 and 3,238, see FIGS. 2A and 2B). That subset includes polynucleotides found to be up-regulated in a pregnant mother carrying a fetus that is T21 when compared to a pregnant mother carrying a normal fetus. That subset also includes polynucleotides found to be down-regulated in a pregnant mother carrying a fetus that is T21 when compared to a pregnant mother carrying a normal fetus.

The panel of biomarkers includes a subset encoded by chromosomes other than chromosome 21, e.g., chromosomes 1-20, 22, and/or x (e.g., SEQ ID NOs: 8-3,027, 3,066-3,227 and 3,239-3,248, see FIGS. 2A and 2B). That subset includes polynucleotides found to be up-regulated in a pregnant mother carrying a fetus that is T21 when compared to a pregnant mother carrying a normal fetus. That subset also includes polynucleotides found to be down-regulated in a pregnant mother carrying a fetus that is T21 when compared to a pregnant mother carrying a normal fetus.

The panel of T21 biomarkers includes a subset that are mRNAs (e.g., SEQ ID NO:8-3,250) and a subset that are small non-coding RNAs (SEQ ID NO:3,251-3,248). An expression level of a T21 biomarker may include polynucleotide expression level information for one polynucleotide chosen from SEQ ID NO:8-3,248, obtained from a biological sample from a subject. A sample expression profile may include polynucleotide expression level information for two or more polynucleotides chosen from SEQ ID NO:8-3,2473, obtained from a biological sample from a subject, for instance, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30. A sample expression profile may include polynucleotide expression level information for no greater than 30 polynucleotides chosen from SEQ ID NO:8-3,273, obtained from a biological sample from a subject, for instance, no greater than 30, no greater than 29, no greater than 28, no greater than 27, no greater than 26, no greater than 25, no greater than 24, no greater than 23, no greater than 22, no greater than 21, no greater than 20, no greater than 19, no greater than 18, no greater than 17, no greater than 16, no greater than 15, no greater than 14, no greater than 13, no greater than 12, no greater than 11, no greater than 10, no greater than 9, no greater than 8, no greater than 7, no greater than 6, or no greater than 5.

The skilled person will recognize that detecting a T21 biomarker present in a subject may not require use of an entire nucleotide sequence disclosed at any of SEQ ID NO: 8-3,273. A nucleotide sequence used in a method provided herein is of a length that is at least substantially unique for a T21 biomarker to specifically hybridize with a RNA, such as a CFP RNA, present in a biological sample. A nucleotide sequence used in a method provided herein may be RNA, DNA, or RNA/DNA hybrid.

In one embodiment, a T21 biomarker present in a biological sample may be a polynucleotide that contains or consists of the sequence which defines the T21 biomarker target or complement thereof, or associated RNA or DNA thereof. The T21 biomarker may be identical to one of SEQ ID NOs: 8-3,248, or can be a complement thereof, sense or antisense, as well as a sequence that hybridizes therewith under suitable conditions. When provided as a DNA sequence, the biomarker also includes the corresponding RNA sequence. When provided as an RNA sequence, the biomarker also includes the corresponding DNA sequence.

In one embodiment, a T21 biomarker used to detect a RNA present in a biological sample, such as a CFP RNA, may be at least 6, at least 15, at least 20, at least 25, at least 30, at least 35, or at least 40 nucleotides in length, and so on, of a sequence selected from SEQ ID NO: 8-3,273, or the complement thereof. In one embodiment, a T21 biomarker may include a sequence selected from SEQ ID NO: 8-3,273, or the complement thereof, that is from 10 nucleotides to the full sequence, from 16 nucleotides to 100 nucleotides, from 17 nucleotides to 50 nucleotides, from 18 nucleotides to 30 nucleotides, from 19 nucleotides to 25 nucleotides, or from 20 to 22 nucleotides. A T21 biomarker selected from SEQ ID NO: 8-3,273 may have perfect identity, at least 95% identity, at least 90% identity, at least 85% identity, or at least 80% identity with a sequence disclosed herein. A T21 biomarker selected from SEQ ID NO: 8-3,273 may have perfect complementarity or at least 95% complementarity, at least 90% complementarity, at least 85% complementarity, or at least 80% complementarity with a sequence disclosed herein. A T21 biomarker may be continuous or it can have one or more bulges or mismatches upon hybridization. A T21 biomarker used to detect a RNA in a biological sample may also include one or more chemical modifications, such as a 2′ carbon modification. A T21 biomarker may or may not form an overhang upon hybridization when detecting a RNA present in a biological sample.

“Hybridization” includes any process by which a strand of a nucleic acid sequence joins with a second nucleic acid sequence strand through base-pairing. Hybridization of polynucleotides is affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringency conditions depend on the length and base composition of the nucleic acid, which can be determined by techniques well known in the art. Generally, stringency can be altered or controlled by, for example, manipulating temperature and salt concentration during hybridization and washing. For example, a combination of high temperature and low salt concentration increases stringency. The degree of stringency may be based, for example, on the calculated (estimated) melting temperature (Tₘ) of the polynucleotide. Calculation of Tₘ is well known in the art. For example, “maximum stringency” typically occurs at around Tₘ−5° C. (5° below the Tₘ of the probe); “high stringency” at around 5-10° below the Tₘ; “intermediate stringency” at around 10-20° below the Tₘ of the probe; and “low stringency” at around 20-25° below the Tₘ. Maximum stringency conditions may be used to identify a polynucleotide present in a biological sample having strict identity or near-strict identity with a T21 biomarker selected from SEQ ID NO:8-3,248; while high stringency conditions are used to identify a polynucleotide present in a biological sample having about 80% or more sequence identity with a T21 biomarker. Such conditions are known to those skilled in the art and can be found in, for example, Strauss, W. M. “Hybridization With Radioactive Probes,” in Current Protocols in Molecular Biology 6.3.1-6.3.6, (John Wiley & Sons, N.Y. 2000). Both aqueous and nonaqueous conditions as described in the art can be used.

Expression levels of any one or more of the T21 biomarkers described herein may be used to determine the presence, absence, or type of T21 in a subject. In one embodiment, expression levels of one or more T21 biomarkers encoded by chromosome 21 may be used to determine the presence, absence, or type of T21 in a subject. In one embodiment, expression levels of one or more T21 biomarkers encoded by the remaining 21 autosomes (chromosomes 1-22 exclusive of chromosome 21) and X may be used to determine the presence, absence, or type of T21 in a subject. In one embodiment, expression levels of one or more T21 biomarkers encoded by any combination of chromosomes 1-22 and X may be used to determine the presence, absence, or type of T21 in a subject. In one embodiment, expression levels of one or more T21 biomarkers encoded by one chromosome selected from 1-22 and X may be used to determine the presence, absence, or type of T21 in a subject.

In one embodiment, expression levels of one or more T21 biomarkers that are mRNAs may be used to determine the presence, absence, or type of T21 in a subject. In one embodiment, expression levels of one or more T21 biomarkers that are small non-coding RNAs may be used to determine the presence, absence, or type of T21 in a subject. In one embodiment, the T21 biomarkers used may be those that are up-regulated in a pregnant mother carrying a fetus that is T21 when compared to a pregnant mother carrying a normal fetus. In one embodiment, the T21 biomarkers used may be those that are down-regulated in a in a pregnant mother carrying a fetus that is T21 when compared to a pregnant mother carrying a normal fetus. In one embodiment, the T21 biomarkers used may be a combination of those that are up-regulated and those that are down-regulated in a pregnant mother carrying a fetus that is T21 when compared to a pregnant mother carrying a normal fetus.

The number of T21 biomarkers used in an assay to determine the presence, absence, or type or T21 in a subject may vary. The skilled person will appreciate that, generally, the more biomarkers examined, the more accurate the determination of the presence, absence, or type of T21 in a subject; however, the skilled person will also appreciate that there is a minimum number of biomarkers useful for an accurate diagnosis of T21. In one embodiment, the number of T21 biomarkers evaluated in practicing a method provided herein may be at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30. In one embodiment, the number of T21 biomarkers evaluated in practicing a method provided herein may be no greater than 30, no greater than 29, no greater than 28, no greater than 27, no greater than 26, no greater than 25, no greater than 24, no greater than 23, no greater than 22, no greater than 21, no greater than 20, no greater than 19, no greater than 18, no greater than 17, no greater than 16, no greater than 15, no greater than 14, no greater than 13, no greater than 12, no greater than 11, no greater than 10, no greater than 9, no greater than 8, no greater than 7, no greater than 6, or no greater than 5. In one embodiment, the number of CFP RNAs detected varies depending upon whether the fetus or subject is normal or abnormal.

All the T21 biomarkers measured in a subject having T21 may not show altered expression levels when compared to a healthy subject. A subject may be considered to have T21 when at least 60%, at least 65%, at least 70%, at least 75%, at least 80%, at least 85%, at least 90%, at least 95%, or 100% of the T21 biomarkers in a sample expression profile from the subject's biological sample show altered expression when compared to those T21 biomarkers in a negative control expression profile from a healthy subject. For instance, in an embodiment where 10 biomarkers are measured in a biological sample from a subject, such as a pregnant mother carrying a fetus with T21, the subject may be considered to have T21 when at least 6 of the biomarkers in a sample expression profile show altered expression when compared to those T21 biomarkers in a control expression profile from a healthy subject. Some of the T21 biomarkers in a subject not having T21 may show altered expression levels when compared to another healthy subject. A subject may be considered not to have T21 when no greater than 40%, no greater than 35%, no greater than 30%, no greater than 25%, no greater than 20%, no greater than 15%, no greater than 10%, no greater than 5%, or none of the T21 biomarkers in a sample expression profile from the subject's biological sample show altered expression when compared to those T21 biomarkers in a control expression profile from another healthy subject. For instance, in an embodiment where 10 biomarkers are measured in a biological sample from a subject, such as a pregnant mother carrying a normal fetus, the subject may be considered to have a normal fetus when no more than 4 of the biomarkers in a sample expression profile show altered expression when compared to the normal range for the population of healthy fetuses.

Whether the expression level or quantity of a biomarker in a subject having T21 is greater than or less than the expression level or quantity of the biomarker in a healthy subject is determined using routine statistical methods by applying accepted confidence levels. The expression level or quantity of a T21 biomarker in a biological sample is considered to be altered if the difference in amount of the biomarker in a test sample is increased or decreased to a statistically significant degree compared to the amount of the biomarker in a control sample. The term “statistically significant” refers to a result, namely a difference in numbers of positive results between a test and a control that is not likely due to chance. The minimum chance level for statistical significance herein is 95% probability that the result is not due to chance, i.e., random variations in the data. A 95% confidence interval means that if the procedure for computing a 95% confidence interval is used over and over, 95% of the time the interval will contain the true parameter value. In one embodiment, the minimum chance level for statistical significance is 97% probability, 99% probability, or 99.9% probability. Various methods, as is known, can be used to calculate statistical significance. Examples include, but are not limited to, binomial probabilities, the Poisson distribution, chi-square, and t-test. The skilled person will recognize that one may use sufficient numbers of results to obtain a confidence interval of at least 95%, or higher, in order to determine statistical significance of a difference in expression level or quantity of a biomarker in a subject having T21 and the expression level or quantity of the biomarker in a healthy subject. However, machine learning protocols can be utilized to recognize patterns with ranking in order to determine if there is a significance of a difference in expression level or quantity of biomarker in a subject having T21 from the expression level or quantity of the biomarker in the health subject.

In one embodiment, a subject is considered to have T21 when comparison of expression of at least one T21 biomarker, or a plurality of T21 biomarkers, with the expression level of the at least one T21 biomarker, or a plurality of T21 biomarkers, in a biological sample from a subject not having T21 shows a difference, and that difference is indicative of the presence of T21 in the subject. In one embodiment, a subject is considered to have T21 when expression of at least one T21 biomarker, or a plurality of T21 biomarkers, is altered to a statistically significant degree or determined by machine learning in a biological sample from the subject compared to a biological sample from a subject not having trisomy 21. In one embodiment, a subject is considered to have T21 when comparison of expression of at least one T21 biomarker with the expression level of the at least one T21 biomarker in a biological sample from a subject not having T21 shows that the expression level or quantity of a biomarker in the subject is outside the 95% confidence interval for the biomarker. In one embodiment, a subject is considered to have T21 when comparison of expression of a plurality of T21 biomarkers with the expression level of the plurality of T21 biomarkers in a biological sample from a subject not having T21 shows that the expression level or quantity of the plurality of biomarker in the subject is outside the 95% confidence interval for the plurality of the biomarkers.

Accordingly, in one embodiment, a method provided herein includes measuring a plurality of T21 biomarkers in a biological sample obtained from a subject, such as a pregnant female. The plurality of T21 biomarkers (e.g., a specific combination) measured may be selected from any combination of a defined group, or a complement thereof, or a portion thereof. The plurality of T21 biomarkers measured may be polynucleotides that hybridize to a sequence selected from any one of SEQ ID NO:8-3,273 under suitable conditions. In one embodiment, a method provided herein includes detecting T21 biomarkers in a biological sample to yield an expression level of each detected T21 biomarker. The T21 biomarkers may be selected from any combination of SEQ ID NO:8-3,273, or a complement thereof, or a portion thereof. The T21 biomarkers detected may be polynucleotides that hybridize to a sequence selected from any one of SEQ ID NO:8-3,273 under suitable conditions. The biological sample may include plasma from a pregnant female. In one embodiment, a method disclosed herein includes detecting T21 biomarkers in a biological sample to yield a sample expression profile. The T21 biomarkers may be selected from any combination of SEQ ID NO:8-3,273, or a complement thereof, or a portion thereof. The T21 biomarkers detected may be selected from SEQ ID NO:8-3,273, or a complement thereof, or a portion thereof. The T21 biomarkers detected may be polynucleotides that hybridize to a sequence selected from any one of SEQ ID NO:8-3,273 under suitable conditions. The biological sample may include plasma from a pregnant female.

In one embodiment, such as one where the subject is a pregnant female, a method disclosed herein may include identifying the fetus as i) having trisomy 21 if expression of the plurality of biomarkers is altered to a statistically significant degree in the biological sample compared to a biological sample from a second pregnant female carrying a fetus not having trisomy 21, or ii) not having trisomy 21 if expression of the plurality of biomarkers is not altered to a statistically significant degree in the biological sample compared to a biological sample from a second pregnant female carrying a fetus not having trisomy 21. In one embodiment, such as one where the subject is a pregnant female, the method may further include comparing the expression level of a detected T21 biomarker to the expression level of the T21 biomarker in pregnant females carrying a fetus without T21, wherein an expression level of a detected T21 biomarker that is outside the 95% confidence interval for that T21 biomarker indicates the expression level of the T21 biomarker is altered. In one embodiment, such as one where the subject is a pregnant female, the method may further include comparing the sample expression profile with a reference expression profile; wherein a difference between the sample expression profile and the reference expression profile is indicative of the presence of trisomy 21 in the fetus. A sample whose expression levels were not different from the standard control would be interpreted to be from a pregnancy unaffected by T21. A significant difference from the standard would lead to the conclusion T21 was present]

In one embodiment, such as one where the fetus is diagnosed as having T21, a method may further include recommending to the pregnant female a genetic test chosen from amniocentesis, cordocentesis, and chorionic villus sampling.

Amounts of T21 biomarkers in a biological sample may be determined in absolute or relative terms. If expressed in relative terms, amounts can be expressed as normalized amounts with reference to one or more normalization sequences present in a biological sample.

It is expected that this method will have a sensitivity (percent of fetuses or subjects having T21 correctly identified, also referred to as detection rate) of at least 98%, at least 99%, or 100% when enough T21 biomarkers present in a biological sample are detected. It is also expected that this method will have a specificity (percent of fetuses or subjects not having T21 correctly identified) of at least 98%, at least 99%, or 100% when enough T21 biomarkers present in a biological sample are detected.

Measuring the expression level or quantity of any single T21 biomarker or a plurality of T21 biomarkers may be accomplished by use of techniques that are known in the art and routine. In one embodiment, the expression level or quantity of a T21 biomarker or a plurality of T21 biomarkers may be monitored directly by detecting RNA present in a biological sample. RNA may be obtained from a biological sample using routine techniques known in the art. In one embodiment, the RNA is cell-free RNA obtained from biological tissue and/or fluid. In one embodiment, the RNA is cell-free plasma RNA obtained from whole blood, blood plasma, or serum. In one embodiment, the RNA is isolated. As used herein, the term “isolated” refers to a polynucleotide that has been removed from its natural environment.

Detecting one or more T21 biomarkers that are present as a RNA polynucleotide may be accomplished by a variety of methods. Some methods are quantitative and allow estimation of the original levels of RNA between the levels present in a test sample and a control, such as a control expression level for a T21 biomarker and/or a control expression profile, whereas other methods are merely qualitative. In one embodiment, a method for detecting one or more T21 biomarkers may include the use of polynucleotides that are in solution, and may be in any format, including, but not limited to, the use of individual tubes or a high throughput device, such as a PCR-card.

Quantitative real-time PCR (QRT-PCR) may be used to measure the differential expression of any T21 biomarker in a test sample and a control. In QRT-PCR, the RNA template is generally reverse transcribed into cDNA, which is then amplified via a PCR reaction. The primers used for amplification may be selected by determining which T21 biomarker(s) described at SEQ ID NO:8-3,273 is to be amplified, and then designing primers using routine methods known in the art. The PCR amplification process is catalyzed by a thermostable DNA polymerase. Non-limiting examples of suitable thermostable DNA polymerases include Taq DNA polymerase, Pfu DNA polymerase, Tli (also known as Vent) DNA polymerase, Tfl DNA polymerase, and Tth DNA polymerase. The PCR process may include three steps (i.e., denaturation, annealing, and extension) or two steps (i.e., denaturation and annealing/extension). The temperature of the annealing or annealing/extension step may vary, depending upon the amplification primers and other parameters such as concentration. The temperature of the annealing or annealing/extending step may range from about 50° C. to about 75° C. The amount of PCR product is followed cycle-by-cycle in real time, which allows for determination of the initial concentrations of mRNA. The reaction may be performed in the presence of a dye that binds to double-stranded DNA, such as SYBR Green. The reaction may also be performed with fluorescent reporter probes, such as TAQMAN probes (Applied Biosystems, Foster City, Calif.) that fluoresce when the quencher is removed during the PCR extension cycle. Fluorescence values are recorded during each cycle and represent the amount of product amplified to that point in the amplification reaction. The cycle when the fluorescent signal is first recorded as statistically significant is the threshold cycle (Ct). To minimize errors and reduce any sample-to-sample variation, QRT-PCR is typically performed using one or more normalization sequences.

Reverse-transcriptase PCR (RT-PCR) may also be used to measure the expression of a T21 biomarker. As described above, the RNA template is reverse transcribed into cDNA, which is then amplified via a typical PCR reaction. After a set number of cycles the amplified DNA products are typically separated by gel electrophoresis. Comparison of the relative amount of PCR product amplified in different samples will reveal whether the expression of a T21 biomarker is altered in a test sample. Accordingly, sequences in the Sequence Listing showing DNA can have the “T” replaced with a “U” to convert to the corresponding RNA, and vice versa.

Expression of a T21 biomarker may also be measured using a nucleic acid microarray (also referred to in the art as a DNA chip or biochip). In this method, single-stranded polynucleotides selected from at least a portion of SEQ ID NO:8-3,273, or a complement thereof, are plated, or arrayed, on a solid support. The solid support may be a material such as, for instance, glass, silica-based, silicon-based, a synthetic polymer, a biological polymer, a copolymer, a metal, or a membrane. The form or shape of the solid support may vary, depending on the application. Suitable examples include, but are not limited to, slides, strips, plates, wells, microparticles, fibers (such as optical fibers), gels, and combinations thereof. The arrayed immobilized sequences are generally hybridized with specific DNA probes obtained from the test sample. As described above, RNA present in a sample, including T21 biomarkers, is generally reverse transcribed into cDNA. Fluorescently labeled cDNA probes may be generated through incorporation of fluorescently labeled deoxynucleotides during the reverse transcription step. The cDNA probes are hybridized to the immobilized nucleic acids on the solid support under highly stringent conditions. After stringent washing to remove non-specifically bound probes, the solid support is scanned using routine methods, for instance, by confocal laser microscopy or by another detection method, such as a CCD camera. Quantitation of hybridization of each arrayed element allows for assessment of corresponding RNA abundance. With multiple color fluorescence, separately labeled cDNA probes may be hybridized pairwise to the array. The relative abundance of the transcripts from the two sources corresponding to each specified T21 biomarker may then be determined simultaneously. Microarray analysis may be performed by commercially available equipment, following manufacturer's protocols, such as by using the Affymetrix GenChip technology, or Incyte's microarray technology.

Differential expression of a T21 biomarker may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe. After washing to remove the non-specifically bound probe, the hybridized labeled species are detected using routine techniques known in the art. The probe may be labeled with, for instance, a radioactive element, a chemical that fluoresces when exposed to ultraviolet light, a tag that is detected with an antibody, or an enzyme that catalyses the formation of a colored or a fluorescent product. A comparison of the relative amounts of RNA detected in a control sample and a test sample will reveal whether the expression of one or more T21 biomarkers or changed in the test sample.

Nuclease protection assays may also be used to monitor the altered expression of a T21 biomarker in a test sample and a control. In nuclease protection assays, an antisense probe hybridizes in solution to a RNA sample. The antisense probe may be labeled with an isotope, a fluorophore, an enzyme, or another tag. Following hybridization, nucleases are added to degrade the single-stranded, unhybridized probe and RNA. An acrylamide gel is used to separate the remaining protected double-stranded fragments, which are then detected using techniques well known in the art. Again, qualitative differences in expression may be detected.

In one embodiment, expression of a T21 biomarker may be examined in vivo in a subject. One or more RNA polynucleotides may be labeled with fluorescent dye, a bioluminescent marker, a fluorescent semiconductor nanocrystal, or a short-lived radioisotope, and then the subject may be imaged or scanned using a variety of techniques, depending upon the type of label.

In one embodiment, the detection of a RNA, such as a CFP RNA, uses the nucleotides of a specific exon as described in SEQ ID NO:8-3,273. Thus, if QRT-PCR is used to detect a specific CFP RNA, the primers used to amplify the CFP RNA will amplify all or a portion of an exon described in SEQ ID NO:8-3,273. If a microarray is used to detect a specific CFP RNA, the arrayed immobilized sequence used to detect the CFP RNA will be based on all or a portion of an exon described in SEQ ID NO:8-3,273, or a complement thereof. A person skilled in the art will know which parameters may be manipulated to optimize detection of a RNA of interest using one or more of the polynucleotides listed at SEQ ID NO:8-3,273.

When determining whether the expression of a T21 biomarker or a plurality of T21 biomarkers are altered in a test sample compared to a control expression level or a control expression profile, it can be helpful to use a normalization sequence. A normalization sequence is a polynucleotide that can be used to normalize the relative amounts of polynucleotides, and/or data obtained from the polynucleotides, from one sample to the next. A normalization sequence can be RNA that has an expression level or quantity that is generally stable under the conditions studied. That is, the normalization sequence can have an expression level or quantity that is substantially unaffected by physiological circumstances present in a subject, and thus the normalization sequence can be used to normalize the amount of polynucleotides in separate samples for comparison. The separate samples can be from different subjects or the same subject at different time points, such as different time points in pregnancy. For example, the normalization sequence can be used to normalize the amount of RNA in QRT-PCR studies, such as by normalizing the amount of a RNA sequence of interest. The normalization sequences described herein can be used alone or in combination and may be used to normalize samples to be assayed for T21 biomarkers. Thus, the normalization sequences provided herein can be for quantification of cell-free RNA, including CFP RNA, present in a biological sample.

It has been determined that previously reported normalization sequences utilized in other tissues for quantification of isolated RNA (e.g., mRNA: 18s RNA, RPLP0, and GAPDH; miRNA: miR-103, miR-146a, and miR-197) were either expressed inconsistently in control plasma samples or were altered by either pregnancy, gestational age or disease (see Dong and Weiner, WO 12/075150, incorporated by reference). The normalization sequences described can include cell-free plasma RNA sequences (including coding sequences, e.g., mRNA, and/or non-coding sequences, e.g., miRNA) that are substantially unchanged by a condition. In one embodiment, the normalization sequences are substantially unchanged during the course of pregnancy.

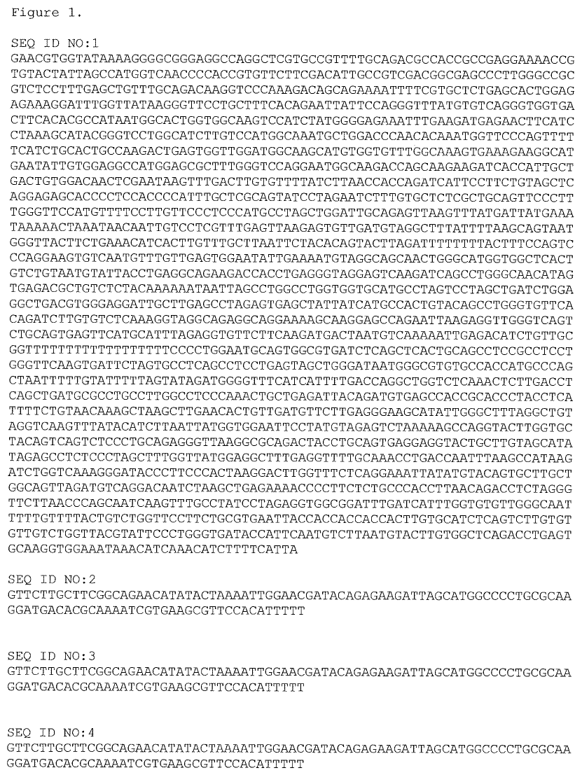

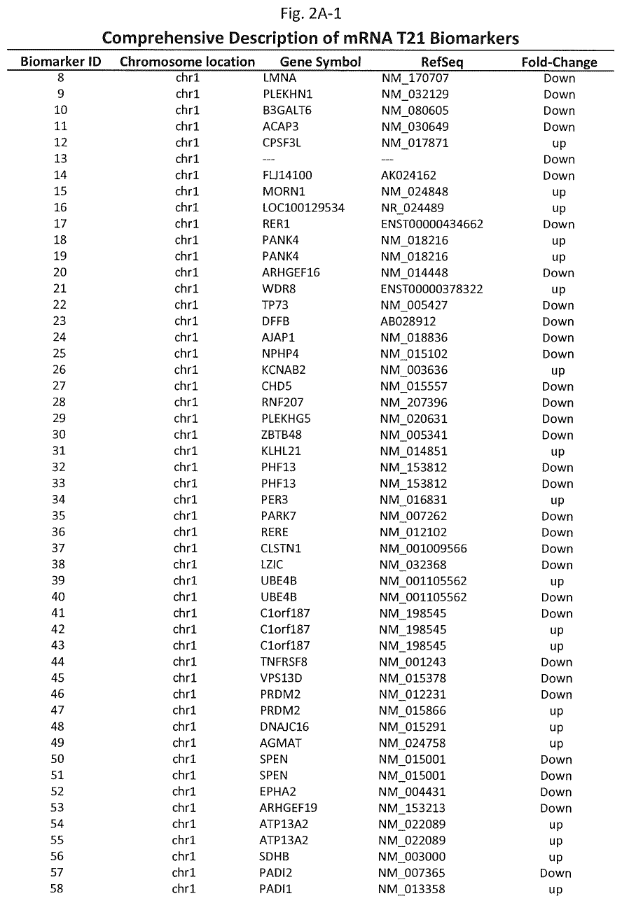

Normalization sequences appropriate for use in the methods provided herein may be identified as described in Dong and Weiner (WO 12/075150, incorporated by reference). In one embodiment, the normalization sequence includes a circulating RNA. Such a normalization sequence can be described as human (i.e., Homo sapiens) peptidylprolyl isomerase A (i.e., cyclophilin A, rotmase A), which is encoded by a PPIA coding region. The normalization sequence can be an mRNA for peptidylprolyl isomerase. An example of a peptidylprolyl isomerase normalization sequence can be found at accession number: NM_021130 and/or NM_001008741. An example of a peptidylprolyl isomerase normalization sequence that may be useful for normalization of mRNA is depicted at SEQ ID NO: 1 (see FIG. 1). In one embodiment, the normalization sequence may include miRNA. Such a normalization sequence may be a Drosophila melanogaster small nuclear RNA, such as snRNA:U6. The snRNA:U6 normalization sequence can be snRNA:U6 at 96Aa, 96: Ab, and/or 96Ac. Examples of these normalization sequences include snRNA:U6:96Aa (SEQ ID NO: 2 for miRNA), snRNA:U6:96Ab (SEQ ID NO: 3 for miRNA), and/or snRNA:U6:96Ac (SEQ ID NO: 4 for miRNA) (see FIG. 1), and can be found at the following accession numbers, respectively: NR 002081 (snRNA:U6:96Aa); NR 002082 (snRNA:U6:96Ab); and NR_002083 (snRNA:U6:96Ac). Accordingly, SEQ ID NO: 1 may be used for normalization of mRNA, and SEQ ID NOs: 2-4 may be used for normalization of miRNA. More than one normalization sequence may be used.

Primers and probes for these sequences can be readily obtained by one of ordinary skill in the art. For example, sequences for the forward primer, reverse primer, and probe for SEQ ID NO: 1 (e.g., for an mRNA normalization sequence) may be: Forward primer: GCTTTGGGTCCAGGAATGG (SEQ NO: 5); ID Reverse primer: GTTGTCCACAGTCAGCAATGGT (SEQ ID NO:6); and Probe: AGACCAGCAAGAAGAT (SEQ ID NO:7). These polynucleotides may also be used as normalization sequences in the methods provided herein.

In one embodiment, a normalization sequence may be a polynucleotide that contains or consists of the sequence. The normalization sequence can be identical to one of SEQ ID NO: 1-7, or can be a complement thereof, sense or antisense, as well as a sequence that hybridizes therewith under suitable conditions. In one embodiment, a normalization sequence may include a sequence selected from SEQ ID NO:1-7, or the complement thereof, that is at least 15 nucleotides, at least 20 nucleotides, at least 25 nucleotides, at least 30 nucleotides, at least 35 nucleotides, at least 40 nucleotides, at least 45 nucleotides, at least 50 nucleotides, or at least 55 nucleotides, to the full sequence. In one embodiment, the normalization sequence can include a sequence of SEQ ID NO:1, 2, 3, 4, 5, 6, or 7. A normalization sequence may have perfect identity, at least 95% identity, at least 90% identity, at least 85% identity, or at least 80% identity with a sequence selected from SEQ ID NO:1-7. A normalization sequence may have perfect complementarity or at least 95% complementarity, at least 90% complementarity, at least 85% complementarity, or at least 80% complementarity with a sequence selected from SEQ ID NO:1-7. A normalization sequence may be continuous or it can have one or more bulges or mismatches upon hybridization. A normalization sequence may also include one or more chemical modifications, such as a 2′ carbon modification. A normalization sequence may or may not form an overhang upon hybridization when detecting a RNA present in a biological sample.

Provided herein is an article that includes a substrate and a plurality of individual polynucleotides. The individual polynucleotides may be selected from SEQ ID NO: 8-3,273, or a complement thereof, or a portion thereof. The polynucleotides are immobilized onto a surface of the substrate. In one embodiment, the polynucleotides are immobilized on the substrate surface to form a microarray.

Provided herein are kits. A kit may include one or more polynucleotides for measuring the expression of at least one T21 biomarker, wherein alteration in the expression of the one or more T21 biomarkers in a subject relative to a control is indicative of the presence, absence, or type of T21. A kit may include one or more polynucleotides that are specific to a selected T21 biomarker

A polynucleotide present in a kit may have a sequence that is identical to a polynucleotide listed at SEQ ID NO:8-3,273, or the complement thereof. In one embodiment, polynucleotide present in a kit may have a portion of a sequence that is identical to a polynucleotide listed at SEQ ID NO:8-3,273, or the complement thereof. The polynucleotides to be used in the measurement of the expression of one or more T21 biomarkers can, depending upon the type of technique to be used. For example, the kit may include polynucleotides useful as primers for QRT-PCR. Polynucleotides useful as probes may be included in a kit and are optionally provided together with a solid substrate, such as but not limited to a bead, a chip, a plate, and a microarray. Polynucleotides may be immobilized on the surface of such a substrate. A kit may also further include a reverse transcriptase, a thermostable DNA polymerase, appropriate buffers and salts, or the combination thereof.

Additional reagents useful in the methods described herein, for example determining the presence, absence, or type of T21 in a subject, may be provided in a kit. Depending on the technique or procedure, the kit may further include one or more additional reagents such as, but not limited to, buffers such as amplification buffers, hybridization buffers, labeling buffers, or any equivalent reagent. Reagents may be supplied in solid (e.g., lyophilized) or liquid form, and these may optionally be provided in individual packages using containers such as vials, packets, bottles and the like, for each individual reagent. Each component can for example be provided in an amount appropriate for direct use or may be provided in a reduced or concentrated form that can be reconstituted.

A kit may further include materials and tools useful for carrying out methods described herein. A kit can be used for example in diagnostic laboratories, clinical settings, or research settings. The kit may further include instructions for use, including for example any procedural protocols and instructions for using the various reagents in the kit for performing different steps of the process. Instructions for using the kit according to one or more methods of the invention may include instructions for processing a biological sample obtained from a subject and/or for performing the test, and instructions for analyzing or interpreting the results. Instructions may be provided in printed form or stored on any computer readable medium including but not limited to DVDs, CDs, hard disk drives, magnetic tape and servers capable of communicating over computer networks. A kit may further include one or more normalization sequences.

It will be understood that generally, components of a kit are conveniently packaged or bound together for ease of handling in commercial distribution and sale.

### Combinations of Nucleic Acid Biomarkers

In some embodiments, a method of detecting a combination of nucleic acid biomarkers in a human subject can include: obtaining a nucleic acid sample from the human subject; selecting the combination of nucleic acid biomarkers; analyzing a transcriptome of the human subject for the combination of nucleic acid biomarkers in the nucleic acid sample from the human subject; detecting in the nucleic acid sample the presence of the combination of nucleic acid biomarkers, wherein each nucleic acid biomarker in the combination of nucleic acid biomarkers has a variation from a transcription standard.

In some embodiments, the method includes providing the transcription standard for each nucleic acid biomarker for the combination of nucleic acid biomarkers.

In some embodiments, the method includes providing the combination of nucleic acid biomarkers as a set of primers and/or probes.

In some embodiments, the method includes obtaining cell free plasma RNA as the nucleic acid sample. In some embodiments, the nucleic acid biomarkers are RNA.

In some embodiments, the method can include generating a report, the report reciting the presence of the combination of nucleic acid biomarkers being present in the nucleic acid sample of the human subject being present in a biomarker amount that is varied from the transcription standard.

In one embodiment, a kit includes purified or isolated nucleic acids, wherein the nucleic acids have the sequences of each of the nucleic acid biomarkers in the combination of biomarkers. As such, each recited combination can be uniquely included in a kit. In some aspects, the nucleic acid biomarkers are attached to a substrate of a biochip, where each nucleic acid biomarker can be in a unique position or a position can include one or more of the nucleic acid biomarkers of the combination.

As used herein, “nucleic acid biomarker” or “biomarker” is defined to be a nucleic acid, such as an RNA, that is present in an abnormal amount compared to a standard or normal amount. The biomarker thereby then serves as a tool to look for changes in the transcription thereof. For example, a biomarker can be present at a normal or standard level when there is no disease state or susceptibility of a disease state, but the biomarker is present at a changed level or a variation from the standard or normal amount. While SNPs may be detected by merely identifying the presence, the nucleic acid biomarkers described herein may always be present, but the change in the transcription thereof or change in the amount or concentration in blood or plasma provides the indication that the subject may have a condition that is marked by the biomarker. Thus, by using the term “biomarker” it is clear that the transcription thereof, amount thereof or concentration thereof is not normal, such that it is changed. Such a changed condition can be compared to subject (e.g., pregnant woman, fetus possibly having T21 whether known or unknown) prior to pregnancy or in early pregnancy (e.g., earlier than 12 weeks or between 16-20 weeks). Thus, by being defined as a biomarker, it is defined that the transcription thereof, amount thereof or concentration thereof is detectably different from a standard or normal person without the condition or the same subject prior to onset of the condition-T21 in the fetus. In some aspects, a biomarker requires at least a fold change relative to the normal or standard amount or concentration or transcription, or at least a 1.3 fold change, or at least a 1.4 fold change, or at last a 1.5 fold change, or at least a 1.6 fold change, or at least a 1.7 fold change, whether the change is up regulation (increased transcription, amount or concentration) or down regulation (decreased transcription, amount or concentration) compared to a standard or normal amount or compared to that of the subject prior to being pregnant or prior to 9 weeks or prior to 12 weeks of gestation (or prior to 7 weeks or prior to 10 weeks implantation).

As used herein, “combination of biomarkers” or “combination of nucleic biomarkers” defines a unique combination of nucleic acids that are biomarkers under the definition of a biomarker provided herein. The combination of biomarkers provides an indication of a T21 disease state in a fetus of a pregnant woman.

The combination of biomarkers can be detected to be present in a biomarker amount by hybridizing the biomarker with a biomarker primer (PCR) or biomarker probe (biochip). The combination of biomarkers can be calculated or quantitated with a normalization nucleic acid during the detection of the biomarker amount thereof. The combination of biomarkers can be tied to a disease state-T21 of a fetus. Once the disease state is identified for the combination of biomarkers, a treatment regimen can be provided to the subject, such as pregnant woman or the fetus thereof, that has the biomarker amount. In one aspect, a further confirmatory diagnostic protocol can be performed to confirm T21. The treatment regimen can then be implemented on the pregnant woman, such as providing a report with the information of abortion as an option or choosing to end the pregnancy. The combination of biomarkers can be present as a kit in the combination. The kit may include instructions identifying the combination of biomarkers and the indication of the disease state thereof.

Transcriptome-typing can be performed with the combination of biomarkers. Transcriptome-typing is equivalent to genotyping for transcribed RNA.

A method for detecting T21 in an asymptomatic subject comprising: (a) subjecting a sample from the subject to a procedure to detect polynucleotides (biomarkers) of specific Groups; (b) detecting T21 by comparing the amount of polynucleotides in a specific biomarker group to the amount of such polynucleotides obtained from a control who does not have T21 wherein the polynucleotides comprise at least one of, or are selected from Group 1, 2, 3, 4, 5, or combination groups thereof, or any other combination of groups described herein.

A method where the procedure comprises detecting Groups of polynucleotides in the sample by contacting the sample with oligonucleotides that hybridize to the polynucleotides (biomarkers); and detecting in the sample levels of nucleic acids that hybridize to the polynucleotides relative to a control, wherein a change or significant difference in the amount or status of the polynucleotides in the sample compared with the amount or status in the control is indicative of T21.

A method wherein the procedure comprises: contacting the sample with the group of biomarkers that specifically bind to the polynucleotides under conditions effective to bind the biomarkers and form complexes; measuring the amount or status of the polynucleotides present in the sample by quantitating the amount of the complexes; and wherein a change or significant difference in the amount or status of polynucleotides in the sample compared with the amount or status obtained from a control subject who does not suffer from T21 is indicative of T21.

Within certain embodiments, the amount of polynucleotides that are RNA are detected via polymerase chain reaction using, for example, oligonucleotide primers that hybridize to one or more combinations of biomarkers, or complements of such combinations of biomarkers. Within other embodiments, the amount of RNA is detected using a hybridization technique, employing oligonucleotide probes that hybridize to one or more combinations of biomarkers, or complements thereof.

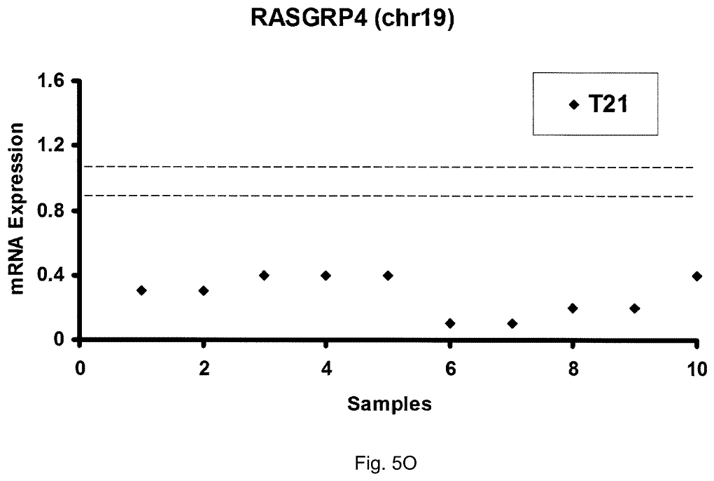

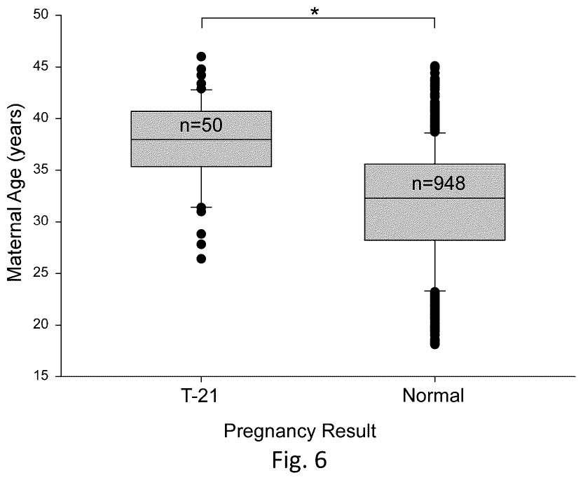

FIG. 6 shows that the maternal age in Normal (euploid fetus) women and those with a Trisomy 21 fetus. The box illustrates the median and the 25th-75 percentile range. The solid circles show the number of women whose age was above the 90th or below the 10th percentile. This data illustrates the well-known increase in Trisomy 21 prevalence with advancing maternal age.

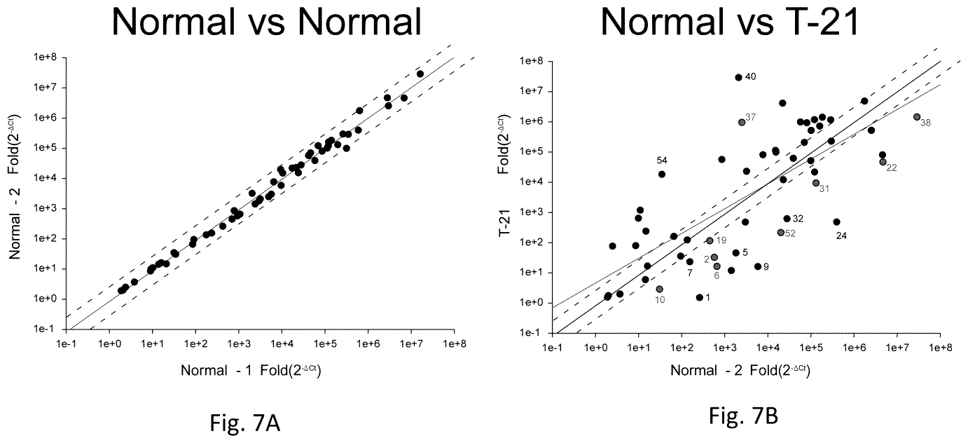

FIGS. 7A-7B show that the protocols provide for high reproducibility of the high throughput assay that is utilized for gene quantification and the differential plasma cell free RNA expression in women with a Trisomy 21 fetus. FIG. 7A shows the Mean RNA expression of a 54 cell free RNAs subset (Group) from the original list of 3,248 plasma cell free RNA markers. This group of 54 cell free RNAs is selected because they had the highest differential expression half in women with a Trisomy 21 fetus. One half of the Normal subjects were selected at random and mean expression for each RNA marker was plotted against the mean expression of the same marker in the second half of Normal women. The solid line represents the correlation between the two groups. FIG. 7B shows the Mean RNA expression for the group of 54 RNA markers for the Trisomy 21 (n=50) (Y axis) plotted against mean expression of the same marker in the Normal subjects (n=948). The light dots identify the 10 variables with the highest p values for differential expression in women with a Trisomy 21 fetus by Mann Whitney U test after adjustment by a Bonferroni correction. The solid line between the dashed lines represents the correlation illustrated on the right, while the solid line that crosses the dashed lines is the correlation between the T21 and Normal groups. Notice the change in slope, which indicates the change obtained with the selected group of 54 cell free RNAs.

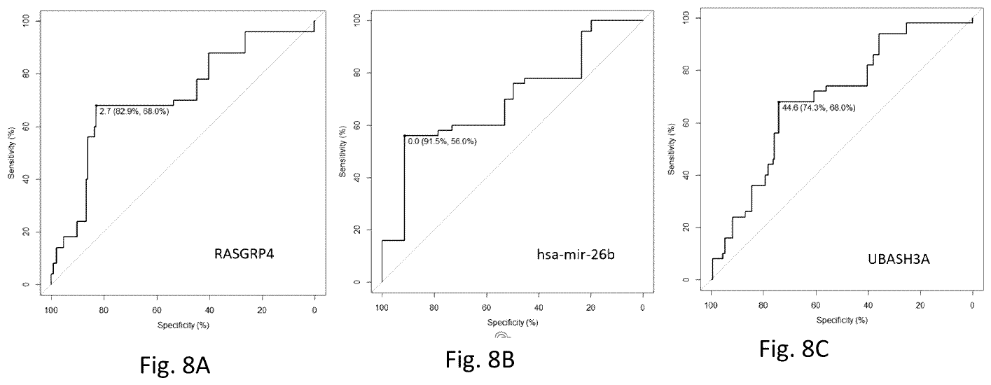

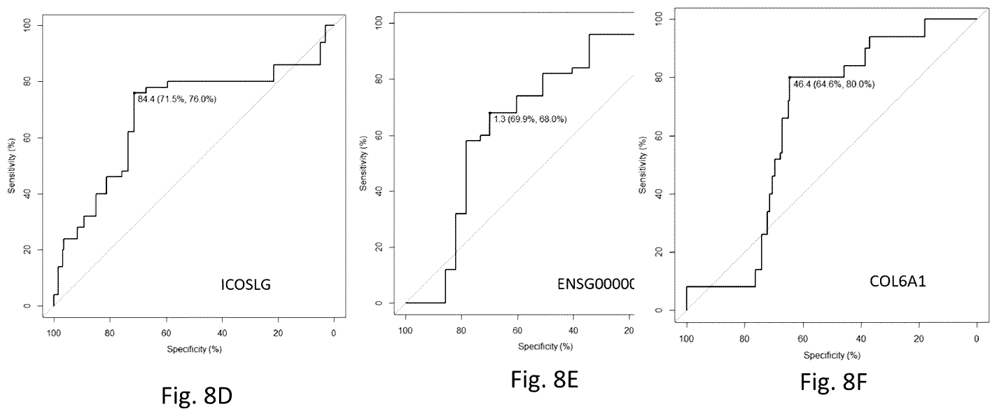

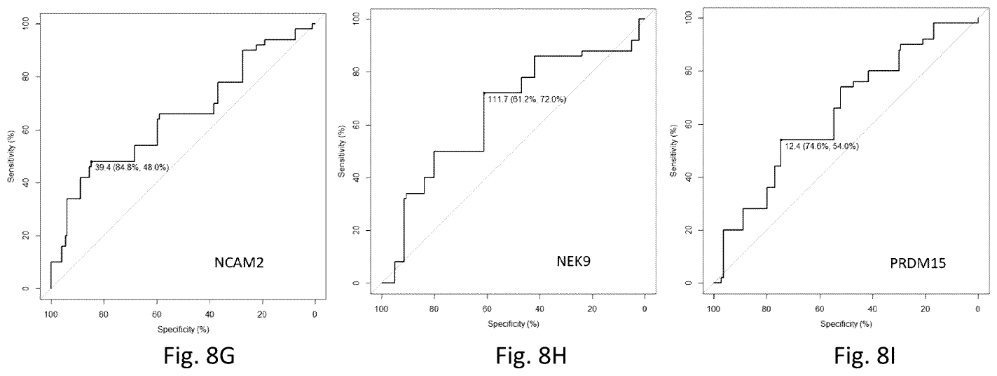

FIGS. 8A-8I show the ROCs for the 9 RNAs shown by the light dots in FIG. 7B with the highest p values. These are a specific subset grouping of the cell free RNAs.

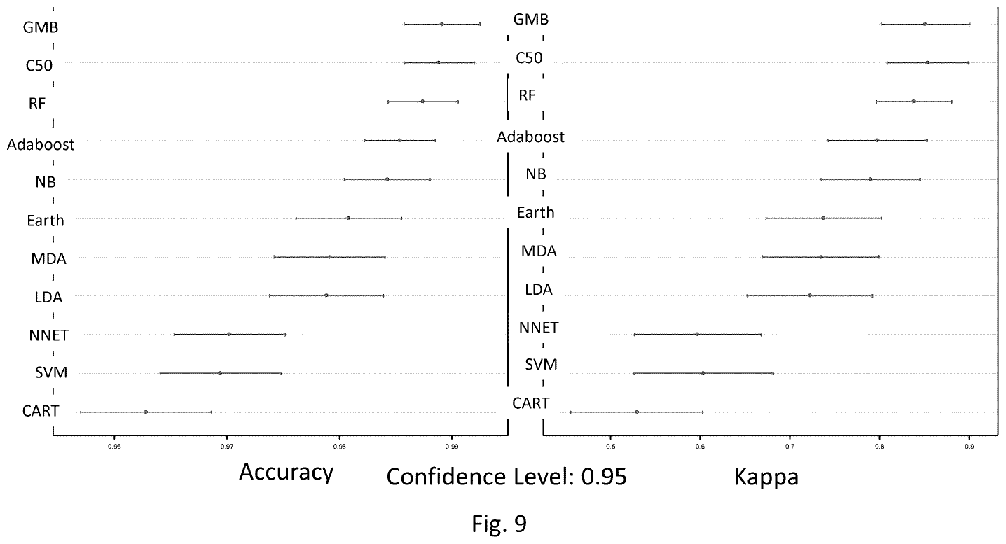

FIG. 9 shows a comparison of 11 Machine Learning (ML) algorithms: Gradient Boosting Machine (GBM), C5.0, Random Forest (FR), Adaboost, Naïve Bayesian (NB), Earth, Mean Decrease in Accuracy (MDA), linear discriminant analysis (LDA), Neural Network (NNET), Support Vector Machine (SVM), and Classification and Regression Trees (CART). GBM and C5.0 proved superior for the detection of Trisomy 21 fetuses with RF close behind using all 54 plasma cell free RNA markers in terms of accuracy and Kappa. These studies support the grouping of the 54 RNA markers.

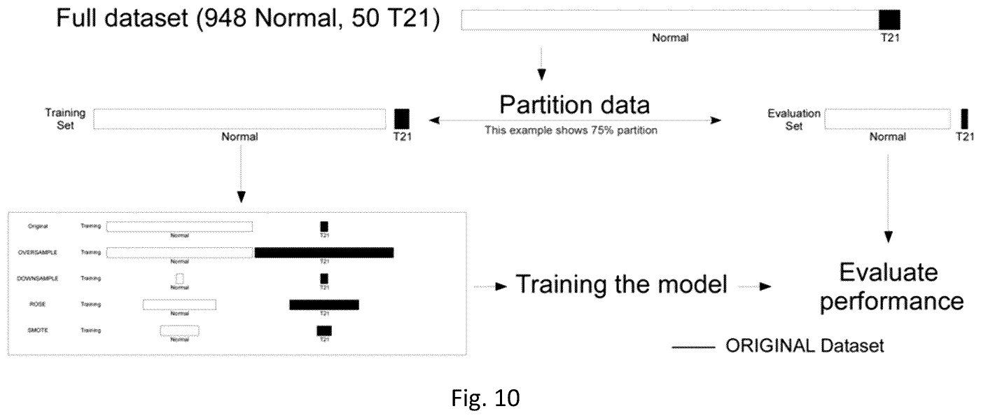

FIG. 10 shows general workflow that leads to the identification of the biomarker subsets that are described herein. The workflow uses artificial intelligence to select the biomarker groups described herein, and the selected biomarker groups can be used in the multiple models in order to identify women whose fetus had/have Trisomy 21.

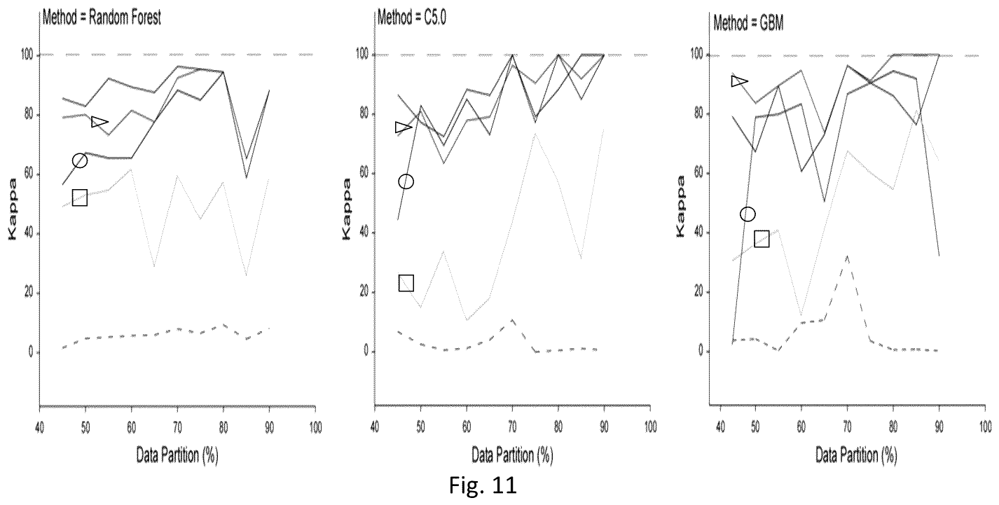

FIG. 11 shows data for the three best performing ML algorithms. The data shows the impact on partitioning whether the protocol uses oversample, down sample, Rose or Smote. Oversampling in each instance provided the highest model Kappa and Accuracy with the optimal performance somewhere between 70-80%.

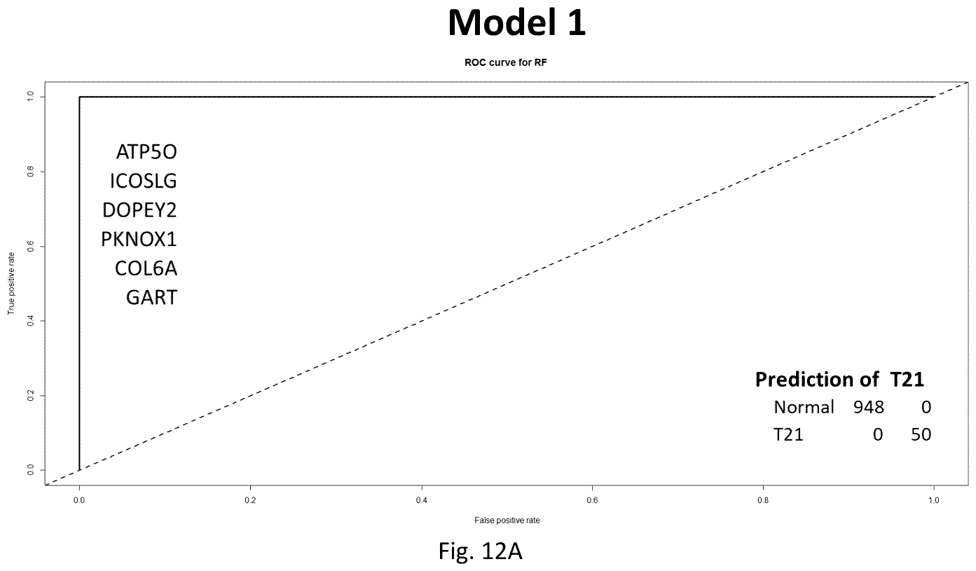

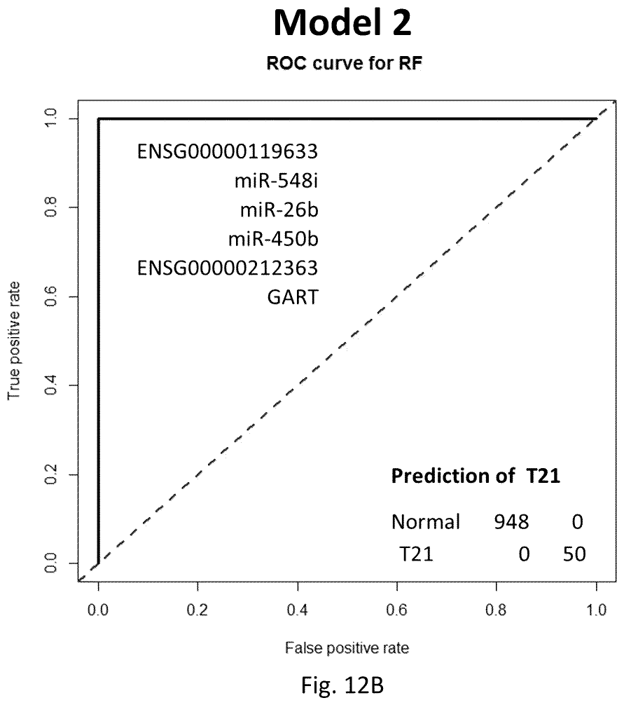

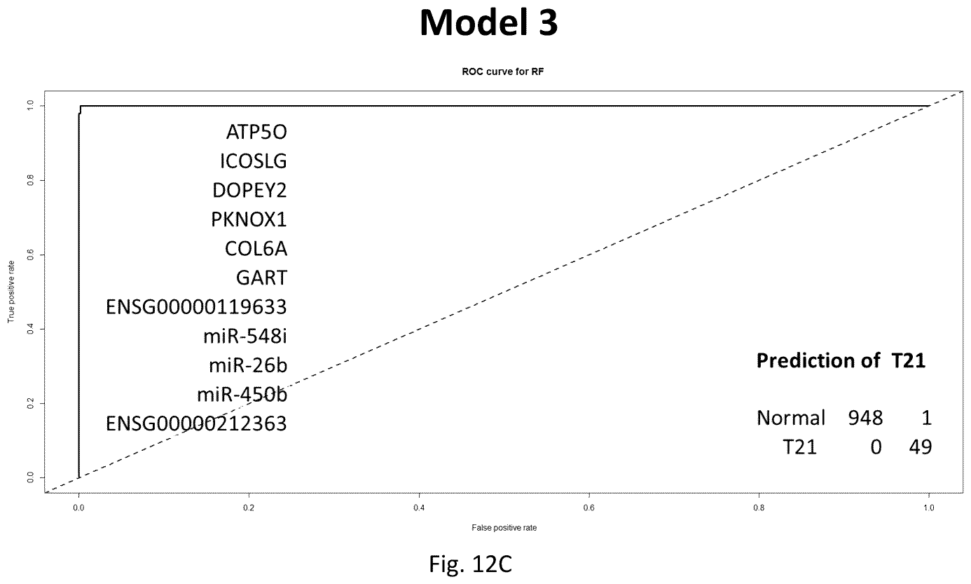

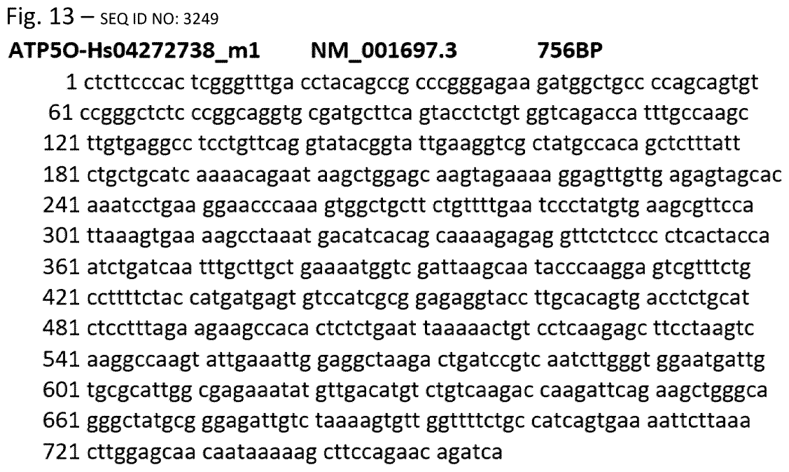

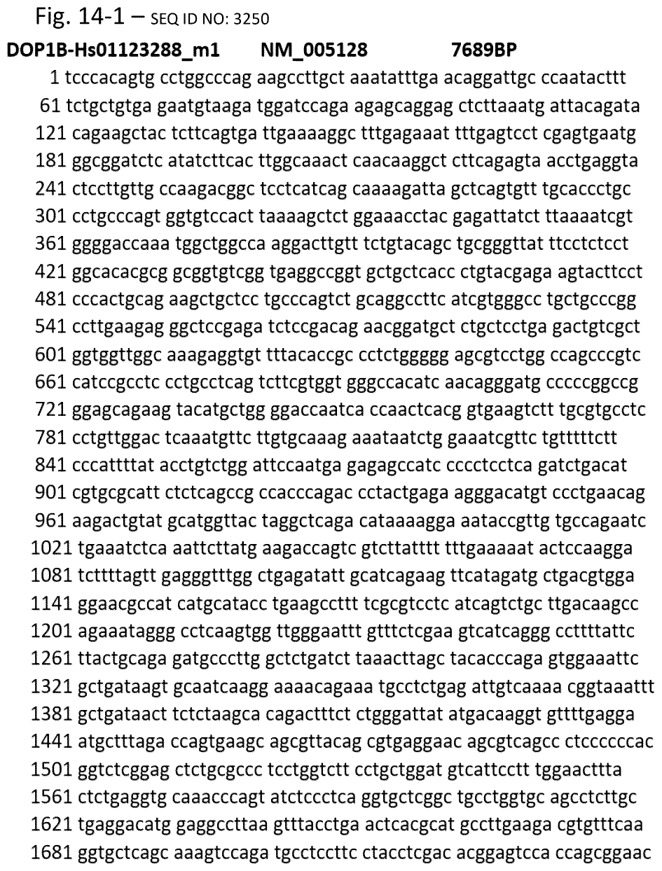

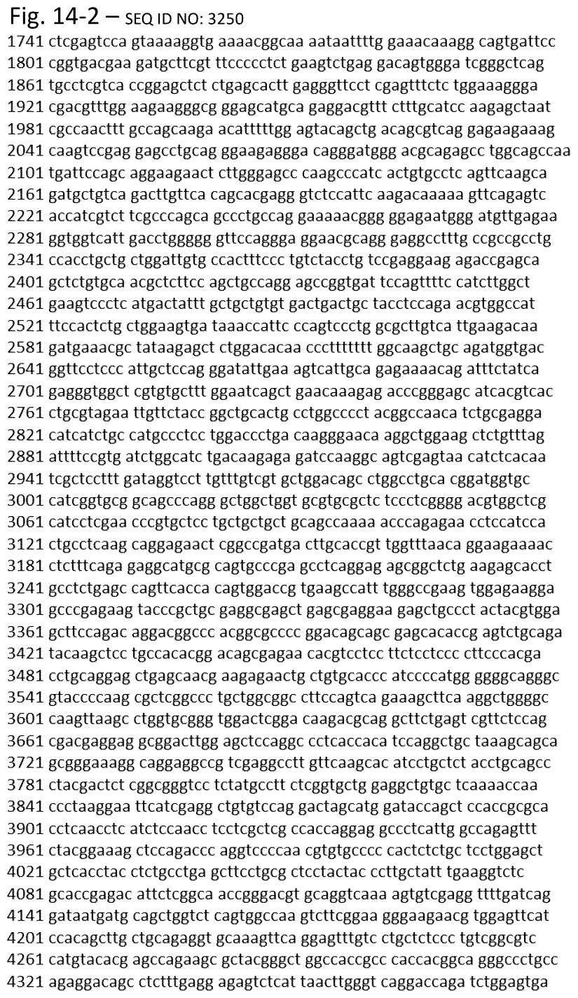

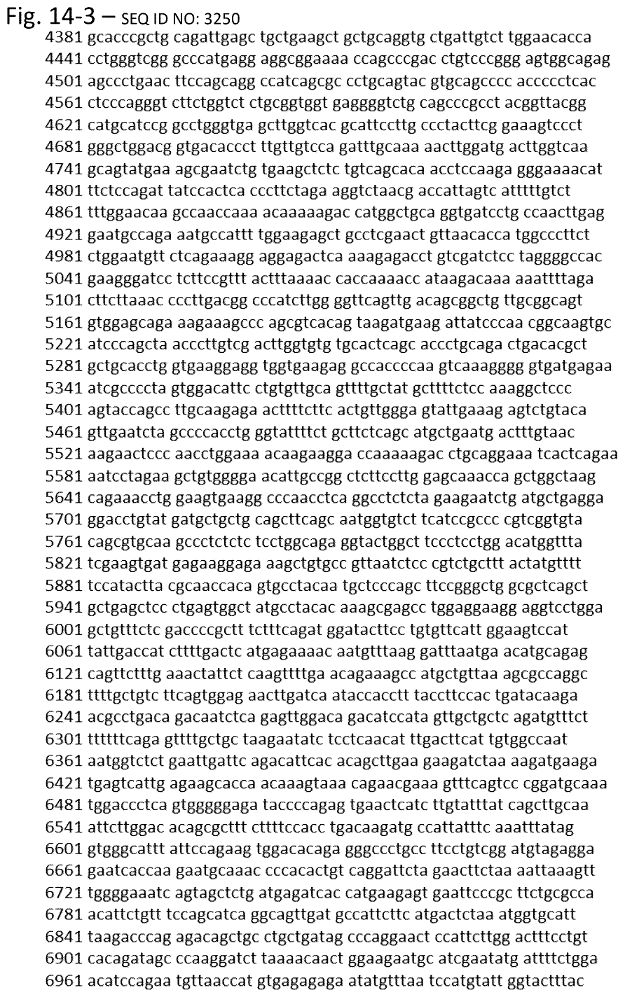

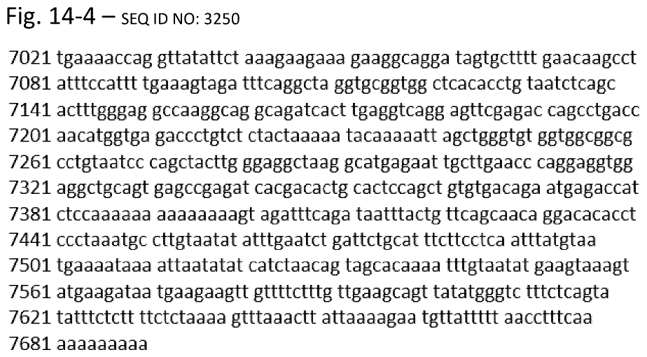

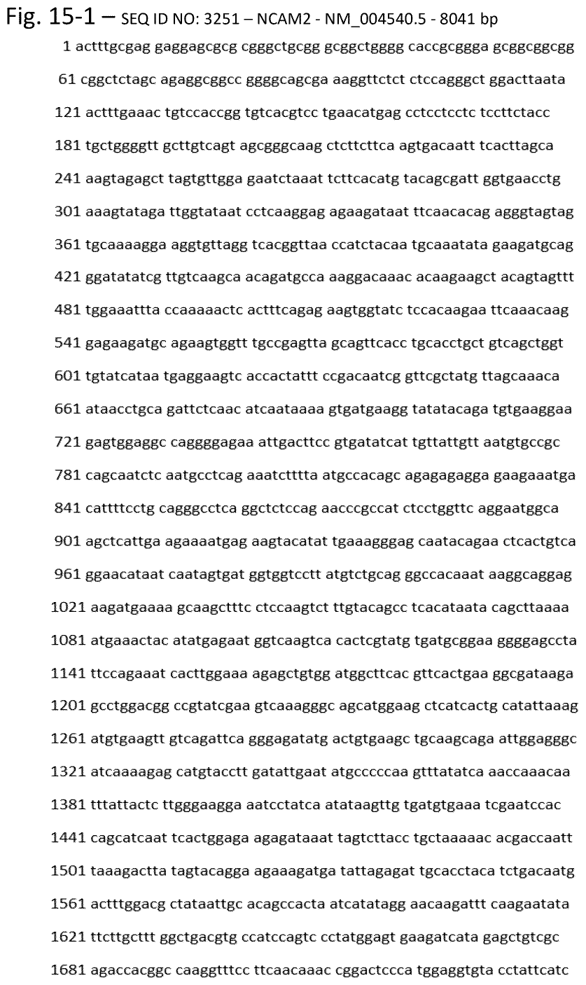

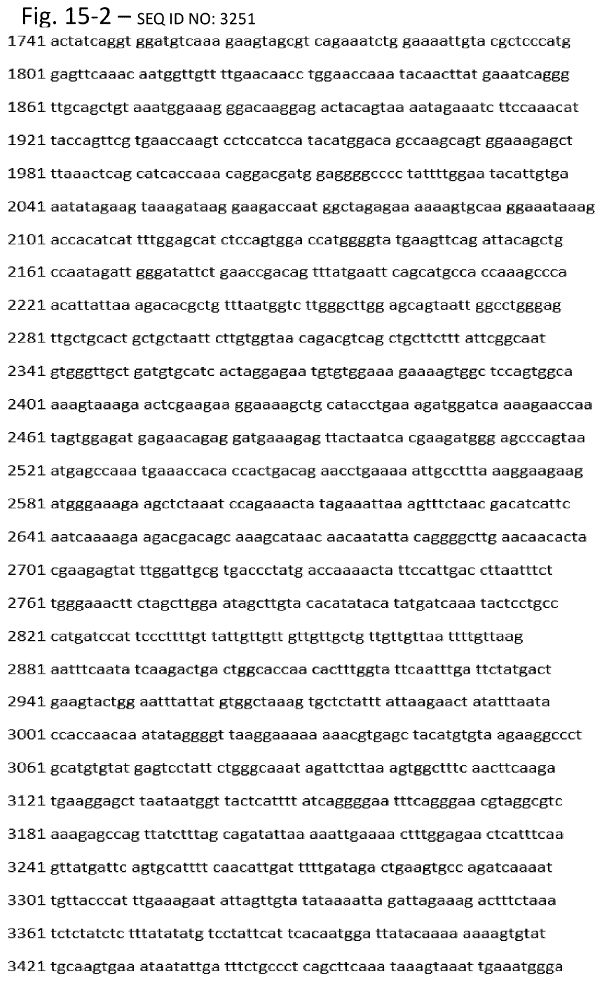

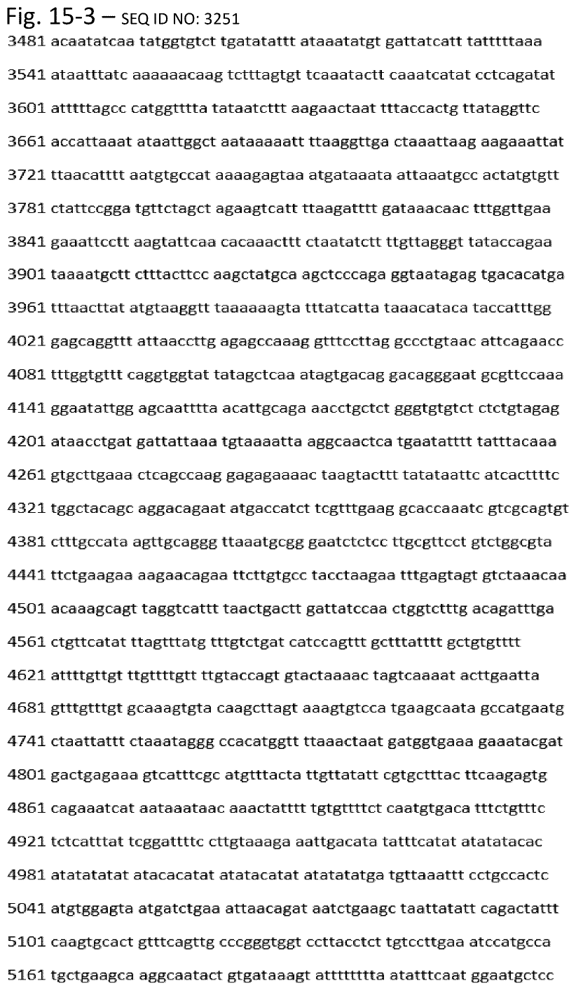

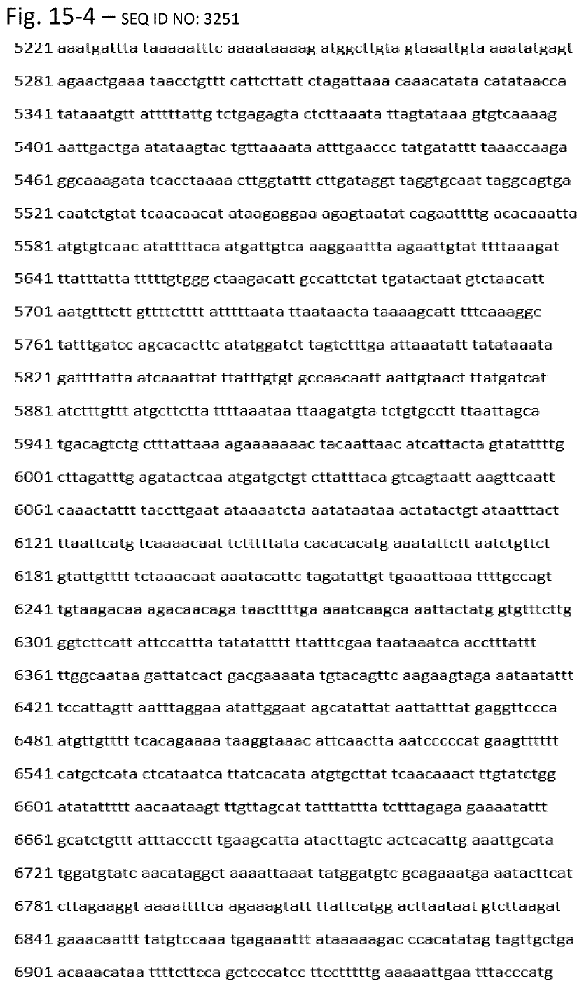

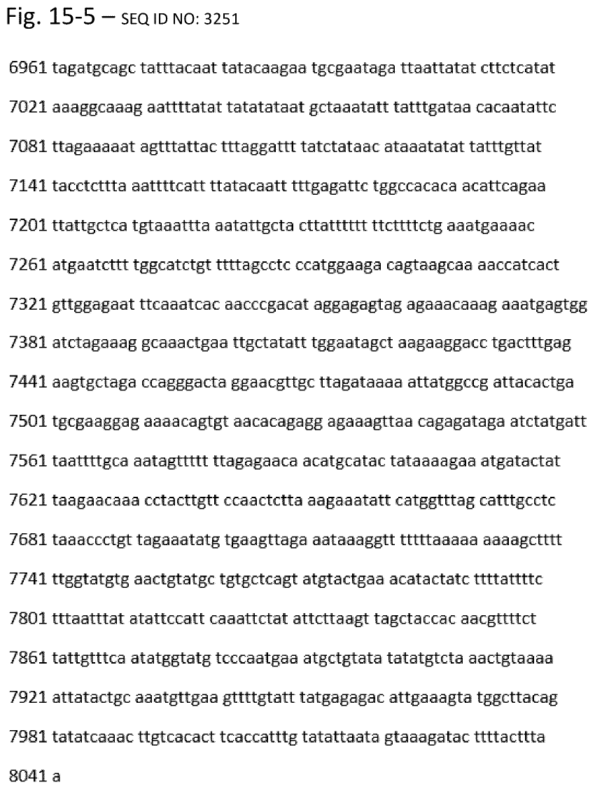

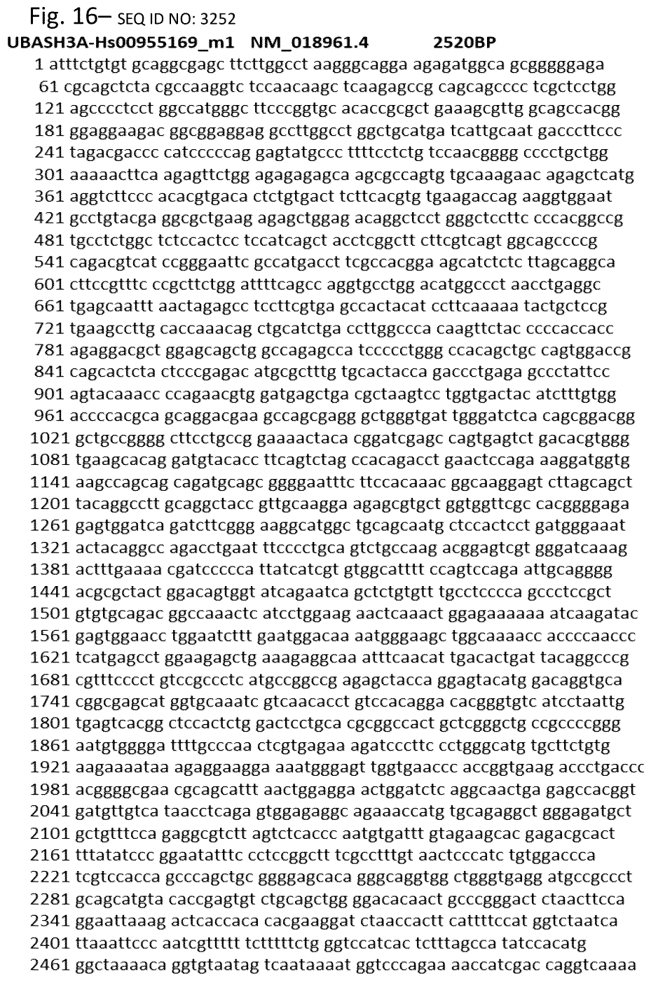

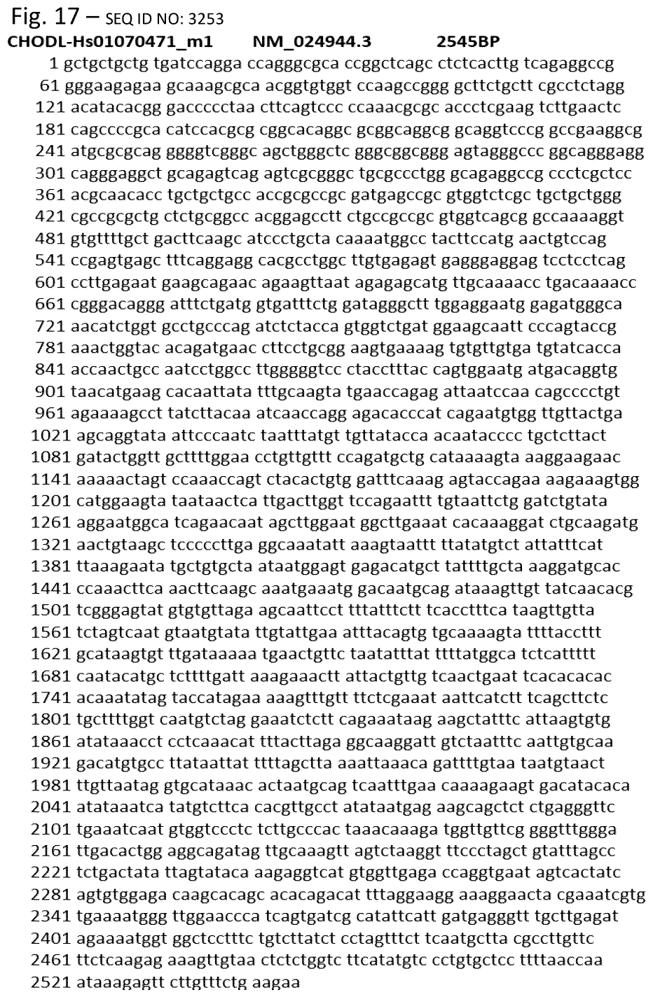

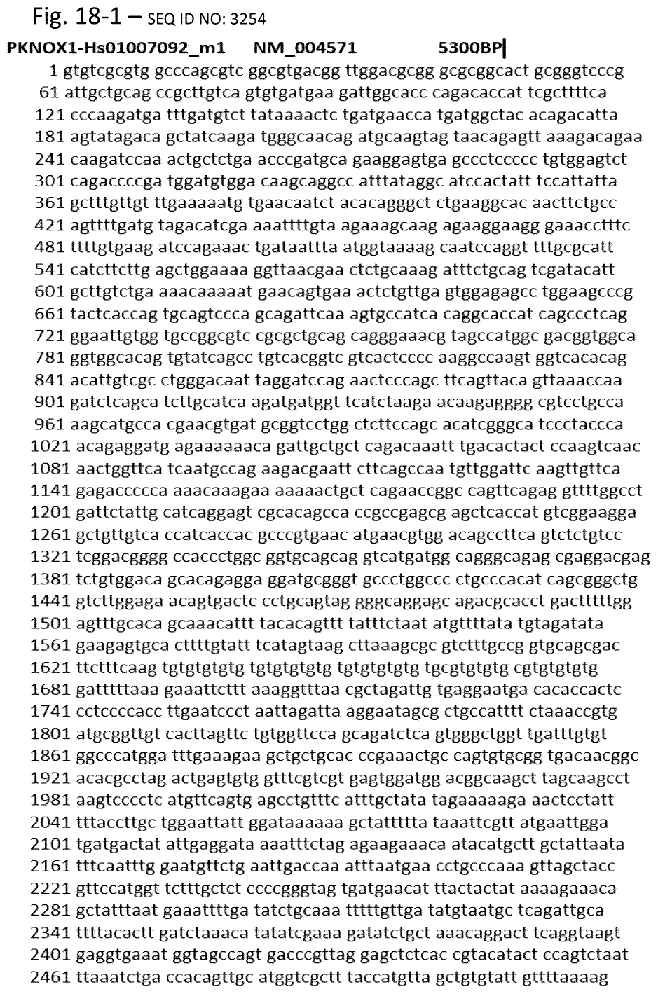

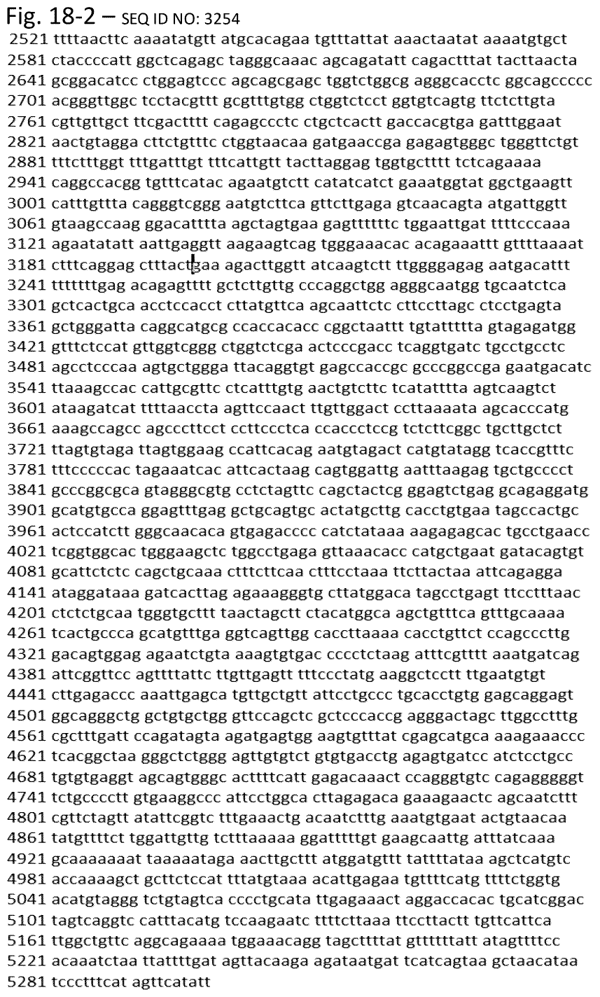

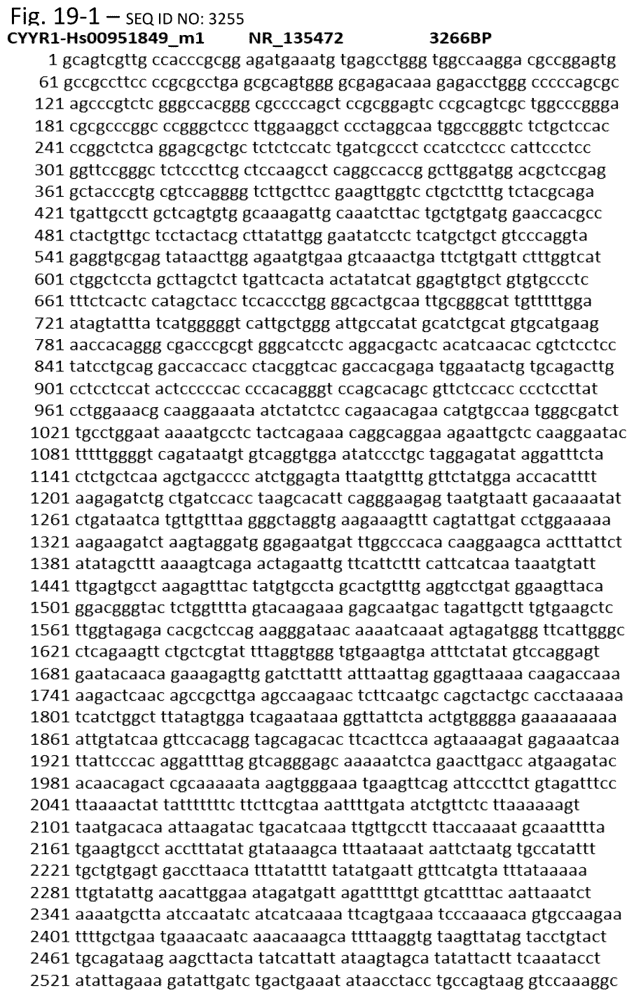

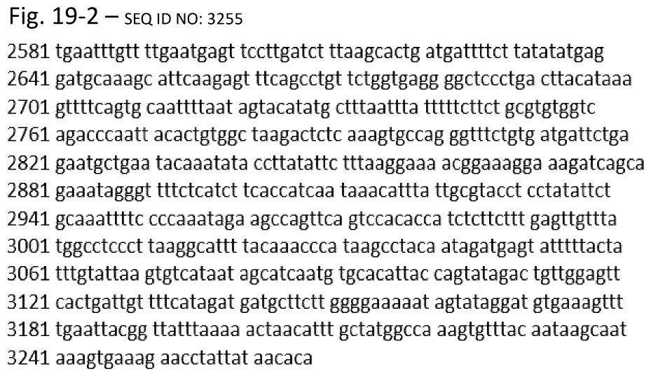

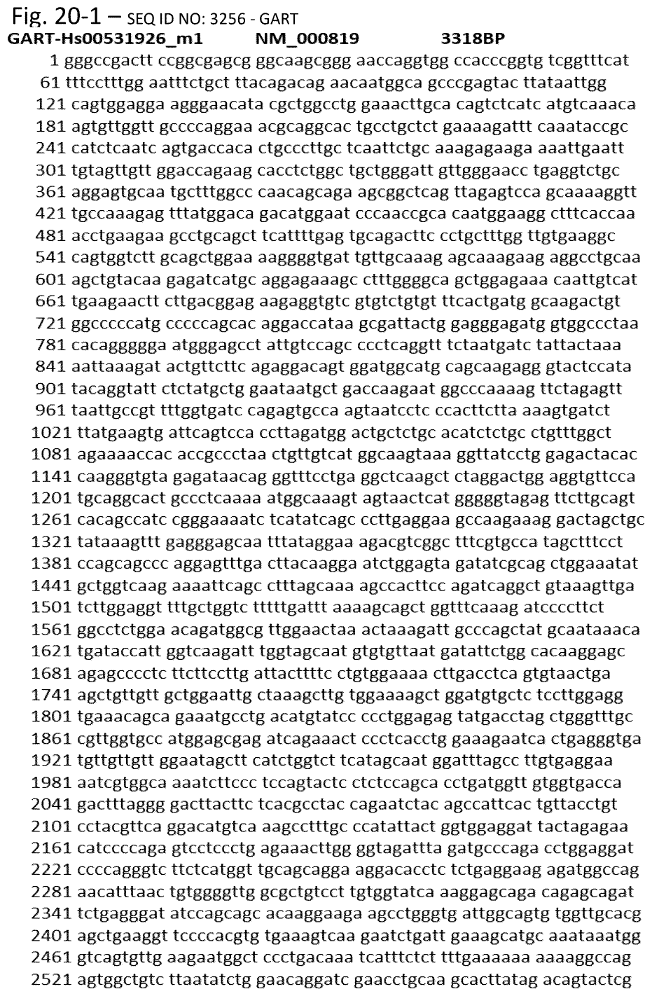

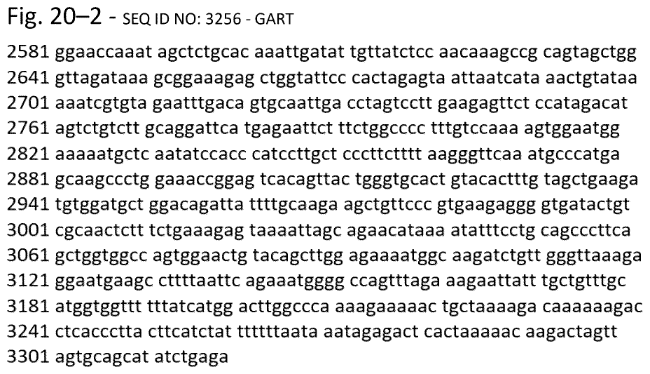

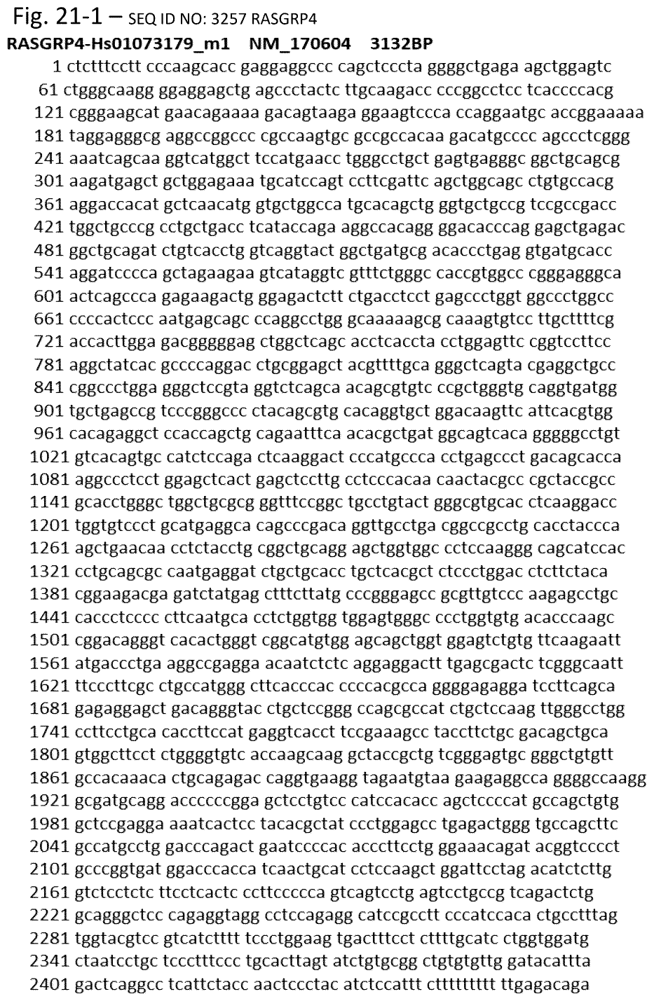

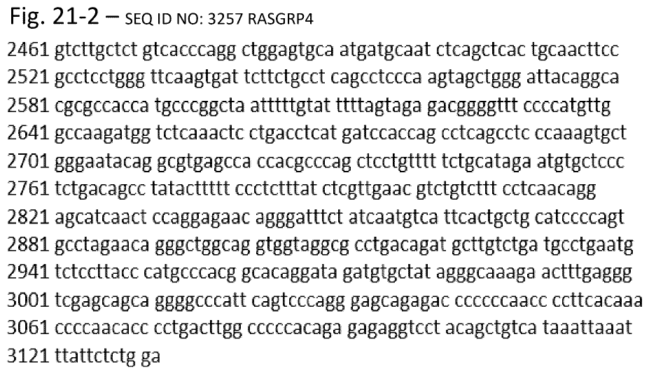

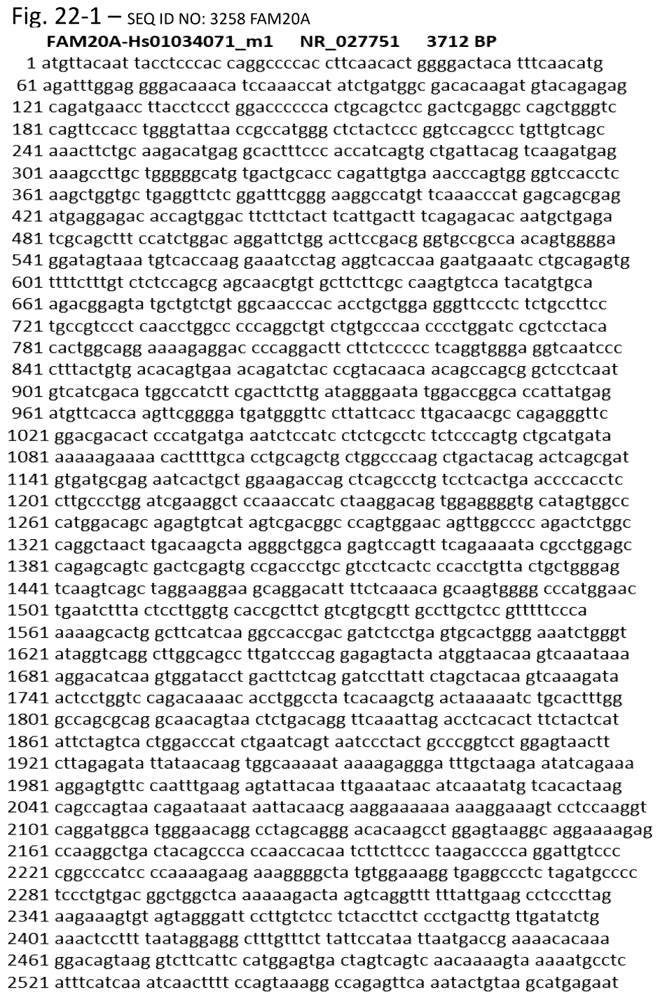

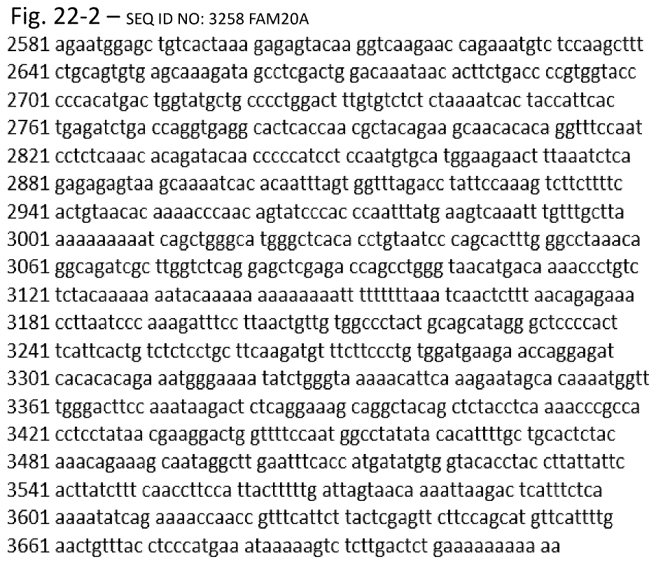

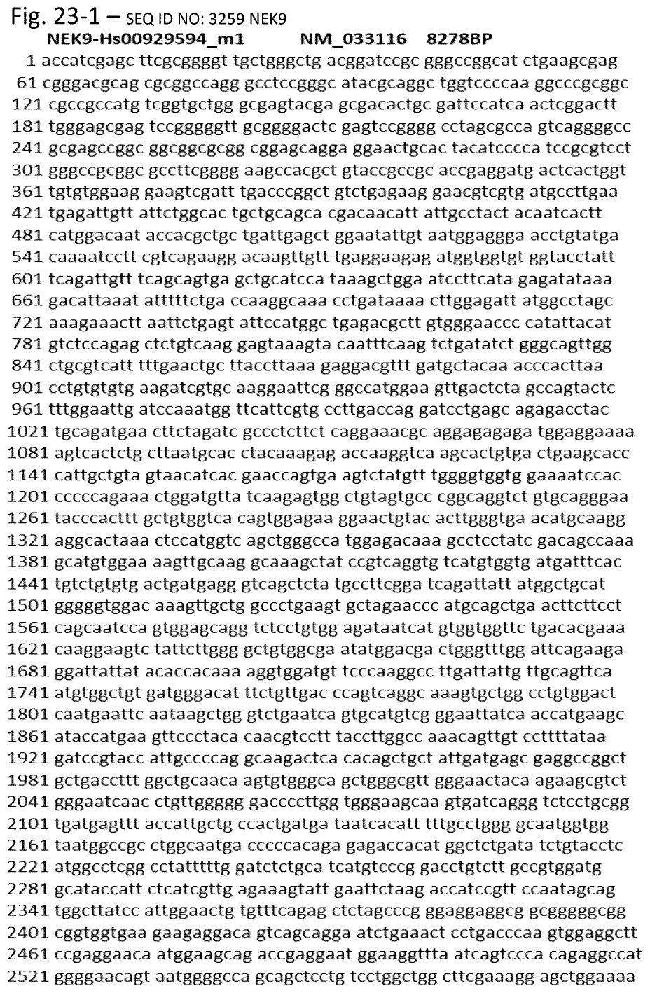

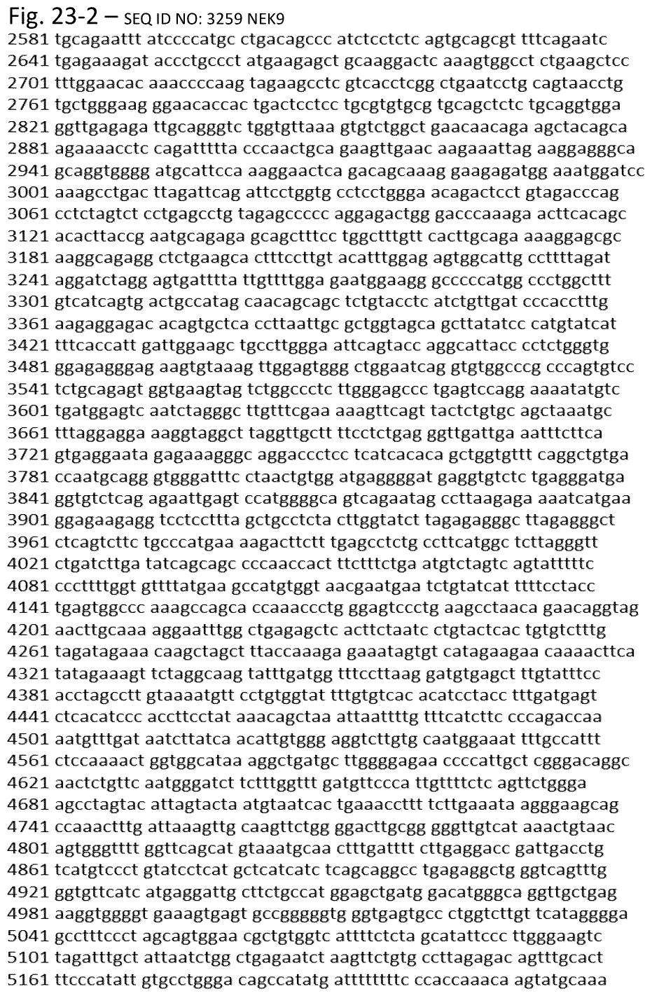

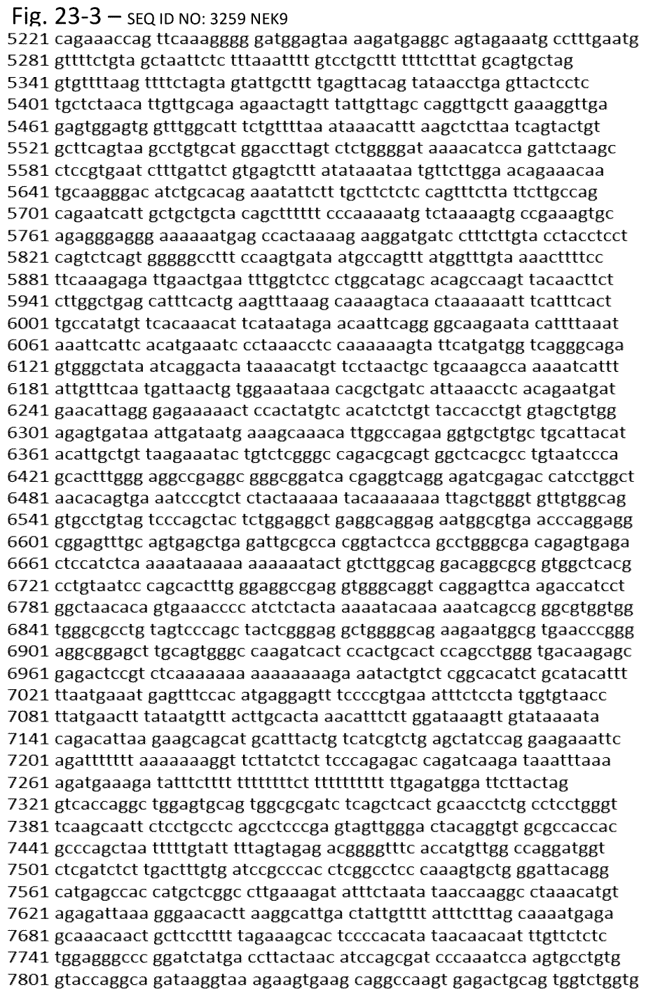

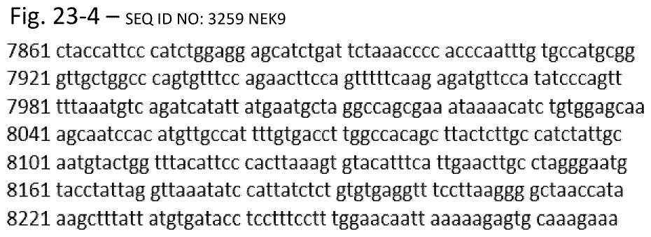

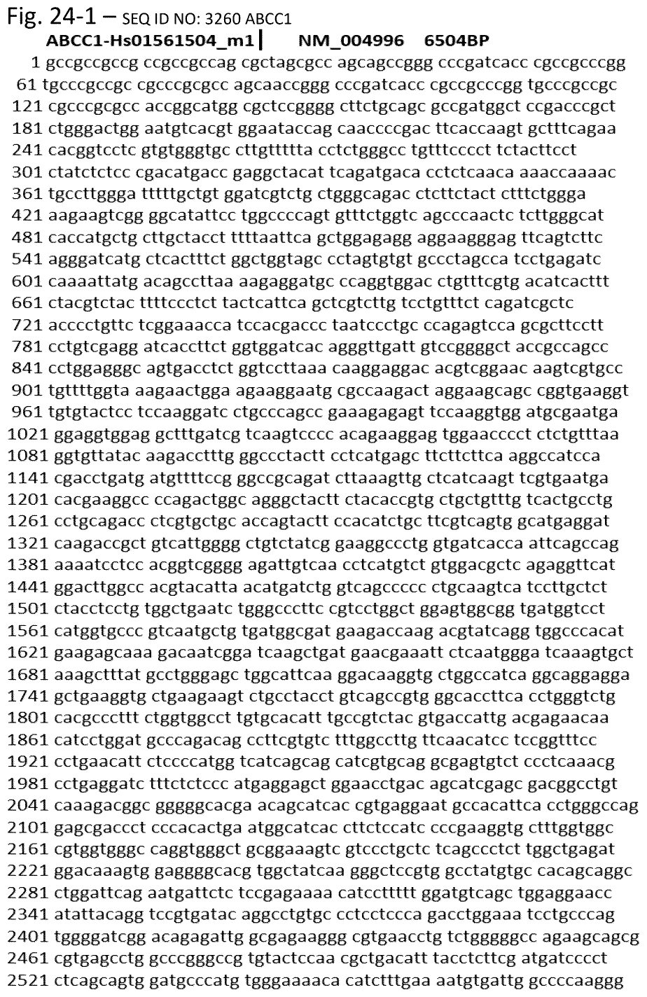

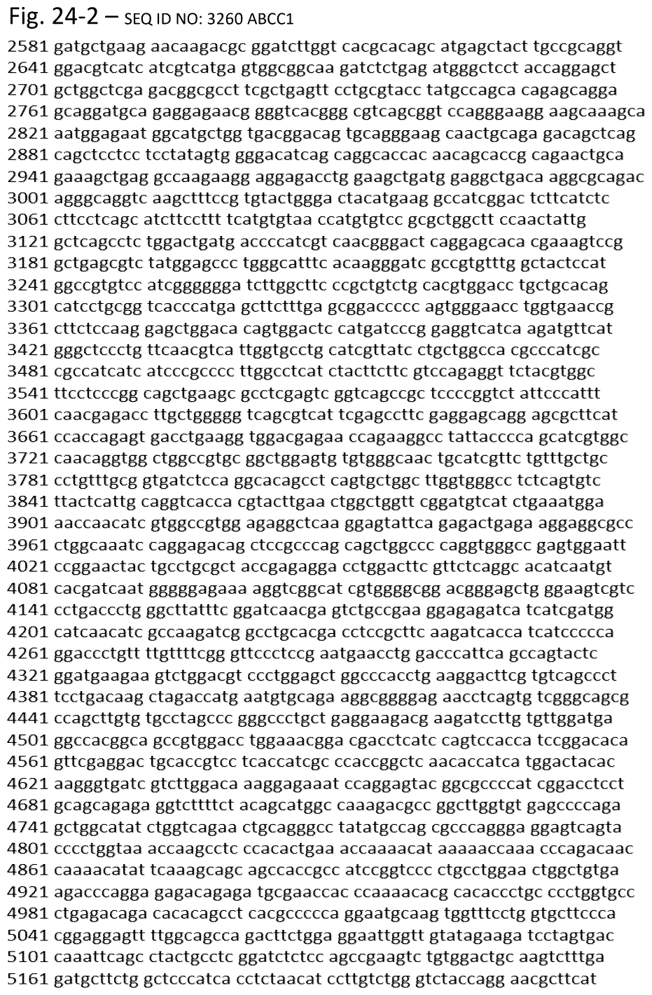

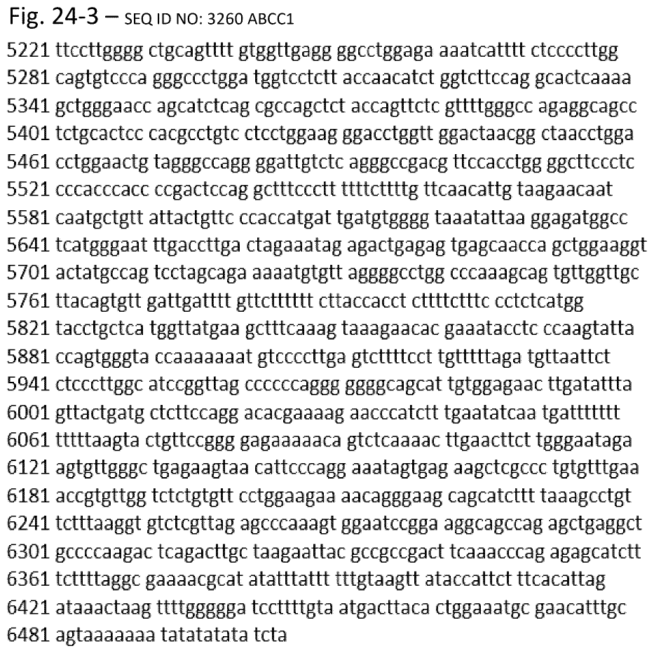

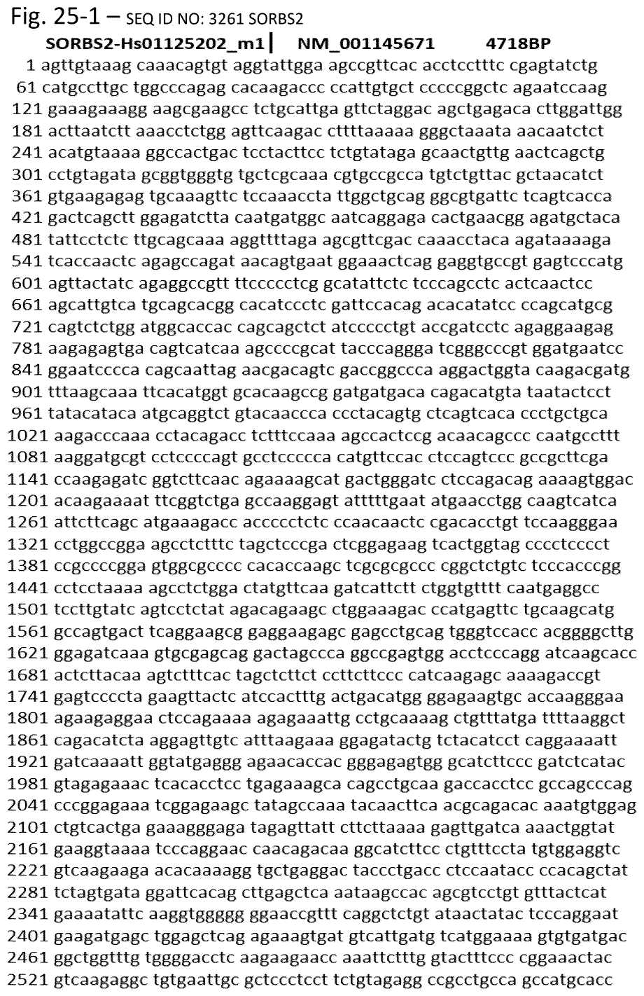

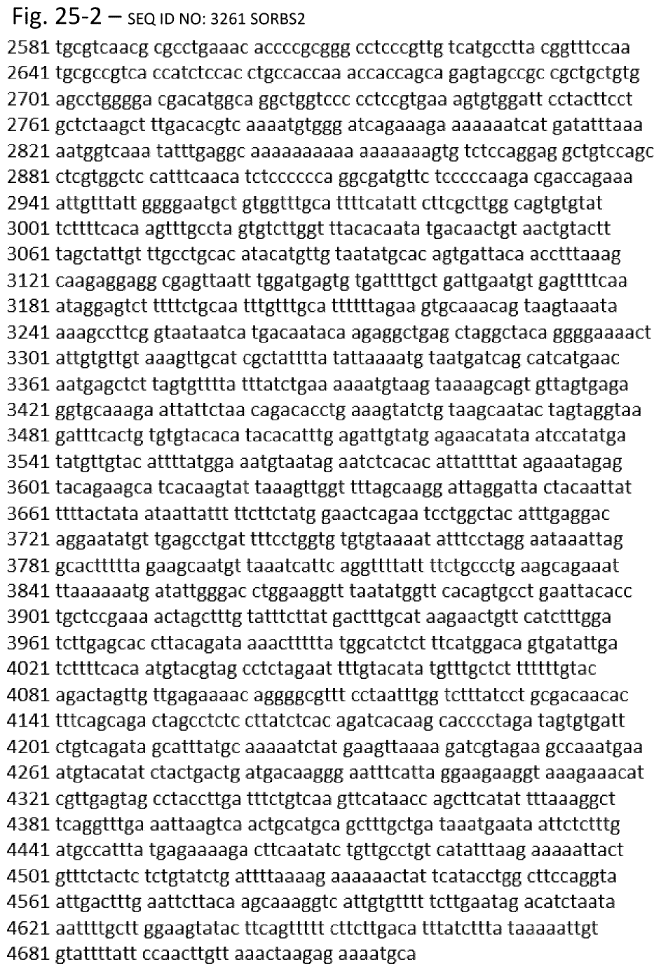

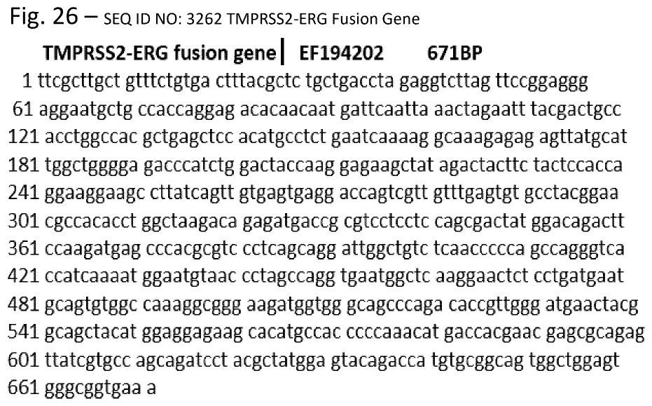

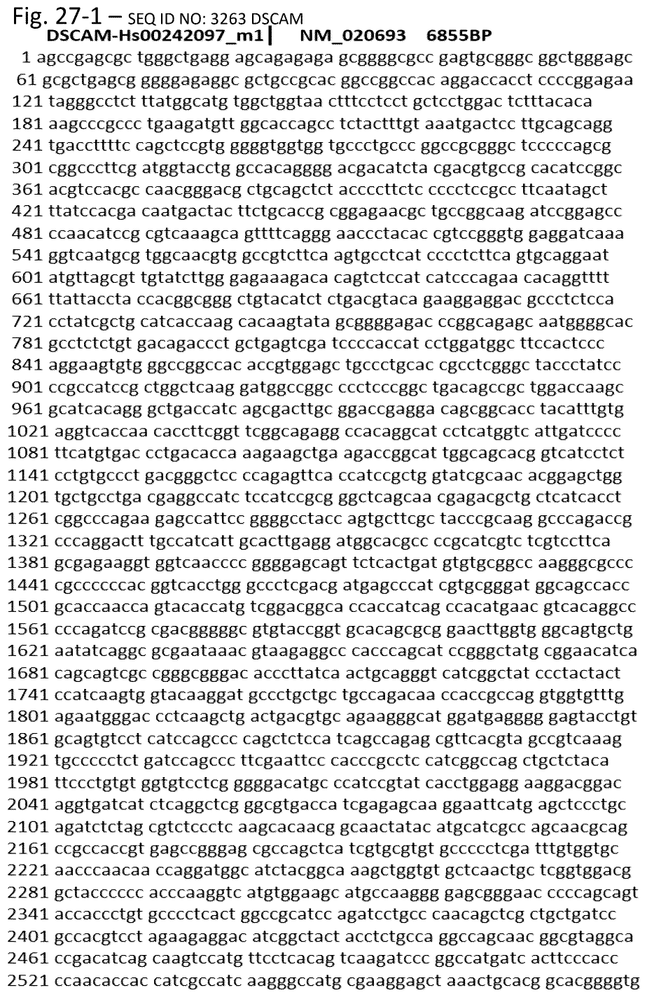

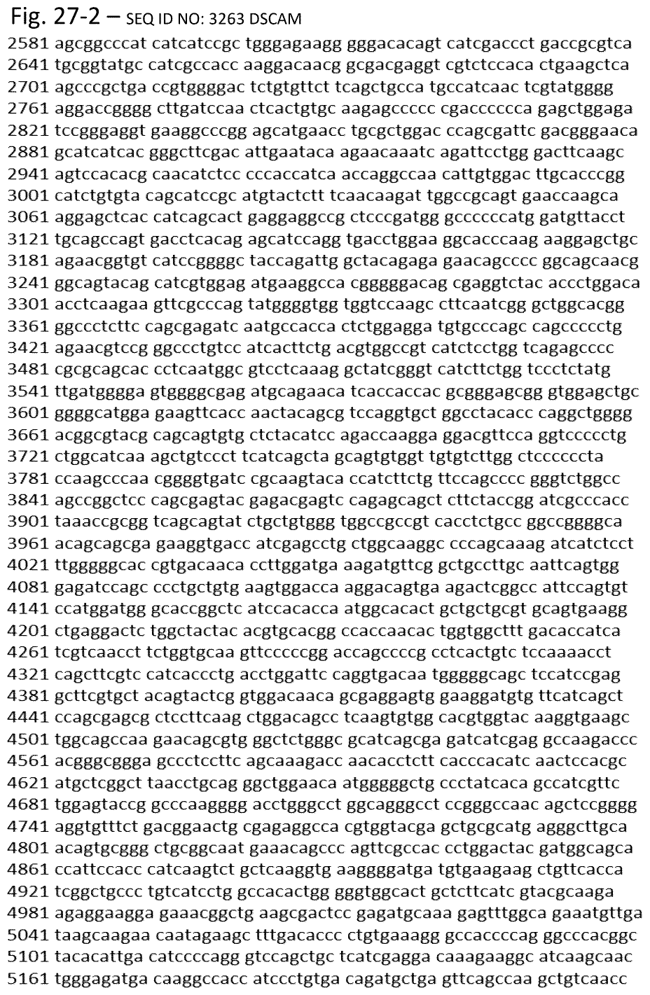

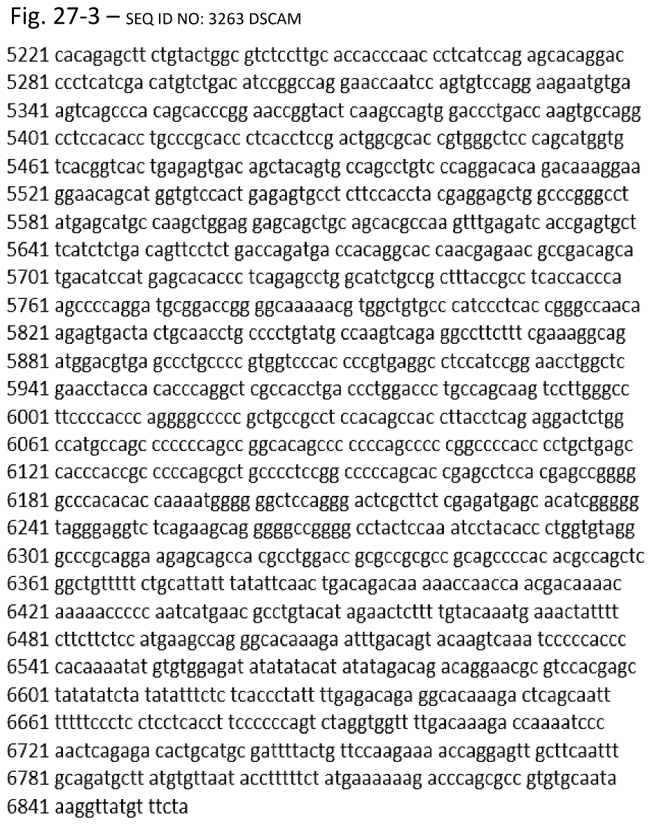

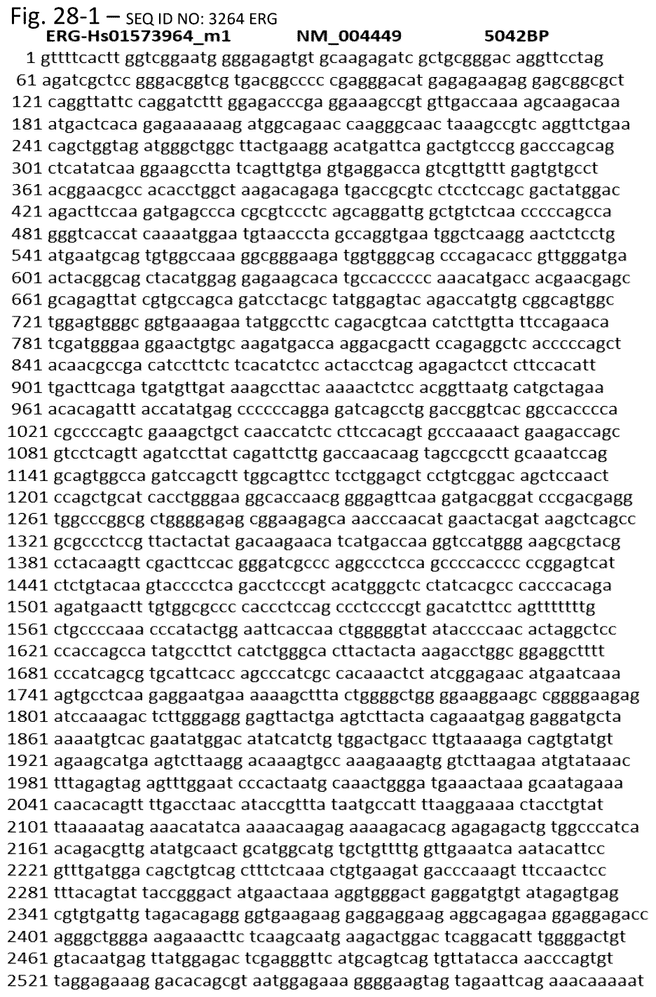

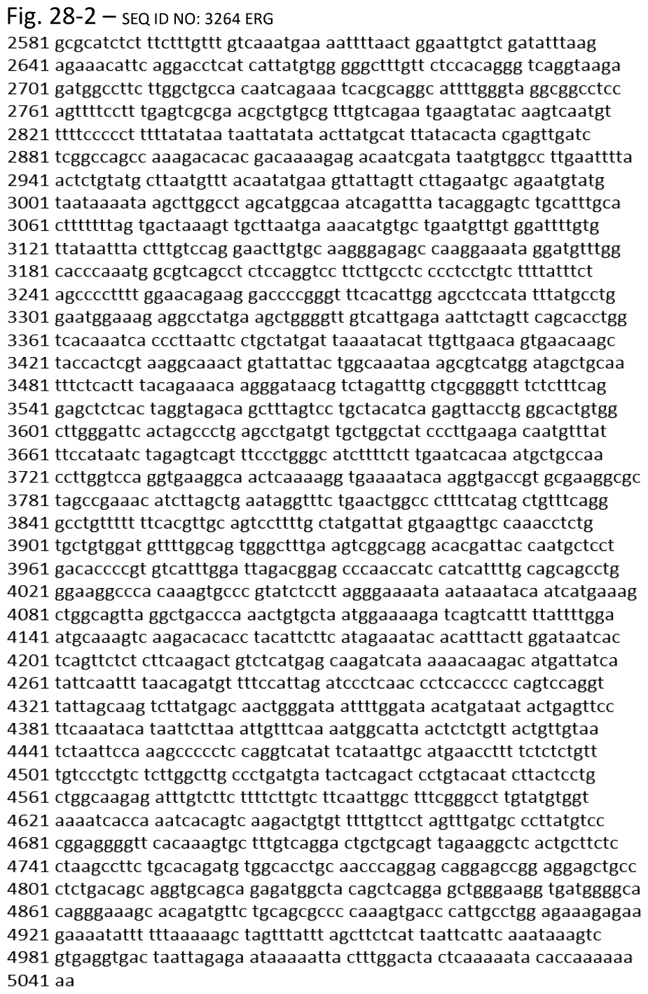

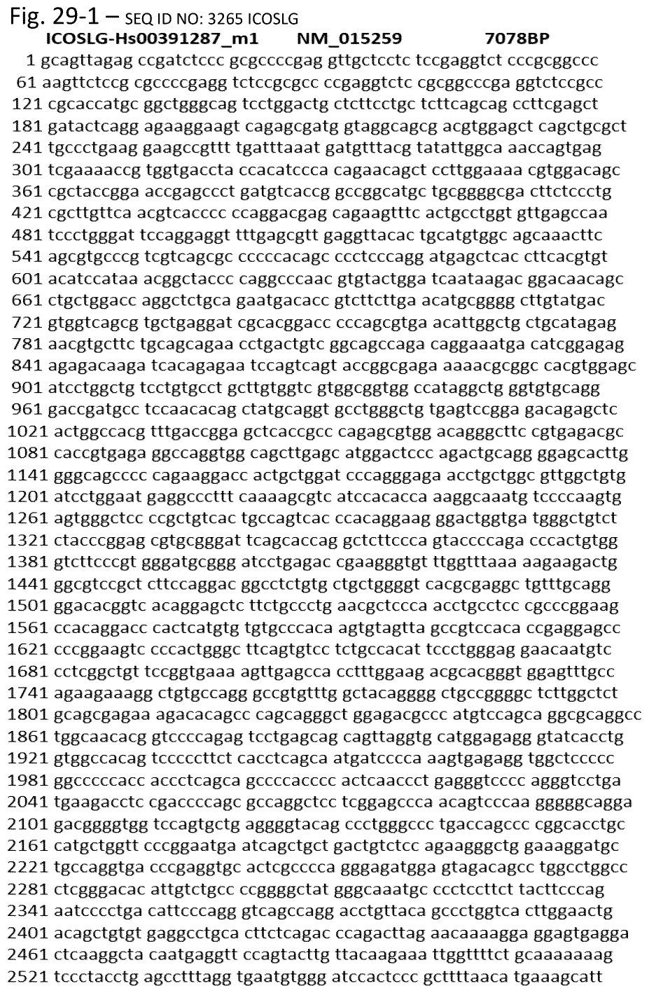

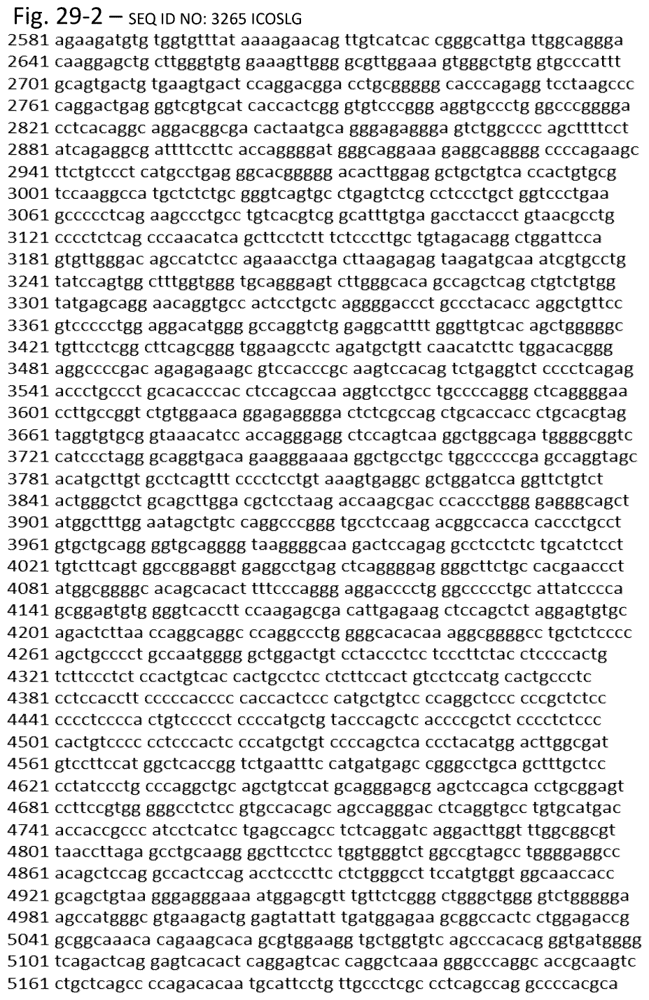

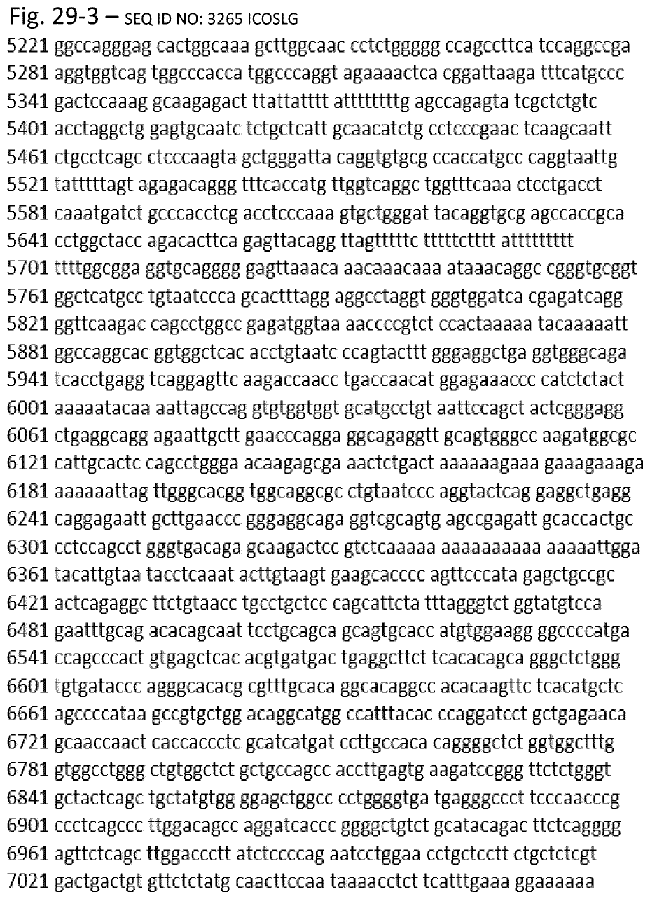

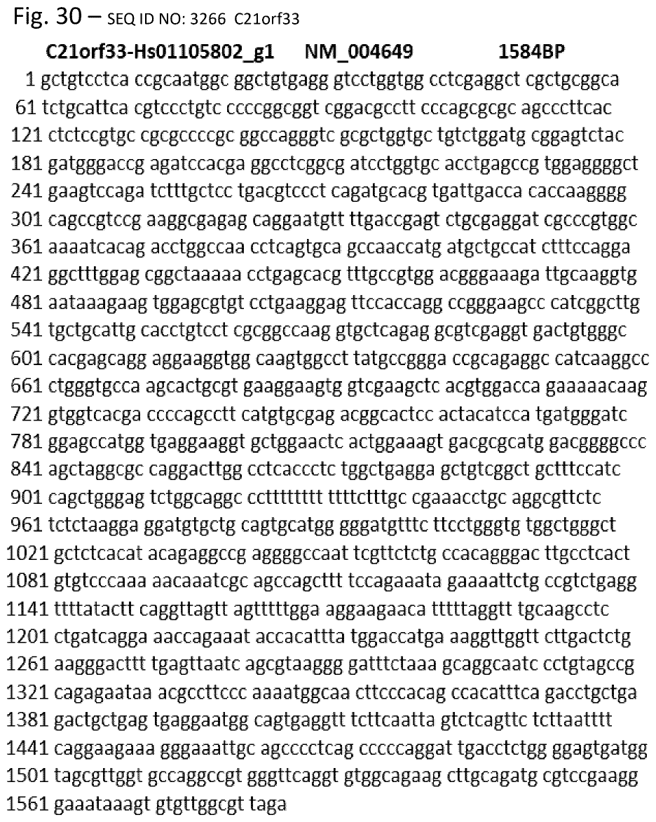

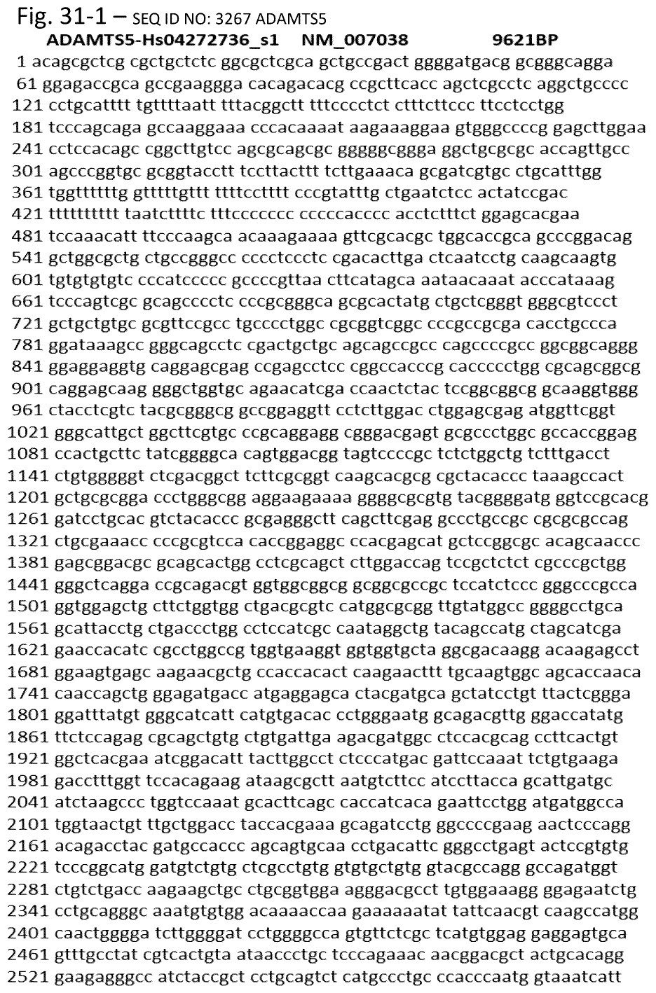

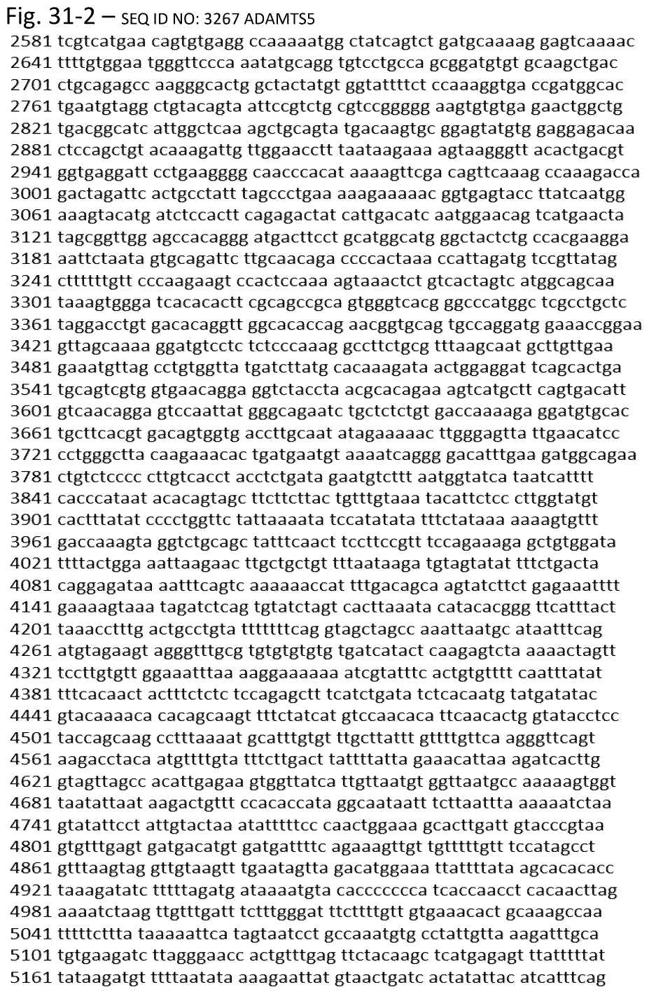

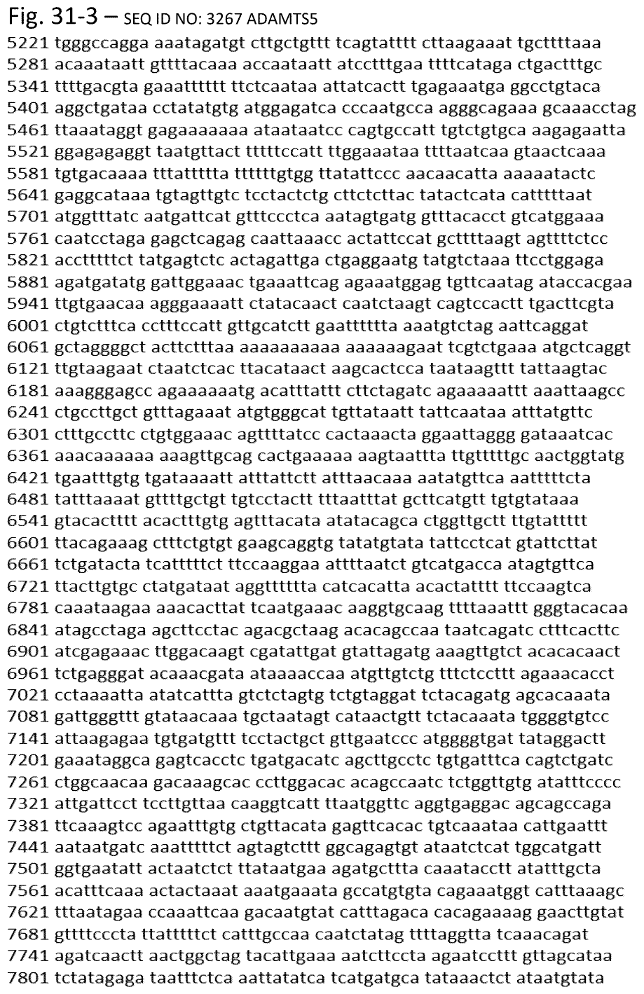

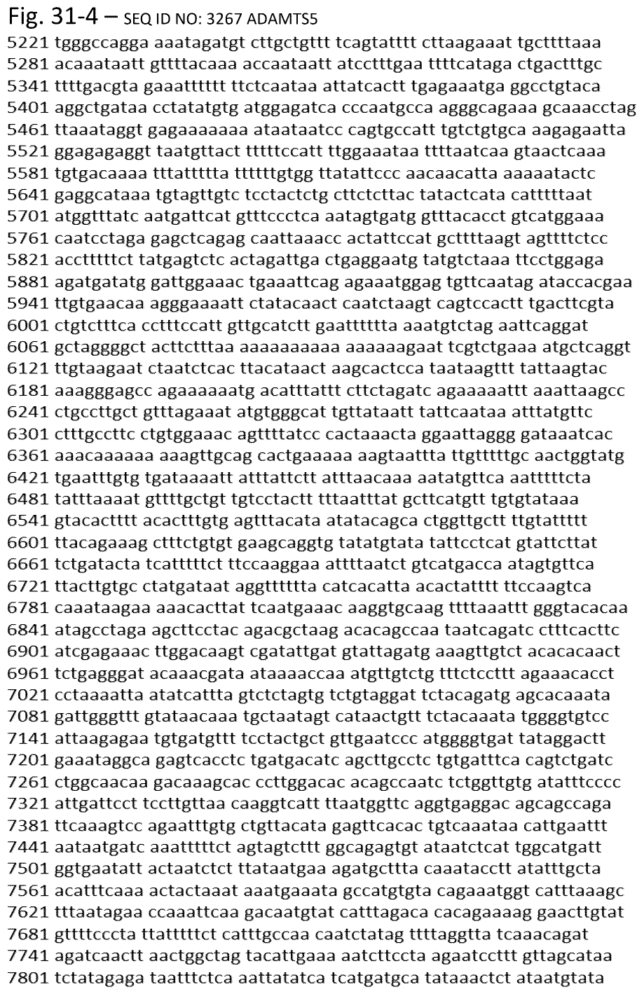

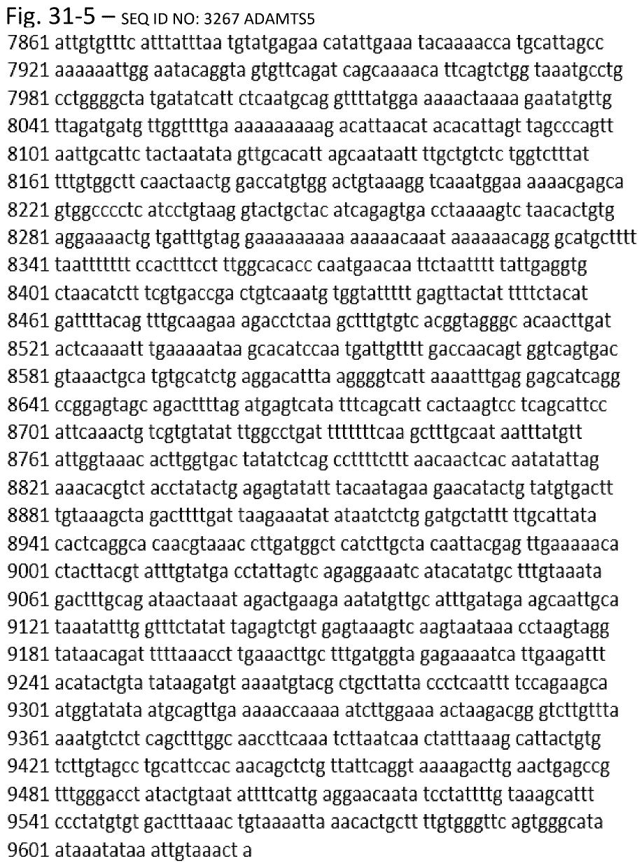

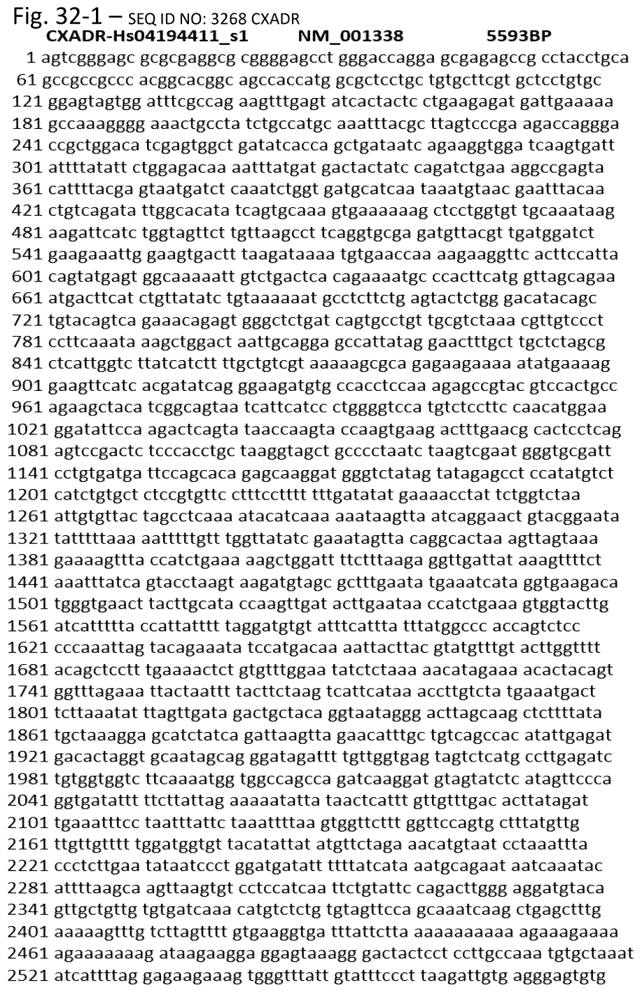

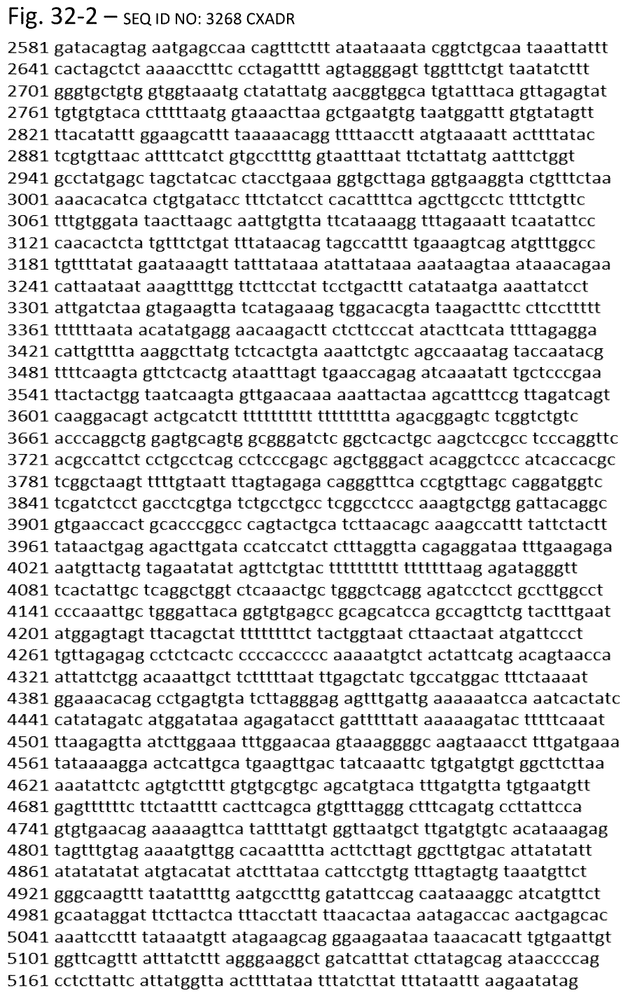

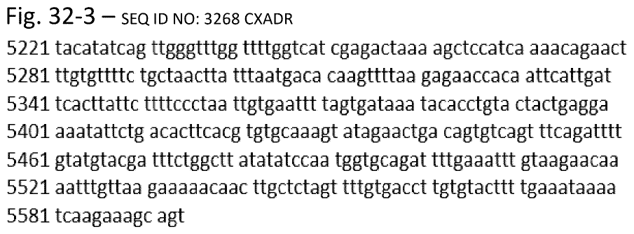

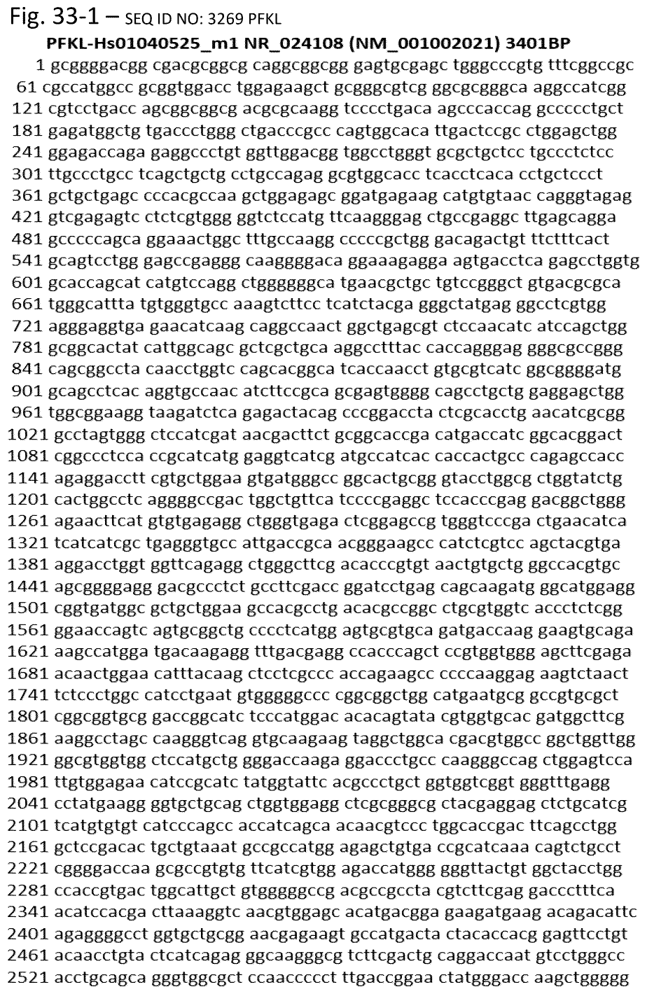

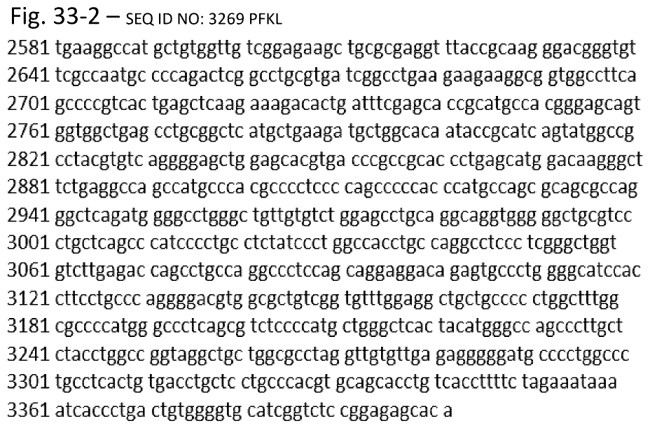

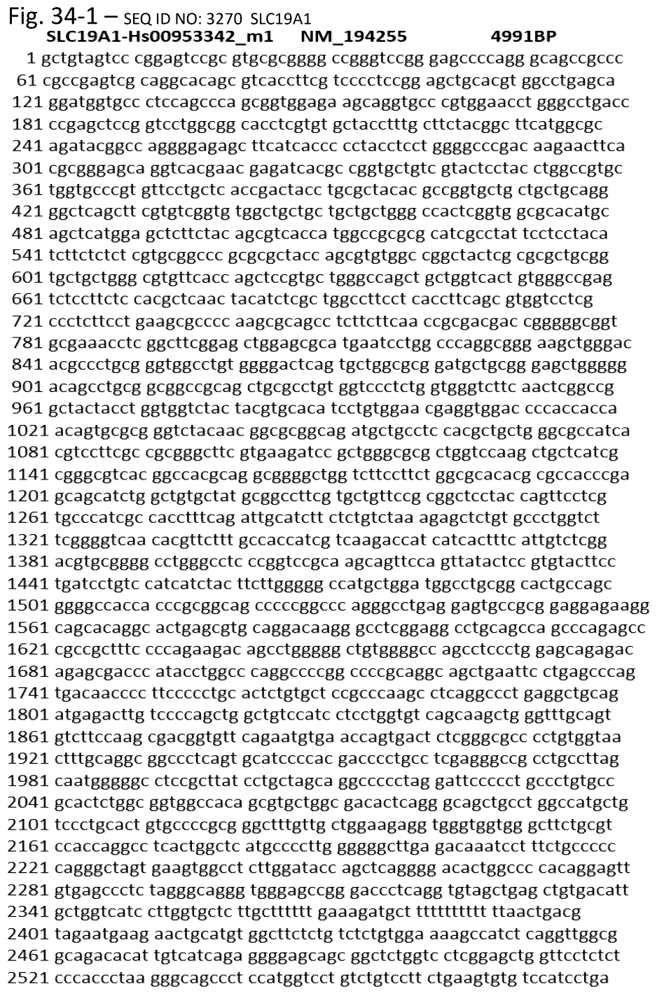

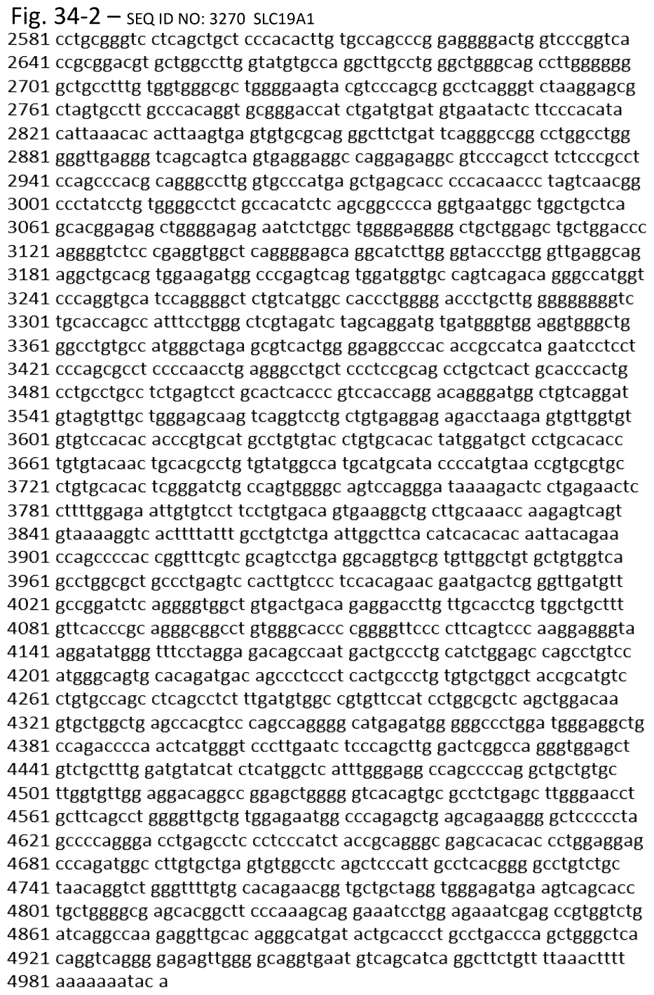

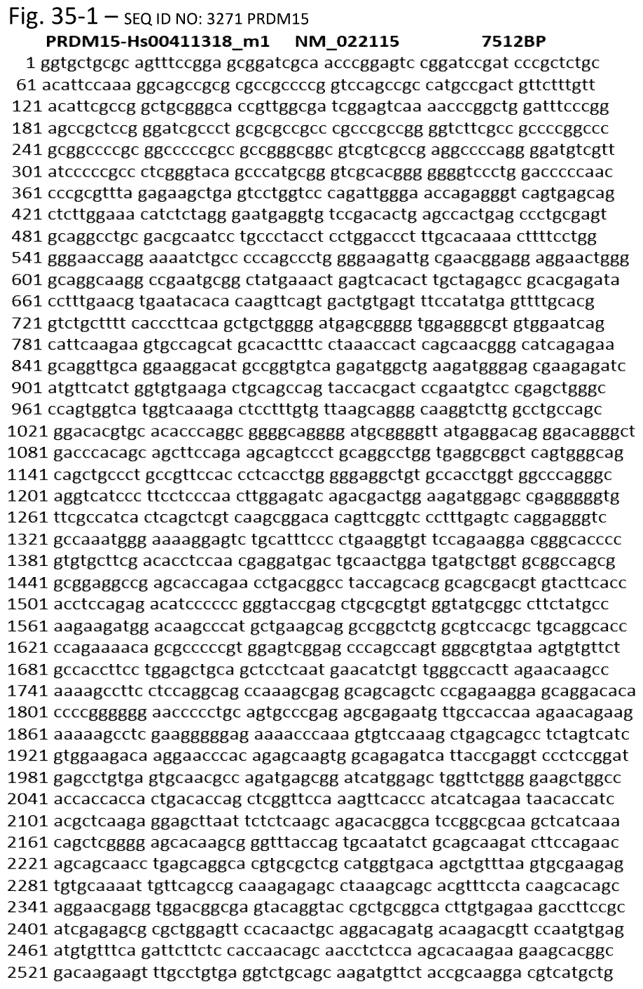

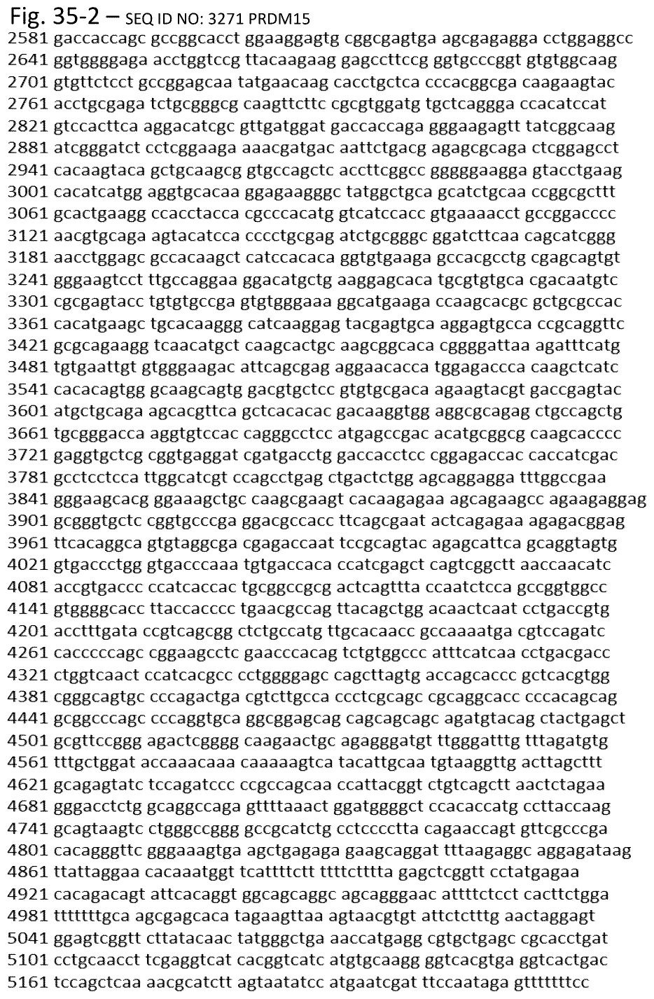

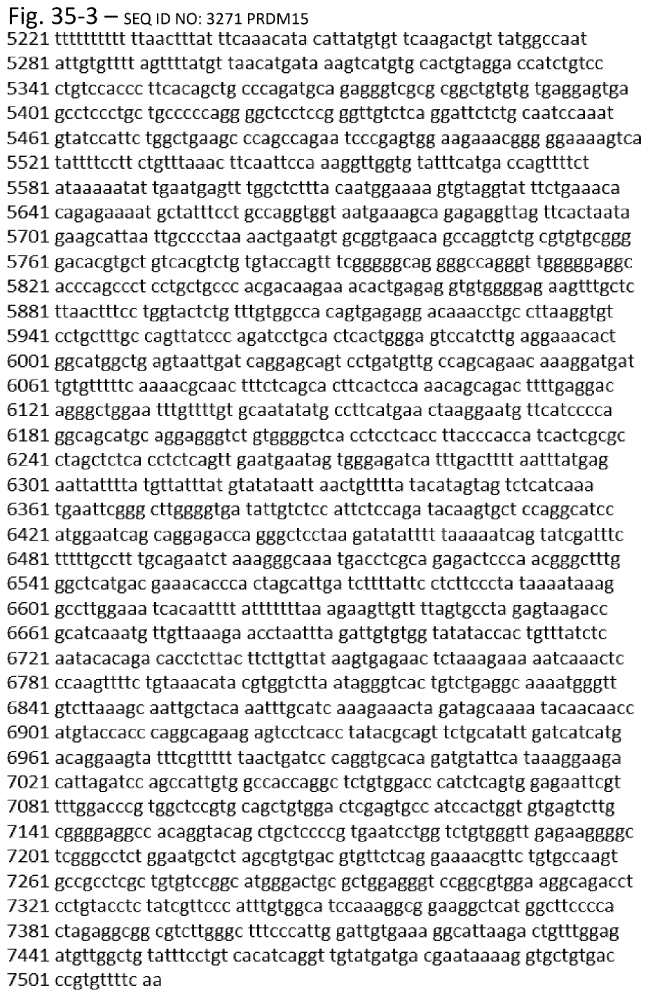

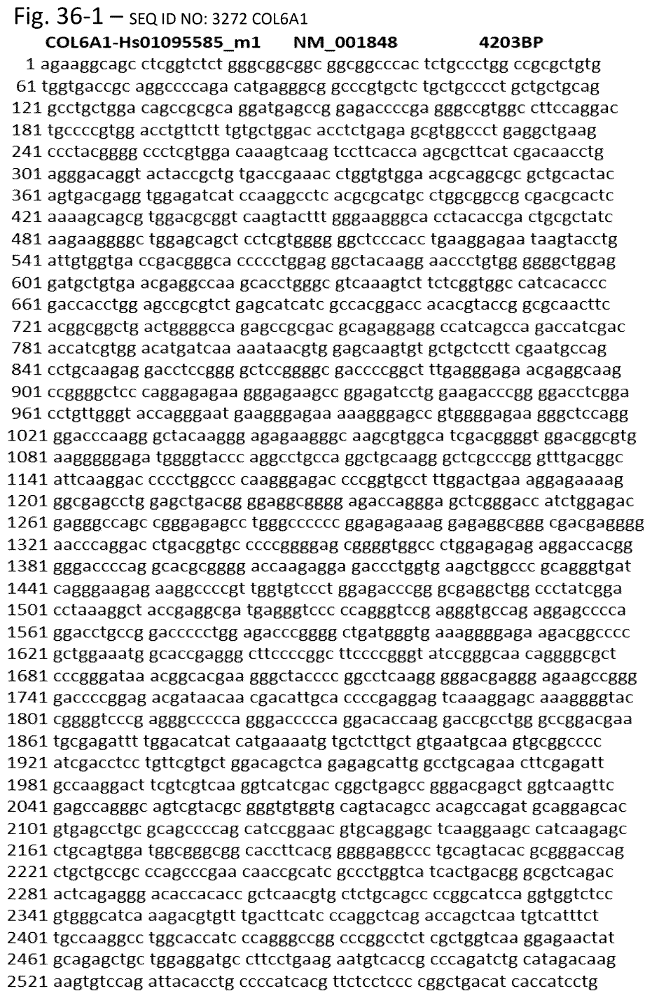

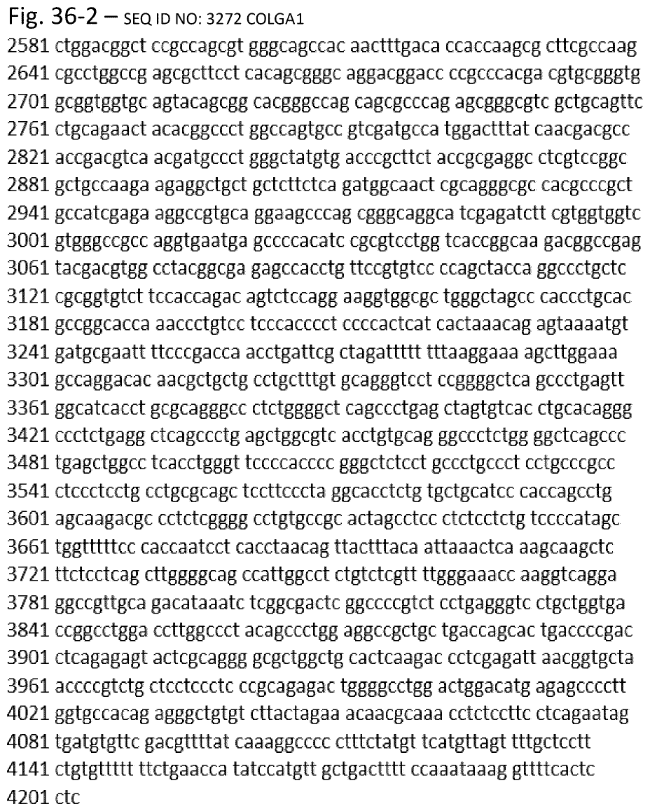

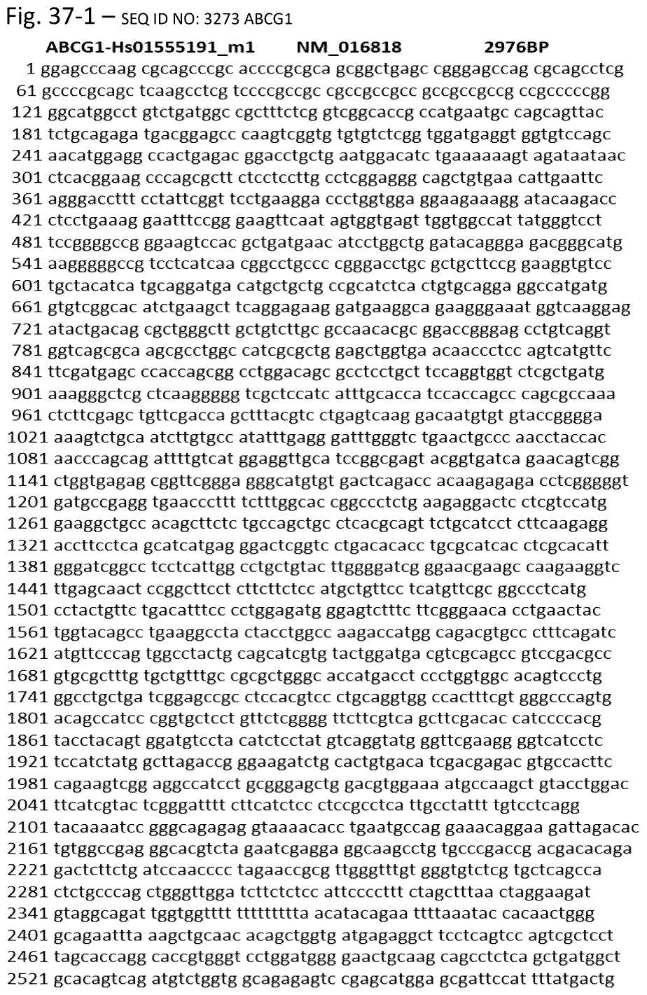

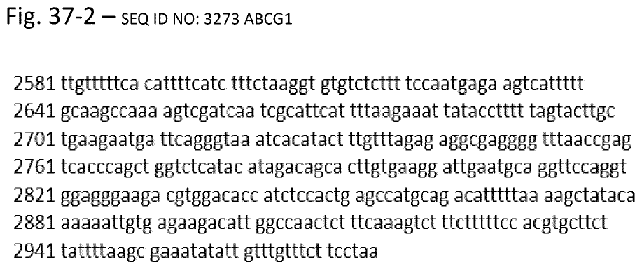

FIGS. 12A-12C shows that the group of 54 plasma cell free RNA markers were tested for the prediction of Trisomy 21 using C5.0 with bagging. The RNAs utilized in the best performing C5.0 models were then entered into Random Forest, and the diagnostic models of FIGS. 12A-12C resulted.

FIG. 12A shows a specific 6 plasma cell free RNA group that happens to consist of mRNA that are products of genes located on the number 21 chromosome. The model's accuracy is diagnostic of Trisomy 21. Thus, a specific group of the 6 plasma cell free RNA is provided for diagnostics: ATP50; ICOSLG; DOPEY2; PKNOX1; COL6A; and GART.

FIG. 12B shows a specific 6 plasma cell free RNA group that consist of 5 small noncoding RNAs produced by genes located on a chromosome other than the number 21, and 1 mRNA that is a product of a gene located on the number 21 chromosome. The model's accuracy is diagnostic of Trisomy 21. Thus, a specific group of the 6 plasma cell free RNA group is provided for diagnostics: ENSG00000119633; miR-548i; miR-26b; miR-450b; ENSG00000212363; and GART.

FIG. 12C show a specific 11 plasma cell free RNA group that consists of the 11 unique RNAs identified with C5.0. The model's accuracy is diagnostic of Trisomy 21. Thus, a specific group of the 11 plasma cell free RNA group is provided for diagnostics: ATP50; ICOSLG; DOPEY2; PKNOX1; COL6A; GART; ENSG00000119633; miR-548i; miR-26b; miR-450b; and ENSG00000212363.

The nucleic acid biomarkers can be useful because they can be detected as a combination of nucleic biomarkers in a human subject. This detected combination of biomarkers when detected to have transcription levels that are outside of normal transcriptional levels provides information about the probability of defined heath scenarios. For example, the specific combinations of the nucleic acid biomarkers having the variation from the transcriptional standard can be used for assessing the likelihood of trisomy 21. Accordingly, methods are described herein for detecting the combination of nucleic biomarkers. The combination of biomarkers being upregulated or downregulated provide an indication that the subject pregnant female carries a fetus having trisomy 21. The results of the combination of biomarkers can be obtained, and the variation for each detected to be: no variation; an upregulation; or a downregulation. A report can be generated to identify the variation of each biomarker in the combination, and the results thereof relative to the patient being sampled for the biomarker combination. The report can further provide a recommendation for further medical evaluations to confirm whether or not the presence of the combination of nucleic acid biomarkers was a true positive result or a false positive result. For example, the presence of the combination of biomarkers can provide an indication of the corresponding fetus having T21, and the report can provide recommendations of specific medical protocols for confirming whether or not the indication is true or false. The methods may also include the performance of the subsequent medical procedure to confirm the indication to be true or false, whereby a report can be generated regarding the indication by the presence of the combination of biomarkers compared to the outcome or results of the subsequent medical procedure.

In some embodiments, a method of detecting a combination of nucleic acid biomarkers in a human subject can include: obtaining a nucleic acid sample from the human subject; analyzing a transcriptome of the human subject for the combination of nucleic acid biomarkers in the nucleic acid sample from the human subject; selecting the combination of nucleic acid biomarkers; detecting in the nucleic acid sample the presence of the combination of nucleic acid biomarkers, wherein each nucleic acid biomarker in the combination of nucleic acid biomarkers has a variation from a transcription standard, wherein the combination of nucleic acid biomarkers includes: ATP50 having a nucleotide sequence of or complementary to SEQ ID NO: 3249; ICOSLG having a nucleotide sequence of or complementary to SEQ ID NO: 3265; DOP1B (also known as DOPEY2) having a nucleotide sequence of or complementary to SEQ ID NO: 3250; PKNOX1 having a nucleotide sequence of or complementary to SEQ ID NO: 3254; COL6A1 having a nucleotide sequence of or complementary to SEQ ID NO: 3272; GART having a nucleotide sequence of or complementary to SEQ ID NO: 3256; ENSG00000199633 F2 having a nucleotide sequence of or complementary to SEQ ID NO: 3217; hsa-mir-548I having a nucleotide sequence of or complementary to SEQ ID NO: 3165; hsa-mir-26b having a nucleotide sequence of or complementary to SEQ ID NO: 3161; hsa-mir-450b having a nucleotide sequence 10 of or complementary to SEQ ID NO: 3246; and ENSG00000212363 having a nucleotide sequence of or complementary to SEQ ID NO: 3170. Table 1 shows this combination of nucleic acid biomarkers—Group 1—as a defined panel where each must be present and detected for a variation of no variation; an upregulation; or a downregulation.

TABLE 1
Group 1-Combination of Biomarkers

| | | | | | | Up or |
|---|---|---|---|---|---|---|
| SEQ ID | Group ID | | | | | Down |
| NO: | NO: | Gene Name | p-value | Type/Ref | Chromosome | Regulation |
| 3249 | 28 | ATP5O | <0.01 | NM_001697.3 | 21 | Down |
| 3265 | 31 | ICOSLG | <0.01 | NM_015259 | 21 | Down |
| 3250 | 33 | DOP1B | <0.01 | NM_005128 | 21 | Down |
| 3254 | 46 | PKNOX1 | <0.01 | NM_004571 | 21 | Up |
| 3272 | 52 | COL6A1 | <0.01 | NM_001848 | 21 | Down |
| 3256 | 54 | GART | <0.01 | NM_000819 | 21 | Down |
| 3217 | 1 | ENSG00000199633 F2 | <0.01 | snoRNA | 15 | Up |
| 3165 | 5 | hsa-mir-548I | <0.01 | miRNA | 3 | Down |
| 3161 | 10 | hsa-mir-26b | <0.01 | miRNA | 2 | Down |
| 3246 | 12 | hsa-mir-450b | <0.01 | miRNA | X | Down |
| 3170 | 13 | ENSG00000212363 | <0.01 | snoRNA | 5 | Down |

The information in the tables provides the original Biomarker SEQ ID NO:, Group ID, Gene name, p value, Reference sequence number, Chromosome of Origin and the direction of gene regulation when the fetus has Trisomy 21.

In some embodiments, the combination of nucleic acid biomarkers includes: ATP50 having a nucleotide sequence of or complementary to SEQ ID NO: 3249 with a transcriptional variation that is downregulated compared to the transcription standard; ICOSLG having a nucleotide sequence of or complementary to SEQ ID NO: 3265 with a transcriptional variation that is downregulated compared to the transcription standard; DOP1B having a nucleotide sequence of or complementary to SEQ ID NO: 3250 with a transcriptional variation that is downregulated compared to the transcription standard; PKNOX1 having a nucleotide sequence of or complementary to SEQ ID NO: 3254 with a transcriptional variation that is upregulated compared to the transcription standard; COL6A1 having a nucleotide sequence of or complementary to SEQ ID NO: 3272 with a transcriptional variation that is downregulated compared to the transcription standard; GART having a nucleotide sequence of or complementary to SEQ ID NO: 3256 with a transcriptional variation that is downregulated compared to the transcription standard; ENSG00000199633 F2 having a nucleotide sequence of or complementary to SEQ ID NO: 3217 with a transcriptional variation that is upregulated compared to the transcription standard; hsa-mir-548I having a nucleotide sequence of or complementary to SEQ ID NO: 3165 with a transcriptional variation that is downregulated compared to the transcription standard; hsa-mir-26b having a nucleotide sequence of or complementary to SEQ ID NO: 3161 with a transcriptional variation that is downregulated compared to the transcription standard; hsa-mir-450b having a nucleotide sequence of or complementary to SEQ ID NO: 3246 with a transcriptional variation that is downregulated compared to the transcription standard; and ENSG00000212363 having a nucleotide sequence of or complementary to SEQ ID NO: 3170 with a variation less than the transcription standard.

In some embodiments, the combination of nucleic acid biomarkers is: ENSG00000199633 F2 having a nucleotide sequence of or complementary to SEQ ID NO: 3217; hsa-mir-548I having a nucleotide sequence of or complementary to SEQ ID NO: 3165; hsa-mir-26b having a nucleotide sequence of or complementary to SEQ ID NO: 3161; hsa-mir-450b having a nucleotide sequence of or complementary to SEQ ID NO: 3246; ENSG00000212363 having a nucleotide sequence of or complementary to SEQ ID NO: 3170; and GART having a nucleotide sequence of or complementary to SEQ ID NO: 3256. Table 2 shows this combination of nucleic acid biomarkers—Group 2—as a defined panel where each must be present and detected for a variation of no variation; an upregulation; or a downregulation.

TABLE 2
Group 2-Combination of Biomarkers

| SEQ ID | Group | | p- | | | Up or Down |
|---|---|---|---|---|---|---|
| NO: | ID NO: | Gene Name | value | Type/Ref | Chromosome | Regulation |
| 3217 | 1 | ENSG00000199633 F2 | <0.01 | snoRNA | 15 | Up |
| 3165 | 5 | hsa-mir-548I | <0.01 | miRNA | 3 | Down |
| 3161 | 10 | hsa-mir-26b | <0.01 | miRNA | 2 | Down |
| 3246 | 12 | hsa-mir-450b | <0.01 | miRNA | X | Down |
| 3170 | 13 | ENSG00000212363 | <0.01 | snoRNA | 5 | Down |
| 3256 | 54 | GART-Hs00531926_m1 | <0.01 | mRNA/NM_000819 | 21 | Down |

In some embodiments, the combination of nucleic acid biomarkers is: ENSG00000199633 F2 having a nucleotide sequence of or complementary to SEQ ID NO: 3217 with a transcriptional variation that is upregulated compared to the transcription standard; hsa-mir-548I having a nucleotide sequence of or complementary to SEQ ID NO: 3165 with a transcriptional variation that is downregulated compared to the transcription standard; hsa-mir-26b having a nucleotide sequence of or complementary to SEQ ID NO: 3161 with a transcriptional variation that is downregulated compared to the transcription standard; hsa-mir-450b having a nucleotide sequence of or complementary to SEQ ID NO: 3246 with a transcriptional variation that is downregulated compared to the transcription standard; and ENSG00000212363 having a nucleotide sequence of or complementary to SEQ ID NO: 3170 with a variation less than the transcription standard; and GART having a nucleotide sequence of or complementary to SEQ ID NO: 3256 with a transcriptional variation that is downregulated compared to the transcription standard.

In some embodiments, the combination of nucleic acid biomarkers is: ATP50 having a nucleotide sequence of or complementary to SEQ ID NO: 3249; ICOSLG having a nucleotide sequence of or complementary to SEQ ID NO: 3265; DOP1B having a nucleotide sequence of or complementary to SEQ ID NO: 3250; PKNOX1 having a nucleotide sequence of or complementary to SEQ ID NO: 3254; COL6A1 having a nucleotide sequence of or complementary to SEQ ID NO: 3272; and GART having a nucleotide sequence of or complementary to SEQ ID NO: 3256. Table 3 shows this combination of nucleic acid biomarkers—Group 3—as a defined panel where each must be present and detected for a variation of no variation; an upregulation; or a downregulation.

TABLE 3
Group 3-Combination of Biomarkers

| | | | | | | Up or |
|---|---|---|---|---|---|---|
| | | | | | | Down |
| SEQ ID NO: | Group ID NO: | Gene Name | p-value | Type/Ref | Chromosome | Regulation |
| 3249 | 28 | ATP5O | <0.01 | NM_001697.3 | 21 | Down |
| 3265 | 31 | ICOSLG | <0.01 | NM_015259 | 21 | Down |
| 3250 | 33 | DOP1B | <0.01 | NM_005128 | 21 | Down |
| 3254 | 46 | PKNOX1 | <0.01 | NM_004571 | 21 | Up |
| 3272 | 52 | COL6A1 | <0.01 | NM_001848 | 21 | Down |
| 3256 | 54 | GART | <0.01 | NM_000819 | 21 | Down |

In some embodiments, the combination of nucleic acid biomarkers is: ATP50 having a nucleotide sequence of or complementary to SEQ ID NO: 3249 with a transcriptional variation that is downregulated compared to the transcription standard; ICOSLG having a nucleotide sequence of or complementary to SEQ ID NO: 3265 with a transcriptional variation that is downregulated compared to the transcription standard; DOP1B having a nucleotide sequence of or complementary to SEQ ID NO: 3250 with a transcriptional variation that is downregulated compared to the transcription standard; PKNOX1 having a nucleotide sequence of or complementary to SEQ ID NO: 3254 with a transcriptional variation that is upregulated compared to the transcription standard; COL6A1 having a nucleotide sequence of or complementary to SEQ ID NO: 3272 with a transcriptional variation that is downregulated compared to the transcription standard; and GART having a nucleotide sequence of or complementary to SEQ ID NO: 3256 with a transcriptional variation that is downregulated compared to the transcription standard.

In some embodiments, the combination of nucleic acid biomarkers in Group 1 (Table 1) further comprises a sub-group of biomarkers (A) to form Group 1A, which Group 1A includes the biomarkers of Group 1 and the following additional sub-group (A) of mRNA biomarkers: RASGRP4 having a nucleotide sequence of or complementary to SEQ ID NO: 3257; FAM20A having a nucleotide sequence of or complementary to SEQ ID NO: 3258; NEK9 having a nucleotide sequence of or complementary to SEQ ID NO: 3259; ABCC1 having a nucleotide sequence of or complementary to SEQ ID NO: 3260; SORBS2 having a nucleotide sequence of or complementary to SEQ ID NO: 3261; TMPRSS2 having a nucleotide sequence of or complementary to SEQ ID NO: 3262; DSCAM having a nucleotide sequence of or complementary to SEQ ID NO: 3263; ERG having a nucleotide sequence of or complementary to SEQ ID NO: 3264; ICOSLG having a nucleotide sequence of or complementary to SEQ ID NO: 3265; C21orf33 having a nucleotide sequence of or complementary to SEQ ID NO: 3266; ADAMTS5 having a nucleotide sequence of or complementary to SEQ ID NO: 3267; CXADR having a nucleotide sequence of or complementary to SEQ ID NO: 3268; PFKL having a nucleotide sequence of or complementary to SEQ ID NO: 3269; SLC19A1 having a nucleotide sequence of or complementary to SEQ ID NO: 3270; PRDM15 having a nucleotide sequence of or complementary to SEQ ID NO: 3271; COL6A1 having a nucleotide sequence of or complementary to SEQ ID NO: 3272; and ABCG1 having a nucleotide sequence of or complementary to SEQ ID NO: 3273.

In some aspects, the combination of nucleic acid biomarkers in Group 1 (Table 1) further comprises a sub-group of biomarkers to form Group 1A, which Group 1A includes the biomarkers of Group 1 and the following additional biomarkers: RASGRP4 having a nucleotide sequence of or complementary to SEQ ID NO: 3257 with a transcriptional variation that is downregulated compared to the transcription standard; FAM20A having a nucleotide sequence of or complementary to SEQ ID NO: 3258 with a transcriptional variation that is downregulated compared to the transcription standard; NEK9 having a nucleotide sequence of or complementary to SEQ ID NO: 3259 with a transcriptional variation that is downregulated or upregulated compared to the transcription standard; ABCC1 having a nucleotide sequence of or complementary to SEQ ID NO: 3260 with a transcriptional variation that is upregulated compared to the transcription standard; SORBS2 having a nucleotide sequence of or complementary to SEQ ID NO: 3261 with a transcriptional variation that is downregulated or upregulated compared to the transcription standard; TMPRSS2 having a nucleotide sequence of or complementary to SEQ ID NO: 3262 with a transcriptional variation that is downregulated compared to the transcription standard; DSCAM having a nucleotide sequence of or complementary to SEQ ID NO: 3263 with a transcriptional variation that is downregulated compared to the transcription standard; ERG having a nucleotide sequence of or complementary to SEQ ID NO: 3264 with a transcriptional variation that is upregulated compared to the transcription standard; ICOSLG having a nucleotide sequence of or complementary to SEQ ID NO: 3265 with a transcriptional variation that is downregulated compared to the transcription standard; C21orf33 having a nucleotide sequence of or complementary to SEQ ID NO: 3266 with a transcriptional variation that is downregulated compared to the transcription standard; ADAMTS5 having a nucleotide sequence of or complementary to SEQ ID NO: 3267 with a transcriptional variation that is downregulated compared to the transcription standard; CXADR having a nucleotide sequence of or complementary to SEQ ID NO: 3268 with a transcriptional variation that is downregulated compared to the transcription standard; PFKL having a nucleotide sequence of or complementary to SEQ ID NO: 3269 with a transcriptional variation that is upregulated compared to the transcription standard; SLC19A1 having a nucleotide sequence of or complementary to SEQ ID NO: 3270 with a transcriptional variation that is upregulated compared to the transcription standard; PRDM15 having a nucleotide sequence of or complementary to SEQ ID NO: 3271 with a transcriptional variation that is downregulated compared to the transcription standard; COL6Al having a nucleotide sequence of or complementary to SEQ ID NO: 3272 with a transcriptional variation that is downregulated compared to the transcription standard; and ABCG1 having a nucleotide sequence of or complementary to SEQ ID NO: 3273 with a transcriptional variation that is downregulated compared to the transcription standard.

In some embodiments, the combination of nucleic acid biomarkers in Group 1 (Table 1) further comprises a second sub-group (B) of biomarkers to form Group 1B, which Group 1B includes the biomarkers of Group 1 and the following additional biomarkers (B) sub-group (B) are small non-coding RNA that can include: ENSG00000199633 F2 having a nucleotide sequence of or complementary to SEQ ID NO: 3217; ENSG00000207147 F2 having a nucleotide sequence of or complementary to SEQ ID NO: 3238; hsa-let-7d Fl having a nucleotide sequence of or complementary to SEQ ID NO: 3189; hsa-mir-569 F1 having a nucleotide sequence of or complementary to SEQ ID NO: 3163; hsa-mir-5481 having a nucleotide sequence of or complementary to SEQ ID NO: 3165; ENSG00000201980 having a nucleotide sequence of or complementary to SEQ ID NO: 3195; ENSG00000202231 having a nucleotide sequence of or complementary to SEQ ID NO: 3243; hsa-mir-216b having a nucleotide sequence of or complementary to SEQ ID NO: 3160; hsa-mir-98 having a nucleotide sequence of or complementary to SEQ ID NO: 3245; hsa-mir-26b having a nucleotide sequence of or complementary to SEQ ID NO: 3161; hsa-mir-581 Fl having a nucleotide sequence of or complementary to SEQ ID NO: 3173; hsa-mir-450b having a nucleotide sequence of or complementary to SEQ ID NO: 3246; ENSG00000212363 having a nucleotide sequence of or complementary to SEQ ID NO: 3170; ENSG00000199282 having a nucleotide sequence of or complementary to SEQ ID NO: 3207; hsa-mir-523 having a nucleotide sequence of or complementary to SEQ ID NO: 3233; hsa-mir-376a-2/1 F2 having a nucleotide sequence of or complementary to SEQ ID NO: 3214; ENSG00000199856 F1 having a nucleotide sequence of or complementary to SEQ ID NO: 3230; and HBII-276 F2 having a nucleotide sequence of or complementary to SEQ ID NO: 3184.

In some aspects, sub-group (B) are small non-coding RNA that can include: ENSG00000199633 F2 having a nucleotide sequence of or complementary to SEQ ID NO: 3217 with a transcriptional variation that is upregulated compared to the transcription standard; ENSG00000207147 F2 having a nucleotide sequence of or complementary to SEQ ID NO: 3238 with a transcriptional variation that is upregulated compared to the transcription standard; hsa-let-7d Fl having a nucleotide sequence of or complementary to SEQ ID NO: 3189 with a transcriptional variation that is upregulated compared to the transcription standard; hsa-mir-569 F1 having a nucleotide sequence of or complementary to SEQ ID NO: 3163 with a transcriptional variation that is downregulated compared to the transcription standard; hsa-mir-5481 having a nucleotide sequence of or complementary to SEQ ID NO: 3165 with a transcriptional variation that is downregulated compared to the transcription standard; ENSG00000201980 having a nucleotide sequence of or complementary to SEQ ID NO: 3195 with a transcriptional variation that is upregulated compared to the transcription standard; ENSG00000202231 having a nucleotide sequence of or complementary to SEQ ID NO: 3243 with a transcriptional variation that is downregulated compared to the transcription standard; hsa-mir-216b having a nucleotide sequence of or complementary to SEQ ID NO: 3160 with a transcriptional variation that is upregulated compared to the transcription standard; hsa-mir-98 having a nucleotide sequence of or complementary to SEQ ID NO: 3245 with a transcriptional variation that is downregulated compared to the transcription standard; hsa-mir-26b having a nucleotide sequence of or complementary to SEQ ID NO: 3161 with a transcriptional variation that is downregulated compared to the transcription standard; hsa-mir-581 Fl having a nucleotide sequence of or complementary to SEQ ID NO: 3173 with a transcriptional variation that is upregulated compared to the transcription standard; hsa-mir-450b having a nucleotide sequence of or complementary to SEQ ID NO: 3246 with a transcriptional variation that is downregulated compared to the transcription standard; ENSG00000212363 having a nucleotide sequence of or complementary to SEQ ID NO: 3170 with a transcriptional variation that is downregulated compared to the transcription standard; ENSG00000199282 having a nucleotide sequence of or complementary to SEQ ID NO: 3207 with a transcriptional variation that is downregulated compared to the transcription standard; hsa-mir-523 having a nucleotide sequence of or complementary to SEQ ID NO: 3233 with a transcriptional variation that is downregulated compared to the transcription standard; hsa-mir-376a-2/1 F2 having a nucleotide sequence of or complementary to SEQ ID NO: 3214 with a transcriptional variation that is downregulated compared to the transcription standard; ENSG00000199856 F1 having a nucleotide sequence of or complementary to SEQ ID NO: 3230 with a transcriptional variation that is downregulated compared to the transcription standard; and HBII-276 F2 having a nucleotide sequence of or complementary to SEQ ID NO: 3184 with a transcriptional variation that is upregulated compared to the transcription standard.

In some embodiments, the combination of nucleic acid biomarkers in Group 1 (Table 1) further comprises the first sub-group of biomarkers (A) and the second sub-group of biomarkers (B) to form Group 1C of biomarkers, which Group 1C includes the RNA biomarkers of Group 1 and the first sub-group (A) mRNA biomarkers and the sub-group (B) of small non-coding RNA biomarkers.

In some embodiments, Group 1A characterized with sub-group D results in Group 1AD. In some embodiments, Group 1C characterized with the sub-group D results in Group 1 and Group 1CD.

In some embodiments, the Group 1 of Table 1 can have one or more of the biomarkers being a specific examples of the combination of nucleic acid biomarkers—Group 1—as a defined panel where each must be present and detected for a variation of no variation; an upregulation; or a downregulation. As such, in view of Table 1, Group 1 can be specified in the following example: ATP50 including ATP50-Hs04272738_m1 with a transcriptional variation that is downregulated compared to the transcription standard; ICOSLG including ICOSLG-Hs00391287_m1 with a transcriptional variation that is downregulated compared to the transcription standard; DOP1B including DOP1B-Hs01123288_m1 with a transcriptional variation that is downregulated compared to the transcription standard; PKNOXI including PKNOX1-Hs01007092_m1 with a transcriptional variation that is upregulated compared to the transcription standard; COL6A1 including COL6A1-Hs01095585_m1 with a transcriptional variation that is downregulated compared to the transcription standard; and GART including GART-Hs00531926_m1 with a transcriptional variation that is downregulated compared to the transcription standard. In some aspects, the recited biomarkers in any of the groups can include the sample in this paragraph of that biomarker.

In some embodiments, any of the groups of biomarkers having GART, can be specified as having the following example of GART including GART-Hs00531926_m1 with a transcriptional variation that is downregulated compared to the transcription standard.

In some embodiments, the combination of nucleic acid biomarkers further comprises: FAM20A including FAM20A-Hs01034071_m1 that is downregulated compared to the transcriptional standard, and FAM20A-Hs01034070_m that is downregulated compared to the transcriptional standard; NEK9 including NEK9-Hs00929602_m1 that is downregulated compared to the transcriptional standard, and NEK9-Hs00929594_m that is upregulated compared to the transcriptional standard; SORBS2 including SORBS2-Hs01125202_m1 that is upregulated compared to the transcriptional standard and SORBS2-Hs00243432 ml that is downregulated compared to the transcriptional standard; DOP1B including DOP1B-Hs01123288_m1 that is downregulated compared to the transcriptional standard and DOP1B-Hs01123267_g1 that is downregulated compared to the transcriptional standard; UBASH3A including UBASH3A-Hs00955169_m1 that is upregulated compared to the transcriptional standard and UBASH3A-Hs00955168_m1 that is downregulated compared to the transcriptional standard; PKNOX1 including PKNOX1-Hs01007098_m1 that is downregulated compared to the transcriptional standard and PKNOX1-Hs01007097_m1 that is downregulated compared to the transcriptional standard and PKNOX1-Hs01007094_m1 that is upregulated compared to the transcriptional standard and PKNOX1-Hs01007093_m1 that is upregulated compared to the transcriptional standard and PKNOX1-Hs01007092_m1 that is upregulated compared to the transcriptional standard and PKNOX1-Hs00231814_m1 that is downregulated compared to the transcriptional standard; and SLC19A1 including SLC19A1-Hs00953342_m1 that is upregulated compared to the transcriptional standard and SLC19A1-Hs00953341_m1 that is downregulated compared to the transcriptional standard.

In some embodiments, the combination of nucleic acid biomarkers includes or consists of: RASGRP4-Hs01073179_m1; FAM20A-Hs01034071_m1; FAM20A-Hs01034070_m1; NEK9-Hs00929602_m1; NEK9-Hs00929594_m1; ABCC1-Hs01561504_m1; SORBS2-Hs01125202_m1; SORBS2-Hs00243432_m1; TMPRSS2-ERG fusion gene; ATP50-Hs04272738_m1; DSCAM-Hs00242097_m1; ERG-Hs01573964_m1; ICOSLG-Hs00391287_m1; DOP1B-Hs01123288_m1; DOP1B-Hs01123267_g1; C21orf33-Hs01105802_g1; ADAMTS5-Hs04272736_s1; CXADR-Hs04194411_s1; NCAM2-Hs01562292_m1; UBASH3A-Hs00955169_m1; UBASH3A-Hs00955168_m1; PFKL-Hs01040525_m1; CHODL-Hs01070471_m1; PKNOX1-Hs01007098_m1; PKNOX1-Hs01007097_m1; PKNOX1-Hs01007094_m1; PKNOX1-PKNOX1-Hs01007093_m1; Hs01007092_m1; PKNOX1-Hs00231814_m1; CYYR1-Hs00951849_m1; SLC19A1-Hs00953342_m1; SLC19A1-Hs00953341_m1; PRDM15-Hs00411318_m1; COL6A1-Hs01095585_m1; ABCG1-Hs01555191_m1; GART-Hs00531926_m1; ENSG00000199633 F2; ENSG00000207147 F2; hsa-let-7d F1; hsa-mir-569 F1; hsa-mir-548I; ENSG00000201980; ENSG00000202231; hsa-mir-216b; hsa-mir-98; hsa-mir-26b; hsa-mir-581 F1; hsa-mir-450b; ENSG00000212363; ENSG00000199282; hsa-mir-523; hsa-mir-376a-2/1 F2; ENSG00000199856 F1; and HBII-276 F2;

In some embodiments, the method of using the combination of nucleic acid biomarkers includes hybridizing each nucleic acid biomarker in the nucleic acid sample with a complementary nucleic acid configured as a primer or a probe, the method comprising detecting the hybridizing. Accordingly, a combination of primers (forward and/or reverse) can be provided for each of the combinations of the specific Groups or sub-groups of the biomarkers. Accordingly, a combination of probes (e.g., labeled, bound to substrate, etc.) can be provided for each of the combinations of the specific Groups or sub-groups of the biomarkers.

In some aspects, the method can include providing the transcription standard for each nucleic acid biomarker for the combination of nucleic acid biomarkers. That is, each biomarker in each combination has a transcription standard across populations without T21. The biological sample of the pregnant mother can be assayed for the combination of nucleic acid biomarkers of one of the Groups to see whether the pregnant woman has the combination of biomarkers in that Group varying from the transcriptional standard. The presence of the combination of biomarkers having the variation from the transcription standard provide for the indication that the fetus of the pregnant mother has T21. Thus, all of the biomarkers in the specific combination of that Group is assayed in the pregnant woman. In some aspects, the method can include obtaining cell free plasma RNA as the nucleic acid sample, wherein the nucleic acid biomarkers are RNA (e.g., having RNA nucleic acids).

In some embodiments, the method can include generating a report, the report reciting the presence of the combination of nucleic acid biomarkers being present in the nucleic acid sample of the human subject being present in a biomarker amount that is varied from the transcription standard. The report can include any of the information provided herein, such as the presence of the combination of nucleic acid biomarkers having the deviation from the transcriptional standard, what such a presence of the Group of biomarkers means for the fetus, and a listing of further medical procedures and actions recommended or options to be taken.

In some embodiments, the combination of nucleic acid biomarkers is the combination defined as Group 4, shown in Table 4. Table 4 shows this combination of nucleic acid biomarkers—Group 4—as a defined panel where each must be present and detected for a variation of no variation; an upregulation; or a downregulation.

TABLE 4
Group 4-Combination of Biomarkers

| | | | | Up or |
|---|---|---|---|---|
| SEQ | Group | | | Down |
| ID | ID | | | Regu- |
| NO: | NO: | Gene Name | Type/Ref | lation |
| 3217 | 1 | ENSG00000199633 F2 | snoRNA | Up |
| 3238 | 2 | ENSG00000207147 F2 | snoRNA | Up |
| 3189 | 3 | hsa-let-7d F1 | miRNA | Up |
| 3163 | 4 | hsa-mir-569 F1 | miRNA | Down |
| 3165 | 5 | hsa-mir-548I | miRNA | Down |
| 3195 | 6 | ENSG00000201980 | snoRNA | Up |
| 3243 | 7 | ENSG00000202231 | snoRNA | Down |
| 3160 | 8 | hsa-mir-216b | miRNA | Up |
| 3245 | 9 | hsa-mir-98 | miRNA | Down |
| 3161 | 10 | hsa-mir-26b | miRNA | Down |
| 3173 | 11 | hsa-mir-581 F1 | miRNA | Up |
| 3246 | 12 | hsa-mir-450b | miRNA | Down |
| 3170 | 13 | ENSG00000212363 | snoRNA | Down |
| 3207 | 14 | ENSG00000199282 | snoRNA | Down |
| 3233 | 15 | hsa-mir-523 | miRNA | Down |
| 3214 | 16 | hsa-mir-376a-2/1 F2 | miRNA | Down |
| 3230 | 17 | ENSG00000199856 F1 | snoRNA | Down |
| 3184 | 18 | HBII-276 F2 | CDBox | Up |
| 3257 | 19 | RASGRP4-Hs01073179_m1 | NM_170604 | Down |
| 3258 | 20 | FAM20A-Hs01034071_m1 | NR_027751 | Down |
| 3259 | 22 | NEK9-Hs00929602_m1 | NM_033116 | Down |
| 3260 | 24 | ABCC1-Hs01561504_m1 | NM_004996 | Up |
| 3261 | 25 | SORBS2-Hs01125202_m1 | NM_001145671 | Up |
| 3262 | 27 | TMPRSS2-ERG fusion gene | NM_002772 | Down |
| 3249 | 28 | ATP5O-Hs04272738_m1 | NM_001697.3 | Down |
| 3263 | 29 | DSCAM-Hs00242097_m1 | NM_020693 | Down |
| 3264 | 30 | ERG-Hs01573964_m1 | NM_004449 | Up |
| 3265 | 31 | ICOSLG-Hs00391287_m1 | NM_015259 | Down |
| 3250 | 32 | DOP1B-Hs01123288_m1 | NM_005128 | Down |
| 3266 | 34 | C21orf33-Hs01105802_g1 | NM_004649 | Down |
| 3267 | 35 | ADAMTS5-Hs04272736_s1 | NM_007038 | Down |
| 3268 | 36 | CXADR-Hs04194411_s1 | NM_001338 | Down |
| 3251 | 37 | NCAM2-Hs01562292_ml | NM_004540.5 | Up |
| 3252 | 38 | UBASH3A-Hs00955169_m1 | NM_018961.4 | Up |
| 3269 | 40 | PFKL-Hs01040525_m1 | NR_024108 | Up |
| 3253 | 41 | CHODL-Hs01070471_m1 | NM_024944.3 | Down |
| 3254 | 46 | PKNOX1-Hs01007092_m1 | | Up |
| 3255 | 48 | CYYR1-Hs00951849_m1 | NR_135472 | Up |
| 3270 | 49 | SLC19A1-Hs00953342_m1 | NM_194255 | Up |
| 3271 | 51 | PRDM15-Hs00411318_m1 | NM_022115 | Down |
| 3272 | 52 | COL6A1-Hs01095585_m1 | NM_001848 | Down |
| 3273 | 53 | ABCG1-Hs01555191_m1 | NM_016818 | Down |
| 3256 | 54 | GART-Hs00531926_m1 | NM_000819 | Down |

In some embodiments, the combination of nucleic acid biomarkers is the combination defined as Group 5, shown in Table 5. Table 5 shows this combination of nucleic acid biomarkers—Group 5—as a defined panel where each must be present and detected for a variation of no variation; an upregulation; or a downregulation.

TABLE 5
Group 5-Combination of Biomarkers

| | | | | Up or |
|---|---|---|---|---|
| SEQ | Group | | | Down |
| ID | ID | | | Regu- |
| NO: | NO: | Gene Name | Type/Ref | lation |
| 3257 | 19 | RASGRP4-Hs01073179_m1 | NM_170604 | Down |
| 3258 | 20 | FAM20A-Hs01034071_m1 | NR_027751 | Down |
| 3259 | 22 | NEK9-Hs00929602_m1 | NM_033116 | Down |
| 3260 | 24 | ABCC1-Hs01561504_m1 | NM_004996 | Up |
| 3261 | 25 | SORBS2-Hs01125202_m1 | NM_001145671 | Up |
| 3262 | 27 | TMPRSS2-ERG fusion gene | NM_002772 | Down |
| 3249 | 28 | ATP5O-Hs04272738_m1 | NM_001697.3 | Down |
| 3263 | 29 | DSCAM-Hs00242097_m1 | NM_020693 | Down |
| 3264 | 30 | ERG-Hs01573964_m1 | NM_004449 | Up |
| 3265 | 31 | ICOSLG-Hs00391287_m1 | NM_015259 | Down |
| 3250 | 32 | DOP1B-Hs01123288_m1 | NM_005128 | Down |
| 3266 | 34 | C21orf33-Hs01105802_g1 | NM_004649 | Down |
| 3267 | 35 | ADAMTS5-Hs04272736_s1 | NM_007038 | Down |
| 3268 | 36 | CXADR-Hs04194411_s1 | NM_001338 | Down |
| 3251 | 37 | NCAM2-Hs01562292_m1 | NM_004540.5 | Up |
| 3252 | 38 | UBASH3A-Hs00955169_m1 | NM_018961.4 | Up |
| 3269 | 40 | PFKL-Hs01040525_m1 | NR_024108 | Up |
| 3253 | 41 | CHODL-Hs01070471_m1 | NM_024944.3 | Down |
| 3254 | 46 | PKNOX1-Hs01007092_m1 | | Up |
| 3255 | 48 | CYYR1-Hs00951849_m1 | NR 135472 | Up |
| 3270 | 49 | SLC19A1-Hs00953342_m1 | NM_194255 | Up |
| 3271 | 51 | PRDM15-Hs00411318_m1 | NM_022115 | Down |
| 3272 | 52 | COL6A1-Hs01095585_m1 | NM_001848 | Down |
| 3273 | 53 | ABCG1-Hs01555191_m1 | NM_016818 | Down |
| 3256 | 54 | GART-Hs00531926_m1 | NM_000819 | Down |

In one embodiment, the present invention includes a method of determining a primer or a probe for a CFP RNA biomarker. Such a method can include analyzing one or more of the sequences of the Sequence Listing or Figures and determining a unique or sufficiently unique specific target sequence that is useful as a primer or a probe therefore. The primers can be readily determined from the sequences of the sequence listing by convention techniques, and may encompass low stringency, medium stringency and high stringency primers, and thereby the primer sequences that are useful can be changed within the sequences provided in the Sequence Listing.

In one embodiment, the CFP RNA biomarkers can be used to indicate whether or not a fetus of a pregnant woman has T21. This determination can be performed by a blood test at least as early as 10 weeks gestation. Accordingly, the biomarkers identified herein can be combined in a mathematical algorithm that can predict likelihood of T21. The mathematics to create the algorithm is well known and not proprietary. Such an algorithm for predicting likelihood of T21 can be run on a computing system, and may be configured as software and/or or hardware. Data can be input into the computing system in order to operate and optimize the T21 prediction algorithm.

The results of a subject's diagnosis (T21) or the information of the Group of the combination of biomarkers, screening, prognosis or monitoring is typically displayed or provided to a user such as a clinician, health care worker or other caregiver, laboratory personnel or the patient. The results may be quantitative information (e.g. the level or amount of a marker compared to a control) or qualitative information (e.g. diagnosis of spontaneous preterm birth) for all biomarkers in the defined Group. The output can comprise guidelines or instructions for interpreting the results, for example, numerical or other limits that indicate the presence or absence of T21. The guidelines may also specify the diagnosis, for example whether there is a high risk of T21. The output can include tools for interpreting the results to arrive at a diagnosis, prognosis or treatment plan, for example, an output may include ranges or cut-offs for abnormal or normal status to arrive at a diagnosis, prognosis, or treatment plan or further diagnostic confirmation procedure. The output can also provide a recommended therapeutic plan, and it may include other clinical information and guidelines and instructions for interpreting the information.

Devices known in the art can be used to transmit the results of a method of the invention. Examples of output devices include without limitation, a visual output device (e.g. a computer screen or a printed paper), an auditory output device (e.g., a speaker), a printer or a patient s electronic medical record. The format of the output providing the results and related information may be a visual output (e.g., paper or a display on a screen), a diagram such as a graph, chart or voltammetric trace, an audible output (e.g. a speaker) or, a numerical value. In an aspect, the output is a numerical value, in particular the amount or relative amount of each biomarker of a specific combination of biomarkers in a subject's sample compared to a control. In an aspect, the output is a graph that indicates a value, such as an amount or relative amount, of the at least one marker in the sample from the subject on a standard curve. In an embodiment, the output (such as a graphical output) shows or provides a cut-off value or level that indicates the presence of high risk of T21. An output may be communicated to a user by physical, audible or electronic means, including mail, telephone, facsimile transmission, email or an electronic medical record.

The analytic methods described herein can be implemented by use of computer systems and methods described below and known in the art. Thus the invention provides computer readable media comprising one or more combinations of biomarkers, and optionally other markers (e.g. markers of T21). “Computer readable media” refers to any medium that can be read and accessed directly by a computer. Thus, the invention contemplates computer readable medium having recorded thereon markers identified for patients and controls. “Recorded” refers to a process for storing information on computer readable medium. The skilled artisan can readily adopt any of the presently known methods for recording information on computer readable medium to generate manufactures comprising information on one or more combinations of biomarkers.

A variety of data processor programs and formats can be used to store information on one or more combinations of biomarkers, and other markers on computer readable medium. Any number of data processor structuring formats (e.g., text file or database) may be adapted in order to obtain computer readable medium having recorded thereon the marker information.

By providing the combination of biomarker information in computer readable form, one can routinely access the information for a variety of purposes. For example, one skilled in the art can use the information in computer readable form to compare marker information obtained during or following therapy with the information stored within the data storage means.

The invention still further provides a system for identifying selected records that identify T21. A system of the invention generally comprises a computer; a database server coupled to the computer; a database coupled to the database server having data stored therein, the data comprising records of data comprising one or more combinations of biomarkers, and a code mechanism for applying queries based upon a desired selection criteria to the data file in the database to produce reports of records which match the desired selection criteria.

The invention contemplates a method for determining whether a subject has T21 comprising: (a) receiving phenotypic and/or clinical information on the subject and information on one or more combinations of biomarkers, associated with samples from the subject; (b) acquiring information from a network corresponding to the one or more combinations of biomarkers; and (c) based on the phenotypic information, information on one or more combinations of biomarkers, and optionally other markers, and acquired information, determining whether the subject has T21; and (d) optionally recommending a procedure or treatment.

### Example 1

### T21 Cell Free Plasma Biomarker Confirmation Protocol

Presented here is an example of an assay that was used to confirm the microarray biomarker identification for those biomarkers identified as altered in pregnant mothers carrying a fetus that has trisomy 21. More than 1 million exon clusters and 1769 non-coding small RNAs were screened by Affymetrix GeneChip Human Exon ST and Affymetrix GeneChip miRNA Arrays, respectively. PCF RNA was extracted, reverse transcribed and run on the microarrays. The mean PCF total RNA extracted per sample was 25.02 ug+/−14 ug (range 9.60-72.63 ug). Of the 232,119 exons read, 2,686 (1.2%) were located on the #21 chromosome. 3,095 of all exons (1.3%) were differentially expressed compared to Normal. Of those differentially expressed, only 38 (1.2%) originated from the #21 chromosome. Of the 1769 small noncoding human RNAs on the microarray, 16 (0.9%) originated from genes on the #21 chromosome. There were 371 small, noncoding RNAs (21%) differentially expressed in T21 compared to Normal. Only 1 differentially expressed small noncoding RNA originated from a gene located on the #21 chromosome (0.3%). After reordering potential marker RNAs by p value and narrowness of distribution, the 36 highest scoring exons representing 36 mRNAs (19 serendipitously originating from a gene on the #21 chromosome) and the 18 highest scoring small noncoding RNAs (including 1 originating from a gene on the #21 chromosome) were confirmed by q-PCR (Table 2). These 54 RNAs were then subject to Validation testing. These data confirm that the microarray analysis functioned as designed and identified RNAs that were informative of the trisomy 21 status of the fetus.

Blood Plasma Cell Free RNA isolation. RNA is obtained using a process based on a phenol/guanidium isothiocyanate/glycerol phase separation.

RNA concentration. RNA concentration was measured by using a Qubit® 2.0 Fluorometer (Life Technologies, Grand Island, NY) as recommended by the manufacturer. Briefly, calibration of the Qubit® 2.0 Fluorometer was done using Standard #1 and #2. Working solution was prepared by diluting the Qubit™ RNA reagent at 1:200 in Qubit™ RNA buffer. Working solution (190 ul) and 10 ul of standard or RNA sample were mixed, then incubate at room temperature for 2 minutes. The RNA concentration was determined.

### Reverse Transcription.

mRNA RT:

The RNA samples were diluted, and a master mix prepared including dNTP mix, Omniscript Reverse Transcriptase and Random Primer (Invitrogen, Carlsbad CA). The mRNA of each sample was converted into cDNA at 37° C. for 60 min per manufacturer instructions.

miRNA RT:

The miRs were polyadenylated using reagents from the Invitrogen NCode miRNA First-Strand cDNA Synthesis Kit (ThermoFisher). The polyadenylated microRNA was reverse transcribed to generate the first strand of cDNA according to the manufactory's protocol.

Preamplification and qPCR:

Multiplex qPCR reactions were performed by SYBR green using the ViiA 7 Real-Time PCR System. The primers for the gene panels were custom designed and synthesized by Integrated DNA Technologies (IDT, Coralville, IA). The probe sets in each reaction well included primers for the biomarker, normalization, and spike genes so that all three genes were run in the same reaction well to minimize assay variation. Information about the primer sequences used is available from the authors. Preamplification was performed, 1 ul RT samples were prepared for the preamplification Mix Reaction and underwent 12 cycles. Two customized probe-based microfluidic PCR Cards with 384 wells were developed for the selected mRNA and small noncoding RNA markers using a proprietary method (Rosetta Signaling Laboratory, Mission Hills, KS). The probe sets in each well included primers for biomarker, normalization, and spike genes so that all three were run in the same reaction well to minimize assay variation. One ul RT samples were prepared with the preamplification Mix Reaction and underwent 12 cycles. Two ul preamplification cDNA samples were diluted into 10 ul PCR reaction mix, followed by RT PCR using SYBR Green Supermix (ThermoFisher). Threshold cycles (Ct values) of qPCR reactions were extracted using QuantStudio™ Software V1.3 (Applied Biosystems, Foster City CA). Potential markers were normalized to housekeeping control sequences and to a spiked-in cDNA, the Cts determined and the relative expression calculated using the 2−ΔΔCt method.

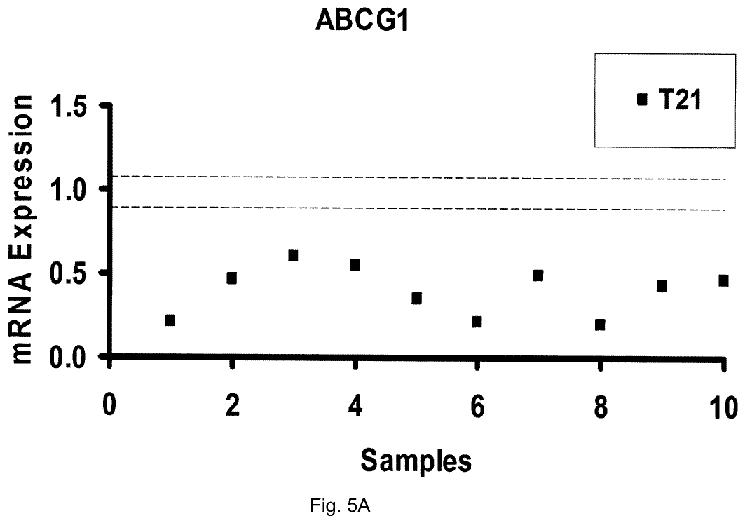

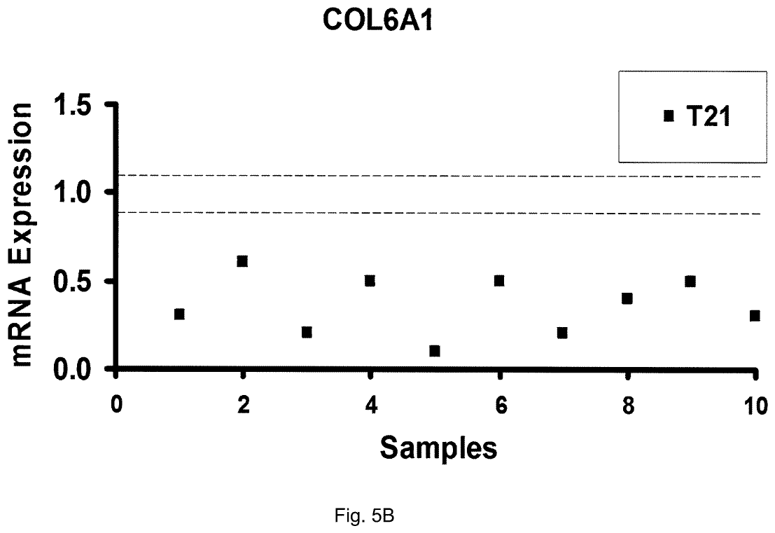

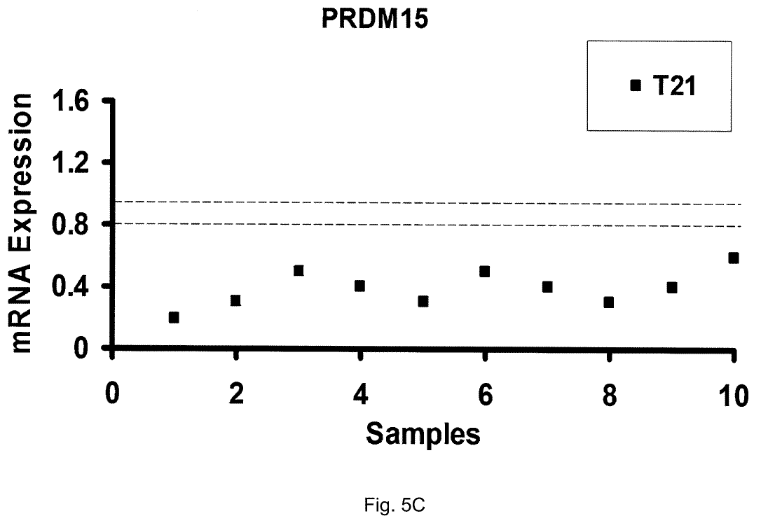

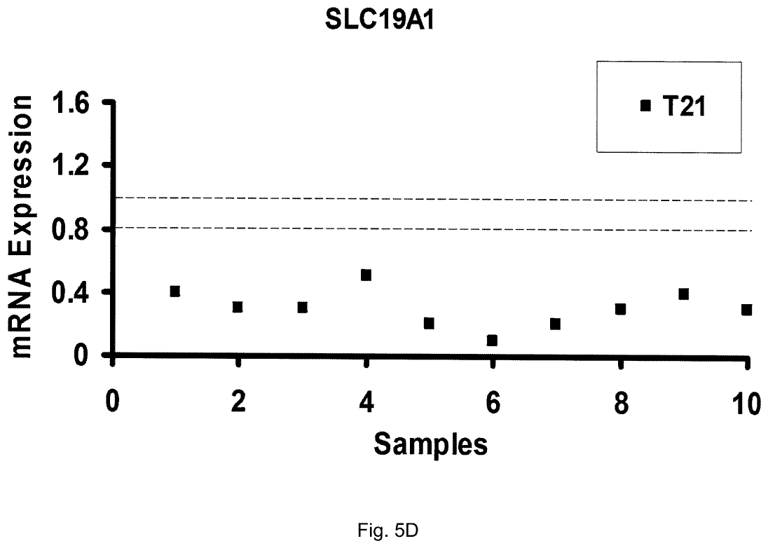

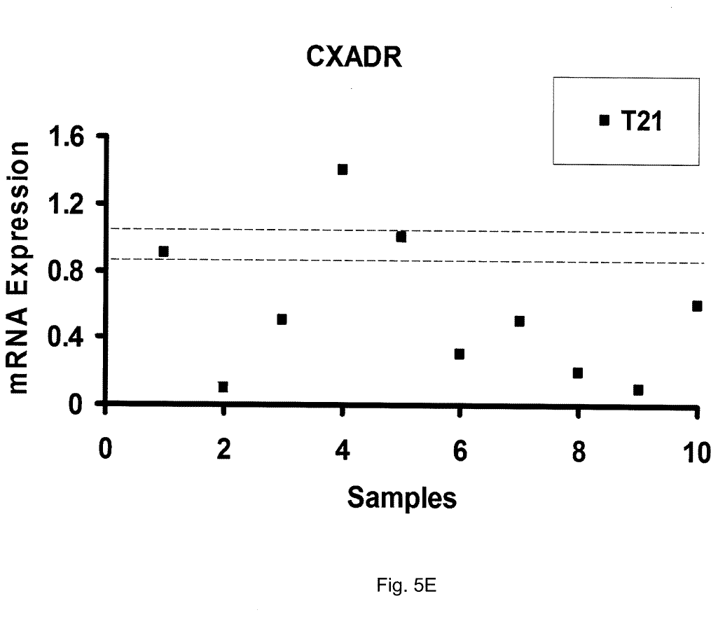

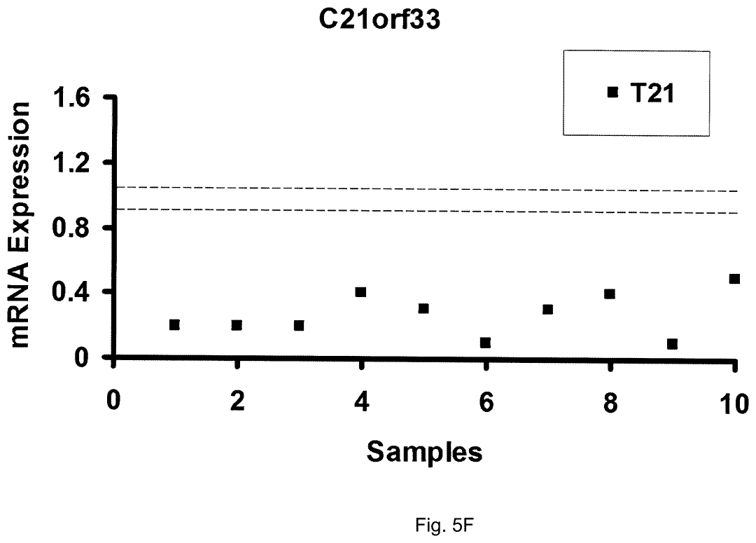

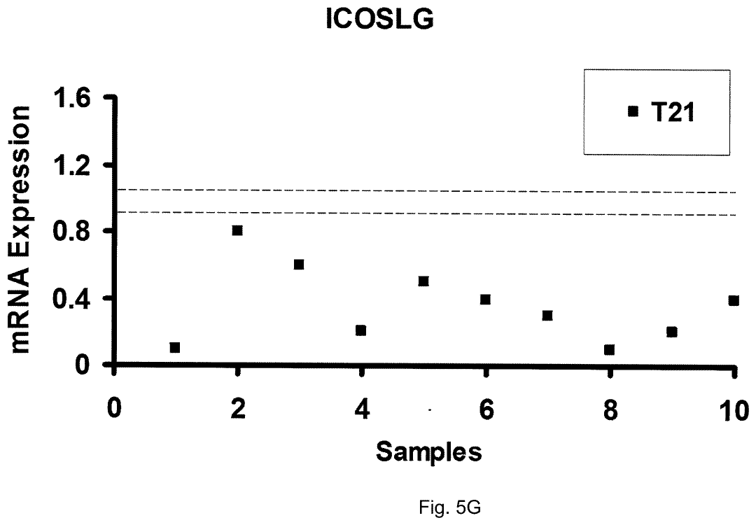

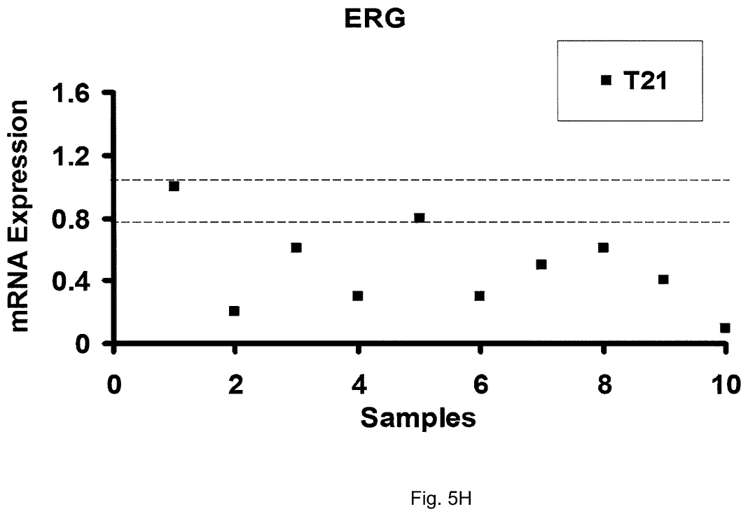

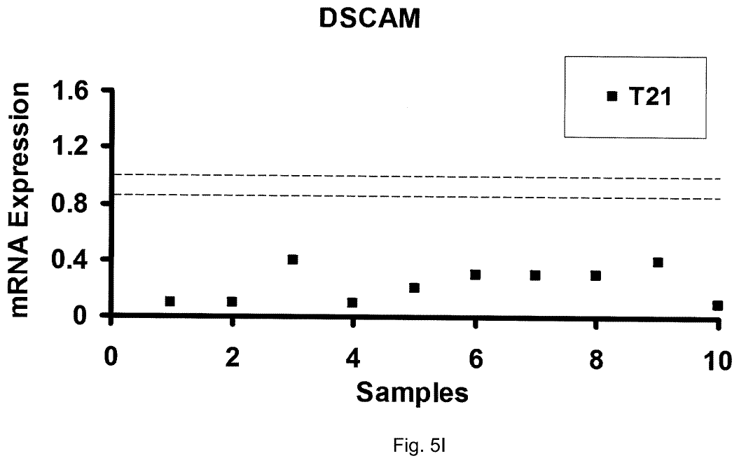

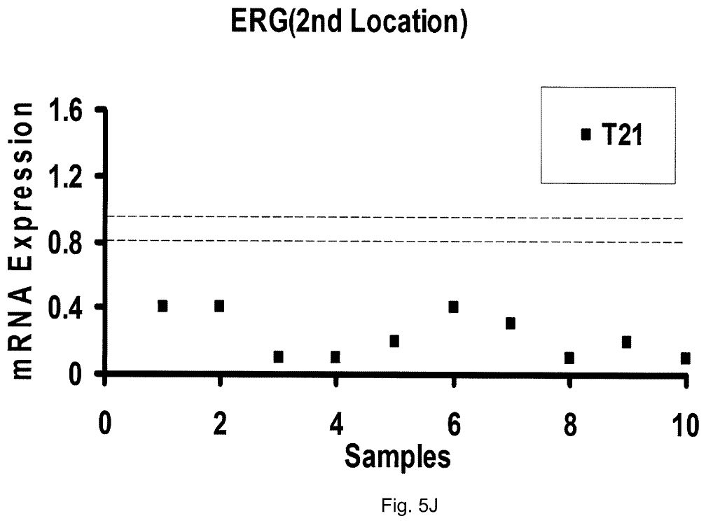

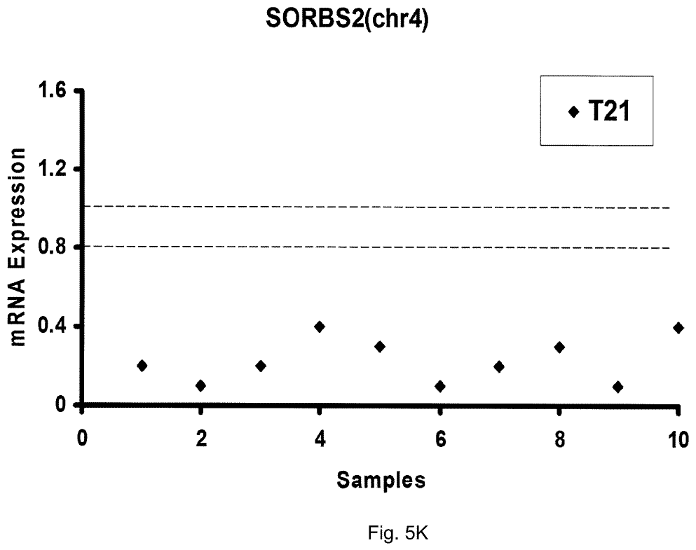

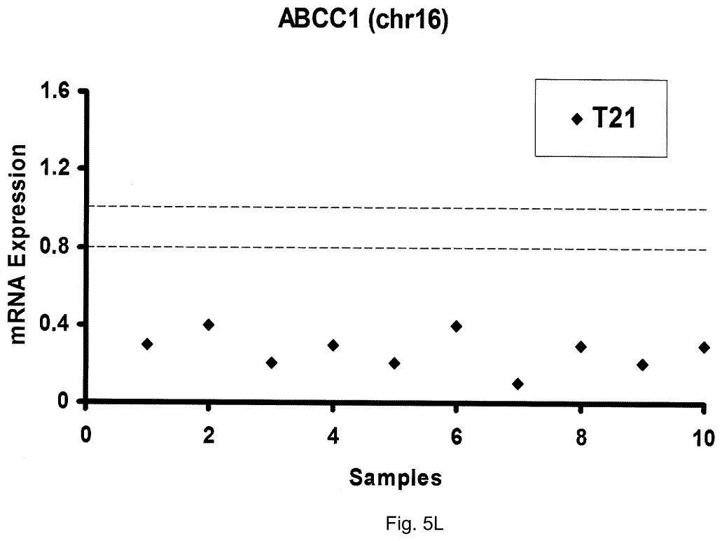

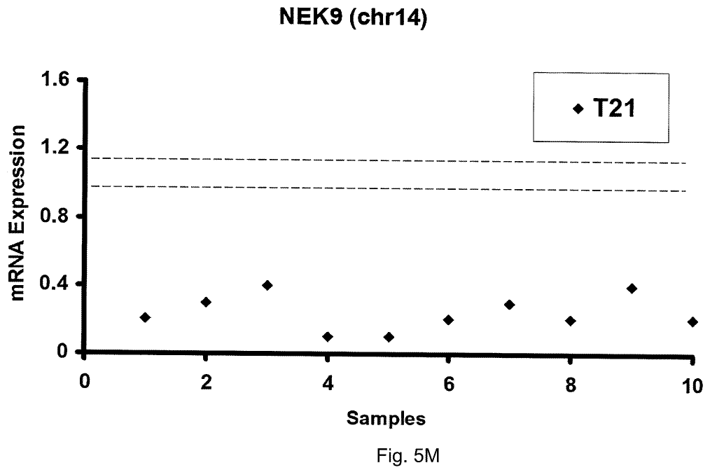

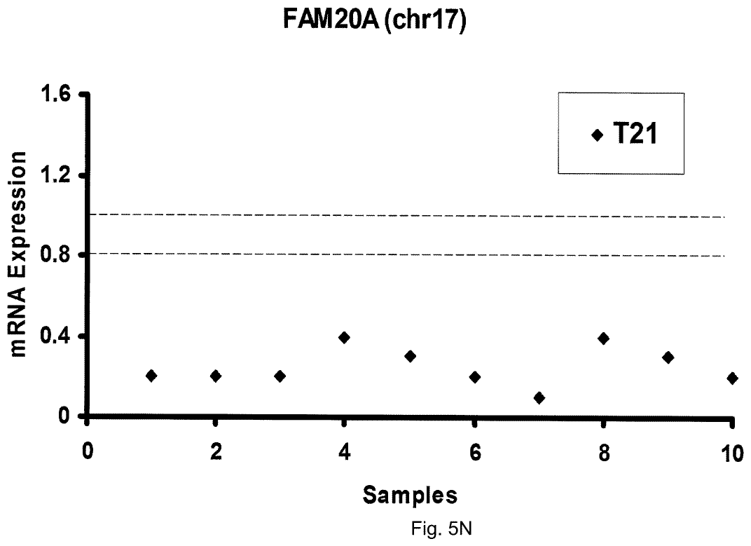

Data analysis. For the initial analysis, the 95% confidence interval for expression (normalized to the described normalization sequences) of each of the selected T21 markers at 12 weeks gestation was calculated (FIG. 5, area between the dotted lines of each graph). Expression levels of the T21 biomarkers were then measured and plotted (squares) against the normal range for affected pregnancies at 12 weeks gestation.

The terms “sample”, “biological sample”, and the like mean a material known or suspected of expressing or containing one or more combinations of biomarkers. A test sample can be used directly as obtained from the source or following a pretreatment to modify the character of the sample. A sample can be derived from any biological source, such as tissues, extracts, or cell cultures, including cells, cell lysates, and physiological fluids, such as, for example, whole blood, plasma, serum, saliva, ocular lens fluid, cerebral spinal fluid, sputum, sweat, urine, milk, ascites fluid, synovial fluid, peritoneal fluid, and the like. A sample can be obtained from animals, preferably mammals, most preferably humans. A sample can be treated prior to use, such as preparing plasma from blood, diluting viscous fluids, and the like. Methods of treatment can involve filtration, distillation, extraction, concentration, inactivation of interfering components, the addition of reagents, and the like.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

A unique segment of a sequence in a sequence listing is a specific sequence segment that is found within the recited sequence of the SEQ ID NO, and substantially absent in the rest of the RNA transciptome. That is, the unique segment of the sequence in the Sequence Listing identified by the SEQ ID NO can be used as a probe or a primer that is specific for that SEQ ID NO. The techniques available for identifying a primer or a probe available to one of ordinary skill in the art can be used to identify one or more unique segments of each SEQ ID NO recited in the Sequence Listing.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The present invention is illustrated by the examples provided herein. It is to be understood that the particular examples, materials, amounts, and procedures are to be interpreted broadly in accordance with the scope and spirit of the invention as set forth herein.

All references cited herein are incorporated herein in their entirety by specific reference.

1. A method of detecting a combination of ribonucleic acid (RNA) biomarkers, the method comprising:
obtaining a plasma sample from a human subject, wherein the human subject is a pregnant female;
obtaining cell free nucleic acids from the plasma sample, wherein the cell free nucleic acids are cell free ribonucleic acids (RNA);
detecting in the cell free RNA the presence of the combination of RNA biomarkers,
wherein the detecting comprises:
contacting the cell free RNA with a combination of primers or probes that are complementary to the combination of RNA biomarkers, and
detecting hybridization between the primers or probes and the combination of RNA biomarkers,
wherein the combination of RNA biomarkers used in the method consists of:
RASGRP4, hsa-mir-26b, UBASH3A, NCAM2, COL6A1, and ISOSLG.

2. The method of claim 1, further comprising detecting a normalization nucleic acid.

3. The method of claim 1, further comprising:
obtaining a plasma sample from a second human subject, wherein the second human subject is a pregnant female carrying a fetus without trisomy 21;
obtaining a second cell free ribonucleic acid sample from the plasma sample; and
detecting in the second cell free ribonucleic acid sample the presence of the combination of RNA biomarkers.

4. The method of claim 3, further comprising:
quantitating the amount of each RNA biomarker in the cell free RNA from the pregnant female; and
quantitating the amount of each RNA biomarker in the second cell free ribonucleic acid sample from the second pregnant female.

5. A method of detecting a combination of ribonucleic acid (RNA) biomarkers, the method comprising:
obtaining a plasma sample from a human subject, wherein the human subject is a pregnant female;
obtaining cell free nucleic acids from the plasma sample, wherein the cell free nucleic acids are cell free ribonucleic acids (RNA);
detecting in the cell free RNA the presence of the combination of RNA biomarkers,
wherein the detecting comprises:
contacting the cell free RNA with a combination of primers or probes that are complementary to the combination of RNA biomarkers, and
detecting hybridization between the primers or probes and the combination of RNA biomarkers,
wherein the combination of RNA biomarkers used in the method consists of:
GART, hsa-mir-26b, hsa-mir-450b, COL6A1, ATP50, ENSG00000199633 F2, and DOP1B.

6. The method of claim 5, further comprising detecting a normalization nucleic acid.

7. The method of claim 5, further comprising:
obtaining a plasma sample from a second human subject, wherein the second human subject is a pregnant female carrying a fetus without trisomy 21;
obtaining a second cell free ribonucleic acid sample from the plasma sample; and
detecting in the second cell free ribonucleic acid sample the presence of the combination of RNA biomarkers.

8. The method of claim 7, further comprising:
quantitating the amount of each RNA biomarker in the cell free RNA from the pregnant female; and
quantitating the amount of each RNA biomarker in the second cell free ribonucleic acid sample from the second pregnant female.

9. A method of detecting a combination of ribonucleic acid (RNA) biomarkers, the method comprising:
obtaining a plasma sample from a human subject, wherein the human subject is a pregnant female;
obtaining cell free nucleic acids from the plasma sample, wherein the cell free nucleic acids are cell free ribonucleic acids (RNA);
detecting in the cell free RNA the presence of the combination of RNA biomarkers,
wherein the detecting comprises:
contacting the cell free RNA with a combination of primers or probes that are complementary to the combination of RNA biomarkers, and
detecting hybridization between the primers or probes and the combination of RNA biomarkers,
wherein the combination of RNA biomarkers used in the method consists of:
hsa-mir-26b, RASGRP4, ICOSLG, UBASH3A, DOP1B, and FAM20A.

10. The method of claim 9, further comprising detecting a normalization nucleic acid.

11. The method of claim 9, further comprising:
obtaining a plasma sample from a second human subject, wherein the second human subject is a pregnant female carrying a fetus without trisomy 21;
obtaining a second cell free ribonucleic acid sample from the plasma sample; and
detecting in the second cell free ribonucleic acid sample the presence of the combination of RNA biomarkers.

12. The method of claim 11, further comprising:
quantitating the amount of each RNA biomarker in the cell free RNA from the pregnant female; and
quantitating the amount of each RNA biomarker in the second cell free ribonucleic acid sample from the second pregnant female.

13. A method of detecting a combination of ribonucleic acid (RNA) biomarkers, the method comprising:
obtaining a plasma sample from a human subject, wherein the human subject is a pregnant female;
obtaining cell free nucleic acids from the plasma sample, wherein the cell free nucleic acids are cell free ribonucleic acids (RNA);
detecting in the cell free RNA the presence of the combination of RNA biomarkers,
wherein the detecting comprises:
contacting the cell free RNA with a combination of primers or probes that are complementary to the combination of RNA biomarkers, and
detecting hybridization between the primers or probes and the combination of RNA biomarkers,
wherein the combination of RNA biomarkers used in the method consists of:
hsa-mir-26b, RASGRP4, NCAM2, ENSG00000207147 F2, UBASH3A, and ENSG00000199856 F1.

14. The method of claim 13, further comprising detecting a normalization nucleic acid.

15. The method of claim 13, further comprising:
obtaining a plasma sample from a second human subject, wherein the second human subject is a pregnant female carrying a fetus without trisomy 21;
obtaining a second cell free ribonucleic acid sample from the plasma sample; and
detecting in the second cell free ribonucleic acid sample the presence of the combination of RNA biomarkers.

16. The method of claim 15, further comprising:
quantitating the amount of each RNA biomarker in the cell free RNA from the pregnant female; and
quantitating the amount of each RNA biomarker in the second cell free ribonucleic acid sample from the second pregnant female.
